(19) United States
(12) Patent Application Publication
(10) Pub. No.: US 2024/0102032 A1
(43) Pub. Date: Mar. 28, 2024

(54) CRISPR-CAS EFFECTOR POLYPEPTIDES AND METHODS OF USE THEREOF

(71) Applicant: The Regents of the University of California, Oakland, CA (US)

(72) Inventors: Jennifer A. Doudna, Berkeley, CA (US); Patrick Pausch, Berkeley, CA (US); Katarzyna Soczek, Berkeley, CA (US); Steven E. Jacobsen, Agoura Hills, CA (US); Zheng Li, Los Angeles, CA (US)

(21) Appl. No.: 18/273,685

(22) PCT Filed: Jan. 24, 2022
(86) PCT No.: PCT/US22/13539

§ 371 Date: Jul. 21, 2023

Related U.S. Application Data
(60) Provisional application No. 63/256,333, filed on Oct. 15, 2021.
(60) Provisional application No. 63/218,711, filed on Jul. 6, 2021.
(60) Provisional application No. 63/159,025, filed on Mar. 10, 2021.
(60) Provisional application No. 63/141,323, filed on Jan. 25, 2021.

(51) Int. Cl. C12N 15/82; C12N 9/22; C12N 15/10; C12N 15/11; C12N 15/90

(52) U.S. Cl. CPC C12N 15/8213; C12N 9/22; C12N 15/102; C12N 15/11; C12N 15/90; C12N 2310/121; C12N 2310/20; C12N 2310/3519

(57) ABSTRACT

The present disclosure provides CRISPR-Cas effector polypeptides that exhibit enhanced gene editing and/or trans cleavage activity, compared to a wild-type CasPhi polypeptide. The present disclosure provides systems and kits comprising such CRISPR-Cas effector polypeptides. The present disclosure provides methods, including gene editing and diagnostic methods, using a CRISPR-Cas effector polypeptide of the present disclosure.

### CROSS-REFERENCE

This application claims the benefit of U.S. Provisional Patent Application No. 63/141,323, filed Jan. 25, 2021, U.S. Provisional Patent Application No. 63/159,025, filed Mar. 10, 2021, U.S. Provisional Patent Application No. 63/218,711, filed Jul. 6, 2021, and U.S. Provisional Patent Application No. 63/256,333, filed Oct. 15, 2021, which applications are incorporated herein by reference in their entirety.

### STATEMENT REGARDING FEDERALLY SPONSORED RESEARCH

This invention was made with government support under Grant No. AI142817 awarded by the National Institutes of Health. The government has certain rights in the invention.

### INCORPORATION BY REFERENCE OF SEQUENCE LISTING PROVIDED AS A TEXT FILE

A Sequence Listing is provided herewith as a text file, “BERK-440WO_SEQ_LIST_ST25.txt” created on Jan. 20, 2022 and having a size of 205 KB. The contents of the text file are incorporated by reference herein in their entirety.

### INTRODUCTION

CRISPR-Cas systems (Clustered Regularly Interspaced Short Falindromic Repeats, CRISPR-associated proteins) include: a) Cas proteins, which are involved in acquisition, targeting and cleavage of foreign DNA or RNA; and b) a guide RNA(s), which includes a segment that binds Cas proteins and a segment that binds to a target nucleic acid. For example, Class 2 CRISPR-Cas systems comprise a single Cas protein bound to a guide RNA, where the Cas protein binds to and cleaves a targeted nucleic acid. The programmable nature of these systems has facilitated their use as a versatile technology for use in modification of target nucleic acid.

There is a need in the art for additional CRISPR-Cas effector proteins that exhibit advantageous properties.

### SUMMARY

The present disclosure provides CRISPR-Cas effector polypeptides that exhibit enhanced gene editing and/or trans cleavage activity, compared to a wild-type CasPhi polypeptide. The present disclosure provides systems and kits comprising such CRISPR-Cas effector polypeptides. The present disclosure provides methods, including gene editing and diagnostic methods, using a CRISPR-Cas effector polypeptide of the present disclosure.

### BRIEF DESCRIPTION OF THE DRAWINGS

FIG. 1A-1B depict cleavage of DNA by a wild-type CasΦ polypeptide (FIG. 1A from top to bottom SEQ ID NOs:1-3).

FIG. 2A-2B depict the domain architecture at the primary structure level (FIG. 2A) and cryogenic electron microscopy (Cryo-EM) structures of wild-type CasΦ in the crRNA- and DNA-bound states (FIG. 2B).

FIG. 3A-3C depict cleavage of DNA by exemplary CRISPR-Cas effector polypeptides (vCasΦ and nCasΦ) of the present disclosure.

FIG. 4A-4B depict secondary structure of a wild-type CasΦ polypeptide (SEQ ID NO:4), illustrating the position of helix alpha 7.

FIG. 5A-5C provide an alignment of amino acid sequences of various CasΦ polypeptides, illustrating the position of helix alpha 7 (from top to bottom SEQ ID NOs:5-13).

FIG. 6 provides an amino acid sequence of a wild-type CasΦ polypeptide (SEQ ID NO:13).

FIG. 7 provides an amino acid sequence of an exemplary variant CRISPR-Cas effector polypeptide (vCasPhi (also referred to as “vCasΦ”)) of the present disclosure (SEQ ID NO:14).

FIG. 8 provides an amino acid sequence of an exemplary variant CRISPR-Cas effector polypeptide (nCasPhi (also referred to as “nCasΦ”)) of the present disclosure (SEQ ID NO:15).

FIG. 9A-9R provide amino acid sequences of examples of CasΦ polypeptides (referred to in the figure as “Cas12J”).

FIG. 10 provides nucleotide sequences of constant region portions of CasPhi guide RNAs (referred to in the figure as “Cas12J” guide RNAs; and depicted as the DNA encoding the RNA). Sequences separated by an “or” are the reverse complement of one another. All of the CasPhi constant regions are in the reverse complement orientation, except for the Cas12J_2071242, Cas12J_10000002_47, Cas12J_877636_12 sequences.

FIG. 11 depicts reverse complement of a CasPhi-binding sequence (constant region) of CasPhi guide RNAs (referred to in the figure as “Cas12J” guide RNAs) (from top to bottom SEQ ID NOs:34-42).

FIG. 12A-12B depict target DNA detection by two different CasPhi variants of the present disclosure.

FIG. 13 provides a plasmid map of pCAMBIA1300 pUBQ10 pco-WTCasΦ U6 PDS3 gR10.

FIG. 14A-14D depict target gene editing efficiencies of CasΦ variants compared to WTCasΦ, using AtPDS3 gRNA8 or gRNA10.

FIG. 15A-15B depict target gene editing efficiencies of CasΦ variants compared to WTCasΦ, using FWA gRNA1, gRNA4, gRNA5, or gRNA6.

FIG. 16A-16B depict target gene editing efficiencies of CasΦ variants compared to WTCasΦ, using plasmid transfection or RNP transfection.

FIG. 17A-17D depict target gene editing efficiencies of CasΦ variants compared to WTCasΦ, under repressive and compact chromatin states.

FIG. 18 provides a plasmid map of pCAMBIA1300 pUBQ10 pco-WTCasΦ CmYLCVp ribozyme PDS3 gR10.

FIG. 19 provides a plasmid map of pCAMBIA1300 pUBQ10 pco-WTCasΦ pUBQ10 ribozyme PDS3 gR10.

FIG. 20A-20B depict target gene editing efficiencies of CasΦ variants compared to WTCasΦ, using plasmid transfection. Guide RNA expression was driven by Pol-II promoter CmYLCV (FIG. 20A) or UBQ10 (FIG. 20B).

FIG. 21 provides the nucleotide sequence of plasmid pCAMBIA1300 pUBQ10 pco-WTCasΦU6 PDS3 gR10 (SEQ ID NO:43; plasmid map provided in FIG. 13).

FIG. 22 provides the nucleotide sequence of Arabidopsis codon-optimized vCasΦ (SEQ ID NO:44).

FIG. 23 provides the nucleotide sequence of Arabidopsis codon-optimized nCasΦ (SEQ ID NO:45).

FIG. 24 provides the nucleotide sequence of the cassette that employed the CmYLCV promoter to drive transcription of AtPDS3 gRNA10 flanked by ribozymes (SEQ ID NO:46).

FIG. 25 provides the nucleotide sequence of the cassette which employs the UBQ10 promoter (pUB10) to drive the transcription of AtPDS3 gRNA10 flanked by ribozymes (SEQ ID NO:47).

FIG. 26 provides the nucleotide sequence of a UBQ10 promoter (SEQ ID NO:48).

FIG. 27 provides the nucleotide sequence of a CmYLCV promoter (SEQ ID NO:49).

FIG. 28 depicts target gene efficiencies of CasΦ variants compared to WTCasΦ in T1 transgenic plants.

FIG. 29 depicts white sectors observed in leaves of vCASΦ and nCASΦ PDS3 gR10 transgenic T1 plants.

FIG. 30 depicts target gene editing efficiencies of CasΦ variants combined with Pol-II promoters and PDS3 gRNA10 flanked by ribozymes, in T1 transgenic plants.

FIG. 31 depicts albino seedlings observed in T2 populations of vCASΦ and nCASΦ PDS3 gR10 transgenic plants.

FIG. 32 depicts data showing that albino seedlings which are transgene free were identified in T2 populations of vCASΦ and nCASΦ PDS3 gR10 transgenic plants.

FIG. 33 depicts total seedling number and albino seedling number from the T2 populations of Arabidopsis transgenic plants transgenic for WTCasΦ, vCasΦ or nCasΦ with PDS3 gRNA10.

FIG. 34 depicts percentage of albino seedlings in T2 populations of Arabidopsis transgenic plants transgenic for WTCasΦ, vCasΦ or nCasΦ with PDS3 gRNA10.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, in the context of this disclosure, a guanine (G) (e.g., of dsRNA duplex of a guide RNA molecule; of a guide RNA base pairing with a target nucleic acid, etc.) is considered complementary to both a uracil (U) and to an adenine (A). For example, when a G/U base-pair can be made at a given nucleotide position of a dsRNA duplex of a guide RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Temperature, wash solution salt concentration, and other conditions may be adjusted as necessary according to factors such as length of the region of complementation and the degree of complementation.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a bulge, a loop structure or hairpin structure, etc.). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656), the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489), and the like.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g., with reference to an RNA-binding domain of a polypeptide, binding to a target nucleic acid, and the like) refers to a non-covalent interaction between macromolecules (e.g., between a protein and a nucleic acid; between a CRISPR-Cas effector polypeptide/guide RNA complex and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{D}) of less than 10⁻⁶ M, less than 10⁻⁴ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{D}.

By “binding domain” it is meant a protein domain that is able to bind non-covalently to another molecule. A binding domain can bind to, for example, a DNA molecule (a DNA-binding domain), an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nih.gov/BLAST, ebi.ac.uk/fools/msa/tcoffee/, ebi.ac.uk/Tools/msa/muscle/, mafft.cbrc.jp/alignment/software/. See, e.g., Altschul et al. (1990), J. Mol. Biol. 215:403-10.

A DNA sequence that “encodes” a particular RNA is a DNA nucleotide sequence that is transcribed into RNA. A DNA polynucleotide may encode an RNA (mRNA) that is translated into protein (and therefore the DNA and the mRNA both encode the protein), or a DNA polynucleotide may encode an RNA that is not translated into protein (e.g. tRNA, rRNA, microRNA (miRNA), a “non-coding” RNA (ncRNA), a guide RNA, etc.).

A “protein coding sequence” or a sequence that encodes a particular protein or polypeptide, is a nucleotide sequence that is transcribed into mRNA (in the case of DNA) and is translated (in the case of mRNA) into a polypeptide in vitro or in vivo when placed under the control of appropriate regulatory sequences.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., a CRISPR-Cas effector polypeptide of the present disclosure) and/or regulate translation of an encoded polypeptide.

As used herein, a “promoter” or a “promoter sequence” is a DNA regulatory region capable of binding RNA polymerase and initiating transcription of a downstream (3′ direction) coding or non-coding sequence. For purposes of the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive expression by the various vectors of the present disclosure.

The term “naturally-occurring” or “unmodified” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism that can be isolated from a source in nature is naturally occurring.

The term “fusion” as used herein as applied to a nucleic acid or polypeptide refers to two components that are defined by structures derived from different sources. For example, where “fusion” is used in the context of a fusion polypeptide (e.g., a fusion CasPhi protein), the fusion polypeptide includes amino acid sequences that are derived from different polypeptides. A fusion polypeptide may comprise either modified or naturally-occurring polypeptide sequences (e.g., a first amino acid sequence from a modified or unmodified CasPhi protein; and a second amino acid sequence from a modified or unmodified protein other than a CasPhi protein, etc.). Similarly, “fusion” in the context of a polynucleotide encoding a fusion polypeptide includes nucleotide sequences derived from different coding regions (e.g., a first nucleotide sequence encoding a modified or unmodified CasPhi protein; and a second nucleotide sequence encoding a polypeptide other than a CasPhi protein).

The term “fusion polypeptide” refers to a polypeptide which is made by the combination (i.e., “fusion”) of two otherwise separated segments of amino acid sequence, usually through human intervention.

“Heterologous,” as used herein, means a nucleotide or polypeptide sequence that is not found in the native nucleic acid or protein, respectively. For example, in some cases, in a variant CRISPR-Cas effector polypeptide of the present disclosure, a portion of variant CRISPR-Cas effector polypeptide of the present disclosure may be fused to a heterologous polypeptide (i.e. an amino acid sequence from a protein other than the variant CRISPR-Cas effector polypeptide). The heterologous polypeptide may exhibit an activity (e.g., enzymatic activity) that will also be exhibited by the fusion protein (e.g., base editing activity; nuclear localization; etc.). A heterologous nucleic acid comprising a nucleotide sequence encoding a heterologous polypeptide may be linked to a nucleic acid comprising a nucleotide sequence encoding a CRISPR-Cas effector polypeptide of the present disclosure (e.g., by genetic engineering) to generate a nucleic acid comprising a nucleotide sequence encoding a fusion polypeptide.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”). Alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. An example of such a case is a DNA (a recombinant) encoding a wild-type protein where the DNA sequence is codon optimized for expression of the protein in a cell (e.g., a eukaryotic cell) in which the protein is not naturally found (e.g., expression of a CRISPR-Cas effector polypeptide of the present disclosure, or a fusion polypeptide of the present disclosure) in a eukaryotic cell). A codon-optimized DNA can therefore be recombinant and non-naturally occurring while the protein encoded by the DNA may have a wild type amino acid sequence.

Thus, the term “recombinant” polypeptide does not necessarily refer to a polypeptide whose amino acid sequence does not naturally occur. Instead, a “recombinant” polypeptide is encoded by a recombinant non-naturally occurring DNA sequence, but the amino acid sequence of the polypeptide can be naturally occurring (“wild type”) or non-naturally occurring (e.g., a variant, a mutant, etc.). Thus, a “recombinant” polypeptide is the result of human intervention, but may have a naturally occurring amino acid sequence.

A “vector” or “expression vector” is a replicon, such as plasmid, phage, virus, artificial chromosome, or cosmid, to which another DNA segment, i.e. an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence (or the coding sequence can also be said to be operably linked to the promoter) if the promoter affects its transcription or expression.

The terms “recombinant expression vector,” or “DNA construct” are used interchangeably herein to refer to a DNA molecule comprising a vector and an insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

A cell has been “genetically modified” or “transformed” or “transfected” by exogenous DNA or exogenous RNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like.

The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

A “target nucleic acid” as used herein is a polynucleotide (e.g., DNA such as genomic DNA) that includes a site (“target site” or “target sequence”) targeted by an RNA-guided CRISPR-Cas effector polypeptide of the present disclosure (a variant CRISPR-Cas effector polypeptide of the present disclosure; or a fusion polypeptide of the present disclosure). The target sequence is the sequence to which the guide sequence of a subject CasPhi guide RNA (e.g., a dual CasPhi guide RNA or a single-molecule CasPhi guide RNA) will hybridize. For example, the target site (or target sequence) 5′-GAGCAUAUC-3′ within a target nucleic acid is targeted by (or is bound by, or hybridizes with, or is complementary to) the sequence 5′-GAUAUGCUC-3′. Suitable hybridization conditions include physiological conditions normally present in a cell. For a double stranded target nucleic acid, the strand of the target nucleic acid that is complementary to and hybridizes with the guide RNA is referred to as the “complementary strand” or “target strand”; while the strand of the target nucleic acid that is complementary to the “target strand” (and is therefore not complementary to the guide RNA) is referred to as the “non-target strand” or “non-complementary strand.”

By “cleavage” it is meant the breakage of the covalent backbone of a target nucleic acid molecule (e.g., RNA, DNA). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events.

“Nuclease” and “endonuclease” are used interchangeably herein to mean an enzyme which possesses catalytic activity for nucleic acid cleavage (e.g., ribonuclease activity (ribonucleic acid cleavage), deoxyribonuclease activity (deoxyribonucleic acid cleavage), etc.).

By “cleavage domain” or “active domain” or “nuclease domain” of a nuclease it is meant the polypeptide sequence or domain within the nuclease which possesses the catalytic activity for nucleic acid cleavage. A cleavage domain can be contained in a single polypeptide chain or cleavage activity can result from the association of two (or more) polypeptides. A single nuclease domain may consist of more than one isolated stretch of amino acids within a given polypeptide.

The term “stem cell” is used herein to refer to a cell (e.g., plant stem cell, vertebrate stem cell) that has the ability both to self-renew and to generate a differentiated cell type (see Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells (described below) can differentiate into lineage-restricted progenitor cells (e.g., mesodermal stem cells), which in turn can differentiate into cells that are further restricted (e.g., neuron progenitors), which can differentiate into end-stage cells (i.e., terminally differentiated cells, e.g., neurons, cardiomyocytes, etc.), which play a characteristic role in a certain tissue type, and may or may not retain the capacity to proliferate further. Stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

PSCs of animals can be derived in a number of different ways. For example, embryonic stem cells (ESCs) are derived from the inner cell mass of an embryo (Thomson et. al, Science. 1998 Nov. 6; 282(5391):1145-7) whereas induced pluripotent stem cells (iPSCs) are derived from somatic cells (Takahashi et. al, Cell. 2007 Nov. 30; 131(5):861-72; Takahashi et. al, Nat Protoc. 2007; 2(12):3081-9; Yu et. al, Science. 2007 Dec. 21; 318(5858):1917-20. Epub 2007 Nov. 20). Because the term PSC refers to pluripotent stem cells regardless of their derivation, the term PSC encompasses the terms ESC and iPSC, as well as the term embryonic germ stem cells (EGSC), which are another example of a PSC. PSCs may be in the form of an established cell line, they may be obtained directly from primary embryonic tissue, or they may be derived from a somatic cell. PSCs can be target cells of the methods described herein.

By “embryonic stem cell” (ESC) is meant a PSC that was isolated from an embryo, typically from the inner cell mass of the blastocyst. ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). Stem cells of interest also include embryonic stem cells from other primates, such as Rhesus stem cells and marmoset stem cells. The stem cells may be obtained from any mammalian species, e.g. human, equine, bovine, porcine, canine, feline, rodent, e.g. mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs typically grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, but not SSEA-1. Examples of methods of generating and characterizing ESCs may be found in, for example, U.S. Pat. Nos. 7,029,913, 5,843,780, and 6,200,806, the disclosures of which are incorporated herein by reference. Methods for proliferating hESCs in the undifferentiated form are described in WO 99/20741, WO 01/51616, and WO 03/020920.

By “embryonic germ stem cell” (EGSC) or “embryonic germ cell” or “EG cell” is meant a PSC that is derived from germ cells and/or germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, the disclosures of which are incorporated herein by reference.

By “induced pluripotent stem cell” or “iPSC” it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPSCs may be found in, for example, U.S. Patent Publication Nos. US20090047263, US20090068742, US20090191159, US20090227032, US20090246875, and US20090304646, the disclosures of which are incorporated herein by reference. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

By “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e. ectoderm, mesoderm and endoderm. For example, somatic cells would include both neurons and neural progenitors, the latter of which may be able to naturally give rise to all or some cell types of the central nervous system but cannot give rise to cells of the mesoderm or endoderm lineages.

By “mitotic cell” it is meant a cell undergoing mitosis. Mitosis is the process by which a eukaryotic cell separates the chromosomes in its nucleus into two identical sets in two separate nuclei. It is generally followed immediately by cytokinesis, which divides the nuclei, cytoplasm, organelles and cell membrane into two cells containing roughly equal shares of these cellular components.

By “post-mitotic cell” it is meant a cell that has exited from mitosis, i.e., it is “quiescent”, i.e. it is no longer undergoing divisions. This quiescent state may be temporary, i.e. reversible, or it may be permanent.

By “meiotic cell” it is meant a cell that is undergoing meiosis. Meiosis is the process by which a cell divides its nuclear material for the purpose of producing gametes or spores. Unlike mitosis, in meiosis, the chromosomes undergo a recombination step which shuffles genetic material between chromosomes. Additionally, the outcome of meiosis is four (genetically unique) haploid cells, as compared with the two (genetically identical) diploid cells produced from mitosis.

In some instances, a component (e.g., a nucleic acid component (e.g., a CasPhi guide RNA); a protein component (e.g., a variant CRISPR-Cas effector polypeptide of the present disclosure or a fusion polypeptide of the present disclosure); fusion polypeptide; etc.); and the like) includes a label moiety. The terms “label”, “detectable label”, or “label moiety” as used herein refer to any moiety that provides for signal detection and may vary widely depending on the particular nature of the assay. Label moieties of interest include both directly detectable labels (direct labels; e.g., a fluorescent label) and indirectly detectable labels (indirect labels; e.g., a binding pair member). A fluorescent label can be any fluorescent label (e.g., a fluorescent dye (e.g., fluorescein, Texas red, rhodamine, ALEXAFLUOR® labels, and the like), a fluorescent protein (e.g., green fluorescent protein (GFP), enhanced GFP (EGFP), yellow fluorescent protein (YFP), red fluorescent protein (RFP), cyan fluorescent protein (CFP), cherry, tomato, tangerine, and any fluorescent derivative thereof), etc.). Suitable detectable (directly or indirectly) label moieties for use in the methods include any moiety that is detectable by spectroscopic, photochemical, biochemical, immunochemical, electrical, optical, chemical, or other means. For example, suitable indirect labels include biotin (a binding pair member), which can be bound by streptavidin (which can itself be directly or indirectly labeled). Labels can also include: a radiolabel (a direct label) (e.g., ³H, ¹²⁵I, ³⁵S, ¹⁴C, or ³²P); an enzyme (an indirect label) (e.g., peroxidase, alkaline phosphatase, galactosidase, luciferase, glucose oxidase, and the like); a fluorescent protein (a direct label) (e.g., green fluorescent protein, red fluorescent protein, yellow fluorescent protein, and any convenient derivatives thereof); a metal label (a direct label); a colorimetric label; a binding pair member; and the like. By “partner of a binding pair” or “binding pair member” is meant one of a first and a second moiety, wherein the first and the second moiety have a specific binding affinity for each other. Suitable binding pairs include, but are not limited to: antigen/antibodies (for example, digoxigenin/anti-digoxigenin, dinitrophenyl (DNP)/anti-DNP, dansyl-X-anti-dansyl, fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Any binding pair member can be suitable for use as an indirectly detectable label moiety.

Any given component, or combination of components can be unlabeled, or can be detectably labeled with a label moiety. In some cases, when two or more components are labeled, they can be labeled with label moieties that are distinguishable from one another.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to an individual organism, e.g., a mammal, including, but not limited to, murines, simians, humans, non-human primates, ungulates, felines, canines, bovines, ovines, mammalian farm animals, mammalian sport animals, and mammalian pets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a variant CRISPR-Cas effector polypeptide” includes a plurality of such polypeptides and reference to “the CasPhi guide RNA” includes reference to one or more CasPhi guide RNAs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides CRISPR-Cas effector polypeptides that exhibit enhanced gene editing and/or trans cleavage activity, compared to a wild-type CasΦ polypeptide. The present disclosure provides systems and kits comprising such CRISPR-Cas effector polypeptides. The present disclosure provides methods, including gene editing and diagnostic methods, using a CRISPR-Cas effector polypeptide of the present disclosure.

### Crispr-Cas Effector Polypeptides

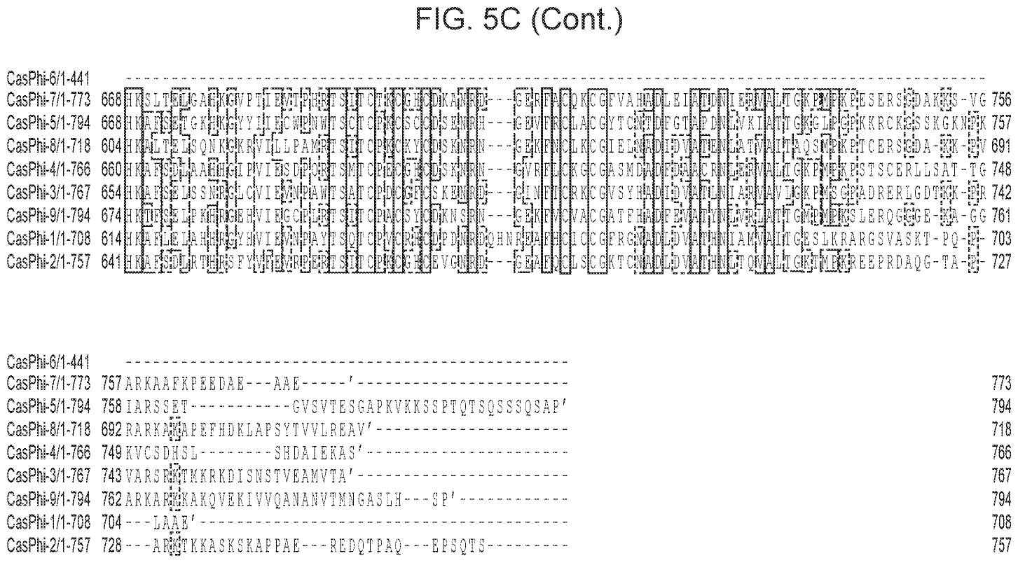

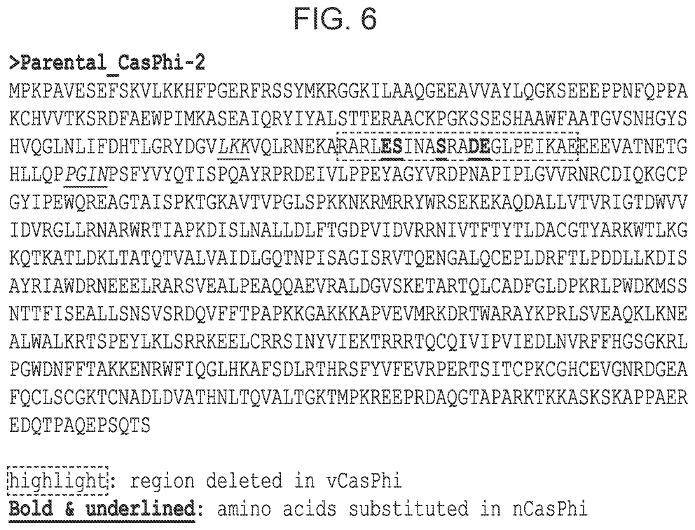

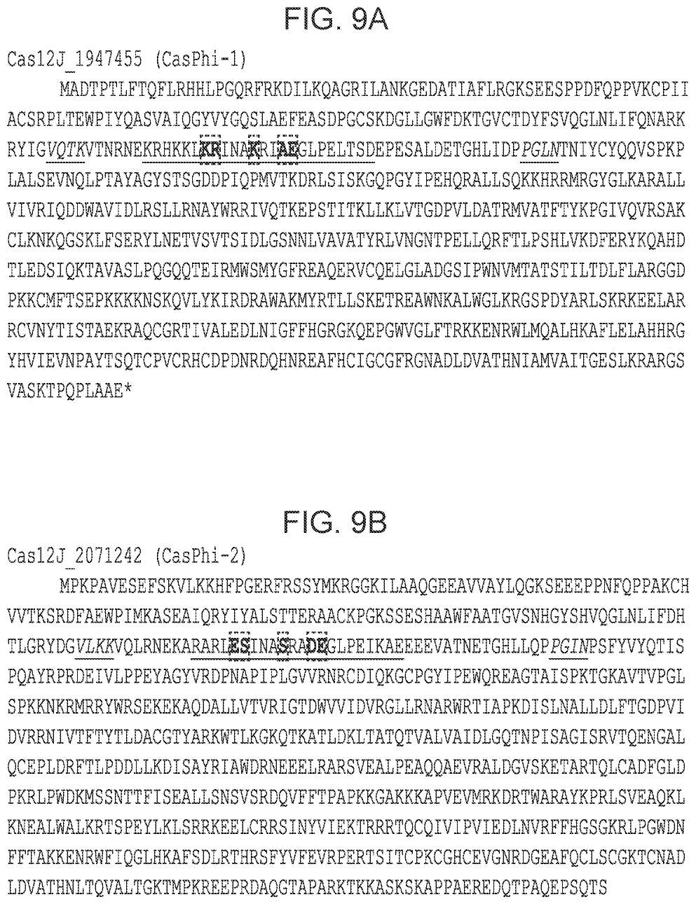

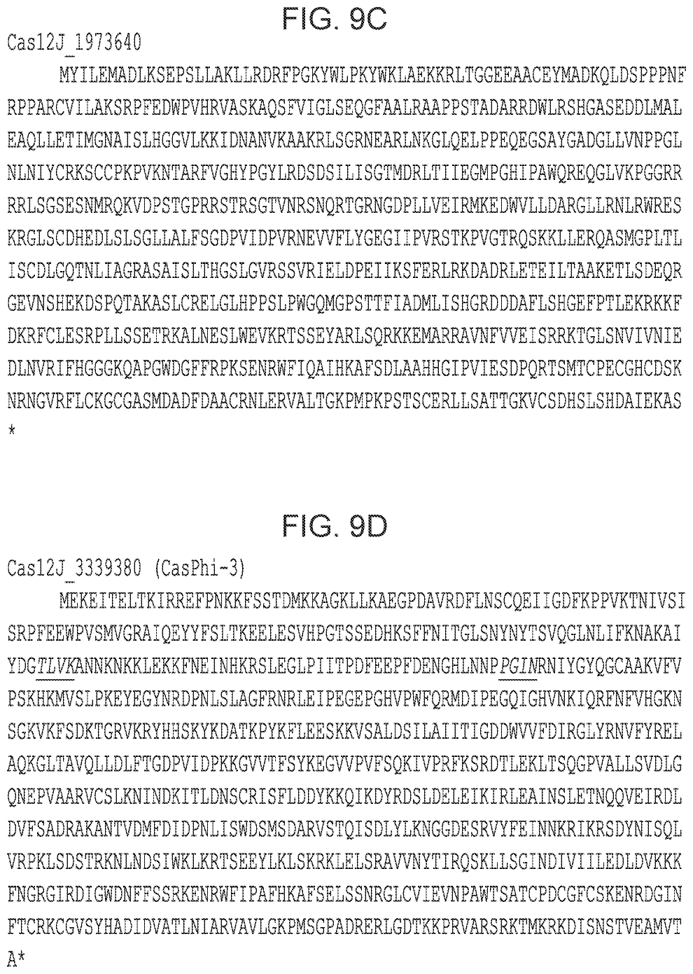

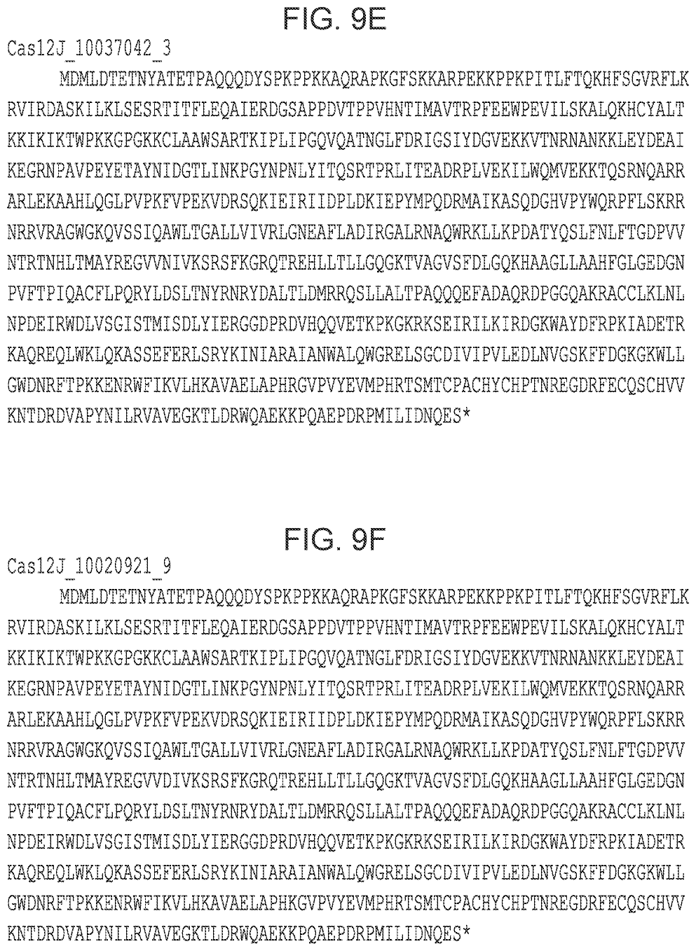

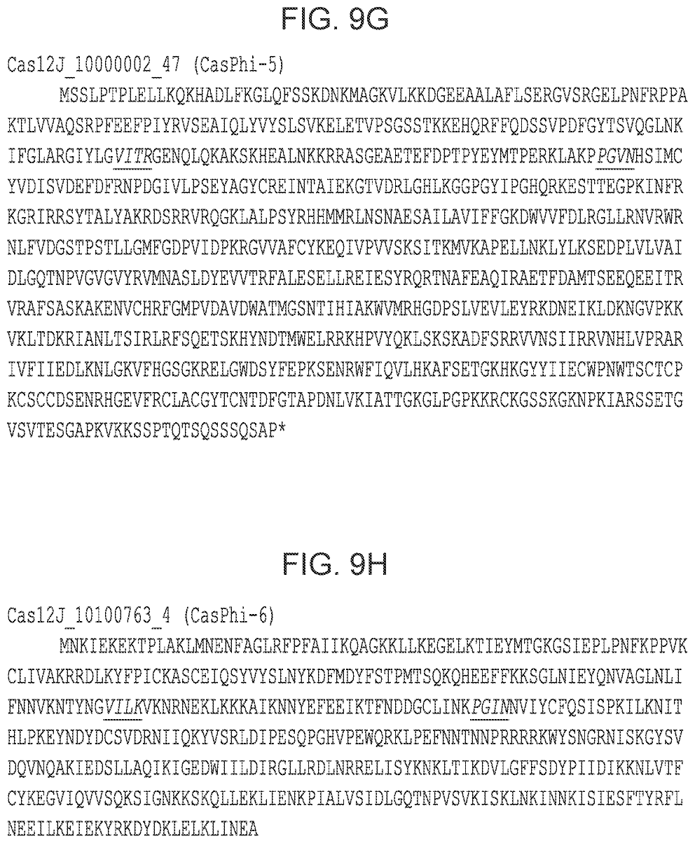

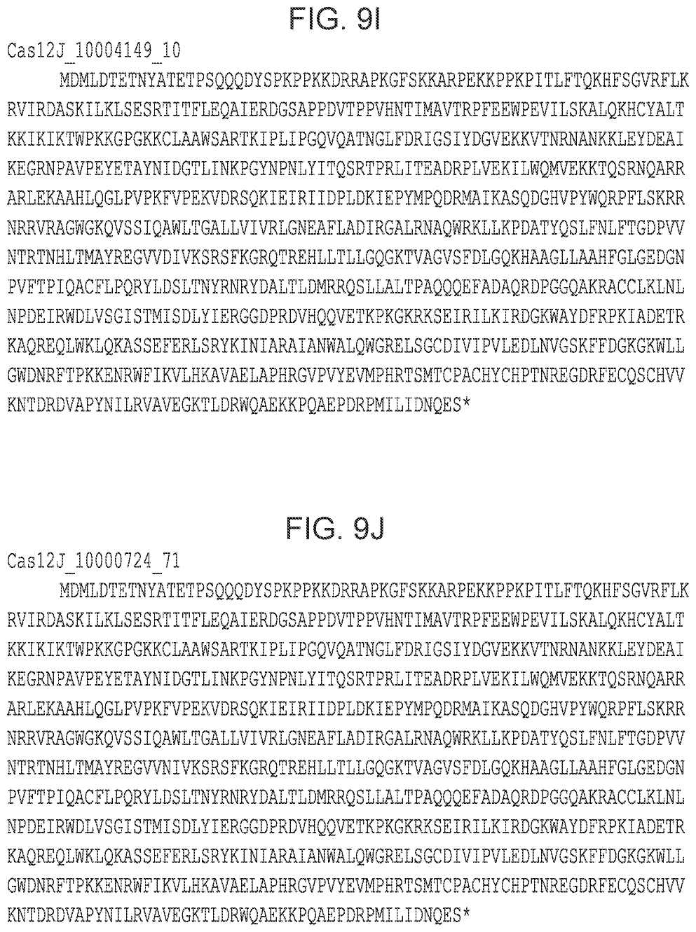

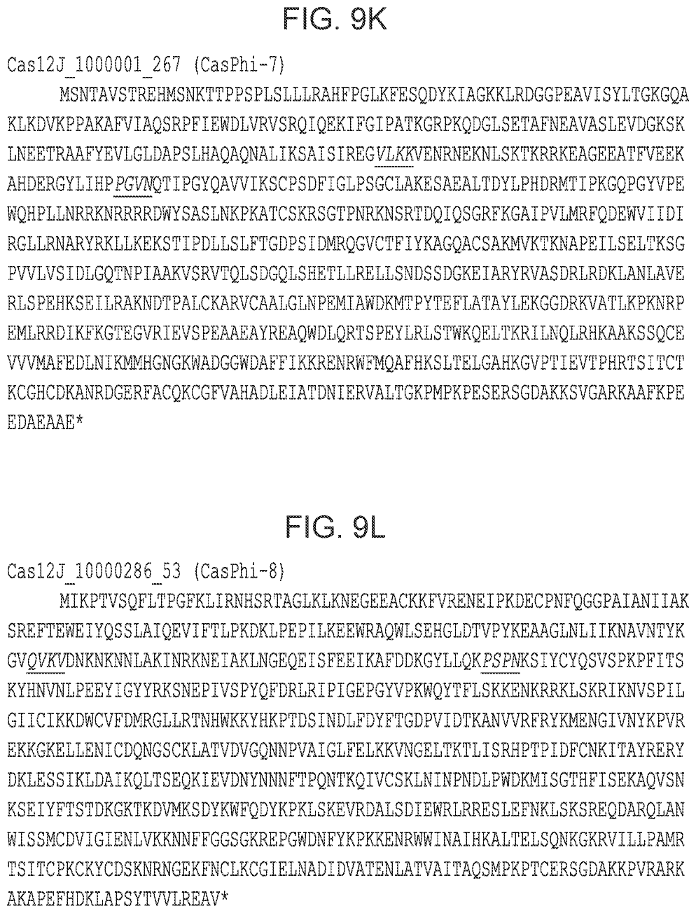

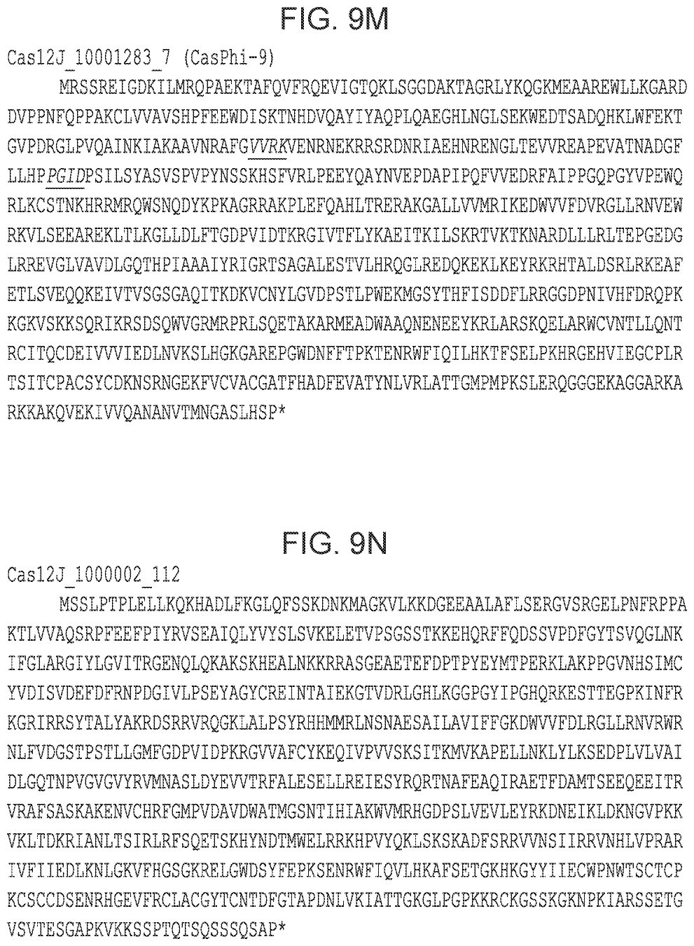

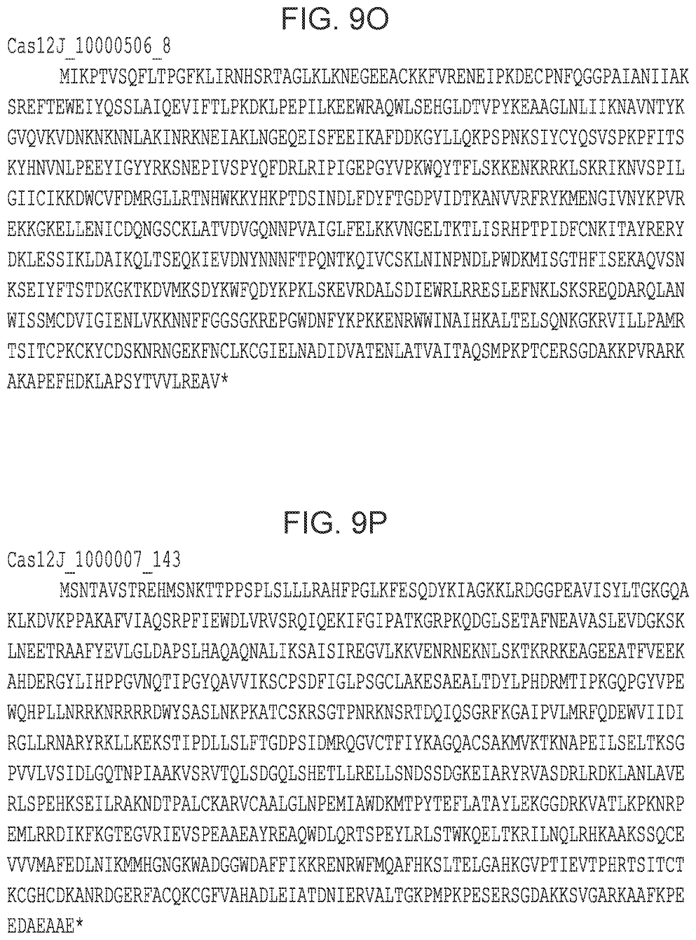

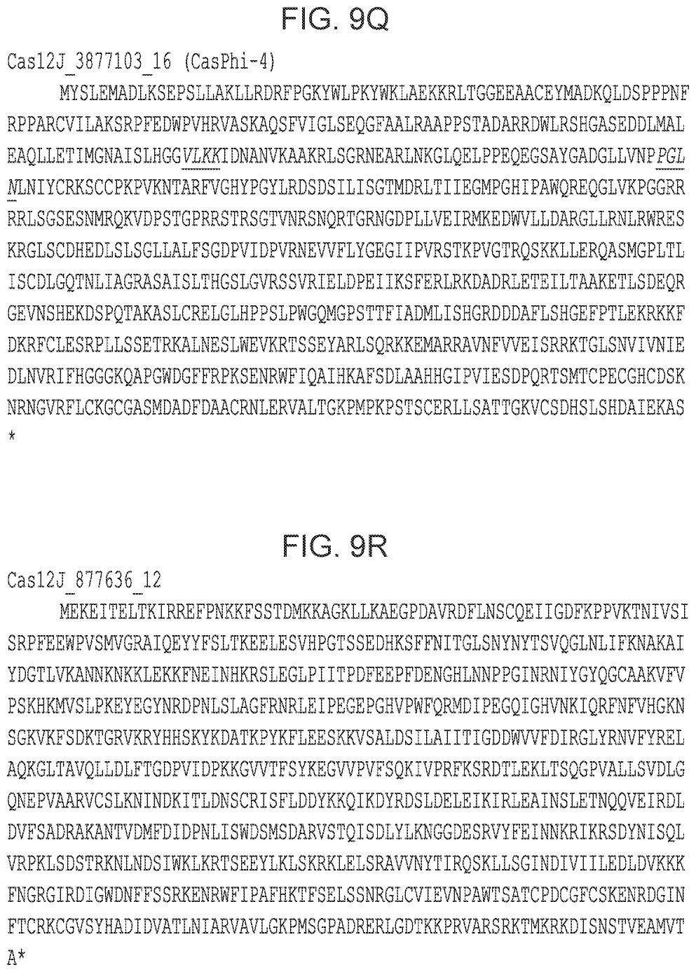

The present disclosure provides CRISPR-Cas effector polypeptides that exhibit enhanced cis- and/or trans cleavage activity, compared to a wild-type CasΦ polypeptide. A “wild-type CasΦ polypeptide” is also referred to herein as “wild-type CasPhi polypeptide.” A CRISPR-Cas effector polypeptide of the present disclosure is also referred to herein as a “variant CRISPR-Cas effector polypeptide” or a “variant CasPhi polypeptide” or a “variant CasΦ polypeptide.” A CRISPR-Cas effector polypeptide of the present disclosure can exhibit enhanced cis-cleavage activity and thus can exhibit enhanced gene editing activity, compared to a wild-type CasPhi polypeptide (e.g., compared to a CasPhi polypeptide comprising the amino acid sequence depicted in FIG. 6, or compared to a CasPhi polypeptide comprising the amino acid sequence depicted in any one of FIG. 9A-9R). A CRISPR-Cas effector polypeptide of the present disclosure can exhibit enhanced trans cleavage activity, compared to a wild-type CasPhi polypeptide (e.g., compared to a CasPhi polypeptide comprising the amino acid sequence depicted in FIG. 6, or compared to a CasPhi polypeptide comprising the amino acid sequence depicted in any one of FIG. 9A-9R); and thus is suitable for use in diagnostic applications.

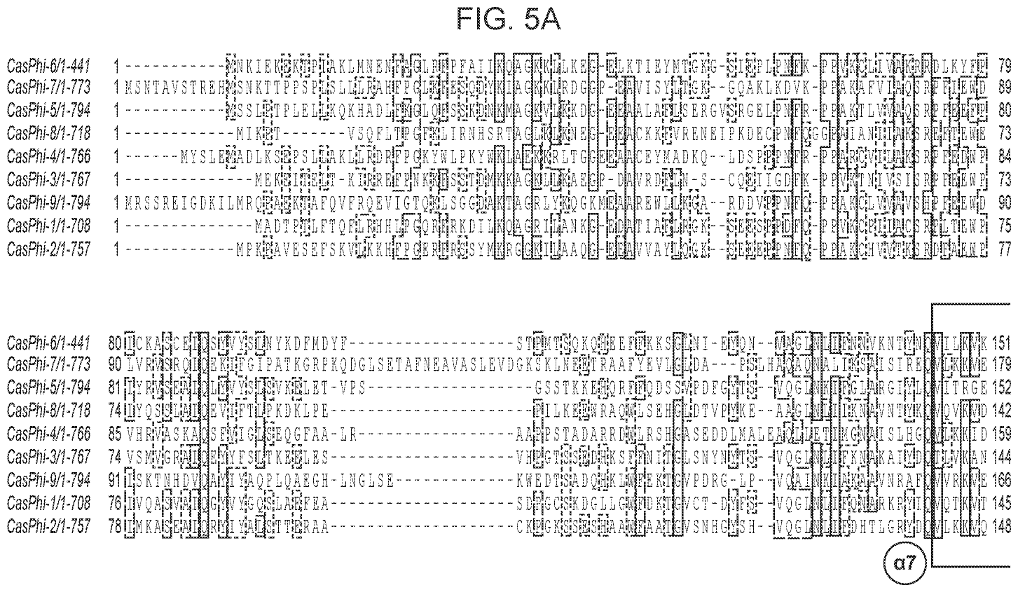

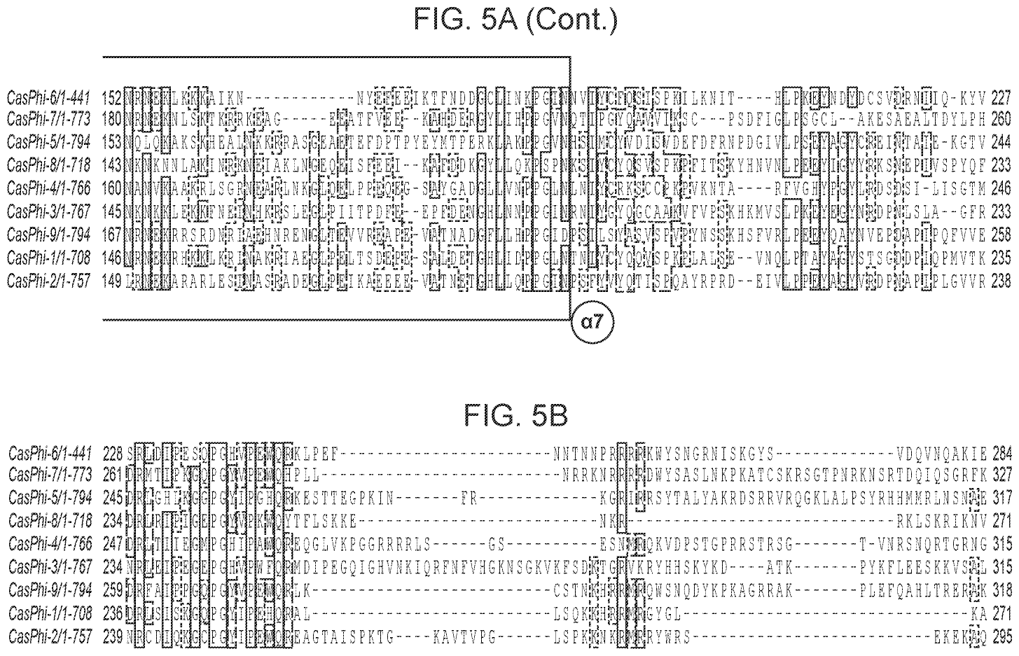

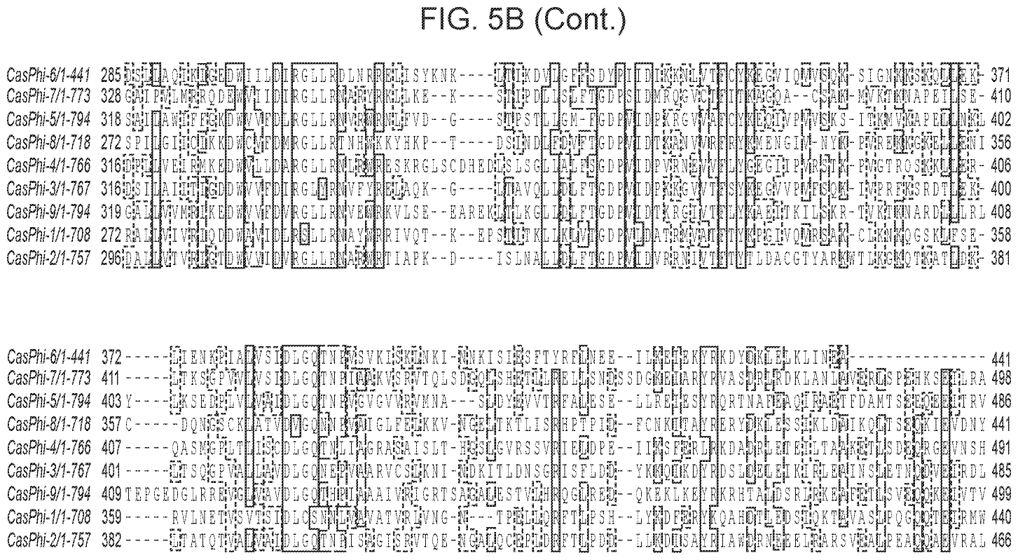

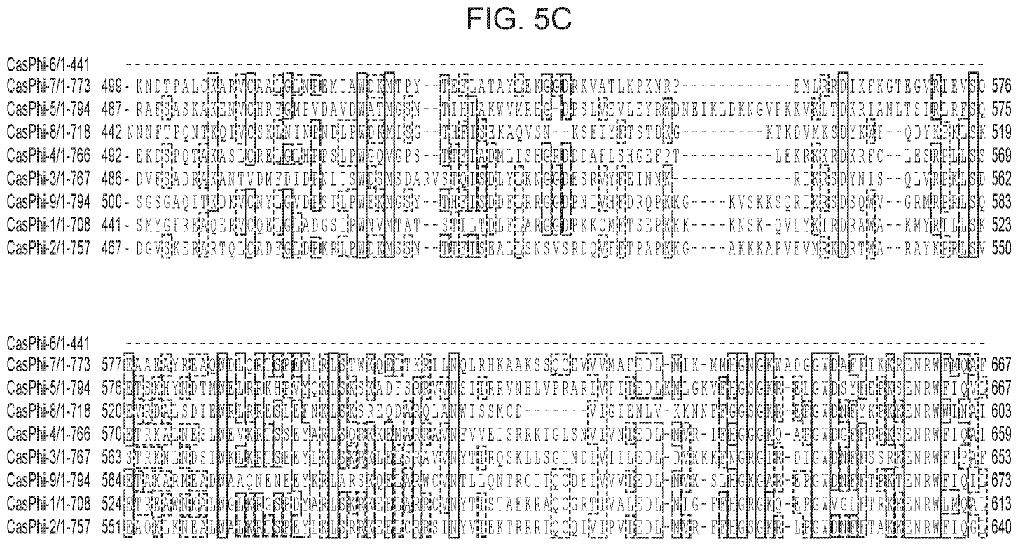

A CRISPR-Cas effector polypeptide of the present disclosure comprises modifications in the amino acid sequence of the helix alpha 7 structure of RecI, relative to a reference CasPhi polypeptide (e.g., compared to a wild-type CasPhi polypeptide comprising the amino acid sequence depicted in any one of FIG. 9A-9R). The location of the helix alpha 7 structure of several CasPhi polypeptides is depicted in FIG. 5A-5C. As shown in FIG. 5A-5C, the helix alpha 7 structure of a CasPhi polypeptide comprising the amino acid sequence depicted in FIG. 6 (CasPhi-2) is amino acids 144-195. Given the alignment provided in FIG. 5A-5C, those skilled in the art could readily determine the location of the helix alpha 7 structure of any of the CasPhi polypeptides depicted in FIG. 9A-9R.

In some cases, a variant CRISPR-Cas effector polypeptide of the present disclosure, when complexed with a guide nucleic acid (e.g., a CasPhi guide RNA) exhibits at least a 10% increased cis- and/or trans-cleavage activity compared to the cis- and/or trans-cleavage activity of a CasPhi polypeptide comprising the amino acid sequence depicted in FIG. 6. For example, in some cases, a variant CRISPR-Cas effector polypeptide of the present disclosure, when complexed with a guide nucleic acid (e.g., a CasPhi guide RNA) exhibits at least a 10%, at least a 25%, at least a 50%, at least a 100% (or 2-fold), at least at least 2.5-fold, at least 3.0-fold, at least 3.5-fold, at least 4.0-fold, at least 4.5-fold, at least 5.0-fold, at least 6-fold, at least 7-fold, at least 8-fold, at least 9-fold, at least 10-fold, or more than 10-fold, increased cis- and/or trans-cleavage activity compared to the cis- and/or trans-cleavage activity of a CasPhi polypeptide comprising the amino acid sequence depicted in FIG. 6. Cis cleavage refers to cleavage of a target nucleic acid that comprises a nucleotide sequence that is complementary to a nucleotide sequence in a guide RNA. Trans cleavage refers to cleavage of non-target nucleic acid that does not comprise a nucleotide sequence that is complementary to a nucleotide sequence in a guide RNA.

In some cases, a variant CRISPR-Cas effector polypeptide of the present disclosure, when complexed with a guide nucleic acid (e.g., a CasPhi guide RNA) cleaves a target nucleic acid more efficiently than a reference CRISPR-Cas effector polypeptide (e.g., a polypeptide comprising the amino acid sequence depicted in FIG. 6; or a polypeptide comprising the amino acid sequence depicted in any one of FIG. 9A-9R). For example, in some cases, a variant CRISPR-Cas effector polypeptide of the present disclosure, when complexed with a guide nucleic acid (e.g., a CasPhi guide RNA) exhibits at least 1.5-fold, at least 2.0-fold, at least 2.5-fold, at least 3.0-fold, at least 3.5-fold, at least 4.0-fold, at least 4.5-fold, at least 5.0-fold, at least 6-fold, at least 7-fold, at least 8-fold, at least 9-fold, at least 10-fold, or more than 10-fold, greater enzymatic activity (e.g., cleavage of a target nucleic acid), compared to the enzymatic activity of a CRISPR-Cas effector polypeptide comprising the amino acid sequence depicted in FIG. 6 (CasPhi-2) when complexed with the same guide nucleic acid, for cleavage of the same target nucleic acid.

In some cases, a variant CRISPR-Cas effector polypeptide of the present disclosure, when complexed with a guide nucleic acid (e.g., a CasPhi guide RNA) cleaves the non-target strand (NTS) of a target nucleic acid more efficiently than a reference CRISPR-Cas effector polypeptide (e.g., a polypeptide comprising the amino acid sequence depicted in FIG. 6; or a polypeptide comprising the amino acid sequence depicted in any one of FIG. 9A-9R). For example, in some cases, a variant CRISPR-Cas effector polypeptide of the present disclosure, when complexed with a guide nucleic acid (e.g., a CasPhi guide RNA) exhibits at least 1.5-fold, at least 2.0-fold, at least 2.5-fold, at least 3.0-fold, at least 3.5-fold, at least 4.0-fold, at least 4.5-fold, at least 5.0-fold, at least 6-fold, at least 7-fold, at least 8-fold, at least 9-fold, at least 10-fold, or more than 10-fold, greater cleavage of the NTS of a target nucleic acid, compared to the cleavage of the NTS of the same target nucleic acid by a CRISPR-Cas effector polypeptide comprising the amino acid sequence depicted in FIG. 6 (CasPhi-2) when complexed with the same guide nucleic acid.

In some cases, a variant CRISPR-Cas effector polypeptide of the present disclosure, when complexed with a guide nucleic acid (e.g., a CasPhi guide RNA) cleaves both the NTS and the target strand (TS) of a target nucleic acid more efficiently than a reference CRISPR-Cas effector polypeptide (e.g., a polypeptide comprising the amino acid sequence depicted in FIG. 6; or a polypeptide comprising the amino acid sequence depicted in any one of FIG. 9A-9R). For example, in some cases, a variant CRISPR-Cas effector polypeptide of the present disclosure, when complexed with a guide nucleic acid (e.g., a CasPhi guide RNA) exhibits at least 1.5-fold, at least 2.0-fold, at least 2.5-fold, at least 3.0-fold, at least 3.5-fold, at least 4.0-fold, at least 4.5-fold, at least 5.0-fold, at least 6-fold, at least 7-fold, at least 8-fold, at least 9-fold, at least 10-fold, or more than 10-fold, greater cleavage of both the NTS and the TS of a target nucleic acid, compared to the cleavage of the NTS and the TS of the same target nucleic acid by a CRISPR-Cas effector polypeptide comprising the amino acid sequence depicted in FIG. 6 (CasPhi-2) when complexed with the same guide nucleic acid.

For example, in some cases, a variant CRISPR-Cas effector polypeptide of the present disclosure, when complexed with a guide nucleic acid (e.g., a CasPhi guide RNA), cleaves 50% of the copies of a target nucleic acid within a time period that is at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, or at least 90% less, than the time period required by a reference CRISPR-Cas effector polypeptide (e.g., a polypeptide comprising the amino acid sequence depicted in FIG. 6; or a polypeptide comprising the amino acid sequence depicted in any one of FIG. 9A-9R). when complexed with the same guide nucleic acid, to cleave 50% of the copies of the same target nucleic acid.

In some cases, a variant CRISPR-Cas effector polypeptide of the present disclosure, when complexed with a guide nucleic acid (e.g., a CasPhi guide RNA), cleaves 50% of the copies of a target nucleic acids within a time period of from about 10 seconds to about 2 minutes (e.g., from about 10 seconds to about 15 seconds, from about 15 seconds to about 30 seconds, from about 30 seconds to about 45 seconds, from about 45 seconds to about 60 seconds, from about 60 seconds to about 75 seconds, from about 75 seconds to about 90 seconds, from about 90 seconds to about 105 seconds, or from about 105 seconds to about 120 seconds. In some cases, a variant CRISPR-Cas effector polypeptide of the present disclosure, when complexed with a guide nucleic acid (e.g., a CasPhi guide RNA), cleaves 50% of the copies of a target nucleic acids within a time period of from about 2 minutes to about 10 minutes (e.g., from about 2 minutes to about 3 minutes, from about 3 minutes to about 4 minutes, from about 4 minutes to about 5 minutes, from about 5 minutes to about 6 minutes, from about 6 minutes to about 7 minutes, from about 7 minutes to about 8 minutes, from about 8 minutes to about 9 minutes, or from about 9 minutes to about 10 minutes. The copies of the target nucleic acids can be in an in vitro, cell-free sample. The copies of the target nucleic acids can be in living cells (e.g., a plurality of living cells, where each cell of the plurality of living cells includes a copy of the target nucleic acid), where the living cells can be in vitro or in vivo.

In some cases, a variant CRISPR-Cas effector polypeptide of the present disclosure, when complexed with a guide nucleic acid (e.g., a CasPhi guide RNA) exhibits enhanced cleavage of a target nucleic acid when the target nucleic acid is in a repressive and compact chromatin state (“inaccessible chromatin”), compared to cleavage of the same target nucleic acid by a reference CRISPR-Cas effector polypeptide (e.g., a polypeptide comprising the amino acid sequence depicted in FIG. 6; or a polypeptide comprising the amino acid sequence depicted in any one of FIG. 9A-9R). For example, in some cases, a variant CRISPR-Cas effector polypeptide of the present disclosure, when complexed with a guide nucleic acid (e.g., a CasPhi guide RNA) exhibits at least 20%, at least 25%, at least 50%, at least 1.5-fold, at least 2.0-fold, at least 2.5-fold, at least 3.0-fold, at least 3.5-fold, at least 4.0-fold, at least 4.5-fold, at least 5.0-fold, at least 6-fold, at least 7-fold, at least 8-fold, at least 9-fold, at least 10-fold, at least 20-fold, at least 50-fold, at least 100-fold, or more than 100-fold, greater cleavage of a target nucleic acid when the target nucleic acid is in a repressive and compact chromatin state, compared to cleavage of the same target nucleic acid by a CRISPR-Cas effector polypeptide comprising the amino acid sequence depicted in FIG. 6 (CasPhi-2) when complexed with the same guide nucleic acid. A target nucleic acid that is present in repressive and compact chromatin is a target nucleic acid that is present in a region of chromatin that is not actively transcribed and is relatively inaccessible to regulatory factors. Those skilled in the art can determine whether a target nucleic acid is present in repressive and compact chromatin. See, e.g., Zhong et al. (2021) Proc. Natl. Acad. Sci. USA 118:e2023347118; Klemm et al. (2019) Nature Reviews Genetics 20:207; and Cusanovich et al. (2015) Science 348:910.

In some cases, a variant CRISPR-Cas effector polypeptide of the present disclosure, when complexed with a guide nucleic acid (e.g., a CasPhi guide RNA) exhibits at least a 10% increased cis-cleavage of a target nucleic acid, when the target nucleic acid is an active and accessible chromatin (“accessible chromatin”), compared to the cis-cleavage of the same target nucleic acid by a CasPhi polypeptide comprising the amino acid sequence depicted in FIG. 6. For example, in some cases, a variant CRISPR-Cas effector polypeptide of the present disclosure, when complexed with a guide nucleic acid (e.g., a CasPhi guide RNA) exhibits at least a 10%, at least a 25%, at least a 50%, at least a 100% (or 2-fold), at least at least 2.5-fold, at least 3.0-fold, at least 3.5-fold, at least 4.0-fold, at least 4.5-fold, at least 5.0-fold, at least 6-fold, at least 7-fold, at least 8-fold, at least 9-fold, at least 10-fold, or more than 10-fold (e.g., at least 15-fold, at least 20-fold, at least 50-fold, at least 100-fold, or more than 100-fold), increased cis-cleavage of a target nucleic acid, when the target nucleic acid is an active and accessible chromatin, compared to the cis-cleavage of the same target nucleic acid by a CRISPR-Cas effector polypeptide comprising the amino acid sequence depicted in FIG. 6 (CasPhi-2) when complexed with the same guide nucleic acid. A target nucleic acid that is present in active and accessible chromatin is generally a target nucleic acid that is actively transcribed and is accessible to proteins such as transcription factors, RNA polymerase, and the like. Those skilled in the art can determine whether a target nucleic acid is present in active and accessible chromatin. See, e.g., Zhong et al. (2021) Proc. Natl. Acad. Sci. USA 118:e2023347118; Klemm et al. (2019) Nature Reviews Genetics 20:207; and Cusanovich et al. (2015) Science 348:910.

In some cases, a variant CRISPR-Cas effector polypeptide of the present disclosure, when complexed with a guide nucleic acid (e.g., a CasPhi guide RNA), and when activated by binding to a target nucleic acid, exhibits more efficient trans cleavage than a reference CRISPR-Cas effector polypeptide (e.g., a polypeptide comprising the amino acid sequence depicted in FIG. 6; or a polypeptide comprising the amino acid sequence depicted in any one of FIG. 9A-9R). For example, in some cases, a variant CRISPR-Cas effector polypeptide of the present disclosure, when complexed with a guide nucleic acid (e.g., a CasPhi guide RNA) and when activated by binding to a target nucleic acid, exhibits at least 1.5-fold, at least 2.0-fold, at least 2.5-fold, at least 3.0-fold, at least 3.5-fold, at least 4.0-fold, at least 4.5-fold, at least 5.0-fold, at least 6-fold, at least 7-fold, at least 8-fold, at least 9-fold, at least 10-fold, or more than 10-fold, greater trans cleavage (cleavage of non-target nucleic acid), compared to the trans cleavage by a CRISPR-Cas effector polypeptide comprising the amino acid sequence depicted in FIG. 6 (CasPhi-2) when complexed with the same guide nucleic acid.

As noted above, a CRISPR-Cas effector polypeptide of the present disclosure comprises modifications in the amino acid sequence of the helix alpha 7 structure of RecI, relative to a reference CasPhi polypeptide (e.g., compared to a wild-type CasPhi polypeptide comprising the amino acid sequence depicted in any one of FIG. 9A-9R). As shown in FIG. 5A-5C, the helix alpha 7 structure of a CasPhi polypeptide comprising the amino acid sequence depicted in FIG. 6 (CasPhi-2) is amino acids 144-195. In some cases, a CRISPR-Cas effector polypeptide of the present disclosure comprises a deletion or a substitution of one or more amino acids in the alpha-7 helix of the Rec I domain, compared to the amino acid sequence depicted in FIG. 6, or a corresponding region of another CasPhi polypeptide.

In some cases, a CRISPR-Cas effector polypeptide of the present disclosure comprises a deletion or a substitution of one or more amino acids within amino acids 144-195 of the amino acid sequence depicted in FIG. 6, or a corresponding region of another CasPhi polypeptide. In some cases, a CRISPR-Cas effector polypeptide of the present disclosure comprises a deletion or a substitution of one or more amino acids within amino acids 150-185 of the amino acid sequence depicted in FIG. 6, or a corresponding region of another CasPhi polypeptide. In some cases, a CRISPR-Cas effector polypeptide of the present disclosure comprises a deletion or a substitution of one or more amino acids within amino acids 155-180 of the amino acid sequence depicted in FIG. 6, or a corresponding region of another CasPhi polypeptide. In some cases, a CRISPR-Cas effector polypeptide of the present disclosure comprises a deletion or a substitution of one or more amino acids within amino acids 155-176 of the amino acid sequence depicted in FIG. 6, or a corresponding region of another CasPhi polypeptide.

In some cases, a CRISPR-Cas effector polypeptide of the present disclosure comprises an amino acid sequence having at least 50% amino acid sequence identity to any one of the amino acid sequences depicted in FIG. 9A-9R; and comprises substitutions of amino acids E159, S160, S164, D167, and E168, compared to the amino acid sequence depicted in FIG. 6, or corresponding amino acids in another CasPhi polypeptide. For example, in some cases, a CRISPR-Cas effector polypeptide of the present disclosure comprises an amino acid sequence having at least 50% amino acid sequence identity to any one of the amino acid sequences depicted in FIG. 9A-9R; where amino acids 159-168 (where the numbering is based on the numbering depicted in FIG. 6) are X₁X₂X₃X₄X₅X₆X₇X₈X₉X₁₀ (SEQ ID NO:50), where X₁ is any amino acid other than Glu; X₂ is any amino acid other than Ser; X₃ is Ile, Arg, Lys, Leu, or Asn; X₄ is Asn, Lys, or Ala; X₅ is Ala, Glu, His, or Lys; X₆ is any amino acid other than Ser; X₇ is Arg, Asn, Ala, or Cys; X₈ is Ala, Ile, Arg, Ser, Leu, or Lys; X₉ is any amino acid other than Asp; and X₁₀ is any amino acid other than Glu. For example, X₁ is Ala, Arg, Asn, Asp, Cys, Gln, Gly, His, Ile, Leu, Lys, Met, Phe, Pro, Ser, Thr, Trp, Tyr, or Val; X₂ is Ala, Arg, Asn, Asp, Cys, Gln, Glu, Gly, His, Ile, Leu, Lys, Met, Phe, Pro, Thr, Trp, Tyr, or Val; X₆ is Ala, Arg, Asn, Asp, Cys, Gln, Glu, Gly, His, Ile, Leu, Lys, Met, Phe, Pro, Thr, Trp, Tyr, or Val; X₉ is Ala, Arg, Asn, Cys, Gln, Glu, Gly, His, Ile, Leu, Lys, Met, Phe, Pro, Ser, Thr, Trp, Tyr, or Val; and X₁₀ is Ala, Arg, Asn, Asp, Cys, Gln, Gly, His, Ile, Leu, Lys, Met, Phe, Pro, Ser, Thr, Trp, Tyr, or Val. In some cases, X₁ is Ala or Gly; X₂ is Ala or Gly; X₃ is Ile; X₄ is Asn; X₅ is Ala; X₆ is Ala or Gly; X₇ is Arg; X₈ is Ala; X₉ is Ala or Gly; and X₁₀ is Ala or Gly. In some cases, X₁ is Ala; X₂ is Ala; X₃ is Ile; X₄ is Asn; X₅ is Ala; X₆ is Ala; X₇ is Arg; X₈ is Ala; X₉ is Ala; and X₁₀ is Ala.

As another example, in some cases, a CRISPR-Cas effector polypeptide of the present disclosure comprises an amino acid sequence having at least 50% amino acid sequence identity to any one of the amino acid sequences depicted in FIG. 9A-9R; where amino acids 159-168 (where the numbering is based on the numbering depicted in FIG. 6) are X₁X₂INSX₃RAX₄X₅(SEQ ID NO:51), where X₁ is any amino acid other than Glu; X₂ is any amino acid other than Ser; X₃ is any amino acid other than Ser; X₄ is any amino acid other than Asp; and X₅ is any amino acid other than Glu. For example, X₁ is Ala, Arg, Asn, Asp, Cys, Gln, Gly, His, Ile, Leu, Lys, Met, Phe, Pro, Ser, Thr, Trp, Tyr, or Val; X₂ is Ala, Arg, Asn, Asp, Cys, Gln, Glu, Gly, His, Ile, Leu, Lys, Met, Phe, Pro, Thr, Trp, Tyr, or Val; X₃ is Ala, Arg, Asn, Asp, Cys, Gln, Glu, Gly, His, Ile, Leu, Lys, Met, Phe, Pro, Thr, Trp, Tyr, or Val; X₄ is Ala, Arg, Asn, Cys, Gln, Glu, Gly, His, Ile, Leu, Lys, Met, Phe, Pro, Ser, Thr, Trp, Tyr, or Val; and X₅ is Ala, Arg, Asn, Asp, Cys, Gin, Gly, His, Ile, Leu, Lys, Met, Phe, Pro, Ser, Thr, Trp, Tyr, or Val. In some cases, X₁ is Ala or Gly; X₂ is Ala or Gly; X₃ is Ala or Gly; X₄ is Ala or Gly; and X₅ is Ala or Gly. In some cases, X₁ is Ala; X₂ is Ala; X₃ is Ala; X₄ is Ala; and X₅ is Ala. In some cases, amino acids 158-168 are AAINAARAAA (SEQ ID NO:52).

In some cases, a CRISPR-Cas effector polypeptide of the present disclosure comprises an amino acid sequence having at least 50% amino acid sequence identity to the amino acid sequence depicted in FIG. 8, where amino acids 159, 160, 164, 167, and 178 are Ala.

In some cases, a contiguous stretch of from about 15 amino acids to about 52 amino acids of amino acids 144-195 of the amino acid sequence depicted in FIG. 6, or a corresponding region of another CasPhi polypeptide are replaced with a heterologous polypeptide of from about 4 amino acids to about 250 amino acid in length, to generate a CRISPR-Cas effector polypeptide of the present disclosure. Thus, for example, in some cases, a CRISPR-Cas effector polypeptide of the present disclosure comprises a deletion of a contiguous stretch of from about 15 amino acids to about 52 amino acids of amino acids within amino acids 144-195 of the amino acid sequence depicted in FIG. 6, or a corresponding region of another CasPhi polypeptide; where the deleted amino acids are replaced with a heterologous polypeptide of from about 4 amino acids to about 250 amino acid in length. In some cases, a CRISPR-Cas effector polypeptide of the present disclosure comprises a deletion of a contiguous stretch of from about 15 amino acids to about 30 amino acids of amino acids within amino acids 150-185 of the amino acid sequence depicted in FIG. 6, or a corresponding region of another CasPhi polypeptide; where the deleted amino acids are replaced with a heterologous polypeptide of from about 4 amino acids to about 250 amino acid in length. In some cases, a CRISPR-Cas effector polypeptide of the present disclosure comprises a deletion of a contiguous stretch of from about 15 amino acids to about 26 amino acids of amino acids within amino acids 155-180 of the amino acid sequence depicted in FIG. 6, or a corresponding region of another CasPhi polypeptide; where the deleted amino acids are replaced with a heterologous polypeptide of from about 4 amino acids to about 250 amino acid in length. In some cases, a CRISPR-Cas effector polypeptide of the present disclosure comprises a deletion of a contiguous stretch of from about 15 amino acids to about 22 amino acids of amino acids within amino acids 155-176 of the amino acid sequence depicted in FIG. 6, or a corresponding region of another CasPhi polypeptide; where the deleted amino acids are replaced with a heterologous polypeptide of from about 4 amino acids to about 250 amino acid in length. The heterologous polypeptide can have a length of from about 4 amino acids (aa) to about 10 aa, from about 4 aa to about 15 aa, from about 4 an to about 20 aa, from about 4 aa to about 25 aa, from about 10 aa to about 15 aa, from about 15 an to about 20 aa, from about 20 aa to about 25 aa, from about 25 aa to about 50 aa, from about 50 an to about 100 aa, from about 100 an to about 150 aa, from about 150 aa to about 200 aa, or from about 200 an to about 250 aa.

In some cases, a contiguous stretch of from about 15 amino acids to about 52 amino acids of amino acids 144-195 of the amino acid sequence depicted in FIG. 6, or a corresponding region of another CasPhi polypeptide are replaced with a heterologous polypeptide of from about 4 amino acids to about 25 amino acid in length, to generate a CRISPR-Cas effector polypeptide of the present disclosure. Thus, for example, in some cases, a CRISPR-Cas effector polypeptide of the present disclosure comprises a deletion of a contiguous stretch of from about 15 amino acids to about 52 amino acids of amino acids within amino acids 144-195 of the amino acid sequence depicted in FIG. 6, or a corresponding region of another CasPhi polypeptide; where the deleted amino acids are replaced with a heterologous polypeptide of from about 4 amino acids to about 25 amino acid in length. In some cases, a CRISPR-Cas effector polypeptide of the present disclosure comprises a deletion of a contiguous stretch of from about 15 amino acids to about 30 amino acids of amino acids within amino acids 150-185 of the amino acid sequence depicted in FIG. 6, or a corresponding region of another CasPhi polypeptide; where the deleted amino acids are replaced with a heterologous polypeptide of from about 4 amino acids to about 25 amino acid in length. In some cases, a CRISPR-Cas effector polypeptide of the present disclosure comprises a deletion of a contiguous stretch of from about 15 amino acids to about 26 amino acids of amino acids within amino acids 155-180 of the amino acid sequence depicted in FIG. 6, or a corresponding region of another CasPhi polypeptide; where the deleted amino acids are replaced with a heterologous polypeptide of from about 4 amino acids to about 25 amino acid in length. In some cases, a CRISPR-Cas effector polypeptide of the present disclosure comprises a deletion of a contiguous stretch of from about 15 amino acids to about 22 amino acids of amino acids within amino acids 155-176 of the amino acid sequence depicted in FIG. 6, or a corresponding region of another CasPhi polypeptide; where the deleted amino acids are replaced with a heterologous polypeptide of from about 4 amino acids to about 25 amino acid in length.

In some cases, a CRISPR-Cas effector polypeptide of the present disclosure comprises an amino acid sequence having at least 50% amino acid sequence identity to any one of the amino acid sequences depicted in FIG. 9A-9R, where: i) a contiguous stretch of amino acids of from about 15 amino acids to about 52 amino acids of amino acids 144-195, or ii) a contiguous stretch of from about 15 amino acids to about 30 amino acids of amino acids within amino acids 150-185, or iii) a contiguous stretch of from about 15 amino acids to about 26 amino acids of amino acids within amino acids 155-180, or iv) a contiguous stretch of from about 15 amino acids to about 22 amino acids of amino acids within amino acids 155-176, of the amino acid sequence depicted in FIG. 6, or a corresponding region of another CasPhi polypeptide, is replaced with a heterologous polypeptide of from about 4 amino acids to about 25 amino acid in length.

The heterologous polypeptide that replaces the deletion of amino acids in the helix alpha 7 structure in some cases has a length of from about 4 amino acids to about 25 amino acids; and can comprise Gly, Ser, or a combination of Gly and Ser. For example, in some cases, the heterologous polypeptide that replaces the deletion of amino acids in the helix alpha 7 structure is (Gly)n (SEQ ID NO:53), where n is an integer from 4 to about 25 (e.g., 4, 5, 6, 7, 8, 9, 10, from 10 to 15, from 15 to 20, or from 20 to 25). As another example, in some cases, the heterologous polypeptide that replaces the deletion of amino acids in the helix alpha 7 structure is (Ser)n (SEQ ID NO:54), where n is an integer from 4 to about 25 (e.g., 4, 5, 6, 7, 8, 9, 10, from 10 to 15, from 15 to 20, or from 20 to 25). As another example, in some cases, the heterologous polypeptide that replaces the deletion of amino acids in the helix alpha 7 structure is (GSSG)n (SEQ ID NO:55), where n is an integer from 1 to 6 (e.g., where n is 1, 2, 3, 4, 5, or 6); and in some cases, n is 1. As another example, in some cases, the heterologous polypeptide that replaces the deletion of amino acids in the helix alpha 7 structure is (GGGGS)n (SEQ ID NO:56), where n is an integer from 1 to 5 (e.g., where n is 1, 2, 3, 4, or 5); and in some cases, n is 1.

The heterologous polypeptide that replaces the deletion of amino acids in the helix alpha 7 structure can be any of a variety of heterologous polypeptides, where suitable heterologous polypeptides include nucleic acid interacting polypeptides; nucleic acid modifying polypeptides; and the like. Any heterologous polypeptide, as described below, can be used to replace the deletion of amino acids in the helix alpha 7 structure in a variant CasPhi polypeptide of the present disclosure.

In some cases, the heterologous polypeptide that replaces the deletion of amino acids in the helix alpha 7 structure exhibits enzymatic activity. For example, the heterologous polypeptide that replaces the deletion of amino acids in the helix alpha 7 structure can be a DNA ligase, a base editor (e.g., a DNA methyltransferase), a DNA nuclease, a DNA helicase, or a DNA kinase. As one example, the heterologous polypeptide that replaces the deletion of amino acids in the helix alpha 7 structure can be a base editor, where suitable base editors are described elsewhere herein. In some cases, the heterologous polypeptide that replaces the deletion of amino acids in the helix alpha 7 structure exhibits nucleic acid binding activity. For example, a suitable heterologous polypeptide is a single-strand binding protein (SSB protein).

In some cases, the heterologous polypeptide that replaces the deletion of amino acids in the helix alpha 7 structure exhibits protein-binding activity, i.e., is a protein-binding polypeptide. Non-limiting examples of such protein-binding polypeptides include, e.g., an ALFA-tag (e.g., a peptide having the amino acid sequence SRLEEELRRRLTE; SEQ ID NO:57; and having a length of about 13 amino acids); an AviTag, e.g., a peptide that provides for biotinylation by the enzyme BirA, e.g., where the peptide has the sequence GLNDIFEAQKIEWHE (SEQ ID NO:58) and has a length of about 15 amino acids; a C-tag, e.g., a peptide comprising the amino acid sequence EPEA (SEQ ID NO:59) and having a length of about 4 amino acids; a calmodulin tag, e.g., a peptide bound by the protein calmodulin, where the peptide can comprise KRRWKKNFIAVSAANRFKKISSSGAL (SEQ ID NO:60), where the peptide can have a length of about 26 amino acids; a polyglutamate tag, e.g., (Glu)n (SEQ ID NO:61), where n is an integer from 4 to 10, e.g., where n is 6; a polyarginine tag, e.g., (Arg)n (SEQ ID NO:62), where n is an integer from 5 to 9; an E-tag, e.g., a peptide having the sequence GAPVPYPDPLEPR (SEQ ID NO:63), where the peptide has a length of about 13 amino acids; a FLAG tag, e.g., a peptide having the sequence DYKDDDDK (SEQ ID NO:64) and having a length of 8 amino acids; a hemagglutinin tag, e.g., a peptide having the sequence YPYDVPDYA (SEQ ID NO:65) and having a length of about 9 amino acids; a histidine tag, e.g., (His)n (SEQ ID NO:66), where n is an integer from 5 to 10; a myc tag, e.g., a peptide having the sequence EQKLISEEDL (SEQ ID NO:67) and having a length of about 10 amino acids; an NE-tag, e.g., a peptide having the sequence TKENPRSNQEESYDDNES (SEQ ID NO:68) and having a length of about 18 amino acids; a Rho1D4 tag, e.g., a peptide having the sequence TETSQVAPA (SEQ ID NO:69) and having a length of about 9 amino acids; an S tag, e.g., a peptide having the sequence KETAAAKFERQHMDS (SEQ ID NO:70) and having a length of about 15 amino acids; an SBP tag, e.g., a peptide having the sequence MDEKTTGWRGGHVVEGLAGELEQLRARLEHHPQGQREP (SEQ ID NO:71) and having a length of about 38 amino acids; a Softag 1, e.g., a peptide having the sequence SLAELLNAGLGGS (SEQ ID NO:72) and having a length of about 13 amino acids; a Softag 3, e.g., a peptide having the sequence TQDPSRVG (SEQ ID NO:73) and having a length of about 8 amino acids; a Strep tag, e.g., a peptide having the sequence WSHPQFEK (SEQ ID NO:74) and having a length of about 8 amino acids; a T7 tag, e.g., a peptide having the sequence MASMTGGQQMG (SEQ ID NO:75) and having a length of about 11 amino acids; a tetracysteine (TC) tag, e.g., a peptide having the sequence CCPGCC (SEQ ID NO:76) and having a length of about 6 amino acids; a Ty tag, e.g., a peptide having the sequence EVHTNQDPLD (SEQ ID NO:77) and having a length of about 10 amino acids; a V5 tag, e.g., a peptide having the sequence GKPIPNPLLGLDST (SEQ ID NO:78) and having a length of about 14 amino acids; a VSV tag, e.g., a peptide having the sequence YTDIEMNRLGK (SEQ ID NO:79) and having a length of about 11 amino acids; or an Xpress tag, e.g., a peptide having the sequence DLYDDDDK (SEQ ID NO:80) and having a length of about 8 amino acids.

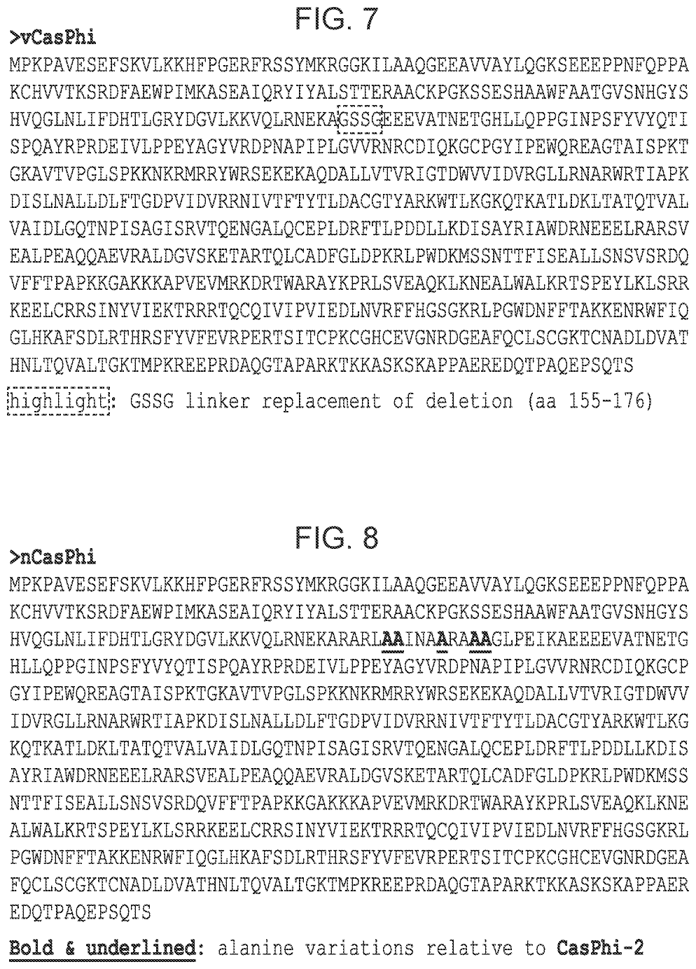

In some cases, a variant CRISPR-Cas effector polypeptide of the present disclosure comprises an amino acid sequence having at least 50% amino acid sequence identity to the amino acid sequence depicted in FIG. 7, where the variant CRISPR-Cas effector polypeptide has a length of from about 730 amino acids to about 740 amino acids.

### Fusion Proteins

As noted above, in some cases, a variant CRISPR-Cas effector polypeptide of the present disclosure is fused to one or more heterologous polypeptides that has an activity of interest (e.g., a catalytic activity of interest, subcellular localization activity, etc.) to form a fusion protein. A heterologous polypeptide to which a variant CRISPR-Cas effector polypeptide of the present disclosure can be fused is referred to herein as a “fusion partner.” Thus, the present disclosure provides a fusion polypeptide comprising: a) a variant CRISPR-Cas effector polypeptide of the present disclosure; and b) one or more heterologous polypeptides.

In some cases, the fusion partner can modulate transcription (e.g., inhibit transcription, increase transcription) of a target DNA. For example, in some cases the fusion partner is a protein (or a domain from a protein) that inhibits transcription (e.g., a transcriptional repressor, a protein that functions via recruitment of transcription inhibitor proteins, modification of target DNA such as methylation, recruitment of a DNA modifier, modulation of histones associated with target DNA, recruitment of a histone modifier such as those that modify acetylation and/or methylation of histones, and the like). In some cases, the fusion partner is a protein (or a domain from a protein) that increases transcription (e.g., a transcription activator, a protein that acts via recruitment of transcription activator proteins, modification of target DNA such as demethylation, recruitment of a DNA modifier, modulation of histones associated with target DNA, recruitment of a histone modifier such as those that modify acetylation and/or methylation of histones, and the like). In some cases, the fusion partner is a reverse transcriptase. In some cases, the fusion partner is a base editor. In some cases, the fusion partner is a deaminase.

In some cases, a fusion polypeptide of the present disclosure includes a heterologous polypeptide that has enzymatic activity that modifies a target nucleic acid (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, or glycosylase activity).

In some cases, a fusion polypeptide of the present disclosure includes a heterologous polypeptide that has enzymatic activity that modifies a polypeptide (e.g., a histone) associated with a target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

Examples of proteins (or fragments thereof) that can be used in increase transcription include but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1, and the like; histone lysine demethylases such as JHDM2a/b, UTX, JMJD3, and the like; histone acetyltransferases such as GCN5, PCAF, CBP, p300, TAF, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TET1CD), TET1, DME, DML1, DML2, ROS1, and the like.

Examples of proteins (or fragments thereof) that can be used in decrease transcription include but are not limited to: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

In some cases, the fusion partner has enzymatic activity that modifies the target nucleic acid (e.g., ssRNA, dsRNA, ssDNA, dsDNA). Examples of enzymatic activity that can be provided by the fusion partner include but are not limited to: nuclease activity such as that provided by a restriction enzyme (e.g., FokI nuclease), methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like); demethylase activity such as that provided by a demethylase (e.g., Ten-Eleven Translocation (TET) dioxygenase 1 (TET1CD), TET1, DME, DML1, DML2, ROS1, and the like), DNA repair activity, DNA damage activity, deamination activity such as that provided by a deaminase (e.g., a cytosine deaminase enzyme such as rat APOBEC1), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity).

In some cases, the fusion partner has enzymatic activity that modifies a protein associated with the target nucleic acid (e.g., ssRNA, dsRNA, ssDNA, dsDNA) (e.g., a histone, an RNA binding protein, a DNA binding protein, and the like). Examples of enzymatic activity (that modifies a protein associated with a target nucleic acid) that can be provided by the fusion partner include but are not limited to: methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, and the like, SET1A, SET1B, MLL1 to 5, ASH1, SYMD2, NSD1, DOT1L, Pr-SET7/8, SUV4-20H1, EZH2, RIZ1), demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3, and the like), acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK, and the like), deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11, and the like), kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

Additional examples of a suitable fusion partners are dihydrofolate reductase (DHFR) destabilization domain (e.g., to generate a chemically controllable fusion polypeptide), and a chloroplast transit peptide. Suitable chloroplast transit peptides include, but are not limited to:

(SEQ ID NO: 81)
MASMISSSAVTTVSRASRGQSAAMAPFGGLKSMTGFPVRKVNTDITSITS
NGGRVKCMQVWPPIGKKKFETLSYLPPLTRDSRA;
(SEQ ID NO: 82)
MASMISSSAVTTVSRASRGQSAAMAPFGGLKSMTGFPVRKVNTDITSITS
NGGRVKS;
(SEQ ID NO: 83)
MASSMLSSATMVASPAQATMVAPFNGLKSSAAFPATRKANNDITSITSNG
GRVNCMQVWPPIEKKKFETLSYLPDLTDSGGRVNC;
(SEQ ID NO: 84)
MAQVSRICNGVQNPSLISNLSKSSQRKSPLSVSLKTQQHPRAYPISSSWG
LKKSGMTLIGSELRPLKVMSSVSTAC;
(SEQ ID NO: 85)
MAQVSRICNGVWNPSLISNLSKSSQRKSPLSVSLKTQQHPRAYPISSSWG
LKKSGMTLIGSELRPLKVMSSVSTAC;
(SEQ ID NO: 86)
MAQINNMAQGIQTLNPNSNFHKPQVPKSSSFLVFGSKKLKNSANSMLVLK
KDSIFMQLFCSFRISASVATAC;
(SEQ ID NO: 87)
MAALVTSQLATSGTVLSVTDRFRRPGFQGLRPRNPADAALGMRTVGASAA
PKQSRKPHRFDRRCLSMVV;
(SEQ ID NO: 88)
MAALTTSQLATSATGFGIADRSAPSSLLRHGFQGLKPRSPAGGDATSLSV
TTSARATPKQQRSVQRGSRRFPSVVVC;
(SEQ ID NO: 89)
MASSVLSSAAVATRSNVAQANMVAPFTGLKSAASFPVSRKQNLDITSIAS
NGGRVQC;
(SEQ ID NO: 90)
MESLAATSVFAPSRVAVPAARALVRAGTVVPTRRTSSTSGTSGVKCSAAV
TPQASPVISRSAAAA;
and
(SEQ ID NO: 91)
MGAAATSMQSLKFSNRLVPPSRRLSPVPNNVTCNNLPKSAAPVRTVKCCA
SSWNSTINGAAATTNGASAASS.

In some case, a fusion polypeptide of the present disclosure comprises: a) a variant CRISPR-Cas effector polypeptide of the present disclosure; and b) a chloroplast transit peptide. Thus, for example, a ribonucleoprotein (RNP) complex, comprising a variant CRISPR-Cas effector polypeptide of the present disclosure and a guide RNA, can be targeted to the chloroplast. In some cases, this targeting may be achieved by the presence of an N-terminal extension, called a chloroplast transit peptide (CTP) or plastid transit peptide. Chromosomal transgenes from bacterial sources must have a sequence encoding a CTP sequence fused to a sequence encoding an expressed polypeptide if the expressed polypeptide is to be compartmentalized in the plant plastid (e.g. chloroplast). Accordingly, localization of an exogenous polypeptide to a chloroplast is often 1 accomplished by means of operably linking a polynucleotide sequence encoding a CTP sequence to the 5′ region of a polynucleotide encoding the exogenous polypeptide. The CTP is removed in a processing step during translocation into the plastid. Processing efficiency may, however, be affected by the amino acid sequence of the CTP and nearby sequences at the amino terminus (NH₂ terminus) of the peptide. Other options for targeting to the chloroplast which have been described are the maize cab-m7 signal sequence (U.S. Pat. No. 7,022,896, WO 97/41228) a pea glutathione reductase signal sequence (WO 97/41228) and the CTP described in US2009029861.

In some cases, a fusion polypeptide of the present disclosure can comprise: a) a variant CRISPR-Cas effector polypeptide of the present disclosure; and b) an endosomal escape peptide. In some cases, an endosomal escape polypeptide comprises the amino acid sequence GLFXALLXLLXSLWXLLLXA (SEQ ID NO:92), wherein each X is independently selected from lysine, histidine, and arginine. In some cases, an endosomal escape polypeptide comprises the amino acid sequence GLFHALLHLLHSLWHLLLHA (SEQ ID NO:93).

For examples of some of the above fusion partners (and more) used in the context of fusions with Cas9, Zinc Finger, and/or TALE proteins (for site specific target nucleic modification, modulation of transcription, and/or target protein modification, e.g., histone modification), see, e.g.: Nomura et al, J Am Chem Soc. 2007 Jul. 18; 129(28):8676-7; Rivenbark et al., Epigenetics. 2012 April; 7(4):350-60; Nucleic Acids Res. 2016 Jul. 8; 44(12):5615-28; Gilbert et al., Cell. 2013 Jul. 18; 154(2):442-51; Kearns et al., Nat Methods. 2015 May; 12(5):401-3; Mendenhall et al., Nat Biotechnol. 2013 December; 31(12):1133-6; Hilton et al., Nat Biotechnol. 2015 May; 33(5):510-7; Gordley et al., Proc Natl Acad Sci USA. 2009 Mar. 31; 106(13):5053-8; Akopian et al., Proc Natl Acad Sci USA. 2003 Jul. 22; 100(15):8688-91; Tan et. al., J Virol. 2006 February; 80(4):1939-48; Tan et al., Proc Natl Acad Sci USA. 2003 Oct. 14; 100(21):11997-2002; Papworth et al., Proc Natl Acad Sci USA. 2003 Feb. 18; 100(4):1621-6; Sanjana et al., Nat Protoc. 2012 Jan. 5; 7(1):171-92; Beerli et al., Proc Natl Acad Sci USA. 1998 Dec. 8; 95(25):14628-33; Snowden et al., Curr Biol. 2002 Dec. 23; 12(24):2159-66; Xu et. al., Xu et al., Cell Discov. 2016 May 3; 2:16009; Komor et al., Nature. 2016 Apr. 20; 533(7603):420-4; Chaikind et al., Nucleic Acids Res. 2016 Aug. 11; Choudhury et. al., Oncotarget. 2016 Jun. 23; Du et al., Cold Spring Harb Protoc. 2016 Jan. 4; Pham et al., Methods Mol Biol. 2016; 1358:43-57; Balboa et al., Stem Cell Reports. 2015 Sep. 8; 5(3):448-59; Hara et al., Sci Rep. 2015 Jun. 9; 5:11221; Piatek et al., Plant Biotechnol J. 2015 May; 13(4):578-89; Hu et al., Nucleic Acids Res. 2014 April; 42(7):4375-90; Cheng et al., Cell Res. 2013 October; 23(10):1163-71; and Maeder et al., Nat Methods. 2013 October; 10(10):977-9.

Additional suitable heterologous polypeptides include, but are not limited to, a polypeptide that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). Non-limiting examples of heterologous polypeptides to accomplish increased or decreased transcription include transcription activator and transcription repressor domains. In some such cases, a fusion polypeptide of the present disclosure is targeted by the guide nucleic acid (guide RNA) to a specific location (i.e., sequence) in the target nucleic acid and exerts locus-specific regulation such as blocking RNA polymerase binding to a promoter (which selectively inhibits transcription activator function), and/or modifying the local chromatin status (e.g., when a fusion sequence is used that modifies the target nucleic acid or modifies a polypeptide associated with the target nucleic acid). In some cases, the changes are transient (e.g., transcription repression or activation). In some cases, the changes are inheritable (e.g., when epigenetic modifications are made to the target nucleic acid or to proteins associated with the target nucleic acid, e.g., nucleosomal histones).

Non-limiting examples of heterologous polypeptides for use when targeting ssRNA target nucleic acids include (but are not limited to): splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; RNA-binding proteins; and the like. It is understood that a heterologous polypeptide can include the entire protein or in some cases can include a fragment of the protein (e.g., a functional domain).

The heterologous polypeptide of a subject fusion polypeptide can be any domain capable of interacting with ssRNA (which, for the purposes of this disclosure, includes intramolecular and/or intermolecular secondary structures, e.g., double-stranded RNA duplexes such as hairpins, stem-loops, etc.), whether transiently or irreversibly, directly or indirectly, including but not limited to an effector domain selected from the group comprising; Endonucleases (for example RNase III, the CRR22 DYW domain, Dicer, and PIN (PilT N-terminus) domains from proteins such as SMG5 and SMG6); proteins and protein domains responsible for stimulating RNA cleavage (for example CPSF, CstF, CFIm and CFIIm); Exonucleases (for example XRN-1 or Exonuclease T); Deadenylases (for example HNT3); proteins and protein domains responsible for nonsense mediated RNA decay (for example UPF1, UPF2, UPF3, UPF3b, RNP S1, Y14, DEK, REF2, and SRm160); proteins and protein domains responsible for stabilizing RNA (for example PABP); proteins and protein domains responsible for repressing translation (for example Ago2 and Ago4); proteins and protein domains responsible for stimulating translation (for example Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (for example PAP1, GLD-2, and Star-PAP); proteins and protein domains responsible for polyuridinylation of RNA (for example CI D1 and terminal uridylate transferase); proteins and protein domains responsible for RNA localization (for example from IMP1, ZBP1, She2p, She3p, and Bicaudal-D); proteins and protein domains responsible for nuclear retention of RNA (for example Rrp6); proteins and protein domains responsible for nuclear export of RNA (for example TAP, NXF1, THO, TREX, REF, and Aly); proteins and protein domains responsible for repression of RNA splicing (for example PTB, Sam68, and hnRNP A1); proteins and protein domains responsible for stimulation of RNA splicing (for example Serine/Arginine-rich (SR) domains); proteins and protein domains responsible for reducing the efficiency of transcription (for example FUS (TLS)); and proteins and protein domains responsible for stimulating transcription (for example CDK7 and HIV Tat). Alternatively, the effector domain may be selected from the group comprising Endonucleases; proteins and protein domains capable of stimulating RNA cleavage; Exonucleases; Deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable heterologous polypeptide is a PUF RNA-binding domain, which is described in more detail in WO2012068627, which is hereby incorporated by reference in its entirety.

Some RNA splicing factors that can be used (in whole or as fragments thereof) as heterologous polypeptides for a fusion polypeptide of the present disclosure have modular organization, with separate sequence-specific RNA binding modules and splicing effector domains. For example, members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP Al binds to exonic splicing silencers (ESSs) through its RRM domains and inhibits exon inclusion through a C-terminal Glycine-rich domain. Some splicing factors can regulate alternative use of splice site (ss) by binding to regulatory sequences between the two alternative sites. For example, ASF/SF2 can recognize ESEs and promote the use of intron proximal sites, whereas hnRNP Al can bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple c\acute{ω}-elements that are located in either the core exon region or the exon extension region (i.e., between the two alternative 5′ splice sites). For more examples, see WO2010075303, which is hereby incorporated by reference in its entirety.

Further suitable fusion partners include, but are not limited to, proteins (or fragments thereof) that are boundary elements (e.g., CTCF), proteins and fragments thereof that provide periphery recruitment (e.g., Lamin A, Lamin B, etc.), protein docking elements (e.g., FKBP/FRB, Pil1/Aby1, etc.).

### Nucleases

In some cases, a subject fusion polypeptide comprises: i) a variant CRISPR-Cas effector polypeptide of the present disclosure; and ii) a heterologous polypeptide (a “fusion partner”), where the heterologous polypeptide is a nuclease. Suitable nucleases include, but are not limited to, a homing nuclease polypeptide; a FokI polypeptide; a transcription activator-like effector nuclease (TALEN) polypeptide; a MegaTAL polypeptide; a meganuclease polypeptide; a zinc finger nuclease (ZFN); an ARCUS nuclease; and the like. The meganuclease can be engineered from an LADLIDADG homing endonuclease (LHE). A megaTAL polypeptide can comprise a TALE DNA binding domain and an engineered meganuclease. See, e.g., WO 2004/067736 (homing endonuclease); Urnov et al. (2005) Nature 435:646 (ZFN); Mussolino et al. (2011) Nucle. Acids Res. 39:9283 (TALE nuclease); Boissel et al. (2013) Nucl. Acids Res. 42:2591 (MegaTAL).

### Reverse Transcriptases

In some cases, a subject fusion polypeptide comprises: i) a variant CRISPR-Cas effector polypeptide of the present disclosure; and ii) a heterologous polypeptide (a “fusion partner”), where the heterologous polypeptide is a reverse transcriptase polypeptide. Suitable reverse transcriptases include, e.g., a murine leukemia virus reverse transcriptase; a Rous sarcoma virus reverse transcriptase; a human immunodeficiency virus type I reverse transcriptase; a Moloney murine leukemia virus reverse transcriptase; and the like.

### Base Editors

In some cases, a fusion polypeptide of the present disclosure comprises: i) a variant CRISPR-Cas effector polypeptide of the present disclosure; and ii) a heterologous polypeptide (a “fusion partner”), where the heterologous polypeptide is a base editor. Suitable base editors include, e.g., an adenosine deaminase; a cytidine deaminase (e.g., an activation-induced cytidine deaminase (AID)); APOBEC3G; and the like); and the like.

A suitable adenosine deaminase is any enzyme that is capable of deaminating adenosine in DNA. In some cases, the deaminase is a TadA deaminase.

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 94)
MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQKKAQSSTD.

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 95)
MRRAFITGVFFLSEVEFSHEYWMRHALTLAKRAWDEREVPVGAVLVHNNR
VIGEGWNRPIGRHDPTAHAEIMALRQGGLVMQNYRLIDATLYVTLEPCVM
CAGAMIHSRIGRVVFGARDAKTGAAGSLMDVLHHPGMNHRVEITEGILAD
ECAALLSDFFRMRRQEIKAQKKAQSSTD.

In some cases, a suitable adenosine deaminase comprises an amino acid sequence having at least 80%, at least 85%, at least 90%, at least 95%, at least 98%, at least 99%, or 100%, amino acid sequence identity to the following Staphylococcus aureus TadA amino acid sequence:

(SEQ ID NO: 96)
MGSHMTNDIYFMTLAIEEAKKAAQLGEVPIGAIITKDDEVIARAHNLRE
TLQQPTAHAEHIAIERAAKVLGSWRLEGCTLYVTLEPCVMCAGTIVMSR
IPRVVYGADDPKGGCSGSLMNLLQQSNFNHRAIVDKGVLKEACSTLLTT
FFK NLRANKKSTN:

In some cases, a suitable adenosine deaminase comprises an amino acid sequence having at least 80%, at least 85%, at least 90%, at least 95%, at least 98%, at least 99%, or 100%, amino acid sequence identity to the following Bacillus subtilis TadA amino acid sequence:

(SEQ ID NO: 97)
MTQDELYMKEAIKEAKKAEEKGEVPIGAVLVINGEIIARAHNLRETEQRS
IAHAEMLVIDEACKALGTWRLEGATLYVTLEPCPMCAGAVVLSRVEKVVF
GAFDPKGGCSGTLMNLLQEERFNHQAEVVSGVLEEECGGMLSAFFRELRK
KKKAARKNLSE

In some cases, a suitable adenosine deaminase comprises an amino acid sequence having at least 80%, at least 85%, at least 90%, at least 95%, at least 98%, at least 99%, or 100%, amino acid sequence identity to the following Salmonella typhimurium TadA:

(SEQ ID NO: 98)
MPPAFITGVTSLSDVELDHEYWMRHALTLAKRAWDEREVPVGAVLVHNHR
VIGEGWNRPIGRHDPTAHAEIMALRQGGLVLQNYRLLDTTLYVTLEPCVM
CAGAMVHSRIGRVVFGARDAKTGAAGSLIDVLHHPGMNHRVEIIEGVLRD
ECATLLSDFFRMRRQEIKALKKADRAEGAGPAV

In some cases, a suitable adenosine deaminase comprises an amino acid sequence having at least 80%, at least 85%, at least 90%, at least 95%, at least 98%, at least 99%, or 100%, amino acid sequence identity to the following Shewanella putrefaciens TadA amino acid sequence:

(SEQ ID NO: 99)
MDEYWMQVAMQMAEKAEAAGEVPVGAVLVKDGQQIATGYNLSISQHDPTA
HAEILCLRSAGKKLENYRLLDATLYITLEPCAMCAGAMVHSRIARVVYGA
RDEKTGAAGTVVNLLQHPAFNHQVEVTSGVLAEACSAQLSRFFKRRRDEK
KALKLAQRAQQGIE

In some cases, a suitable adenosine deaminase comprises an amino acid sequence having at least 80%, at least 85%, at least 90%, at least 95%, at least 98%, at least 99%, or 100%, amino acid sequence identity to the following Haemophilus influenzae F3031 TadA amino acid sequence:

(SEQ ID NO: 100)
MDAAKVRSEFDEKMMRYALELADKAEALGEIPVGAVLVDDARNIIGEGW
NLSIVQSDPTAHAEIIALRNGAKNIQNYRLLNSTLYVTLEPCTMCAGAI
LHSRIKRLVFGASDYKTGAIGSRFHFFDDYKMNHTLEITSGVLAEECSQ
KLS TFFQKRREEKKIEKALLKSLSDK

In some cases, a suitable adenosine deaminase comprises an amino acid sequence having at least 80%, at least 85%, at least 90%, at least 95%, at least 98%, at least 99%, or 100%, amino acid sequence identity to the following Caulobacter crescentus TadA amino acid sequence:

(SEQ ID NO: 101)
MRTDESEDQDHRMMRLALDAARAAAEAGETPVGAVILDPSTGEVIATAGN
GPIAAHDPTAHAEIAAMRAAAAKLGNYRLTDLTLVVTLEPCAMCAGAISH
ARIGRVVFGADDPKGGAVVHGPKFFAQPTCHWRPEVTGGVLADESADLLR
GFFRARRKAKI

In some cases, a suitable adenosine deaminase comprises an amino acid sequence having at least 80%, at least 85%, at least 90%, at least 95%, at least 98%, at least 99%, or 100%, amino acid sequence identity to the following Geobacter sulfurreducens TadA amino acid sequence:

(SEQ ID NO: 102)
MSSLKKTPIRDDAYWMGKAIREAAKAAARDEVPIGAVIVRDGAVIGRGHN
LREGSNDPSAHAEMIAIRQAARRSANWRLTGATLYVTLEPCLMCMGAIIL
ARLERVVFGCYDPKGGAAGSLYDLSADPRLNHQVRLSPGVCQEECGTMLS
DFFRDLRRRKKAKATPALFIDERKVPPEP

Cytidine deaminases suitable for inclusion in a CRISPR/Cas effector polypeptide fusion polypeptide include any enzyme that is capable of deaminating cytidine in DNA.

In some cases, the cytidine deaminase is a deaminase from the apolipoprotein B mRNA-editing complex (APOBEC) family of deaminases. In some cases, the APOBEC family deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase. In some cases, the cytidine deaminase is an activation induced deaminase (AID).

In some cases, a suitable cytidine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 103)
MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKDYFYCWNT
FVENHERTFKAWEGLHENSVRLSRQLRRILLPLYEVDDLRDAFRTLGL

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK FLYQFKNVRW AKGRRETYLC YVVKRRDSAT SFSLDFGYLR NKNGCHVELL FLRYISDWDL DPGRCYRVTW FTSWSPCYDC ARHVADFLRG NPNLSLRIFT ARLYFCEDRK AEPEGLRRLH RAGVQIAIMT FKENHERTFK AWEGLHENSV RLSRQLRRIL LPLYEVDDLR DAFRTLGL (SEQ ID NO:104).

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK FLYQFKNVRW AKGRRETYLC YVVKRRDSAT SFSLDFGYLR NKNGCHVELL FLRYISDWDL DPGRCYRVTW FTSWSPCYDC ARHVADFLRG NPNLSLRIFT ARLYFCEDRK AEPEGLRRLH RAGVQIAIMT FKDYFYCWNT FVENHERTFK AWEGLHENSV RLSRQLRRIL LPLYEVDDLR DAFRTLGL (SEQ ID NO:103).

### Transcription Factors

In some cases, a fusion polypeptide of the present disclosure comprises: i) a variant CRISPR-Cas effector polypeptide of the present disclosure; and ii) a heterologous polypeptide (a “fusion partner”), where the heterologous polypeptide is a transcription factor. A transcription factor can include: i) a DNA binding domain; and ii) a transcription activator. A transcription factor can include: i) a DNA binding domain; and ii) a transcription repressor. Suitable transcription factors include polypeptides that include a transcription activator or a transcription repressor domain (e.g., the Kruppel associated box (KRAB or SKD); the Mad mSIN3 interaction domain (SID); the ERF repressor domain (ERD), etc.); zinc-finger-based artificial transcription factors (see, e.g., Sera (2009) Adv. Drug Deliv. 61:513); TALE-based artificial transcription factors (see, e.g., Liu et al. (2013) Nat. Rev. Genetics 14:781); and the like. In some cases, the transcription factor comprises a VP64 polypeptide (transcriptional activation). In some cases, the transcription factor comprises a Krüppel-associated box (KRAB) polypeptide (transcriptional repression). In some cases, the transcription factor comprises a Mad mSIN3 interaction domain (SID) polypeptide (transcriptional repression). In some cases, the transcription factor comprises an ERF repressor domain (ERD) polypeptide (transcriptional repression). For example, in some cases, the transcription factor is a transcriptional activator, where the transcriptional activator is GAL4-VP16.

### Recombinases

In some cases, a fusion polypeptide of the present disclosure comprises: i) a variant CRISPR-Cas effector polypeptide of the present disclosure; and ii) a heterologous polypeptide (a “fusion partner”), where the heterologous polypeptide is a recombinase. Suitable recombinases include, e.g., a Cre recombinase; a Hin recombinase; a Tre recombinase; a FLP recombinase; and the like.

Examples of various additional suitable heterologous polypeptide (or fragments thereof) for a subject fusion polypeptide include, but are not limited to, those described in the following applications (which publications are related to other CRISPR endonucleases such as Cas9, but the described fusion partners can also be used with a variant CRISPR-Cas effector polypeptide of the present disclosure instead): PCT patent applications: WO2010075303, WO2012068627, and WO2013155555, and can be found, for example, in U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

In some cases, a heterologous polypeptide (a fusion partner) provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some cases, a CasPhi fusion polypeptide does not include an NLS so that the protein is not targeted to the nucleus (which can be advantageous, e.g., when the target nucleic acid is an RNA that is present in the cytosol). In some cases, the heterologous polypeptide can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like).

In some cases, a fusion polypeptide of the present disclosure comprises: a) variant CRISPR-Cas effector of the present disclosure; and b) one or more nuclear localization signals (NLSs) (e.g., in some cases 2 or more, 3 or more, 4 or more, or 5 or more NLSs). Thus, in some cases, a fusion polypeptide of the present disclosure includes one or more NLSs (e.g., 2 or more, 3 or more, 4 or more, or 5 or more NLSs). In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus.

In some cases, a fusion polypeptide of the present disclosure comprises: a) a variant CRISPR-Cas effector polypeptide of the present disclosure; and b) between 1 and 10 NLSs (e.g., 1-9, 1-8, 1-7, 1-6, 1-5, 2-10, 2-9, 2-8, 2-7, 2-6, or 2-5 NLSs). In some cases, a fusion polypeptide of the present disclosure comprises: a) a variant CRISPR-Cas effector polypeptide of the present disclosure; and b) between 2 and 5 NLSs (e.g., 2-4, or 2-3 NLSs).

Non-limiting examples of NLSs include an NLS sequence derived from: the NLS of the SV40 virus large T-antigen, having the amino acid sequence PKKKRKV (SEQ ID NO:105); the NLS from nucleoplasmin (e.g., the nucleoplasmin bipartite NLS with the sequence KRPAATKKAGQAKKKK (SEQ ID NO:106)); the c-myc NLS having the amino acid sequence PAAKRVKLD (SEQ ID NO:107) or RQRRNELKRSP (SEQ ID NO:108); the hRNPA1 M9 NLS having the sequence NQSSNFGPMKGGNFGGRSSGPYGGGGQYFAKPRNQGGY (SEQ ID NO:109); the sequence RMRIZFKNKGKDTAELRRRRVEVSVELRKAKKDEQILKRRNV (SEQ ID NO:110) of the IBB domain from importin-alpha; the sequences VSRKRPRP (SEQ ID NO:111) and PPKKARED (SEQ ID NO:112) of the myoma T protein; the sequence PQPKKKPL (SEQ ID NO:113) of human p53; the sequence SALIKKKKKMAP (SEQ ID NO:114) of mouse c-abl IV; the sequences DRLRR (SEQ ID NO:115) and PKQKKRK (SEQ ID NO:116) of the influenza virus NS1; the sequence RKLKKKIKKL (SEQ ID NO:117) of the Hepatitis virus delta antigen; the sequence REKKKFLKRR (SEQ ID NO:118) of the mouse Mx1 protein; the sequence KRKGDEVDGVDEVAKKKSKK (SEQ ID NO:119) of the human poly(ADP-ribose) polymerase; and the sequence RKCLQAGMNLEARKTKK (SEQ ID NO:120) of the steroid hormone receptors (human) glucocorticoid. In general, NLS (or multiple NLSs) are of sufficient strength to drive accumulation of the variant CRISPR-Cas effector polypeptide in a detectable amount in the nucleus of a eukaryotic cell. Detection of accumulation in the nucleus may be performed by any suitable technique. For example, a detectable marker may be fused to the variant CRISPR-Cas effector polypeptide such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

In some cases, a variant CRISPR-Cas effector polypeptide of the present disclosure includes a “Protein Transduction Domain” or PTD (also known as a CPP—cell penetrating peptide), which refers to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the amino terminus a polypeptide (e.g., linked to a wild type CasPhi to generate a fusion protein, or linked to a variant CRISPR-Cas effector polypeptide of the present disclosure. In some embodiments, a PTD is covalently linked to the carboxyl terminus of a variant CRISPR-Cas effector polypeptide of the present disclosure. In some cases, the PTD is inserted internally in the variant CRISPR-Cas effector polypeptide (i.e., is not at the N- or C-terminus of the variant CRISPR-Cas effector polypeptide) at a suitable insertion site. In some cases, a subject fusion polypeptide includes: a) a variant CRISPR-Cas effector polypeptide of the present disclosure; and b) one or more PTDs (e.g., two or more, three or more, four or more PTDs). In some cases, a PTD includes a nuclear localization signal (NLS) (e.g., in some cases 2 or more, 3 or more, 4 or more, or 5 or more NLSs). Thus, in some cases, a fusion polypeptide of the present disclosure includes one or more NLSs (e.g., 2 or more, 3 or more, 4 or more, or 5 or more NLSs). In some cases, a PTD is covalently linked to a nucleic acid (e.g., a CasPhi guide nucleic acid, a polynucleotide encoding a CasPhi guide nucleic acid, a polynucleotide encoding a fusion polypeptide, a donor polynucleotide, etc.). Examples of PTDs include but are not limited to a minimal undecapeptide protein transduction domain (corresponding to residues 47-57 of HIV-1 TAT comprising YGRKKRRQRRR; SEQ ID NO:121); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:122); Transportan GWTLNSAGYLLGKINLKALAALAKKIL (SEQ ID NO:123); KALAWEAKLAKALAKALAKHLAKALAKALKCEA (SEQ ID NO:124); and RQIKIWFQNRRMKWKK (SEQ ID NO:125). Exemplary PTDs include but are not limited to, YGRKKRRQRRR (SEQ ID NO:121), RKKRRQRRR (SEQ ID NO:126); an arginine homopolymer of from 3 arginine residues to 50 arginine residues; Exemplary PTD domain amino acid sequences include, but are not limited to, any of the following: YGRKKRRQRRR (SEQ ID NO:121); RKKRRQRR (SEQ ID NO:127); YARAAARQARA (SEQ ID NO:128); THRLPRRRRRR (SEQ ID NO:129); and GGRRARRRRRR (SEQ ID NO:130). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

### Linkers (e.g., for Fusion Partners)

In some embodiments, a variant CRISPR-Cas effector polypeptide of the present disclosure can fused to a fusion partner via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

Examples of linker polypeptides include glycine polymers (G)ₙ where n is an integer of at least one; glycine-serine polymers (including, for example, (GS)ₙ, (GSGGS)ₙ (SEQ ID NO:131), (GGSGGS)ₙ (SEQ ID NO:132), (GGGGS)n (SEQ ID NO:133), and (GGGS)ₙ (SEQ ID NO:134), where n is an integer of at least one; e.g., where n is 1, 2, 3, 4, 5, 6, 7, 8, 9, or 10); glycine-alanine polymers; and alanine-serine polymers. Exemplary linkers can comprise amino acid sequences including, but not limited to, GGSG (SEQ ID NO:135), GGSGG (SEQ ID NO:136), GSGSG (SEQ ID NO:137), GSGGG (SEQ ID NO:138), GGGSG (SEQ ID NO:139), GSSSG (SEQ ID NO:140), GGGGS (SEQ ID NO:141), and the like. The ordinarily skilled artisan will recognize that design of a peptide conjugated to any desired element can include linkers that are all or partially flexible, such that the linker can include a flexible linker as well as one or more portions that confer less flexible structure.

### Detectable Labels

In some cases, a variant CRISPR-Cas effector polypeptide of the present disclosure, or a fusion polypeptide of the present disclosure, comprises a detectable label. Suitable detectable labels and/or moieties that can provide a detectable signal can include, but are not limited to, an enzyme, a radioisotope, a member of a specific binding pair; a fluorophore; a fluorescent protein; a quantum dot; and the like.

Suitable fluorescent proteins include, but are not limited to, green fluorescent protein (GFP) or variants thereof, a blue fluorescent protein (BFP), a cyan fluorescent (CFP), a yellow fluorescent protein (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

### Protospacer Adjacent Motif (PAM)

A variant CRISPR-Cas effector of the present disclosure binds to target DNA at a target sequence defined by the region of complementarity between the DNA-targeting RNA and the target DNA. As is the case for many CRISPR endonucleases, site-specific binding (and/or cleavage) of a double stranded target DNA occurs at locations determined by both (i) base-pairing complementarity between the guide RNA and the target DNA; and (ii) a short motif [referred to as the protospacer adjacent motif (PAM)] in the target DNA.

In some cases, the PAM for a CasPhi protein is immediately 5′ of the target sequence of the non-complementary strand of the target DNA (the complementary strand: (i) hybridizes to the guide sequence of the guide RNA, while the non-complementary strand does not directly hybridize with the guide RNA; and (ii) is the reverse complement of the non-complementary strand).

In some cases, the PAM sequence of the non-complementary strand is 5′-VTTR-3′ (where V is G, A, or C and R is A or G). Thus, in some cases, suitable PAMs can include GTTA, GTTG, ATTA, ATTG, CTTA, and CTTG.

In some cases, the PAM sequence of the non-complementary strand is 5′-TBN-3′ (where B is T, C, or G). Thus, in some cases, suitable PAMs can include TTA, TTC, TTT, TTG, TCA, TCC, TCT, TCG, TGA, TGC, TGT, and TGG. In some cases, the PAM sequence of the non-complementary strand is 5′-TNN-3′.

In some cases, the PAM sequence of the non-complementary strand is 5′-VTTB-3′ (where V is G, A, or C and where B is T, C, or G). Thus, in some cases, suitable PAMs can include GTTT, GTTC, GTTG, ATTT, ATTC, ATTG, CTTT, CTTC, CTTG. In some cases, the PAM sequence of the non-complementary strand is 5′-NTTN-3′. In some cases, the PAM sequence of the non-complementary strand is 5′-VTTN-3′ (where V is G, A, or C). In some cases, the PAM sequence of the non-complementary strand is 5′-VTTC-3′.

For a particular variant CRISPR-Cas effector polypeptide of choice, the PAM sequence preference may be different than the sequences described above. Various methods (including in silico and/or wet lab methods) for identification of the appropriate PAM sequence are known in the art and are routine, and any convenient method can be used. For example, PAM sequences described herein were identified using a PAM depletion assay (e.g., see working examples below), but could also have been identified using a variety of different methods (including computational analysis of sequencing data—as known in the art).

### CasPhi Guide RNA

A nucleic acid that binds to a variant CRISPR-Cas effector polypeptide of the present disclosure, forming a ribonucleoprotein complex (RNP), and targets the complex to a specific location within a target nucleic acid (e.g., a target DNA) is referred to herein as a “CasPhi guide RNA” or simply as a “guide RNA.” It is to be understood that in some cases, a hybrid DNA/RNA can be made such that a CasPhi guide RNA includes DNA bases in addition to RNA bases, but the term “CasPhi guide RNA” is still used to encompass such a molecule herein.

A CasPhi guide RNA can be said to include two segments, a targeting segment and a protein-binding segment. The protein-binding segment is also referred to herein as the “constant region” of the guide RNA. The targeting segment of a CasPhi guide RNA includes a nucleotide sequence (a guide sequence) that is complementary to (and therefore hybridizes with) a specific sequence (a target site) within a target nucleic acid (e.g., a target dsDNA, a target ssRNA, a target ssDNA, the complementary strand of a double stranded target DNA, etc.). The protein-binding segment (or “protein-binding sequence”) interacts with (binds to) a variant CRISPR-Cas effector polypeptide of the present disclosure. The protein-binding segment of a subject CasPhi guide RNA can include two complementary stretches of nucleotides that hybridize to one another to form a double stranded RNA duplex (dsRNA duplex). Site-specific binding and/or cleavage of a target nucleic acid (e.g., genomic DNA, ds DNA, RNA, etc.) can occur at locations (e.g., target sequence of a target locus) determined by base-pairing complementarity between the CasPhi guide RNA (the guide sequence of the CasPhi guide RNA) and the target nucleic acid. In many cases, the targeting segment is heterologous to the protein-binding segment; i.e., the targeting segment comprises a nucleotide sequence that is not found in nature in the same guide RNA as the protein-binding segment.

A CasPhi guide RNA and a variant CRISPR-Cas effector polypeptide of the present disclosure form a complex (e.g., bind via non-covalent interactions). The CasPhi guide RNA provides target specificity to the complex by including a targeting segment, which includes a guide sequence (a nucleotide sequence that is complementary to a sequence of a target nucleic acid). The variant CRISPR-Cas effector polypeptide of the complex provides the site-specific activity (e.g., cleavage activity provided by the variant CRISPR-Cas effector polypeptide and/or an activity provided by the fusion partner in the case of a fusion protein). In other words, the variant CRISPR-Cas effector polypeptide is guided to a target nucleic acid sequence (e.g. a target sequence) by virtue of its association with the CasPhi guide RNA.

The “guide sequence” also referred to as the “targeting sequence” of a CasPhi guide RNA can be modified so that the CasPhi guide RNA can target a variant CRISPR-Cas effector polypeptide of the present disclosure, or a fusion polypeptide of the present disclosure, to any desired sequence of any desired target nucleic acid, with the exception (e.g., as described herein) that the PAM sequence can be taken into account. Thus, for example, a CasPhi guide RNA can have a guide sequence with complementarity to (e.g., can hybridize to) a sequence in a nucleic acid in a eukaryotic cell, e.g., a viral nucleic acid, a eukaryotic nucleic acid (e.g., a eukaryotic chromosome, chromosomal sequence, a eukaryotic RNA, etc.), and the like.

### Guide Sequence of a CasPhi Guide RNA

A subject CasPhi guide RNA includes a guide sequence (i.e., a targeting sequence), which is a nucleotide sequence that is complementary to a sequence (a target site) in a target nucleic acid. In other words, the guide sequence of a CasPhi guide RNA can interact with a target nucleic acid (e.g., double stranded DNA (dsDNA), single stranded DNA (ssDNA), single stranded RNA (ssRNA), or double stranded RNA (dsRNA)) in a sequence-specific manner via hybridization (i.e., base pairing). The guide sequence of a CasPhi guide RNA can be modified (e.g., by genetic engineering)/designed to hybridize to any desired target sequence (e.g., while taking the PAM into account, e.g., when targeting a dsDNA target) within a target nucleic acid (e.g., a eukaryotic target nucleic acid such as genomic DNA).

In some cases, the percent complementarity between the guide sequence and the target site of the target nucleic acid is 60% or more (e.g., 65% or more, 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid is 80% or more (e.g., 85% or more, 90% or more, 95% or more, 97% or more, 98% or more, 99% or more, or 100%). In some cases, the percent complementarity between the guide sequence and the target site of the target nucleic acid is 90% or more (e.g., 95% or more, 97% or more, 98% or more, 99% or more, or 100%). In some cases, the percent complementarity between the guide sequence and the target site of the target nucleic acid is 100%.

In some cases, the percent complementarity between the guide sequence and the target site of the target nucleic acid is 100% over the seven contiguous 3′-most nucleotides of the target site of the target nucleic acid.

In some cases, the percent complementarity between the guide sequence and the target site of the target nucleic acid is 60% or more (e.g., 70% or more, 75% or more, 80% or more, 85% or more, 90% or more, 95% or more, 97% or more, 98% or more, 99% or more, or 100%) over 17 or more (e.g., 18 or more, 19 or more, 20 or more, 21 or more, 22 or more) contiguous nucleotides. In some cases, the percent complementarity between the guide sequence and the target site of the target nucleic acid is 80% or more (e.g., 85% or more, 90% or more, 95% or more, 97% or more, 98% or more, 99% or more, or 100%) over 17 or more (e.g., 18 or more, 19 or more, 20 or more, 21 or more, 22 or more) contiguous nucleotides. In some cases, the percent complementarity between the guide sequence and the target site of the target nucleic acid is 90% or more (e.g., 95% or more, 97% or more, 98% or more, 99% or more, or 100%) over 17 or more (e.g., 18 or more, 19 or more, 20 or more, 21 or more, 22 or more) contiguous nucleotides. In some cases, the percent complementarity between the guide sequence and the target site of the target nucleic acid is 100% over 17 or more (e.g., 18 or more, 19 or more, 20 or more, 21 or more, 22 or more) contiguous nucleotides.

In some cases, the percent complementarity between the guide sequence and the target site of the target nucleic acid is 60% or more (e.g., 70% or more, 75% or more, 80% or more, 85% or more, 90% or more, 95% or more, 97% or more, 98% or more, 99% or more, or 100%) over 19 or more (e.g., 20 or more, 21 or more, 22 or more) contiguous nucleotides. In some cases, the percent complementarity between the guide sequence and the target site of the target nucleic acid is 80% or more (e.g., 85% or more, 90% or more, 95% or more, 97% or more, 98% or more, 99% or more, or 100%) over 19 or more (e.g., 20 or more, 21 or more, 22 or more) contiguous nucleotides. In some cases, the percent complementarity between the guide sequence and the target site of the target nucleic acid is 90% or more (e.g., 95% or more, 97% or more, 98% or more, 99% or more, or 100%) over 19 or more (e.g., 20 or more, 21 or more, 22 or more) contiguous nucleotides. In some cases, the percent complementarity between the guide sequence and the target site of the target nucleic acid is 100% over 19 or more (e.g., 20 or more, 21 or more, 22 or more) contiguous nucleotides.

In some cases, the percent complementarity between the guide sequence and the target site of the target nucleic acid is 60% or more (e.g., 70% or more, 75% or more, 80% or more, 85% or more, 90% or more, 95% or more, 97% or more, 98% or more, 99% or more, or 100%) over 17-25 contiguous nucleotides. In some cases, the percent complementarity between the guide sequence and the target site of the target nucleic acid is 80% or more (e.g., 85% or more, 90% or more, 95% or more, 97% or more, 98% or more, 99% or more, or 100%) over 17-25 contiguous nucleotides. In some cases, the percent complementarity between the guide sequence and the target site of the target nucleic acid is 90% or more (e.g., 95% or more, 97% or more, 98% or more, 99% or more, or 100%) over 17-25 contiguous nucleotides. In some cases, the percent complementarity between the guide sequence and the target site of the target nucleic acid is 100% over 17-25 contiguous nucleotides.

In some cases, the percent complementarity between the guide sequence and the target site of the target nucleic acid is 60% or more (e.g., 70% or more, 75% or more, 80% or more, 85% or more, 90% or more, 95% or more, 97% or more, 98% or more, 99% or more, or 100%) over 19-25 contiguous nucleotides. In some cases, the percent complementarity between the guide sequence and the target site of the target nucleic acid is 80% or more (e.g., 85% or more, 90% or more, 95% or more, 97% or more, 98% or more, 99% or more, or 100%) over 19-25 contiguous nucleotides. In some cases, the percent complementarity between the guide sequence and the target site of the target nucleic acid is 90% or more (e.g., 95% or more, 97% or more, 98% or more, 99% or more, or 100%) over 19-25 contiguous nucleotides. In some cases, the percent complementarity between the guide sequence and the target site of the target nucleic acid is 100% over 19-25 contiguous nucleotides.

In some cases, the guide sequence has a length in a range of from 17-30 nucleotides (nt) (e.g., from 17-25, 17-22, 17-20, 19-30, 19-25, 19-22, 19-20, 20-30, 20-25, or 20-22 nt). In some cases, the guide sequence has a length in a range of from 17-25 nucleotides (nt) (e.g., from 17-22, 17-20, 19-25, 19-22, 19-20, 20-25, or 20-22 nt). In some cases, the guide sequence has a length of 17 or more nt (e.g., 18 or more, 19 or more, 20 or more, 21 or more, or 22 or more nt; 19 nt, 20 nt, 21 nt, 22 nt, 23 nt, 24 nt, 25 nt, etc.). In some cases, the guide sequence has a length of 19 or more nt (e.g., 20 or more, 21 or more, or 22 or more nt; 19 nt, 20 nt, 21 nt, 22 nt, 23 nt, 24 nt, 25 nt, etc.). In some cases, the guide sequence has a length of 17 nt. In some cases, the guide sequence has a length of 18 nt. In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt.

In some cases, the guide sequence (also referred to as a “spacer sequence”) has a length of from 15 to 50 nucleotides (e.g., from 15 nucleotides (nt) to 20 nt, from 20 nt to 25 nt, from 25 nt to 30 nt, from 30 nt to 35 nt, from 35 nt to 40 nt, from 40 nt to 45 nt, or from 45 nt to 50 nt).

### Protein-Binding Segment of a CasPhi Guide RNA

The protein-binding segment (the “constant region”) of a subject CasPhi guide RNA interacts with a variant CRISPR-Cas effector polypeptide of the present disclosure. The CasPhi guide RNA guides the bound variant CRISPR-Cas effector polypeptide to a specific nucleotide sequence within target nucleic acid via the above-mentioned guide sequence. The protein-binding segment of a CasPhi guide RNA can include two stretches of nucleotides that are complementary to one another and hybridize to form a double stranded RNA duplex (dsRNA duplex). Thus, in some cases, the protein-binding segment includes a dsRNA duplex.

In some cases, the dsRNA duplex region includes a range of from 5-25 base pairs (bp) (e.g., from 5-22, 5-20, 5-18, 5-15, 5-12, 5-10, 5-8, 8-25, 8-22, 8-18, 8-15, 8-12, 12-25, 12-22, 12-18, 12-15, 13-25, 13-22, 13-18, 13-15, 14-25, 14-22, 14-18, 14-15, 15-25, 15-22, 15-18, 17-25, 17-22, or 17-18 bp, e.g., 5 bp, 6 bp, 7 bp, 8 bp, 9 bp, 10 bp, etc.). In some cases, the dsRNA duplex region includes a range of from 6-15 base pairs (bp) (e.g., from 6-12, 6-10, or 6-8 bp, e.g., 6 bp, 7 bp, 8 bp, 9 bp, 10 bp, etc.). In some cases, the duplex region includes 5 or more bp (e.g., 6 or more, 7 or more, or 8 or more bp). In some cases, the duplex region includes 6 or more bp (e.g., 7 or more, or 8 or more bp). In some cases, not all nucleotides of the duplex region are paired, and therefore the duplex forming region can include a bulge. The term “bulge” herein is used to mean a stretch of nucleotides (which can be one nucleotide) that do not contribute to a double stranded duplex, but which are surround 5′ and 3′ by nucleotides that do contribute, and as such a bulge is considered part of the duplex region. In some cases, the dsRNA includes 1 or more bulges (e.g., 2 or more, 3 or more, 4 or more bulges). In some cases, the dsRNA duplex includes 2 or more bulges (e.g., 3 or more, 4 or more bulges). In some cases, the dsRNA duplex includes 1-5 bulges (e.g., 1-4, 1-3, 2-5, 2-4, or 2-3 bulges).

Thus, in some cases, the stretches of nucleotides that hybridize to one another to form the dsRNA duplex have 70%-100% complementarity (e.g., 75%-100%, 80%-10%, 85%-100%, 90%-100%, 95%-100% complementarity) with one another. In some cases, the stretches of nucleotides that hybridize to one another to form the dsRNA duplex have 70%-100% complementarity (e.g., 75%-100%, 80%-10%, 85%-100%, 90%-100%, 95%-100% complementarity) with one another. In some cases, the stretches of nucleotides that hybridize to one another to form the dsRNA duplex have 85%-100% complementarity (e.g., 90%-100%, 95%-100% complementarity) with one another. In some cases, the stretches of nucleotides that hybridize to one another to form the dsRNA duplex have 70%-95% complementarity (e.g., 75%-95%, 80%-95%, 85%-95%, 90%-95% complementarity) with one another.

In other words, in some cases, the dsRNA duplex includes two stretches of nucleotides that have 70%-100% complementarity (e.g., 75%-100%, 80%-10%, 85%-100%, 90%-100%, 95%-100% complementarity) with one another. In some cases, the dsRNA duplex includes two stretches of nucleotides that have 85%-100% complementarity (e.g., 90%-100%, 95%-100% complementarity) with one another. In some cases, the dsRNA duplex includes two stretches of nucleotides that have 70%-95% complementarity (e.g., 75%-95%, 80%-95%, 85%-95%, 90%-95% complementarity) with one another.

The duplex region of a subject CasPhi guide RNA can include one or more (1, 2, 3, 4, 5, etc) mutations relative to a naturally occurring duplex region. For example, in some cases a base pair can be maintained while the nucleotides contributing to the base pair from each segment can be different. In some cases, the duplex region of a subject CasPhi guide RNA includes more paired bases, less paired bases, a smaller bulge, a larger bulge, fewer bulges, more bulges, or any convenient combination thereof, as compared to a naturally occurring duplex region (of a naturally occurring CasPhi guide RNA).

Examples of various Cas9 guide RNAs can be found in the art, and in some cases variations similar to those introduced into Cas9 guide RNAs can also be introduced into CasPhi guide RNAs of the present disclosure (e.g., mutations to the dsRNA duplex region, extension of the 5′ or 3′ end for added stability for to provide for interaction with another protein, and the like). For example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et. al, Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et. at., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

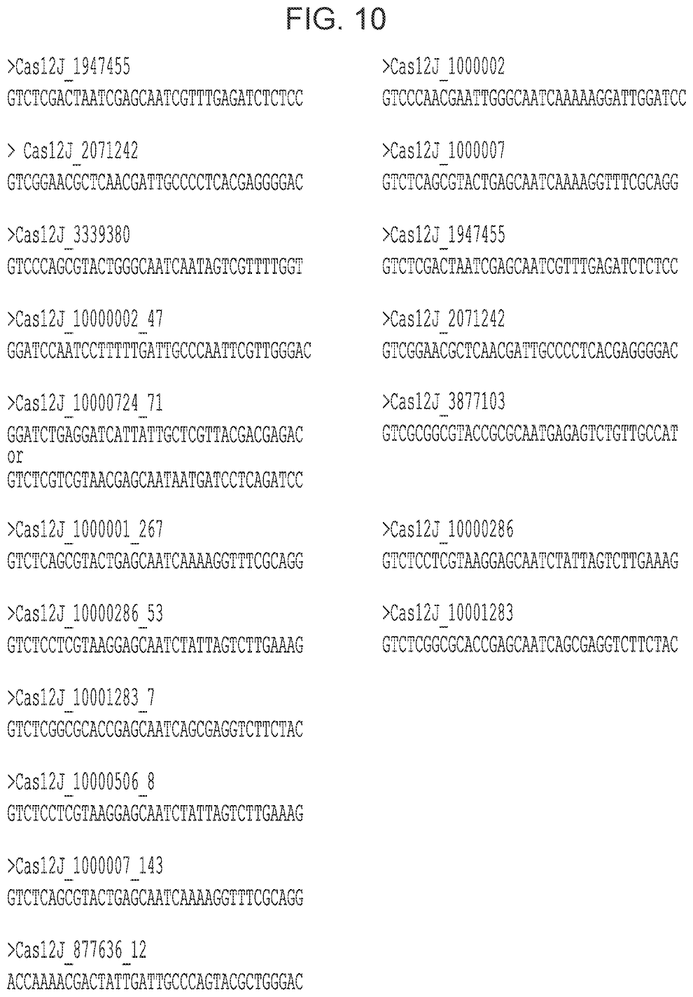

Examples of constant regions suitable for inclusion in a CasPhi guide RNA are provided in FIG. 10 (e.g., where T is substituted with U). A CasPhi guide RNA can include a constant region having from 1 to 5 nucleotide substitutions compared to any one of the nucleotide sequences depicted in FIG. 10. As one example, the constant region of a CasPhi guide RNA can comprise the reverse complement of the nucleotide sequence: GUCUCGACUAAUCGAGCAAUCGUUUGAGAUCUCUCC (SEQ ID NO:37). As another example, the constant region of a CasPhi guide RNA can comprise the nucleotide sequence: GUCGGAACGCUCAACGAUUGCCCCUCACGAGGGGAC (SEQ ID NO:142). As another example, the constant region of a CasPhi guide RNA can comprise the reverse complement of the nucleotide sequence: GUCCCAGCGUACUGGGCAAUCAAUAGTCGUUUUGGU (SEQ ID NO:143). As another example, the constant region of a CasPhi guide RNA can comprise the nucleotide sequence: CACAGGAGAGAUCUCAAACGAUUGCUCGAUUAGUCGAGAC (SEQ ID NO:144). As another example, the constant region of a CasPhi guide RNA can comprise the nucleotide sequence: UAAUGUCGGAACGCUCAACGAUUGCCCCUCACGAGGGGAC (SEQ ID NO:145). As another example, the constant region of a CasPhi guide RNA can comprise the nucleotide sequence: AUUAACCAAAACGACUAUUGAUUGCCCAGUACGCUGGGAC (SEQ ID NO:146).

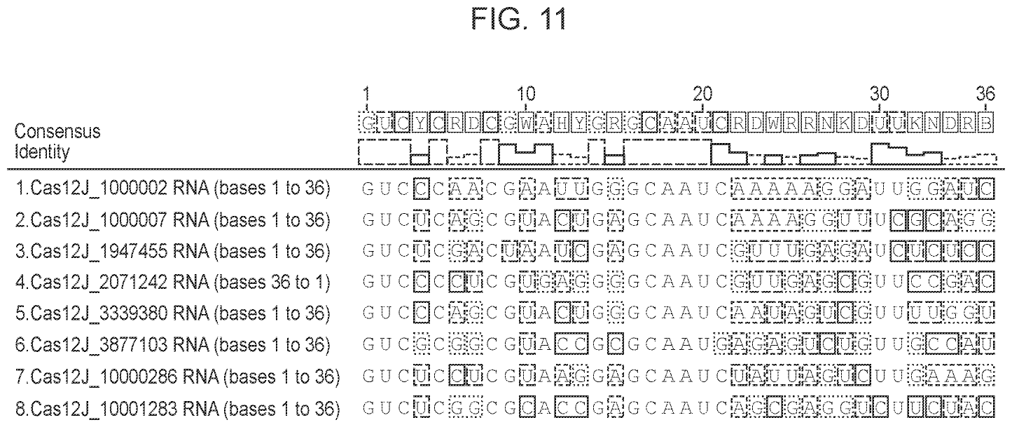

A CasPhi guide RNA constant region can include the reverse complement of any one of the nucleotide sequences depicted in FIG. 11. A CasPhi guide RNA constant region can include the reverse complement of a nucleotide sequence comprising the consensus sequence(s) depicted in FIG. 11.

The nucleotide sequences (with T substituted with U) can be combined with a spacer sequence (where the spacer sequence comprises a target nucleic acid-binding sequence (“guide sequence”)) of choice that is from 15 to 50 nucleotides (e.g., from 15 nucleotides (nt) to 20 nt, from 20 nt to 25 nt, from 25 nt to 30 nt, from 30 nt to 35 nt, from 35 nt to 40 nt, from 40 nt to 45 nt, or from 45 nt to 50 nt in length). In some cases, the spacer sequence is 35-38 nucleotides in length. For example, any one of the nucleotide sequences (with T substituted with U) depicted in FIG. 10 can be included in a guide RNA comprising (N)n-constant region, where N is any nucleotide and n is an integer from 15 to 50 (e.g., from 15 to 20, from 20 to 25, from 25 to 30, from 30 to 35, from 35 to 38, from 35 to 40, from 40 to 45, or from 45 to 50). The reverse complement of any one of the nucleotide sequences depicted in FIG. 10 (but with T substituted with U) can be included in a guide RNA comprising constant region-(N)n, where N is any nucleotide and n is an integer from 15 to 50 (e.g., from 15 to 20, from 20 to 25, from 25 to 30, from 30 to 35, from 35 to 38, from 35 to 40, from 40 to 45, or from 45 to 50).

The spacer (target-binding) region is 3′ of a repeat (CasPhi-binding) region in a CasPhi guide RNA. As one example, a guide RNA can have the reverse complement of the following nucleotide sequence: NNNNNNNNNNNNNNNNNNNNNNNNNNNNNNNNNNNGUCUCGACUAAUCGAGCAAUCGU UUGAGAUCUCUCC (SEQ ID NO:147), where N is any nucleotide, e.g., where the stretch of Ns includes a target nucleic acid-binding sequence. As another example, a guide RNA can have the reverse complement of the following nucleotide sequence: NNNNNNNNNNNNNNNNNNNNNNNNNNNNNNNNNNNGUCGGAACGCUCAACGAUUGCCCC UCACGAGGGGAC (SEQ ID NO:148), where N is any nucleotide, e.g., where the stretch of Ns includes a target nucleic acid-binding sequence.

As one example, a guide RNA can have the following nucleotide sequence: GUCUCGACUAAUCGAGCAAUCGUUUGAGAUCUCUCC (SEQ ID NO:37)—‘guide sequence’ (e.g., GUCUCGACUAAUCGAGCAAUCGUUUGAGAUCUCUCCNNNNNNNNNNNNNNNNNNNNNNN NNNNNNNNNNNN (SEQ ID NO:149), where the stretch of Ns represents the guide sequence/targeting sequence and N is any nucleotide). As another example, a guide RNA can have the following nucleotide sequence: GGAGAGAUCUCAAACGAUUGCUCGAUUAGUCGAGAC (SEQ ID NO:150)—‘guide sequence’ (e.g., GGAGAGAUCUCAAACGAUUGCUCGAUUAGUCGAGACNNNNNNNNNNNNNNNNNNNNNN NNNNNNNNNNNNN (SEQ ID NO:151), where the stretch of Ns represents the guide sequence/targeting sequence and N is any nucleotide).

As another example, a guide RNA can have the following nucleotide sequence: GUCGGAACGCUCAACGAUUGCCCCUCACGAGGGGAC (SEQ ID NO:142)—‘guide sequence’ (e.g., GUCGGAACGCUCAACGAUUGCCCCUCACGAGGGGACNNNNNNNNNNNNNNNNNNNNNNN NNNNNNNNNNNN (SEQ ID NO:152), where the stretch of Ns represents the guide sequence/targeting sequence and N is any nucleotide). As another example, a guide RNA can have the following nucleotide sequence: GUCCCCUCGUGAGGGGCAAUCGUUGAGCGUUCCGAC (SEQ ID NO:38)—‘guide sequence’ (e.g., GUCCCCUCGUGAGGGGCAAUCGUUGAGCGUUCCGACNNNNNNNNNNNNNNNNNNNNNNN NNNNNNNNNNNN (SEQ ID NO:153), where the stretch of Ns represents the guide sequence/targeting sequence and N is any nucleotide).

As another example, a guide RNA can have the following nucleotide sequence: CACAGGAGAGAUCUCAAACGAUUGCUCGAUUAGUCGAGAC (SEQ ID NO:144)—‘guide sequence’ (e.g., CACAGGAGAGAUCUCAAACGAUUGCUCGAUUAGUCGAGACNNNNNNNNNNNNNNNNNNN NNNNNNNNNNNNNNNN (SEQ ID NO:154), where the stretch of Ns represents the guide sequence/targeting sequence and N is any nucleotide). As another example, a guide RNA can have the following nucleotide sequence: UAAUGUCGGAACGCUCAACGAUUGCCCCUCACGAGGGGAC (SEQ ID NO:145)—‘guide sequence’ (e.g., UAAUGUCGGAACGCUCAACGAUUGCCCCUCACGAGGGGACNNNNNNNNNNNNNNNNNNN NNNNNNNNNNNNNNNN (SEQ ID NO:155), where the stretch of Ns represents the guide sequence/targeting sequence and N is any nucleotide). As another example, a guide RNA can have the following nucleotide sequence: AUUAACCAAAACGACUAUUGAUUGCCCAGUACGCUGGGAC (SEQ ID NO:146)—‘guide sequence’ (e.g., AUUAACCAAAACGACUAUUGAUUGCCCAGUACGCUGGGACNNNNNNNNNNNNNNNNNNN NNNNNNNNNNNNNNNN (SEQ ID NO:156), where the stretch of Ns represents the guide sequence/targeting sequence and N is any nucleotide).

### CasPhi Guide Polynucleotides

In some cases, a nucleic acid that binds to a variant CRISPR-Cas effector polypeptide of the present disclosure (or a fusion polypeptide of the present disclosure), forming a nucleic acid/polypeptide complex, and that targets the complex to a specific location within a target nucleic acid (e.g., a target DNA) comprises ribonucleotides only, deoxyribonucleotides only, or a mixture of ribonucleotides and deoxyribonucleotides. In some cases, a guide polynucleotide comprises ribonucleotides only, and is referred to herein as a “guide RNA.” In some cases, a guide polynucleotide comprises deoxyribonucleotides only, and is referred to herein as a “guide DNA.” In some cases, a guide polynucleotide comprises both ribonucleotides and deoxyribonucleotides. A guide polynucleotide can comprise combinations of ribonucleotide bases, deoxyribonucleotide bases, nucleotide analogs, modified nucleotides, and the like; and may further include naturally occurring backbone residues and/or linkages and/or non-naturally occurring backbone residues and/or linkages.

### Systems

The present disclosure provides a variant CRISPR-Cas effector polypeptide system (also referred to herein as a “variant CRISPR-Cas effector system”). A system of the present disclosure can comprise: a) a variant CRISPR-Cas effector polypeptide of the present disclosure and a CasPhi guide RNA; b) a variant CRISPR-Cas effector polypeptide of the present disclosure, a CasPhi guide RNA, and a donor template nucleic acid; c) a fusion polypeptide of the present disclosure and a CasPhi guide RNA; d) a fusion polypeptide of the present disclosure, a CasPhi guide RNA, and a donor template nucleic acid; e) an mRNA encoding a variant CRISPR-Cas effector polypeptide of the present disclosure; and a CasPhi guide RNA; f) an mRNA encoding a variant CRISPR-Cas effector polypeptide of the present disclosure, a CasPhi guide RNA, and a donor template nucleic acid; g) an mRNA encoding a fusion polypeptide of the present disclosure; and a CasPhi guide RNA; h) an mRNA encoding a fusion polypeptide of the present disclosure, a CasPhi guide RNA, and a donor template nucleic acid; i) a recombinant expression vector comprising a nucleotide sequence encoding a variant CRISPR-Cas effector polypeptide of the present disclosure and a nucleotide sequence encoding a CasPhi guide RNA; j) a recombinant expression vector comprising a nucleotide sequence encoding a variant CRISPR-Cas effector polypeptide of the present disclosure, a nucleotide sequence encoding a CasPhi guide RNA, and a nucleotide sequence encoding a donor template nucleic acid; k) a recombinant expression vector comprising a nucleotide sequence encoding a fusion polypeptide of the present disclosure and a nucleotide sequence encoding a CasPhi guide RNA; l) a recombinant expression vector comprising a nucleotide sequence encoding a fusion polypeptide of the present disclosure, a nucleotide sequence encoding a CasPhi guide RNA, and a nucleotide sequence encoding a donor template nucleic acid; m) a first recombinant expression vector comprising a nucleotide sequence encoding a variant CRISPR-Cas effector polypeptide of the present disclosure, and a second recombinant expression vector comprising a nucleotide sequence encoding a CasPhi guide RNA; n) a first recombinant expression vector comprising a nucleotide sequence encoding a variant CRISPR-Cas effector polypeptide of the present disclosure, and a second recombinant expression vector comprising a nucleotide sequence encoding a CasPhi guide RNA; and a donor template nucleic acid; o) a first recombinant expression vector comprising a nucleotide sequence encoding a fusion polypeptide of the present disclosure, and a second recombinant expression vector comprising a nucleotide sequence encoding a CasPhi guide RNA; p) a first recombinant expression vector comprising a nucleotide sequence encoding a fusion polypeptide of the present disclosure, and a second recombinant expression vector comprising a nucleotide sequence encoding a CasPhi guide RNA; and a donor template nucleic acid; q) a recombinant expression vector comprising a nucleotide sequence encoding a variant CRISPR-Cas effector polypeptide of the present disclosure, a nucleotide sequence encoding a first CasPhi guide RNA, and a nucleotide sequence encoding a second CasPhi guide RNA; or r) a recombinant expression vector comprising a nucleotide sequence encoding a fusion polypeptide of the present disclosure, a nucleotide sequence encoding a first CasPhi guide RNA, and a nucleotide sequence encoding a second CasPhi guide RNA; or some variation of one of (a) through (r).

The present disclosure provides an implantable device comprising a system of the present disclosure. In some cases, the system is within a matrix. In some cases, the system is within a reservoir. The present disclosure provides a container comprising a system of the present disclosure. In some cases, the container is a syringe. In some cases, the container is sterile.

### Compositions

The present disclosure provides compositions comprising a variant CRISPR-Cas effector polypeptide of the present disclosure or a fusion polypeptide of the present disclosure. A composition of the present disclosure can comprise: a) a variant CRISPR-Cas effector polypeptide of the present disclosure, or a fusion polypeptide of the present disclosure; and b) one or more of: a salt, a buffer, a protease inhibitor, a detergent, a lipid, and the like. In some cases, a composition of the present disclosure comprises: a) a variant CRISPR-Cas effector polypeptide of the present disclosure; and b) a CasPhi guide RNA. The variant CRISPR-Cas effector polypeptide and the CasPhi guide RNA can form an RNP. Thus, the present disclosure provides an RNP comprising: a) a variant CRISPR-Cas effector polypeptide of the present disclosure; and b) a CasPhi guide RNA. In some cases, a composition of the present disclosure comprises: a) a fusion polypeptide of the present disclosure; and b) a CasPhi guide RNA. The fusion polypeptide and the CasPhi guide RNA can form an RNP. Thus, the present disclosure provides an RNP comprising: a) a fusion polypeptide of the present disclosure; and b) a CasPhi guide RNA.

### Nucleic Acids

The present disclosure provides one or more nucleic acids comprising one or more of: a donor polynucleotide sequence, a nucleotide sequence encoding a variant CRISPR-Cas effector polypeptide of the present disclosure, a fusion polypeptide of the present disclosure, a CasPhi guide RNA, and a nucleotide sequence encoding a CasPhi guide RNA. The present disclosure provides a nucleic acid comprising a nucleotide sequence encoding a fusion polypeptide of the present disclosure. The present disclosure provides a recombinant expression vector that comprises a nucleotide sequence encoding a variant CRISPR-Cas effector polypeptide of the present disclosure. The present disclosure provides a recombinant expression vector that comprises a nucleotide sequence encoding a fusion polypeptide of the present disclosure. The present disclosure provides a recombinant expression vector that comprises: a) a nucleotide sequence encoding a variant CRISPR-Cas effector polypeptide of the present disclosure; and b) a nucleotide sequence encoding a CasPhi guide RNA(s). The present disclosure provides a recombinant expression vector that comprises: a) a nucleotide sequence encoding a fusion polypeptide of the present disclosure; and b) a nucleotide sequence encoding a CasPhi guide RNA(s). In some cases, the nucleotide sequence encoding the variant CRISPR-Cas effector polypeptide and/or the nucleotide sequence encoding the CasPhi guide RNA is operably linked to a promoter that is operable in a cell type of choice (e.g., a prokaryotic cell, a eukaryotic cell, a plant cell, an animal cell, a mammalian cell, a primate cell, a rodent cell, a human cell, etc.).

In some cases, a nucleotide sequence encoding a variant CRISPR-Cas effector polypeptide of the present disclosure is codon optimized. This type of optimization can entail a mutation of a variant CRISPR-Cas effector polypeptide-encoding nucleotide sequence to mimic the codon preferences of the intended host organism or cell while encoding the same protein. Thus, the codons can be changed, but the encoded protein remains unchanged. For example, if the intended target cell was a human cell, a human codon-optimized variant CRISPR-Cas effector polypeptide-encoding nucleotide sequence could be used. As another non-limiting example, if the intended host cell were a mouse cell, then a mouse codon-optimized variant CRISPR-Cas effector polypeptide-encoding nucleotide sequence could be generated. As another non-limiting example, if the intended host cell were a plant cell, then a plant codon-optimized variant CRISPR-Cas effector polypeptide-encoding nucleotide sequence could be generated. As another non-limiting example, if the intended host cell were an insect cell, then an insect codon-optimized variant CRISPR-Cas effector polypeptide-encoding nucleotide sequence could be generated.

Codon usage tables are readily available, for example, at the “Codon Usage Database” available at www[dot]kazusa[dot]or[dot]p[forwardslash]codon. In some cases, a nucleic acid of the present disclosure comprises a variant CRISPR-Cas effector polypeptide-encoding nucleotide sequence that is codon optimized for expression in a eukaryotic cell. In some cases, a nucleic acid of the present disclosure comprises a variant CRISPR-Cas effector polypeptide-encoding nucleotide sequence that is codon optimized for expression in an animal cell. In some cases, a nucleic acid of the present disclosure comprises a variant CRISPR-Cas effector polypeptide-encoding nucleotide sequence that is codon optimized for expression in a fungus cell. In some cases, a nucleic acid of the present disclosure comprises a variant CRISPR-Cas effector polypeptide-encoding nucleotide sequence that is codon optimized for expression in a plant cell. In some cases, a nucleic acid of the present disclosure comprises a variant CRISPR-Cas effector polypeptide-encoding nucleotide sequence that is codon optimized for expression in a monocotyledonous plant species. In some cases, a nucleic acid of the present disclosure comprises a variant CRISPR-Cas effector polypeptide-encoding nucleotide sequence that is codon optimized for expression in a dicotyledonous plant species. In some cases, a nucleic acid of the present disclosure comprises a variant CRISPR-Cas effector polypeptide-encoding nucleotide sequence that is codon optimized for expression in a gymnosperm plant species. In some cases, a nucleic acid of the present disclosure comprises a variant CRISPR-Cas effector polypeptide-encoding nucleotide sequence that is codon optimized for expression in an angiosperm plant species. In some cases, a nucleic acid of the present disclosure comprises a variant CRISPR-Cas effector polypeptide-encoding nucleotide sequence that is codon optimized for expression in a corn cell. In some cases, a nucleic acid of the present disclosure comprises a variant CRISPR-Cas effector polypeptide-encoding nucleotide sequence that is codon optimized for expression in a soybean cell. In some cases, a nucleic acid of the present disclosure comprises a variant CRISPR-Cas effector polypeptide-encoding nucleotide sequence that is codon optimized for expression in a rice cell. In some cases, a nucleic acid of the present disclosure comprises a variant CRISPR-Cas effector polypeptide-encoding nucleotide sequence that is codon optimized for expression in a wheat cell. In some cases, a nucleic acid of the present disclosure comprises a variant CRISPR-Cas effector polypeptide-encoding nucleotide sequence that is codon optimized for expression in a cotton cell. In some cases, a nucleic acid of the present disclosure comprises a variant CRISPR-Cas effector polypeptide-encoding nucleotide sequence that is codon optimized for expression in a sorghum cell. In some cases, a nucleic acid of the present disclosure comprises a variant CRISPR-Cas effector polypeptide-encoding nucleotide sequence that is codon optimized for expression in an alfalfa cell. In some cases, a nucleic acid of the present disclosure comprises a variant CRISPR-Cas effector polypeptide-encoding nucleotide sequence that is codon optimized for expression in a sugar cane cell. In some cases, a nucleic acid of the present disclosure comprises a variant CRISPR-Cas effector polypeptide-encoding nucleotide sequence that is codon optimized for expression in an Arabidopsis cell. In some cases, a nucleic acid of the present disclosure comprises a variant CRISPR-Cas effector polypeptide-encoding nucleotide sequence that is codon optimized for expression in a tomato cell. In some cases, a nucleic acid of the present disclosure comprises a variant CRISPR-Cas effector polypeptide-encoding nucleotide sequence that is codon optimized for expression in a cucumber cell. In some cases, a nucleic acid of the present disclosure comprises a variant CRISPR-Cas effector polypeptide-encoding nucleotide sequence that is codon optimized for expression in a potato cell. In some cases, a nucleic acid of the present disclosure comprises a variant CRISPR-Cas effector polypeptide-encoding nucleotide sequence that is codon optimized for expression in an algal cell.

The present disclosure provides one or more recombinant expression vectors that include (in different recombinant expression vectors in some cases, and in the same recombinant expression vector in some cases): (i) a nucleotide sequence of a donor template nucleic acid (where the donor template comprises a nucleotide sequence having homology to a target sequence of a target nucleic acid (e.g., a target genome)); (ii) a nucleotide sequence that encodes a CasPhi guide RNA that hybridizes to a target sequence of the target locus of the targeted genome (e.g., operably linked to a promoter that is operable in a target cell such as a eukaryotic cell); and (iii) a nucleotide sequence encoding a variant CRISPR-Cas effector protein (e.g., operably linked to a promoter that is operable in a target cell such as a eukaryotic cell). The present disclosure provides one or more recombinant expression vectors that include (in different recombinant expression vectors in some cases, and in the same recombinant expression vector in some cases): (i) a nucleotide sequence of a donor template nucleic acid (where the donor template comprises a nucleotide sequence having homology to a target sequence of a target nucleic acid (e.g., a target genome)); and (ii) a nucleotide sequence that encodes a CasPhi guide RNA that hybridizes to a target sequence of the target locus of the targeted genome (e.g., operably linked to a promoter that is operable in a target cell such as a eukaryotic cell). The present disclosure provides one or more recombinant expression vectors that include (in different recombinant expression vectors in some cases, and in the same recombinant expression vector in some cases): (i) a nucleotide sequence that encodes a CasPhi guide RNA that hybridizes to a target sequence of the target locus of the targeted genome (e.g., operably linked to a promoter that is operable in a target cell such as a eukaryotic cell); and (ii) a nucleotide sequence encoding a variant CRISPR-Cas effector protein (e.g., operably linked to a promoter that is operable in a target cell such as a eukaryotic cell).

Suitable expression vectors include viral expression vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (AAV) (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, a recombinant expression vector of the present disclosure is a recombinant adeno-associated virus (AAV) vector. In some cases, a recombinant expression vector of the present disclosure is a recombinant lentivirus vector. In some cases, a recombinant expression vector of the present disclosure is a recombinant retroviral vector.

For plant applications, viral vectors based on Tobamoviruses, Potexviruses, Potyviruses, Tobraviruses, Tombusviruses, Geminiviruses, Bromoviruses, Carmoviruses, Alfamoviruses, or Cucumoviruses can be used. See, e.g., Peyret and Lomonossoff (2015) Plant Biotechnol. J. 13:1121. Suitable Tobamovirus vectors include, for example, a tomato mosaic virus (ToMV) vector, a tobacco mosaic virus (TMV) vector, a tobacco mild green mosaic virus (TMGMV) vector, a pepper mild mottle virus (PMMoV) vector, a paprika mild mottle virus (PaMMV) vector, a cucumber green mottle mosaic virus (CGMMV) vector, a kyuri green mottle mosaic virus (KGMMV) vector, a hibiscus latent fort pierce virus (HLFPV) vector, an odontoglossum ringspot virus (ORSV) vector, a rehmannia mosaic virus (ReMV) vector, a Sammon's opuntia virus (SOV) vector, a wasabi mottle virus (WMoV) vector, a youcai mosaic virus (YoMV) vector, a sunn-hemp mosaic virus (SHMV) vector, and the like. Suitable Potexvirus vectors include, for example, a potato virus X (PVX) vector, a potato aucubamosaicvirus (PAMV) vector, an Alstroemeria virus X (AlsVX) vector, a cactus virus X (CVX) vector, a Cymbidium mosaic virus (CymMV) vector, a hosta virus X (HVX) vector, a lily virus X (LVX) vector, a Narcissus mosaic virus (NMV) vector, a Nerine virus X (NVX) vector, a Plantago asiatica mosaic virus (PIAMV) vector, a strawberry mild yellow edge virus (SMYEV) vector, a tulip virus X (TVX) vector, a white clover mosaic virus (WClMV) vector, a bamboo mosaic virus (BaMV) vector, and the like. Suitable Potyvirus vectors include, for example, a potato virus Y (PVY) vector, a bean common mosaic virus (BCMV) vector, a clover yellow vein virus (ClYVV) vector, an East Asian Passiflora virus (EAPV) vector, a Freesia mosaic virus (FreMV) vector, a Japanese yam mosaic virus (JYMV) vector, a lettuce mosaic virus (LMV) vector, a Maize dwarf mosaic virus (MDMV) vector, an onion yellow dwarf virus (OYDV) vector, a papaya ringspot virus (PRSV) vector, a pepper mottle virus (PepMoV) vector, a Perilla mottle virus (PerMoV) vector, a plum pox virus (PPV) vector, a potato virus A (PVA) vector, a sorghum mosaic virus (SrMV) vector, a soybean mosaic virus (SMV) vector, a sugarcane mosaic virus (SCMV) vector, a tulip mosaic virus (TulMV) vector, a turnip mosaic virus (TuMV) vector, a watermelon mosaic virus (WMV) vector, a zucchini yellow mosaic virus (ZYMV) vector, a tobacco etch virus (TEV) vector, and the like. Suitable Tobravirus vectors include, for example, a tobacco rattle virus (TRV) vector and the like. Suitable Tombusvirus vectors include, for example, a tomato bushy stunt virus (TBSV) vector, an eggplant mottled crinkle virus (EMCV) vector, a grapevine Algerian latent virus (GALV) vector, and the like. Suitable Cucumovirus vectors include, for example, a cucumber mosaic virus (CMV) vector, a peanut stunt virus (PSV) vector, a tomato aspermy virus (TAV) vector, and the like. Suitable Bromovirus vectors include, for example, a brome mosaic virus (BMV) vector, a cowpea chlorotic mottle virus (CCMV) vector, and the like. Suitable Carmovirus vectors include, for example, a carnation mottle virus (CarMV) vector, a melon necrotic spot virus (MNSV) vector, a pea stem necrotic virus (PSNV) vector, a turnip crinkle virus (TCV) vector, and the like. Suitable Alfamovirus vectors include, for example, an alfalfa mosaic virus (AMV) vector, and the like.

Depending on the host/vector system utilized, any of a number of suitable transcription and translation control elements, including constitutive and inducible promoters, transcription enhancer elements, transcription terminators, etc. may be used in the expression vector.

In some cases, a nucleotide sequence encoding a CasPhi guide RNA is operably linked to a control element, e.g., a transcriptional control element, such as a promoter. In some embodiments, a nucleotide sequence encoding a variant CRISPR-Cas effector protein or a fusion polypeptide of the present disclosure is operably linked to a control element, e.g., a transcriptional control element, such as a promoter.

The transcriptional control element can be a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the transcriptional control element can be functional in eukaryotic cells, e.g., hematopoietic stem cells (e.g., mobilized peripheral blood (mPB) CD34(+) cell, bone marrow (BM) CD34(+) cell, etc.).

Non-limiting examples of eukaryotic promoters (promoters functional in a eukaryotic cell) include EF1α, those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, fluorescent protein, etc.) that can be fused to the variant CRISPR-Cas effector protein, thus resulting in a fusion polypeptide.

In some cases, a nucleotide sequence encoding a CasPhi guide RNA and/or a variant CRISPR-Cas effector of the present disclosure and/or a fusion polypeptide of the present disclosure is operably linked to an inducible promoter. In some embodiments, a nucleotide sequence encoding a CasPhi guide RNA and/or a variant CRISPR-Cas effector of the present disclosure and/or a fusion protein of the present disclosure is operably linked to a constitutive promoter.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a Rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

In some cases, a nucleotide sequence encoding a CasPhi guide RNA is operably linked to (under the control of) a promoter operable in a eukaryotic cell (e.g., a U6 promoter, an enhanced U6 promoter, an H1 promoter, and the like). As would be understood by one of ordinary skill in the art, when expressing an RNA (e.g., a guide RNA) from a nucleic acid (e.g., an expression vector) using a U6 promoter (e.g., in a eukaryotic cell), or another PolIII promoter, the RNA may need to be mutated if there are several Ts in a row (coding for Us in the RNA). This is because a string of Ts (e.g., 5 Ts) in DNA can act as a terminator for polymerase III (PolIII). Thus, in order to ensure transcription of a guide RNA in a eukaryotic cell it may sometimes be necessary to modify the sequence encoding the guide RNA to eliminate runs of Ts. In some cases, a nucleotide sequence encoding a variant CRISPR-Cas effector polypeptide or a fusion polypeptide of the present disclosure is operably linked to a promoter operable in a eukaryotic cell (e.g., a CMV promoter, an EF1α promoter, an estrogen receptor-regulated promoter, and the like).

In some cases, expression of a nucleic acid of the present disclosure may be driven (in operable linkage) with a U6 promoter. In some cases, expression of a nucleic acid of the present disclosure may be driven (in operable linkage) with a promoter having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the following U6 promoter sequence:

(SEQ ID NO: 157)
AAGGTCGGGCAGGAAGAGGGCCTATTTCCCATGATTCCTTCATATTTGCA
TATACGATACAAGGCTGTTAGAGAGATAATTAGAATTAATTTGACTGTAA
ACACAAAGATATTAGTACAAAATACGTGACGTAGAAAGTAATAATTTCTT
GGGTAGTTTGCAGTTTTAAAATTATGTTTTAAAATGGACTATCATATGCT
TACCGTAACTTGAAAGTATTTCGATTTCTTGGCTTTATATATCTTGTGGA
AAGGACG

A U6 promoter can have the following nucleotide sequence:

(SEQ ID NO: 157)
AAGGTCGGGCAGGAAGAGGGCCTATTTCCCATGATTCCTTCATATTTGCA
TATACGATACAAGGCTGTTAGAGAGATAATTAGAATTAATTTGACTGTAA
ACACAAAGATATTAGTACAAAATACGTGACGTAGAAAGTAATAATTTCTT
GGGTAGTTTGCAGTTTTAAAATTATGTTTTAAAATGGACTATCATATGCT
TACCGTAACTTGAAAGTATTTCGATTTCTTGGCTTTATATATCTTGTGGA
AAGGACG.

In some cases, a nucleic acid comprises a nucleotide sequence that is operably linked to a U6 promoter having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the following AtU626 promoter sequence:

(SEQ ID NO: 158)
AAGCTTCGTTGAACAACGGAAACTCGACTTGCCTTCCGCACAATACATCA
TTTCTTCTTAGCTTTTTTTCTTCTTCTTCGTTCATACAGTTTTTTTTTGT
TTATCAGCTTACATTTTCTTGAACCGTAGCTTTCGTTTTCTTCTTTTTAA
CTTTCCATTCGGAGTTTTTGTATCTTGTTTCATAGTTTGTCCCAGGATTA
GAATGATTAGGCATCGAACCTTCAAGAATTTGATTGAATAAAACATCTTC
ATTCTTAAGATATGAAGATAATCTTCAAAAGGCCCCTGGGAATCTGAAAG
AAGAGAAGCAGGCCCATTTATATGGGAAAGAACAATAGTATTTCTTATAT
AGGCCCATTTAAGTTGAAAACAATCTTCAAAAGTCCCACATCGCTTAGAT
AAGAAAACGAAGCTGAGTTTATATACAGCTAGAGTCGAAGTAGTGATT.

In some cases, a nucleotide sequence encoding a CasPhi guide RNA is operably linked to a Pol-II promoter. Suitable Pol-II promoters include, e.g., a UBQ10 promoter, a 35S promoter, a Nos-P promoter, and a UBQ1 promoter.

In some cases, expression of a nucleic acid of the present disclosure may be driven (in operable linkage) with a UBQ10 promoter. In some cases, expression of a nucleic acid of the present disclosure may be driven (in operable linkage) with a promoter having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the following UBQ10 promoter sequence:

(SEQ ID NO: 159)
CGACGAGTCAGTAATAAACGGCGTCAAAGTGGTTGCAGCCGGCACACACG
AGTCGTGTTTATCAACTCAAAGCACAAATACTTTTCCTCAACCTAAAAAT
AAGGCAATTAGCCAAAAACAACTTTGCGTGTAAACAACGCTCAATACACG
TGTCATTTTATTATTAGCTATTGCTTCACCGCCTTAGCTTTCTCGTGACC
TAGTCGTCCTCGTCTTTTCTTCTTCTTCTTCTATAAAACAATACCCAAAG
AGCTCTTCTTCTTCACAATTCAGATTTCAATTTCTCAAAATCTTAAAAAC
TTTCTCTCAATTCTCTCTACCGTGATCAAGGTAAATTTCTGTGTTCCTTA
TTCTCTCAAAATCTTCGATTTTGTTTTCGTTCGATCCCAATTTCGTATAT
GTTCTTTGGTTTAGATTCTGTTAATCTTAGATCGAAGACGATTTTCTGGG
TTTGATCGTTAGATATCATCTTAATTCTCGATTAGGGTTTCATAGATATC
ATCCGATTTGTTCAAATAATTTGAGTTTTGTCGAATAATTACTCTTCGAT
TTGTGATTTCTATCTAGATCTGGTGTTAGTTTCTAGTTTGTGCGATCGAA
TTTGTAGATTAATCTGAGTTTTTCTGATTAACA.

In some cases, a UBQ10 promoter comprises the following amino acid sequence:

(SEQ ID NO: 159)
cgacgagtcagtaataaacggcgtcaaagtggttgcagccggcacacacg
agtcgtgtttatcaactcaaagcacaaatacttttcctcaacctaaaaat
aaggcaattagccaaaaacaactttgcgtgtaaacaacgctcaatacacg
tgtcattttattattagctattgcttcaccgccttagctttctcgtgacc
tagtcgtcctcgtcttttcttcttcttcttctataaaacaatacccaaag
agctcttcttcttcacaattcagatttcaatttctcaaaatcttaaaaac
tttctctcaattctctctaccgtgatcaaggtaaatttctgtgttcctta
ttctctcaaaatcttcgattttgttttcgttcgatcccaatttcgtatat
gttctttggtttagattctgttaatcttagatcgaagacgattttctggg
tttgatcgttagatatcatcttaattctcgattagggtttcatagatatc
atccgatttgttcaaataatttgagttttgtcgaataattactcttcgat
ttgtgatttctatctagatctggtgttagtttctagtttgtgcgatcgaa
tttgtagattaatctgagtttttctgattaaca.

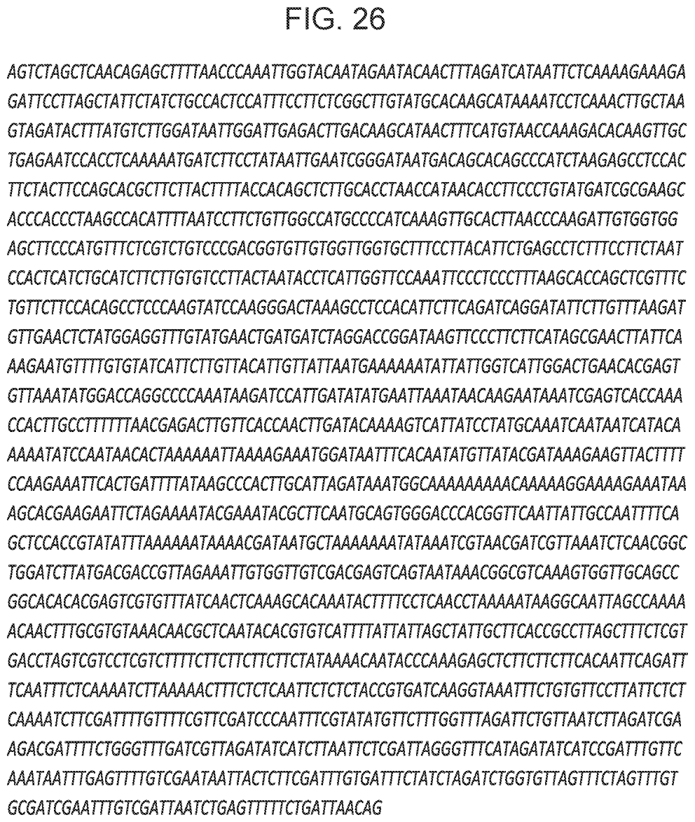

In some cases, a suitable Pol-II promoter comprises a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, nucleotide sequence identity with a contiguous stretch of from about 1500 nucleotides (nt) to about 1986 nt (e.g., from about 1500 nt to about 1600 nt, from about 1600 nt to about 1700 nt, from about 1700 nt to about 1800 nt, from about 1800 nt to about 1900 nt, or from about 1900 nt to about 1986 nt) of the UBQ10 promoter nucleotide sequence depicted in FIG. 26. In some cases, a UBQ10 promoter has a length of from about 1500 nucleotides (nt) to about 1986 nt (e.g., from about 1500 nt to about 1600 nt, from about 1600 nt to about 1700 nt, from about 1700 nt to about 1800 nt, from about 1800 nt to about 1900 nt, or from about 1900 nt to about 1986 nt).

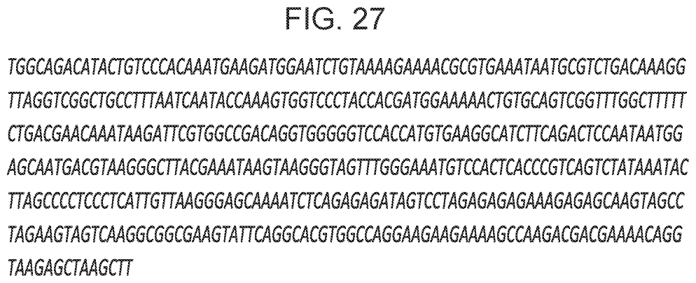

In some cases, a suitable Pol-II promoter comprises a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, nucleotide sequence identity with a contiguous stretch of from about 350 nucleotides (nt) to about 465 nt (e.g., from about 350 nt to about 375 nt, from about 375 nt to about 400 nt, from about 400 nt to about 425 nt, from about 425 nt to about 450 nt, or from about 450 nt to about 465 nt) of the CmYLCV promoter nucleotide sequence depicted in FIG. 27. In some cases, a CmYLCV promoter has a length of from about 350 nucleotides (nt) to about 465 nt (e.g., from about 350 nt to about 375 nt, from about 375 nt to about 400 nt, from about 400 nt to about 425 nt, from about 425 nt to about 450 nt, or from about 450 nt to about 465 nt).

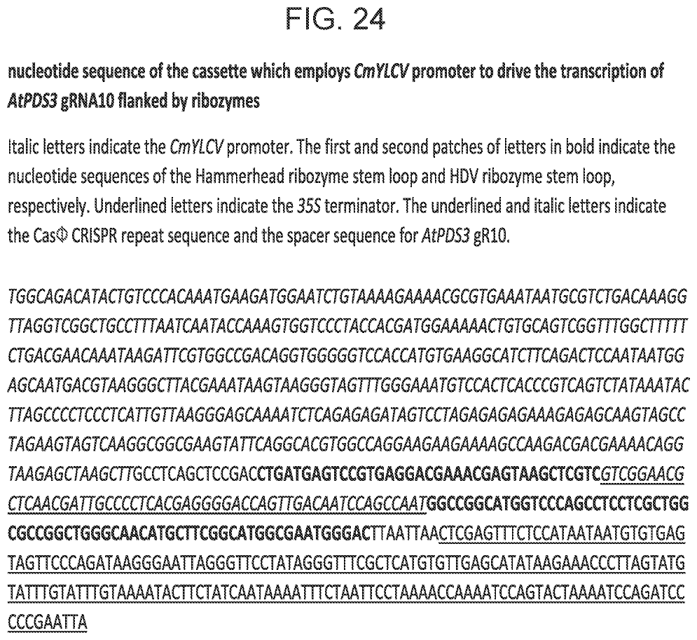

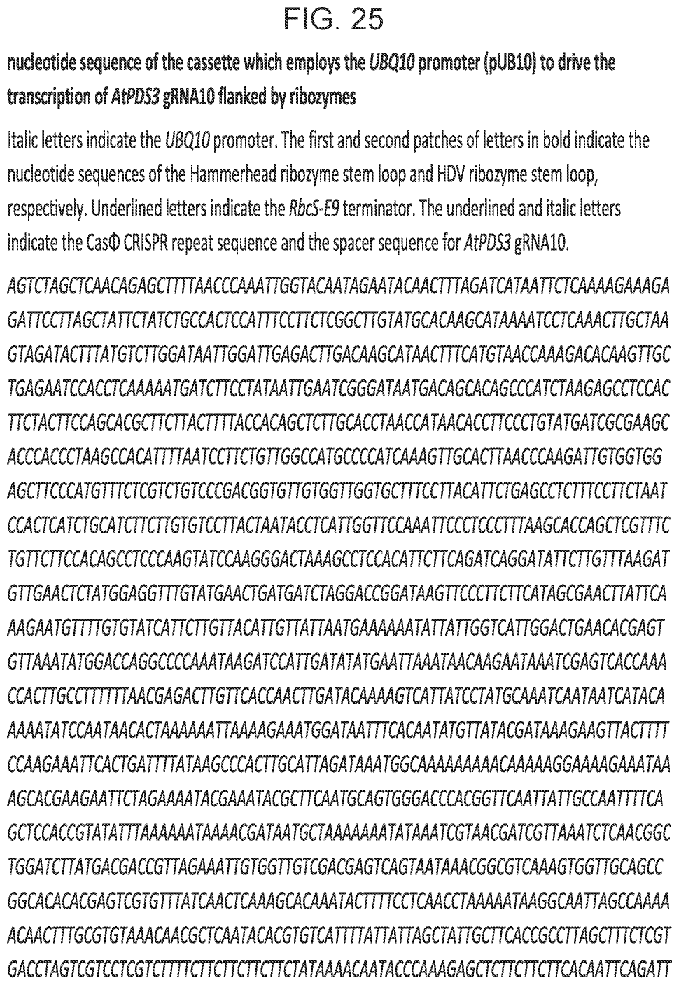

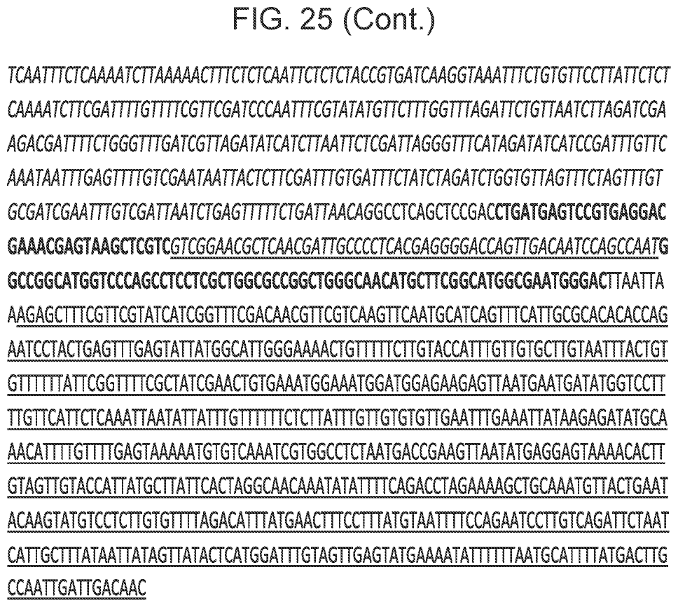

In some cases, a DNA molecule comprising a nucleotide sequence encoding a guide RNA (gRNA) (e.g., a single-molecule gRNA or “sgRNA”) comprises a Pol-III promoter operably linked to the nucleotide sequence encoding the sgRNA. In some cases, a DNA molecule comprising a nucleotide sequence encoding a sgRNA comprises a Pol-II promoter operably linked to the nucleotide sequence encoding the sgRNA. In some cases, the nucleotide sequence encoding the sgRNA is flanked by nucleotide sequences encoding ribozymes. For example, in some cases, a DNA molecule comprising a nucleotide sequence encoding a sgRNA comprises, in order from 5′ to 3′: i) a Pol-II promoter; ii) a nucleotide sequence encoding a first ribozyme stem loop; iii) the nucleotide sequence encoding the sgRNA; and iv) a nucleotide sequence encoding a second ribozyme stem loop. The first and the second ribozyme stem loop-encoding sequences can be the same or different. Examples of suitable nucleotide sequences encoding ribozyme stem loop are provided in FIG. 24 and FIG. 25. In some cases, the DNA molecule further comprise a terminator, e.g., as depicted in FIG. 24 and FIG. 25.

Examples of inducible promoters include, but are not limited toT7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; estrogen and/or an estrogen analog; IPTG; etc.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used as long as the promoter is functional in the targeted host cell (e.g., eukaryotic cell; prokaryotic cell).

In some cases, the promoter is a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

RNA polymerase III (Pol III) promoters can be used to drive the expression of non-protein coding RNA molecules (e.g., guide RNAs). In some cases, a suitable promoter is a Pol III promoter. In some cases, a Pol III promoter is operably linked to a nucleotide sequence encoding a guide RNA (gRNA). In some cases, a Pol III promoter is operably linked to a nucleotide sequence encoding a single-guide RNA (sgRNA). In some cases, a Pol III promoter is operably linked to a nucleotide sequence encoding a CRISPR RNA (crRNA). In some cases, a Pol III promoter is operably linked to a nucleotide sequence encoding a tracrRNA.

Non-limiting examples of Pol III promoters include a U6 promoter, an Hl promoter, a 5S promoter, an Adenovirus 2 (Ad2) VAI promoter, a tRNA promoter, and a 7SK promoter. See, for example, Schramm and Hernandez (2002) Genes & Development 16:2593-2620. In some cases, a Pol III promoter is selected from the group consisting of a U6 promoter, an Hl promoter, a 5S promoter, an Adenovirus 2 (Ad2) VAI promoter, a tRNA promoter, and a 7SK promoter. In some cases, a guide RNA-encoding nucleotide sequence is operably linked to a promoter selected from the group consisting of a U6 promoter, an Hl promoter, a 5S promoter, an Adenovirus 2 (Ad2) VAI promoter, a tRNA promoter, and a 7SK promoter. In some cases, a single-guide RNA-encoding nucleotide sequence is operably linked to a promoter selected from the group consisting of a U6 promoter, an Hl promoter, a 5S promoter, an Adenovirus 2 (Ad2) VAI promoter, a tRNA promoter, and a 7SK promoter.

Examples describing a promoter that can be used herein in connection with expression in plants, plant tissues, and plant cells include, but are not limited to, promoters described in: U.S. Pat. No. 6,437,217 (maize RS81 promoter), U.S. Pat. No. 5,641,876 (rice actin promoter), U.S. Pat. No. 6,426,446 (maize RS324 promoter), U.S. Pat. No. 6,429,362 (maize PR-1 promoter), U.S. Pat. No. 6,232,526 (maize A3 promoter), U.S. Pat. No. 6,177,611 (constitutive maize promoters), U.S. Pat. Nos. 5,322,938, 5,352,605, 5,359,142 and 5,530,196 (35S promoter), U.S. Pat. No. 6,433,252 (maize L3 oleosin promoter), U.S. Pat. No. 6,429,357 (rice actin 2 promoter as well as a rice actin 2 intron), U.S. Pat. No. 5,837,848 (root specific promoter), U.S. Pat. No. 6,294,714 (light inducible promoters), U.S. Pat. No. 6,140,078 (salt inducible promoters), U.S. Pat. No. 6,252,138 (pathogen inducible promoters), U.S. Pat. No. 6,175,060 (phosphorus deficiency inducible promoters), U.S. Pat. No. 6,635,806 (gamma-coixin promoter), and U.S. patent application Ser. No. 09/757,089 (maize chloroplast aldolase promoter). Additional promoters that can find use include a nopaline synthase (NOS) promoter (Ebert et al., 1987), the octopine synthase (OCS) promoter (which is carried on tumor-inducing plasmids of Agrobacterium tumefaciens), the caulimovirus promoters such as the cauliflower mosaic virus (CaMV) 19S promoter (Lawton et al. Plant Molecular Biology (1987) 9: 315-324), the CaMV 35S promoter (Odell et al., Nature (1985) 313: 810-812), the figwort mosaic virus 35S-promoter (U.S. Pat. Nos. 6,051,753; 5,378,619), the sucrose synthase promoter (Yang and Russell, Proceedings of the National Academy of Sciences, USA (1990) 87: 4144-4148), the R gene complex promoter (Chandler et al., Plant Cell (1989) 1: 1175-1183), and the chlorophyll a/b binding protein gene promoter, PC1SV (U.S. Pat. No. 5,850,019), and AGRtu.nos (GenBank Accession V00087; Depicker et al., Journal of Molecular and Applied Genetics (1982) 1: 561-573; Bevan et al., 1983) promoters.

Examples of suitable tissue specific promoters for use in plants, plant tissues, or plant cells include, for example, a lectin promoter, a corn alcohol dehydrogenase 1 promoter, a corn light harvesting complex promoter, a corn heat shock protein promoter, a pea small subunit RuBP carboxylase promoter, a Ti plasmid mannopine synthase promoter, a Ti plasmid nopaline synthase promoter, a petunia chalcone isomerase promoter, a bean glycine rich protein 1 promoter, a truncated CaMV 35s promoter, a potato patatin promoter, a root cell promoter, a maize zein promoter, a globulin-1 promoter, an α-tubulin promoter, a cab promoter, a PEPCase promoter, a R gene complex-associated promoter, and a chalcone synthase promoter.

Methods of introducing a nucleic acid (e.g., a nucleic acid comprising a donor polynucleotide sequence, one or more nucleic acids encoding a variant CRISPR-Cas effector protein of the present disclosure and/or a fusion polypeptide of the present disclosure and/or a CasPhi guide RNA, and the like) into a host cell are known in the art, and any convenient method can be used to introduce a nucleic acid (e.g., an expression construct) into a cell. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

Introducing the recombinant expression vector into cells can occur in any culture media and under any culture conditions that promote the survival of the cells. Introducing the recombinant expression vector into a target cell can be carried out in vivo or ex vivo. Introducing the recombinant expression vector into a target cell can be carried out in vitro.

In some cases, a variant CRISPR-Cas effector protein can be provided as RNA encoding the variant CRISPR-Cas effector protein. The RNA can be provided by direct chemical synthesis or may be transcribed in vitro from a DNA (e.g., encoding the variant CRISPR-Cas effector protein). Once synthesized, the RNA may be introduced into a cell by any of the well-known techniques for introducing nucleic acids into cells (e.g., microinjection, electroporation, transfection, etc.).

Nucleic acids may be provided to the cells using well-developed transfection techniques; see, e.g. Angel and Yanik (2010) PLoS One 5(7): e11756, and the commercially available TransMessenger® reagents from Qiagen, Stemfect™ RNA Transfection Kit from Stemgent, and TransIT®-mRNA Transfection Kit from Mirus Bio LLC. See also Beumer et al. (2008) PNAS 105(50):19821-19826.

Vectors may be provided directly to a target host cell. In other words, the cells are contacted with vectors comprising the subject nucleic acids (e.g., recombinant expression vectors having the donor template sequence and encoding the CasPhi guide RNA; recombinant expression vectors encoding the variant CRISPR-Cas effector protein; etc.) such that the vectors are taken up by the cells. Methods for contacting cells with nucleic acid vectors that are plasmids, include electroporation, calcium chloride transfection, microinjection, and lipofection are well known in the art. For viral vector delivery, cells can be contacted with viral particles comprising the subject viral expression vectors.

Retroviruses, for example, lentiviruses, are suitable for use in methods of the present disclosure. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

Vectors used for providing the nucleic acids encoding CasPhi guide RNA and/or a variant CRISPR-Cas effector polypeptide and/or a fusion polypeptide to a target host cell can include suitable promoters for driving the expression, that is, transcriptional activation, of the nucleic acid of interest. In other words, in some cases, the nucleic acid of interest will be operably linked to a promoter. This may include ubiquitously acting promoters, for example, the CMV-β-actin promoter, or inducible promoters, such as promoters that are active in particular cell populations or that respond to the presence of drugs such as tetracycline. By transcriptional activation, it is intended that transcription will be increased above basal levels in the target cell by 10 fold, by 100 fold, more usually by 1000 fold. In addition, vectors used for providing a nucleic acid encoding a CasPhi guide RNA and/or a variant CRISPR-Cas effector protein to a cell may include nucleic acid sequences that encode for selectable markers in the target cells, so as to identify cells that have taken up the CasPhi guide RNA and/or CasPhi protein.

A nucleic acid comprising a nucleotide sequence encoding a variant CRISPR-Cas effector polypeptide, or a fusion polypeptide, is in some cases an RNA. Thus, a fusion protein of the present disclosure can be introduced into cells as RNA encoding the fusion protein. Methods of introducing RNA into cells are known in the art and may include, for example, direct injection, transfection, or any other method used for the introduction of DNA. A variant CRISPR-Cas effector protein may instead be provided to cells as a polypeptide. Such a polypeptide may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza hemagglutinin (HA) domain; and other polypeptides that aid in production, e.g. IF2 domain, GST domain, GRPE domain, and the like. The polypeptide may be formulated for improved stability. For example, the peptides may be PEGylated, where the polyethyleneoxy group provides for enhanced lifetime in the blood stream.

Additionally or alternatively, a variant CRISPR-Cas effector polypeptide of the present disclosure, or a fusion polypeptide of the present disclosure may be fused to a polypeptide permeant domain to promote uptake by the cell. A number of permeant domains are known in the art and may be used in the non-integrating polypeptides of the present disclosure, including peptides, peptidomimetics, and non-peptide carriers. For example, a permeant peptide may be derived from the third alpha helix of Drosophila melanogaster transcription factor Antennapedia, referred to as penetratin, which comprises the amino acid sequence RQIKIWFQNRRMKWKK (SEQ ID NO:125). As another example, the permeant peptide comprises the HIV-1 tat basic region amino acid sequence, which may include, for example, amino acids 49-57 of naturally occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-9 and 446; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002). The site at which the fusion is made may be selected in order to optimize the biological activity, secretion or binding characteristics of the polypeptide. The optimal site will be determined by routine experimentation.

As noted above, in some cases, the target cell is a plant cell. Numerous methods for transforming chromosomes or plastids in a plant cell with a recombinant nucleic acid are known in the art, which can be used according to methods of the present application to produce a transgenic plant cell and/or a transgenic plant. Any suitable method or technique for transformation of a plant cell known in the art can be used. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants. Other methods for plant transformation, such as microinjection, electroporation, vacuum infiltration, pressure, sonication, silicon carbide fiber agitation, PEG-mediated transformation, etc., are also known in the art. Transgenic plants produced by these transformation methods can be chimeric or non-chimeric for the transformation event depending on the methods and explants used.

Methods of transforming plant cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958. Additional methods for transforming plants can be found in, for example, Compendium of Transgenic Crop Plants (2009) Blackwell Publishing. Any appropriate method known to those skilled in the art can be used to transform a plant cell with any of the nucleic acids provided herein.

A variant CRISPR-Cas effector polypeptide of the present disclosure, or a fusion polypeptide of the present disclosure may be produced in vitro or by eukaryotic cells or by prokaryotic cells, and it may be further processed by unfolding, e.g. heat denaturation, dithiothreitol reduction, etc. and may be further refolded, using methods known in the art.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also suitable for inclusion in embodiments of the present disclosure are nucleic acids (e.g., encoding a CasPhi guide RNA, encoding a fusion protein, encoding a variant CRISPR-Cas effector of the present disclosure, etc.) and proteins that have been modified using ordinary molecular biological techniques and synthetic chemistry so as to improve their resistance to proteolytic degradation, to change the target sequence specificity, to optimize solubility properties, to alter protein activity (e.g., transcription modulatory activity, enzymatic activity, etc.) or to render them more suitable. Analogs of such polypeptides include those containing residues other than naturally occurring L-amino acids, e.g. D-amino acids or non-naturally occurring synthetic amino acids. D-amino acids may be substituted for some or all of the amino acid residues.

A variant CRISPR-Cas effector polypeptide of the present disclosure may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus, e.g., cysteines can be used to make thioethers, histidines for linking to a metal ion complex, carboxyl groups for forming amides or esters, amino groups for forming amides, and the like.

A variant CRISPR-Cas effector polypeptide of the present disclosure may also be isolated and purified in accordance with conventional methods of recombinant synthesis. A lysate may be prepared of the expression host and the lysate purified using high performance liquid chromatography (HPLC), exclusion chromatography, gel electrophoresis, affinity chromatography, or other purification technique. For the most part, the compositions which are used will comprise 20% or more by weight of the desired product, more usually 75% or more by weight, preferably 95% or more by weight, and for therapeutic purposes, usually 99.5% or more by weight, in relation to contaminants related to the method of preparation of the product and its purification. Usually, the percentages will be based upon total protein. Thus, in some cases, a variant CRISPR-Cas effector polypeptide, or a fusion polypeptide, of the present disclosure is at least 80% pure, at least 85% pure, at least 90% pure, at least 95% pure, at least 98% pure, or at least 99% pure (e.g., free of contaminants, proteins other than the variant CRISPR-Cas effector or fusion protein, or other macromolecules, etc.).

To induce cleavage or any desired modification to a target nucleic acid (e.g., genomic DNA), or any desired modification to a polypeptide associated with target nucleic acid, the CasPhi guide RNA and/or the variant CRISPR-Cas effector polypeptide of the present disclosure and/or the donor template sequence, whether they be introduced as nucleic acids or polypeptides, are provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The agent(s) may be provided to the subject cells one or more times, e.g. one time, twice, three times, or more than three times, and the cells allowed to incubate with the agent(s) for some amount of time following each contacting event e.g. 16-24 hours, after which time the media is replaced with fresh media and the cells are cultured further.

In cases in which two or more different targeting complexes are provided to the cell (e.g., two different CasPhi guide RNAs that are complementary to different sequences within the same or different target nucleic acid), the complexes may be provided simultaneously (e.g. as two polypeptides and/or nucleic acids), or delivered simultaneously. Alternatively, they may be provided consecutively, e.g. the targeting complex being provided first, followed by the second targeting complex, etc. or vice versa.

To improve the delivery of a DNA vector into a target cell, the DNA can be protected from damage and its entry into the cell facilitated, for example, by using lipoplexes and polyplexes. Thus, in some cases, a nucleic acid of the present disclosure (e.g., a recombinant expression vector of the present disclosure) can be covered with lipids in an organized structure like a micelle or a liposome. When the organized structure is complexed with DNA it is called a lipoplex. There are three types of lipids, anionic (negatively-charged), neutral, or cationic (positively-charged). Lipoplexes that utilize cationic lipids have proven utility for gene transfer. Cationic lipids, due to their positive charge, naturally complex with the negatively charged DNA. Also, as a result of their charge, they interact with the cell membrane. Endocytosis of the lipoplex then occurs, and the DNA is released into the cytoplasm. The cationic lipids also protect against degradation of the DNA by the cell.

Complexes of polymers with DNA are called polyplexes. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis) such as inactivated adenovirus must occur. However, this is not always the case; polymers such as polyethylenimine have their own method of endosome disruption as does chitosan and trimethylchitosan.

Dendrimers, a highly branched macromolecule with a spherical shape, may be also be used to genetically modify stem cells. The surface of the dendrimer particle may be functionalized to alter its properties. In particular, it is possible to construct a cationic dendrimer (i.e., one with a positive surface charge). When in the presence of genetic material such as a DNA plasmid, charge complementarity leads to a temporary association of the nucleic acid with the cationic dendrimer. On reaching its destination, the dendrimer-nucleic acid complex can be taken up into a cell by endocytosis.

In some cases, a nucleic acid of the disclosure (e.g., an expression vector) includes an insertion site for a guide sequence of interest. For example, a nucleic acid can include an insertion site for a guide sequence of interest, where the insertion site is immediately adjacent to a nucleotide sequence encoding the portion of a CasPhi guide RNA that does not change when the guide sequence is changed to hybridized to a desired target sequence (e.g., sequences that contribute to the variant CRISPR-Cas effector polypeptide-binding aspect of the guide RNA, e.g., the sequences that contribute to the dsRNA duplex(es) of the CasPhi guide RNA—this portion of the guide RNA can also be referred to as the ‘scaffold’ or ‘constant region’ of the guide RNA). Thus, in some cases, a subject nucleic acid (e.g., an expression vector) includes a nucleotide sequence encoding a CasPhi guide RNA, except that the portion encoding the guide sequence portion of the guide RNA is an insertion sequence (an insertion site). An insertion site is any nucleotide sequence used for the insertion of the desired sequence. “Insertion sites” for use with various technologies are known to those of ordinary skill in the art and any convenient insertion site can be used. An insertion site can be for any method for manipulating nucleic acid sequences. For example, in some cases the insertion site is a multiple cloning site (MCS) (e.g., a site including one or more restriction enzyme recognition sequences), a site for ligation independent cloning, a site for recombination based cloning (e.g., recombination based on att sites), a nucleotide sequence recognized by a CRISPR/Cas (e.g. Cas9) based technology, and the like.

An insertion site can be any desirable length, and can depend on the type of insertion site (e.g., can depend on whether (and how many) the site includes one or more restriction enzyme recognition sequences, whether the site includes a target site for a variant CRISPR/Cas protein of the present disclosure, etc.). In some cases, an insertion site of a subject nucleic acid is 3 or more nucleotides (nt) in length (e.g., 5 or more, 8 or more, 10 or more, 15 or more, 17 or more, 18 or more, 19 or more, 20 or more or 25 or more, or 30 or more nt in length). In some cases, the length of an insertion site of a subject nucleic acid has a length in a range of from 2 to 50 nucleotides (nt) (e.g., from 2 to 40 nt, from 2 to 30 nt, from 2 to 25 nt, from 2 to 20 nt, from 5 to 50 nt, from 5 to 40 nt, from 5 to 30 nt, from 5 to 25 nt, from 5 to 20 nt, from 10 to 50 nt, from 10 to 40 nt, from 10 to 30 nt, from 10 to 25 nt, from 10 to 20 nt, from 17 to 50 nt, from 17 to 40 nt, from 17 to 30 nt, from 17 to 25 nt). In some cases, the length of an insertion site of a subject nucleic acid has a length in a range of from 5 to 40 nt.

### Nucleic Acid Modifications

In some embodiments, a subject nucleic acid (e.g., a CasPhi guide RNA) has one or more modifications, e.g., a base modification, a backbone modification, etc., to provide the nucleic acid with a new or enhanced feature (e.g., improved stability). A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Suitable nucleic acid modifications include, but are not limited to: 2′Omethyl modified nucleotides, 2′ Fluoro modified nucleotides, locked nucleic acid (LNA) modified nucleotides, peptide nucleic acid (PNA) modified nucleotides, nucleotides with phosphorothioate linkages, and a 5′ cap (e.g., a 7-methylguanylate cap (m7G)). Additional details and additional modifications are described below.

A 2′-O-Methyl modified nucleotide (also referred to as 2′-O-Methyl RNA) is a naturally occurring modification of RNA found in tRNA and other small RNAs that arises as a post-transcriptional modification. Oligonucleotides can be directly synthesized that contain 2′-O-Methyl RNA. This modification increases Tm of RNA:RNA duplexes but results in only small changes in RNA:DNA stability. It is stabile with respect to attack by single-stranded ribonucleases and is typically 5 to 10-fold less susceptible to DNases than DNA. It is commonly used in antisense oligos as a means to increase stability and binding affinity to the target message.

2′ Fluoro modified nucleotides (e.g., 2′ Fluoro bases) have a fluorine modified ribose which increases binding affinity (Tm) and also confers some relative nuclease resistance when compared to native RNA. These modifications are commonly employed in ribozymes and siRNAs to improve stability in serum or other biological fluids.

LNA bases have a modification to the ribose backbone that locks the base in the C3′-endo position, which favors RNA A-type helix duplex geometry. This modification significantly increases Tm and is also very nuclease resistant. Multiple LNA insertions can be placed in an oligo at any position except the 3′-end. Applications have been described ranging from antisense oligos to hybridization probes to SNP detection and allele specific polymerase chain reaction (PCR). Due to the large increase in Tm conferred by LNAs, they also can cause an increase in primer dimer formation as well as self-hairpin formation. In some cases, the number of LNAs incorporated into a single oligo is 10 bases or less.

The phosphorothioate (PS) bond (i.e., a phosphorothioate linkage) substitutes a sulfur atom for a non-bridging oxygen in the phosphate backbone of a nucleic acid (e.g., an oligo). This modification renders the internucleotide linkage resistant to nuclease degradation. Phosphorothioate bonds can be introduced between the last 3-5 nucleotides at the 5′- or 3′-end of the oligo to inhibit exonuclease degradation. Including phosphorothioate bonds within the oligo (e.g., throughout the entire oligo) can help reduce attack by endonucleases as well.

In some cases, a subject nucleic acid has one or more nucleotides that are 2′-O-Methyl modified nucleotides. In some embodiments, a subject nucleic acid (e.g., a dsRNA, a siNA, etc.) has one or more 2′ Fluoro modified nucleotides. In some cases, a subject nucleic acid (e.g., a dsRNA, a siNA, etc.) has one or more LNA bases. In some cases, a subject nucleic acid (e.g., a dsRNA, a siNA, etc.) has one or more nucleotides that are linked by a phosphorothioate bond (i.e., the subject nucleic acid has one or more phosphorothioate linkages). In some cases, a subject nucleic acid (e.g., a dsRNA, a siNA, etc.) has a 5′ cap (e.g., a 7-methylguanylate cap (m7G)). In some cases, a subject nucleic acid (e.g., a dsRNA, a siNA, etc.) has a combination of modified nucleotides. For example, a subject nucleic acid (e.g., a dsRNA, a siNA, etc.) can have a 5′ cap (e.g., a 7-methylguanylate cap (m7G)) in addition to having one or more nucleotides with other modifications (e.g., a 2′-O-Methyl nucleotide and/or a 2′ Fluoro modified nucleotide and/or a LNA base and/or a phosphorothioate linkage).

### Modified Backbones and Modified Internucleoside Linkages

Examples of suitable nucleic acids (e.g., a CasPhi guide RNA) containing modifications include nucleic acids containing modified backbones or non-natural internucleoside linkages. Nucleic acids having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some cases, a subject nucleic acid comprises one or more phosphorothioate and/or heteroatom internucleoside linkages, in particular —CH₂—NHO—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677, the disclosure of which is incorporated herein by reference in its entirety. Suitable amide internucleoside linkages are disclosed in U.S. Pat. No. 5,602,240, the disclosure of which is incorporated herein by reference in its entirety.

Also suitable are nucleic acids having morpholino backbone structures as described in, e.g., U.S. Pat. No. 5,034,506. For example, in some embodiments, a subject nucleic acid comprises a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Mimetics

A subject nucleic acid can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262, the disclosures of which are incorporated herein by reference in their entirety.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506, the disclosure of which is incorporated herein by reference in its entirety. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602, the disclosure of which is incorporated herein by reference in its entirety). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2 (Singh et al., Chem. Commun., 1998, 4, 455-456, the disclosure of which is incorporated herein by reference in its entirety). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (e.g., Wahlestedt et al., Proc. Natl. Acad. Sci. U.S.A., 2000, 97, 5633-5638, the disclosure of which is incorporated herein by reference in its entirety).

The synthesis and preparation of the LNA monomers adenine, cytosine, guanine, 5-methyl-cytosine, thymine and uracil, along with their oligomerization, and nucleic acid recognition properties have been described (e.g., Koshkin et al., Tetrahedron, 1998, 54, 3607-3630, the disclosure of which is incorporated herein by reference in its entirety). LNAs and preparation thereof are also described in WO 98/39352 and WO 99/14226, as well as U.S. applications 20120165514, 20100216983, 20090041809, 20060117410, 20040014959, 20020094555, and 20020086998, the disclosures of which are incorporated herein by reference in their entirety.

### Modified Sugar Moieties

A subject nucleic acid can also include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂)ₙO)ₘH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504, the disclosure of which is incorporated herein by reference in its entirety) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—O CH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

A subject nucleic acid may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993; the disclosures of which are incorporated herein by reference in their entirety. Certain of these nucleobases are useful for increasing the binding affinity of an oligomeric compound. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278; the disclosure of which is incorporated herein by reference in its entirety) and are suitable base substitutions, e.g., when combined with 2′-O-methoxyethyl sugar modifications.

Conjugates

Another possible modification of a subject nucleic acid involves chemically linking to the polynucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N. Y Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277,923-937).

A conjugate may include a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle (e.g., the nucleus). In some embodiments, a PTD is covalently linked to the 3′ end of an exogenous polynucleotide. In some embodiments, a PTD is covalently linked to the 5′ end of an exogenous polynucleotide. Exemplary PTDs include but are not limited to a minimal undecapeptide protein transduction domain (corresponding to residues 47-57 of HIV-1 TAT comprising YGRKKRRQRRR; SEQ ID NO:121); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:122); Transportan GWTLNSAGYLLGKINLKALAALAKKIL (SEQ ID NO:123); KALAWEAKLAKALAKALAKHLAKALAKALKCEA (SEQ ID NO:124); and RQIKIWFQNRRMKWKK (SEQ ID NO:125). Exemplary PTDs include but are not limited to, YGRKKRRQRRR (SEQ ID NO:121), RKKRRQRRR (SEQ ID NO:126); an arginine homopolymer of from 3 arginine residues to 50 arginine residues; Exemplary PTD domain amino acid sequences include, but are not limited to, any of the following: YGRKKRRQRRR (SEQ ID NO:121); RKKRRQRR (SEQ ID NO:127); YARAAARQARA (SEQ ID NO:128); THRLPRRRRRR (SEQ ID NO:129); and GGRRARRRRRR (SEQ ID NO:130). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

Introducing Components into a Target Cell

A CasPhi guide RNA (or a nucleic acid comprising a nucleotide sequence encoding same) and/or a variant CRISPR-Cas effector polypeptide of the present disclosure (or a nucleic acid comprising a nucleotide sequence encoding same) and/or a fusion polypeptide of the present disclosure (or a nucleic acid that includes a nucleotide sequence encoding a fusion polypeptide of the present disclosure) and/or a donor polynucleotide (donor template) can be introduced into a host cell by any of a variety of well-known methods.

Any of a variety of compounds and methods can be used to deliver to a target cell a variant CRISPR-Cas effector system of the present disclosure (e.g., where a variant CRISPR-Cas effector system comprises: a) a variant CRISPR-Cas effector polypeptide of the present disclosure and a CasPhi guide RNA; b) a variant CRISPR-Cas effector polypeptide of the present disclosure, a CasPhi guide RNA, and a donor template nucleic acid; c) a fusion polypeptide of the present disclosure and a CasPhi guide RNA; d) a fusion polypeptide of the present disclosure, a CasPhi guide RNA, and a donor template nucleic acid; e) an mRNA encoding a variant CRISPR-Cas effector polypeptide of the present disclosure; and a CasPhi guide RNA; f) an mRNA encoding a variant CRISPR-Cas effector polypeptide of the present disclosure, a CasPhi guide RNA, and a donor template nucleic acid; g) an mRNA encoding a fusion polypeptide of the present disclosure; and a CasPhi guide RNA; h) an mRNA encoding a fusion polypeptide of the present disclosure, a CasPhi guide RNA, and a donor template nucleic acid; i) a recombinant expression vector comprising a nucleotide sequence encoding a variant CRISPR-Cas effector polypeptide of the present disclosure and a nucleotide sequence encoding a CasPhi guide RNA; j) a recombinant expression vector comprising a nucleotide sequence encoding a variant CRISPR-Cas effector polypeptide of the present disclosure, a nucleotide sequence encoding a CasPhi guide RNA, and a nucleotide sequence encoding a donor template nucleic acid; k) a recombinant expression vector comprising a nucleotide sequence encoding a fusion polypeptide of the present disclosure and a nucleotide sequence encoding a CasPhi guide RNA; l) a recombinant expression vector comprising a nucleotide sequence encoding a fusion polypeptide of the present disclosure, a nucleotide sequence encoding a CasPhi guide RNA, and a nucleotide sequence encoding a donor template nucleic acid; m) a first recombinant expression vector comprising a nucleotide sequence encoding a variant CRISPR-Cas effector polypeptide of the present disclosure, and a second recombinant expression vector comprising a nucleotide sequence encoding a CasPhi guide RNA; n) a first recombinant expression vector comprising a nucleotide sequence encoding a variant CRISPR-Cas effector polypeptide of the present disclosure, and a second recombinant expression vector comprising a nucleotide sequence encoding a CasPhi guide RNA; and a donor template nucleic acid; o) a first recombinant expression vector comprising a nucleotide sequence encoding a fusion polypeptide of the present disclosure, and a second recombinant expression vector comprising a nucleotide sequence encoding a CasPhi guide RNA; p) a first recombinant expression vector comprising a nucleotide sequence encoding a fusion polypeptide of the present disclosure, and a second recombinant expression vector comprising a nucleotide sequence encoding a CasPhi guide RNA; and a donor template nucleic acid; q) a recombinant expression vector comprising a nucleotide sequence encoding a variant CRISPR-Cas effector polypeptide of the present disclosure, a nucleotide sequence encoding a first CasPhi guide RNA, and a nucleotide sequence encoding a second CasPhi guide RNA; or r) a recombinant expression vector comprising a nucleotide sequence encoding a fusion polypeptide of the present disclosure, a nucleotide sequence encoding a first CasPhi guide RNA, and a nucleotide sequence encoding a second CasPhi guide RNA; or some variation of one of (a) through (r). As a non-limiting example, a CasPhi system of the present disclosure can be combined with a lipid. As another non-limiting example, a CasPhi system of the present disclosure can be combined with a particle, or formulated into a particle.

Methods of introducing a nucleic acid into a host cell are known in the art, and any convenient method can be used to introduce a subject nucleic acid (e.g., an expression construct/vector) into a target cell (e.g., prokaryotic cell, eukaryotic cell, plant cell, animal cell, mammalian cell, human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: S0169-409X(12)00283-9. doi: 10.1016/j.addr.2012.09.023), and the like.

In some cases, a variant CRISPR-Cas effector polypeptide of the present disclosure is provided as a nucleic acid (e.g., an mRNA, a DNA, a plasmid, an expression vector, a viral vector, etc.) that encodes the variant CRISPR-Cas effector polypeptide. In some cases, the variant CRISPR-Cas effector polypeptide of the present disclosure is provided directly as a protein (e.g., without an associated guide RNA or with an associate guide RNA, i.e., as a ribonucleoprotein complex). A variant CRISPR-Cas effector polypeptide of the present disclosure can be introduced into a cell (provided to the cell) by any convenient method; such methods are known to those of ordinary skill in the art. As an illustrative example, a variant CRISPR-Cas effector polypeptide of the present disclosure can be injected directly into a cell (e.g., with or without a CasPhi guide RNA or nucleic acid encoding a CasPhi guide RNA, and with or without a donor polynucleotide). As another example, a preformed complex of a variant CRISPR-Cas effector polypeptide of the present disclosure and a CasPhi guide RNA (an RNP) can be introduced into a cell (e.g, eukaryotic cell) (e.g., via injection, via nucleofection; via a protein transduction domain (PTD) conjugated to one or more components, e.g., conjugated to the variant CRISPR-Cas effector protein, conjugated to a guide RNA, conjugated to a variant CRISPR-Cas effector polypeptide of the present disclosure and a guide RNA; etc.).

In some cases, a fusion polypeptide (e.g., a variant CRISPR-Cas effector fused to a fusion partner) of the present disclosure is provided as a nucleic acid (e.g., an mRNA, a DNA, a plasmid, an expression vector, a viral vector, etc.) that encodes the fusion polypeptide. In some cases, the fusion polypeptide of the present disclosure is provided directly as a protein (e.g., without an associated guide RNA or with an associate guide RNA, i.e., as a ribonucleoprotein complex). A fusion polypeptide of the present disclosure can be introduced into a cell (provided to the cell) by any convenient method; such methods are known to those of ordinary skill in the art. As an illustrative example, a fusion polypeptide of the present disclosure can be injected directly into a cell (e.g., with or without nucleic acid encoding a CasPhi guide RNA and with or without a donor polynucleotide). As another example, a preformed complex of a fusion polypeptide of the present disclosure and a CasPhi guide RNA (an RNP) can be introduced into a cell (e.g., via injection, via nucleofection; via a protein transduction domain (PTD) conjugated to one or more components, e.g., conjugated to the fusion protein, conjugated to a guide RNA, conjugated to a fusion polypeptide of the present disclosure and a guide RNA; etc.).

In some cases, a nucleic acid (e.g., a CasPhi guide RNA; a nucleic acid comprising a nucleotide sequence encoding a variant CRISPR-Cas effector polypeptide of the present disclosure; etc.) is delivered to a cell (e.g., a target host cell) and/or a polypeptide (e.g., a variant CRISPR-Cas effector polypeptide; a fusion polypeptide) in a particle, or associated with a particle. In some cases, a variant CRISPR-Cas effector system of the present disclosure is delivered to a cell in a particle, or associated with a particle. The terms “particle” and nanoparticle” can be used interchangeable, as appropriate. A recombinant expression vector comprising a nucleotide sequence encoding a variant CRISPR-Cas effector polypeptide of the present disclosure and/or a CasPhi guide RNA, an mRNA comprising a nucleotide sequence encoding a variant CRISPR-Cas effector polypeptide of the present disclosure, and guide RNA may be delivered simultaneously using particles or lipid envelopes; for instance, a variant CRISPR-Cas effector polypeptide and a CasPhi guide RNA, e.g., as a complex (e.g., a ribonucleoprotein (RNP) complex), can be delivered via a particle, e.g., a delivery particle comprising lipid or lipidoid and hydrophilic polymer, e.g., a cationic lipid and a hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG); and/or wherein the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DMPC 0, PEG 5, Cholesterol 5). For example, a particle can be formed using a multistep process in which a variant CRISPR-Cas effector polypeptide and a CasPhi guideRNA are mixed together, e.g., at a 1:1 molar ratio, e.g., at room temperature, e.g., for 30 minutes, e.g., in sterile, nuclease free 1× phosphate-buffered saline (PBS); and separately, DOTAP, DMPC, PEG, and cholesterol as applicable for the formulation are dissolved in alcohol, e.g., 100% ethanol; and, the two solutions are mixed together to form particles containing the complexes).

A variant CRISPR-Cas effector polypeptide of the present disclosure (or an mRNA comprising a nucleotide sequence encoding a variant CRISPR-Cas effector polypeptide of the present disclosure; or a recombinant expression vector comprising a nucleotide sequence encoding a variant CRISPR-Cas effector polypeptide of the present disclosure) and/or CasPhi guide RNA (or a nucleic acid such as one or more expression vectors encoding the CasPhi guide RNA) may be delivered simultaneously using particles or lipid envelopes. For example, a biodegradable core-shell structured nanoparticle with a poly (β-amino ester) (PBAE) core enveloped by a phospholipid bilayer shell can be used. In some cases, particles/nanoparticles based on self-assembling bioadhesive polymers are used; such particles/nanoparticles may be applied to oral delivery of peptides, intravenous delivery of peptides and nasal delivery of peptides, e.g., to the brain. Other embodiments, such as oral absorption and ocular delivery of hydrophobic drugs are also contemplated. A molecular envelope technology, which involves an engineered polymer envelope which is protected and delivered to the site of the disease, can be used. Doses of about 5 mg/kg can be used, with single or multiple doses, depending on various factors, e.g., the target tissue.

Lipidoid compounds (e.g., as described in US patent application 20110293703) are also useful in the administration of polynucleotides, and can be used to deliver a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure (e.g., where a variant CRISPR-Cas effector system comprises: a) a variant CRISPR-Cas effector polypeptide of the present disclosure and a CasPhi guide RNA; b) a variant CRISPR-Cas effector polypeptide of the present disclosure, a CasPhi guide RNA, and a donor template nucleic acid; c) a fusion polypeptide of the present disclosure and a CasPhi guide RNA; d) a fusion polypeptide of the present disclosure, a CasPhi guide RNA, and a donor template nucleic acid; e) an mRNA encoding a variant CRISPR-Cas effector polypeptide of the present disclosure; and a CasPhi guide RNA; f) an mRNA encoding a variant CRISPR-Cas effector polypeptide of the present disclosure, a CasPhi guide RNA, and a donor template nucleic acid; g) an mRNA encoding a fusion polypeptide of the present disclosure; and a CasPhi guide RNA; h) an mRNA encoding a fusion polypeptide of the present disclosure, a CasPhi guide RNA, and a donor template nucleic acid; i) a recombinant expression vector comprising a nucleotide sequence encoding a variant CRISPR-Cas effector polypeptide of the present disclosure and a nucleotide sequence encoding a CasPhi guide RNA; j) a recombinant expression vector comprising a nucleotide sequence encoding a variant CRISPR-Cas effector polypeptide of the present disclosure, a nucleotide sequence encoding a CasPhi guide RNA, and a nucleotide sequence encoding a donor template nucleic acid; k) a recombinant expression vector comprising a nucleotide sequence encoding a fusion polypeptide of the present disclosure and a nucleotide sequence encoding a CasPhi guide RNA; l) a recombinant expression vector comprising a nucleotide sequence encoding a fusion polypeptide of the present disclosure, a nucleotide sequence encoding a CasPhi guide RNA, and a nucleotide sequence encoding a donor template nucleic acid; m) a first recombinant expression vector comprising a nucleotide sequence encoding a variant CRISPR-Cas effector polypeptide of the present disclosure, and a second recombinant expression vector comprising a nucleotide sequence encoding a CasPhi guide RNA; n) a first recombinant expression vector comprising a nucleotide sequence encoding a variant CRISPR-Cas effector polypeptide of the present disclosure, and a second recombinant expression vector comprising a nucleotide sequence encoding a CasPhi guide RNA; and a donor template nucleic acid; o) a first recombinant expression vector comprising a nucleotide sequence encoding a fusion polypeptide of the present disclosure, and a second recombinant expression vector comprising a nucleotide sequence encoding a CasPhi guide RNA; p) a first recombinant expression vector comprising a nucleotide sequence encoding a fusion polypeptide of the present disclosure, and a second recombinant expression vector comprising a nucleotide sequence encoding a CasPhi guide RNA; and a donor template nucleic acid; q) a recombinant expression vector comprising a nucleotide sequence encoding a variant CRISPR-Cas effector polypeptide of the present disclosure, a nucleotide sequence encoding a first CasPhi guide RNA, and a nucleotide sequence encoding a second CasPhi guide RNA; or r) a recombinant expression vector comprising a nucleotide sequence encoding a fusion polypeptide of the present disclosure, a nucleotide sequence encoding a first CasPhi guide RNA, and a nucleotide sequence encoding a second CasPhi guide RNA; or some variation of one of (a) through (r). In one aspect, the aminoalcohol lipidoid compounds are combined with an agent to be delivered to a cell or a subject to form microparticles, nanoparticles, liposomes, or micelles. The aminoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

A poly(beta-amino alcohol) (PBAA) can be used to deliver a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell. US Patent Publication No. 20130302401 relates to a class of poly(beta-amino alcohols) (PBAAs) that has been prepared using combinatorial polymerization.

Sugar-based particles may be used, for example GalNAc, as described with reference to WO2014118272 (incorporated herein by reference) and Nair, J K et al., 2014, Journal of the American Chemical Society 136 (49), 16958-16961) can be used to deliver a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell.

In some cases, lipid nanoparticles (LNPs) are used to deliver a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell. Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). Preparation of LNPs and is described in, e.g., Rosin et al. (2011) Molecular Therapy 19:1286-2200). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), and R-3-[(.omega.-methoxy-poly(ethylene glycol)2000) carbamoyl]-1,2-dimyristyloxlpropyl-3-amine (PEG-C-DOMG) may be used. A nucleic acid (e.g., a CasPhi guide RNA; a nucleic acid of the present disclosure; etc.) may be encapsulated in LNPs containing DLinDAP, DLinDMA, DLinK-DMA, and DLinKC2-DMA (cationic lipid:DSPC:CHOL: PEGS-DMG or PEG-C-DOMG at 40:10:40:10 molar ratios). In some cases, 0.2% SP-DiOC18 is incorporated.

Spherical Nucleic Acid (SNA™) constructs and other nanoparticles (particularly gold nanoparticles) can be used to deliver a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell. See, e.g., Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19): 7625-7630, Jensen et al., Sci. Transl. Med. 5, 209ra152 (2013) and Mirkin, et al., Small, 10:186-192.

Self-assembling nanoparticles with RNA may be constructed with polyethyleneimine (PEI) that is PEGylated with an Arg-Gly-Asp (RGD) peptide ligand attached at the distal end of the polyethylene glycol (PEG).

In general, a “nanoparticle” refers to any particle having a diameter of less than 1000 nm. In some cases, nanoparticles suitable for use in delivering a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell have a diameter of 500 nm or less, e.g., from 25 nm to 35 nm, from 35 nm to 50 nm, from 50 nm to 75 nm, from 75 nm to 100 nm, from 100 nm to 150 nm, from 150 nm to 200 nm, from 200 nm to 300 nm, from 300 nm to 400 nm, or from 400 nm to 500 nm. In some cases, nanoparticles suitable for use in delivering a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell have a diameter of from 25 nm to 200 nm. In some cases, nanoparticles suitable for use in delivering a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell have a diameter of 100 nm or less In some cases, nanoparticles suitable for use in delivering a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell have a diameter of from 35 nm to 60 nm.

Nanoparticles suitable for use in delivering a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically below 10 nm) that quantization of electronic energy levels occurs. Such nanoscale particles are used in biomedical applications as drug carriers or imaging agents and may be adapted for similar purposes in the present disclosure.

Semi-solid and soft nanoparticles are also suitable for use in delivering a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell. A prototype nanoparticle of semi-solid nature is the liposome.

In some cases, an exosome is used to deliver a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell. Exosomes are endogenous nano-vesicles that transport RNAs and proteins, and which can deliver RNA to the brain and other target organs.

In some cases, a liposome is used to deliver a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell. Liposomes are spherical vesicle structures composed of a uni- or multilamellar lipid bilayer surrounding internal aqueous compartments and a relatively impermeable outer lipophilic phospholipid bilayer. Liposomes can be made from several different types of lipids; however, phospholipids are most commonly used to generate liposomes. Although liposome formation is spontaneous when a lipid film is mixed with an aqueous solution, it can also be expedited by applying force in the form of shaking by using a homogenizer, sonicator, or an extrusion apparatus. Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. A liposome formulation may be mainly comprised of natural phospholipids and lipids such as 1,2-distearoyl-sn-glycero-3-phosphatidyl choline (DSPC), sphingomyelin, egg phosphatidylcholines and monosialoganglioside.

A stable nucleic-acid-lipid particle (SNALP) can be used to deliver a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell. The SNALP formulation may contain the lipids 3-N-[(methoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio. The SNALP liposomes may be prepared by formulating D-Lin-DMA and PEG-C-DMA with distearoylphosphatidylcholine (DSPC), Cholesterol and siRNA using a 25:1 lipid/siRNA ratio and a 48/40/10/2 molar ratio of Cholesterol/D-Lin-DMA/DSPC/PEG-C-DMA. The resulting SNALP liposomes can be about 80-100 nm in size. A SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol)2000)carbamoyl]-1,2-dimyrestyloxypropylamine, and cationic 1,2-dilinoleyloxy-3-N,N dimethylaminopropane. A SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinoleyloxy-3-(N;N-dimethyl)aminopropane (DLinDMA).

Other cationic lipids, such as amino lipid 2,2-dilinoleyl-4-dimethylaminoethyl-[1,3]-dioxolane (DLin-KC2-DMA) can be used to deliver a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell. A preformed vesicle with the following lipid composition may be contemplated: amino lipid, distearoylphosphatidylcholine (DSPC), cholesterol and (R)-2,3-bis(octadecyloxy) propyl-1-(methoxy poly(ethylene glycol)2000)propylcarbamate (PEG-lipid) in the molar ratio 40/10/40/10, respectively, and a FVII siRNA/total lipid ratio of approximately 0.05 (w/w). To ensure a narrow particle size distribution in the range of 70-90 nm and a low polydispersity index of 0.11.+−0.0.04 (n=56), the particles may be extruded up to three times through 80 nm membranes prior to adding the guide RNA. Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

Lipids may be formulated with a variant CRISPR-Cas effector system of the present disclosure or component(s) thereof or nucleic acids encoding the same to form lipid nanoparticles (LNPs). Suitable lipids include, but are not limited to, DLin-KC2-DMA4, C12-200 and colipids disteroylphosphatidyl choline, cholesterol, and PEG-DMG may be formulated with a CasPhi system, or component thereof, of the present disclosure, using a spontaneous vesicle formation procedure. The component molar ratio may be about 50/10/38.5/1.5 (DLin-KC2-DMA or C12-200/disteroylphosphatidyl choline/cholesterol/PEG-DMG).

A variant CRISPR-Cas effector system of the present disclosure, or a component thereof, may be delivered encapsulated in PLGA microspheres such as that further described in US published applications 20130252281 and 20130245107 and 20130244279.

Supercharged proteins can be used to deliver a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell. Supercharged proteins are a class of engineered or naturally occurring proteins with unusually high positive or negative net theoretical charge. Both supernegatively and superpositively charged proteins exhibit the ability to withstand thermally or chemically induced aggregation. Superpositively charged proteins are also able to penetrate mammalian cells. Associating cargo with these proteins, such as plasmid DNA, RNA, or other proteins, can enable the functional delivery of these macromolecules into mammalian cells both in vitro and in vivo.

Cell Penetrating Peptides (CPPs) can be used to deliver a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell. CPPs typically have an amino acid composition that either contains a high relative abundance of positively charged amino acids such as lysine or arginine or has sequences that contain an alternating pattern of polar/charged amino acids and non-polar, hydrophobic amino acids.

An implantable device can be used to deliver a variant CRISPR-Cas effector polypeptide of the present disclosure, a fusion polypeptide of the present disclosure, an RNP of the present disclosure, a nucleic acid of the present disclosure (e.g., a CasPhi guide RNA, a nucleic acid encoding a CasPhi guide RNA, a nucleic acid encoding variant CRISPR-Cas effector polypeptide, a donor template, and the like), or a variant CRISPR-Cas effector system of the present disclosure, to a target cell (e.g., a target cell in vivo, where the target cell is a target cell in circulation, a target cell in a tissue, a target cell in an organ, etc.). An implantable device suitable for use in delivering a variant CRISPR-Cas effector polypeptide of the present disclosure, a fusion polypeptide of the present disclosure, an RNP of the present disclosure, a nucleic acid of the present disclosure, or a variant CRISPR-Cas effector system of the present disclosure, to a target cell (e.g., a target cell in vivo, where the target cell is a target cell in circulation, a target cell in a tissue, a target cell in an organ, etc.) can include a container (e.g., a reservoir, a matrix, etc.) that comprises the variant CRISPR-Cas effector polypeptide, the fusion polypeptide, the RNP, or the variant CRISPR-Cas effector system (or component thereof, e.g., a nucleic acid of the present disclosure).

A suitable implantable device can comprise a polymeric substrate, such as a matrix for example, that is used as the device body, and in some cases additional scaffolding materials, such as metals or additional polymers, and materials to enhance visibility and imaging. An implantable delivery device can be advantageous in providing release locally and over a prolonged period, where the polypeptide and/or nucleic acid to be delivered is released directly to a target site, e.g., the extracellular matrix (ECM), the vasculature surrounding a tumor, a diseased tissue, etc. Suitable implantable delivery devices include devices suitable for use in delivering to a cavity such as the abdominal cavity and/or any other type of administration in which the drug delivery system is not anchored or attached, comprising a biostable and/or degradable and/or bioabsorbable polymeric substrate, which may for example optionally be a matrix. In some cases, a suitable implantable drug delivery device comprises degradable polymers, wherein the main release mechanism is bulk erosion. In some cases, a suitable implantable drug delivery device comprises non degradable, or slowly degraded polymers, wherein the main release mechanism is diffusion rather than bulk erosion, so that the outer part functions as membrane, and its internal part functions as a drug reservoir, which practically is not affected by the surroundings for an extended period (for example from about a week to about a few months). Combinations of different polymers with different release mechanisms may also optionally be used. The concentration gradient at the can be maintained effectively constant during a significant period of the total releasing period, and therefore the diffusion rate is effectively constant (termed “zero mode” diffusion). By the term “constant” it is meant a diffusion rate that is maintained above the lower threshold of therapeutic effectiveness, but which may still optionally feature an initial burst and/or may fluctuate, for example increasing and decreasing to a certain degree. The diffusion rate can be so maintained for a prolonged period, and it can be considered constant to a certain level to optimize the therapeutically effective period, for example the effective silencing period.

In some cases, the implantable delivery system is designed to shield the nucleotide based therapeutic agent from degradation, whether chemical in nature or due to attack from enzymes and other factors in the body of the subject.

The site for implantation of the device, or target site, can be selected for maximum therapeutic efficacy. For example, a delivery device can be implanted within or in the proximity of a tumor environment, or the blood supply associated with a tumor. The target location can be, e.g.: 1) the brain at degenerative sites like in Parkinson or Alzheimer disease at the basal ganglia, white and gray matter; 2) the spine, as in the case of amyotrophic lateral sclerosis (ALS); 3) uterine cervix; 4) active and chronic inflammatory joints; 5) dermis as in the case of psoriasis; 7) sympathetic and sensoric nervous sites for analgesic effect; 7) a bone; 8) a site of acute or chronic infection; 9) Intra vaginal; 10) Inner ear—auditory system, labyrinth of the inner ear, vestibular system; 11) Intra tracheal; 12) Intra-cardiac; coronary, epicardiac; 13) urinary tract or bladder; 14) biliary system; 15) parenchymal tissue including and not limited to the kidney, liver, spleen; 16) lymph nodes; 17) salivary glands; 18) dental gums; 19) Intra-articular (into joints); 20) Intra-ocular; 21) Brain tissue; 22) Brain ventricles; 23) Cavities, including abdominal cavity (for example but without limitation, for ovary cancer); 24) Intra esophageal; and 25) Intra rectal; and 26) into the vasculature.

The method of insertion, such as implantation, may optionally already be used for other types of tissue implantation and/or for insertions and/or for sampling tissues, optionally without modifications, or alternatively optionally only with non-major modifications in such methods. Such methods optionally include but are not limited to brachytherapy methods, biopsy, endoscopy with and/or without ultrasound, such as stereotactic methods into the brain tissue, laparoscopy, including implantation with a laparoscope into joints, abdominal organs, the bladder wall and body cavities.

### Modified Host Cells

The present disclosure provides a modified cell comprising a variant CRISPR-Cas effector polypeptide of the present disclosure and/or a nucleic acid comprising a nucleotide sequence encoding a variant CRISPR-Cas effector polypeptide of the present disclosure. The present disclosure provides a modified cell (e.g., a genetically modified cell) comprising nucleic acid comprising a nucleotide sequence encoding a variant CRISPR-Cas effector polypeptide of the present disclosure. The present disclosure provides a genetically modified cell that is genetically modified with an mRNA comprising a nucleotide sequence encoding a variant CRISPR-Cas effector polypeptide of the present disclosure. The present disclosure provides a genetically modified cell that is genetically modified with a recombinant expression vector comprising a nucleotide sequence encoding a variant CRISPR-Cas effector polypeptide of the present disclosure. The present disclosure provides a genetically modified cell that is genetically modified with a recombinant expression vector comprising: a) a nucleotide sequence encoding a variant CRISPR-Cas effector polypeptide of the present disclosure; and b) a nucleotide sequence encoding a CasPhi guide RNA of the present disclosure. The present disclosure provides a genetically modified cell that is genetically modified with a recombinant expression vector comprising: a) a nucleotide sequence encoding a variant CRISPR-Cas effector polypeptide of the present disclosure; b) a nucleotide sequence encoding a CasPhi guide RNA of the present disclosure; and c) a nucleotide sequence encoding a donor template.

The present disclosure provides a modified cell comprising a fusion polypeptide of the present disclosure and/or a nucleic acid comprising a nucleotide sequence encoding a fusion polypeptide of the present disclosure. The present disclosure provides a modified cell (e.g., a genetically modified cell) comprising nucleic acid comprising a nucleotide sequence encoding a fusion polypeptide of the present disclosure. The present disclosure provides a genetically modified cell that is genetically modified with an mRNA comprising a nucleotide sequence encoding a fusion polypeptide of the present disclosure. The present disclosure provides a genetically modified cell that is genetically modified with a recombinant expression vector comprising a nucleotide sequence encoding a fusion polypeptide of the present disclosure. The present disclosure provides a genetically modified cell that is genetically modified with a recombinant expression vector comprising: a) a nucleotide sequence encoding a fusion polypeptide of the present disclosure; and b) a nucleotide sequence encoding a CasPhi guide RNA of the present disclosure. The present disclosure provides a genetically modified cell that is genetically modified with a recombinant expression vector comprising: a) a nucleotide sequence encoding a fusion polypeptide of the present disclosure; b) a nucleotide sequence encoding a CasPhi guide RNA of the present disclosure; and c) a nucleotide sequence encoding a donor template.

A cell that serves as a recipient for a variant CRISPR-Cas effector polypeptide of the present disclosure and/or a nucleic acid comprising a nucleotide sequence encoding a variant CRISPR-Cas effector polypeptide of the present disclosure and/or a CasPhi guide RNA of the present disclosure, and/or a fusion polypeptide of the present disclosure, can be any of a variety of cells, including, e.g., in vitro cells; in vivo cells; ex vivo cells; primary cells; cancer cells; animal cells; plant cells; algal cells; fungal cells; etc. A cell that serves as a recipient for a variant CRISPR-Cas effector polypeptide of the present disclosure and/or a nucleic acid comprising a nucleotide sequence encoding a variant CRISPR-Cas effector polypeptide of the present disclosure and/or a CasPhi guide RNA of the present disclosure and/or a fusion polypeptide of the present disclosure is referred to as a “host cell” or a “target cell.” A host cell or a target cell can be a recipient of a variant CRISPR-Cas effector system of the present disclosure. A host cell or a target cell can be a recipient of an RNP of the present disclosure. A host cell or a target cell can be a recipient of a single component of a variant CRISPR-Cas effector system of the present disclosure.

Non-limiting examples of cells (target cells) include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomonas reinhardtii, Nannochloropsis gaditana, Chlorella pyrenoidosa, Sargassum patens, C. agardhii, and the like), seaweeds (e.g. kelp) a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some cases, the cell is a cell that does not originate from a natural organism (e.g., the cell can be a synthetically made cell; also referred to as an artificial cell).

A cell can be an in vitro cell (e.g., established cultured cell line). A cell can be an ex vivo cell (cultured cell from an individual). A cell can be and in vivo cell (e.g., a cell in an individual). A cell can be an isolated cell. A cell can be a cell inside of an organism. A cell can be an organism. A cell can be a cell in a cell culture (e.g., in vitro cell culture). A cell can be one of a collection of cells. A cell can be a prokaryotic cell or derived from a prokaryotic cell. A cell can be a bacterial cell or can be derived from a bacterial cell. A cell can be an archaeal cell or derived from an archaeal cell. A cell can be a eukaryotic cell or derived from a eukaryotic cell. A cell can be a plant cell or derived from a plant cell. A cell can be an animal cell or derived from an animal cell. A cell can be an invertebrate cell or derived from an invertebrate cell. A cell can be a vertebrate cell or derived from a vertebrate cell. A cell can be a mammalian cell or derived from a mammalian cell. A cell can be a rodent cell or derived from a rodent cell. A cell can be a human cell or derived from a human cell. A cell can be a microbe cell or derived from a microbe cell. A cell can be a fungi cell or derived from a fungi cell. A cell can be an insect cell. A cell can be an arthropod cell. A cell can be a protozoan cell. A cell can be a helminth cell.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGC1A.

In some instances, the stem cell is a hematopoietic stem cell (HSC). HSCs are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other instances, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other instances, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

A cell is in some cases a plant cell. A plant cell can be a cell of a monocotyledon. A cell can be a cell of a dicotyledon.

In some cases, the cell is a plant cell. For example, the cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, chinese artichoke (crosnes), chinese cabbage, chinese celery, chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (chinese mustard), gailon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf—green), lettuce (oak leaf—red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

In some cases, the plant cell is a cell of a plant component such as a leaf, a stem, a root, a seed, a flower, pollen, an anther, an ovule, a pedicel, a fruit, a meristem, a cotyledon, a hypocotyl, a pod, an embryo, endosperm, an explant, a callus, or a shoot.

A cell is in some cases an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopterygota, Plecoptera, Embioptera, Orthoptera, Zoraptera, Dermaptera, Dictyoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

A cell is in some cases an insect cell. For example, in some cases, the cell is a cell of a mosquito, a grasshopper, a true bug, a fly, a flea, a bee, a wasp, an ant, a louse, a moth, or a beetle.

### Kits

The present disclosure provides a kit comprising a variant CRISPR-Cas effector system of the present disclosure, or a component of a variant CRISPR-Cas effector system of the present disclosure.

A kit of the present disclosure can comprise: a) a variant CRISPR-Cas effector polypeptide of the present disclosure and a CasPhi guide RNA; b) a variant CRISPR-Cas effector polypeptide of the present disclosure, a CasPhi guide RNA, and a donor template nucleic acid; c) a fusion polypeptide of the present disclosure and a CasPhi guide RNA; d) a fusion polypeptide of the present disclosure, a CasPhi guide RNA, and a donor template nucleic acid; e) an mRNA encoding a variant CRISPR-Cas effector polypeptide of the present disclosure; and a CasPhi guide RNA; f) an mRNA encoding a variant CRISPR-Cas effector polypeptide of the present disclosure, a CasPhi guide RNA, and a donor template nucleic acid; g) an mRNA encoding a fusion polypeptide of the present disclosure; and a CasPhi guide RNA; h) an mRNA encoding a fusion polypeptide of the present disclosure, a CasPhi guide RNA, and a donor template nucleic acid; i) a recombinant expression vector comprising a nucleotide sequence encoding a variant CRISPR-Cas effector polypeptide of the present disclosure and a nucleotide sequence encoding a CasPhi guide RNA; j) a recombinant expression vector comprising a nucleotide sequence encoding a variant CRISPR-Cas effector polypeptide of the present disclosure, a nucleotide sequence encoding a CasPhi guide RNA, and a nucleotide sequence encoding a donor template nucleic acid; k) a recombinant expression vector comprising a nucleotide sequence encoding a fusion polypeptide of the present disclosure and a nucleotide sequence encoding a CasPhi guide RNA; l) a recombinant expression vector comprising a nucleotide sequence encoding a fusion polypeptide of the present disclosure, a nucleotide sequence encoding a CasPhi guide RNA, and a nucleotide sequence encoding a donor template nucleic acid; m) a first recombinant expression vector comprising a nucleotide sequence encoding a variant CRISPR-Cas effector polypeptide of the present disclosure, and a second recombinant expression vector comprising a nucleotide sequence encoding a CasPhi guide RNA; n) a first recombinant expression vector comprising a nucleotide sequence encoding a variant CRISPR-Cas effector polypeptide of the present disclosure, and a second recombinant expression vector comprising a nucleotide sequence encoding a CasPhi guide RNA; and a donor template nucleic acid; o) a first recombinant expression vector comprising a nucleotide sequence encoding a CasPhi fusion polypeptide of the present disclosure, and a second recombinant expression vector comprising a nucleotide sequence encoding a CasPhi guide RNA; p) a first recombinant expression vector comprising a nucleotide sequence encoding a fusion polypeptide of the present disclosure, and a second recombinant expression vector comprising a nucleotide sequence encoding a CasPhi guide RNA; and a donor template nucleic acid; q) a recombinant expression vector comprising a nucleotide sequence encoding a variant CRISPR-Cas effector polypeptide of the present disclosure, a nucleotide sequence encoding a first CasPhi guide RNA, and a nucleotide sequence encoding a second CasPhi guide RNA; or r) a recombinant expression vector comprising a nucleotide sequence encoding a fusion polypeptide of the present disclosure, a nucleotide sequence encoding a first CasPhi guide RNA, and a nucleotide sequence encoding a second CasPhi guide RNA; or some variation of one of (a) through (r).

A kit of the present disclosure can comprise: a) a component, as described above, of a variant CRISPR-Cas effector system of the present disclosure, or can comprise a variant CRISPR-Cas effector system of the present disclosure; and b) one or more additional reagents, e.g., i) a buffer; ii) a protease inhibitor; iii) a nuclease inhibitor; iv) a reagent required to develop or visualize a detectable label; v) a positive and/or negative control target DNA; vi) a positive and/or negative control CasPhi guide RNA; and the like. A kit of the present disclosure can comprise: a) a component, as described above, of a variant CRISPR-Cas effector system of the present disclosure, or can comprise a variant CRISPR-Cas effector system of the present disclosure; and b) a therapeutic agent.

A kit of the present disclosure can comprise a recombinant expression vector comprising: a) an insertion site for inserting a nucleic acid comprising a nucleotide sequence encoding a portion of a CasPhi guide RNA that hybridizes to a target nucleotide sequence in a target nucleic acid; and b) a nucleotide sequence encoding the variant CRISPR-Cas effector polypeptide-binding portion of a CasPhi guide RNA. A kit of the present disclosure can comprise a recombinant expression vector comprising: a) an insertion site for inserting a nucleic acid comprising a nucleotide sequence encoding a portion of a CasPhi guide RNA that hybridizes to a target nucleotide sequence in a target nucleic acid; b) a nucleotide sequence encoding the variant CRISPR-Cas effector polypeptide-binding portion of a CasPhi guide RNA; and c) a nucleotide sequence encoding a variant CRISPR-Cas effector polypeptide of the present disclosure.

### Utility

A variant CRISPR-Cas effector polypeptide of the present disclosure, or a fusion polypeptide of the present disclosure, finds use in a variety of methods (e.g., in combination with a CasPhi guide RNA and in some cases further in combination with a donor template). For example, a variant CRISPR-Cas effector polypeptide of the present disclosure can be used to (i) modify (e.g., cleave, e.g., nick; methylate; etc.) target nucleic acid (DNA or RNA; single stranded or double stranded); (ii) modulate transcription of a target nucleic acid; (iii) label a target nucleic acid; (iv) bind a target nucleic acid (e.g., for purposes of isolation, labeling, imaging, tracking, etc.); (v) modify a polypeptide (e.g., a histone) associated with a target nucleic acid; and the like. Thus, the present disclosure provides a method of modifying a target nucleic acid. In some cases, a method of the present disclosure for modifying a target nucleic acid comprises contacting the target nucleic acid with: a) a variant CRISPR-Cas effector polypeptide of the present disclosure; and b) one or more (e.g., two) CasPhi guide RNAs. In some cases, a method of the present disclosure for modifying a target nucleic acid comprises contacting the target nucleic acid with: a) a variant CRISPR-Cas effector polypeptide of the present disclosure; b) a CasPhi guide RNA; and c) a donor nucleic acid (e.g., a donor template). In some cases, the contacting step is carried out in a cell in vitro. In some cases, the contacting step is carried out in a cell in vivo. In some cases, the contacting step is carried out in a cell ex vivo.

Because a method that uses a variant CRISPR-Cas effector polypeptide or fusion polypeptide of the present disclosure includes binding of the variant CRISPR-Cas effector polypeptide or fusion polypeptide to a particular region in a target nucleic acid (by virtue of being targeted there by an associated CasPhi guide RNA), the methods can be generally referred to herein as methods of binding (e.g., a method of binding a target nucleic acid). However, it is to be understood that in some cases, while a method of binding may result in nothing more than binding of the target nucleic acid, in other cases, the method can have different final results (e.g., the method can result in modification of the target nucleic acid, e.g., cleavage/methylation/etc., modulation of transcription from the target nucleic acid; modulation of translation of the target nucleic acid; genome editing; modulation of a protein associated with the target nucleic acid; isolation of the target nucleic acid; etc.).

For examples of suitable methods, see, for example, Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al, Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et. at., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; each of which is hereby incorporated by reference in its entirety.

For example, the present disclosure provides (but is not limited to) methods of cleaving a target nucleic acid; methods of editing a target nucleic acid; methods of modulating transcription from a target nucleic acid; methods of isolating a target nucleic acid, methods of binding a target nucleic acid, methods of imaging a target nucleic acid, methods of modifying a target nucleic acid, and the like.

As used herein, the terms/phrases “contact a target nucleic acid” and “contacting a target nucleic acid”, for example, with a variant CRISPR-Cas effector polypeptide or with a fusion polypeptide, etc., encompass all methods for contacting the target nucleic acid. For example, a variant CRISPR-Cas effector polypeptide can be provided to a cell as protein, RNA (encoding the variant CRISPR-Cas effector polypeptide), or DNA (encoding the variant CRISPR-Cas effector polypeptide); while a CasPhi guide RNA can be provided as a guide RNA or as a nucleic acid encoding the guide RNA. As such, when, for example, performing a method in a cell (e.g., inside of a cell in vitro, inside of a cell in vivo, inside of a cell ex vivo), a method that includes contacting the target nucleic acid encompasses the introduction into the cell of any or all of the components in their active/final state (e.g., in the form of a protein(s) for variant CRISPR-Cas effector polypeptide; in the form of a protein for a fusion polypeptide; in the form of an RNA in some cases for the guide RNA), and also encompasses the introduction into the cell of one or more nucleic acids encoding one or more of the components (e.g., nucleic acid(s) comprising nucleotide sequence(s) encoding a variant CRISPR-Cas effector polypeptide or a fusion polypeptide, nucleic acid(s) comprising nucleotide sequence(s) encoding guide RNA(s), nucleic acid comprising a nucleotide sequence encoding a donor template, and the like). Because the methods can also be performed in vitro outside of a cell, a method that includes contacting a target nucleic acid, (unless otherwise specified) encompasses contacting outside of a cell in vitro, inside of a cell in vitro, inside of a cell in vivo, inside of a cell ex vivo, etc.

In some cases, a method of the present disclosure for modifying a target nucleic acid comprises introducing into a target cell a CasPhi locus, e.g., a nucleic acid comprising a nucleotide sequence encoding a variant CRISPR-Cas effector polypeptide as well as nucleotide sequences of about 1 kilobase (kb) to 5 kb in length surrounding the CasPhi-encoding nucleotide sequence from a cell (e.g., in some cases a cell that in its natural state (the state in which it occurs in nature) comprises a CasPhi locus) comprising a CasPhi locus, where the target cell does not normally (in its natural state) comprise a CasPhi locus. However, one or more spacer sequences, encoding guide sequences for the encoded crRNA(s), can be modified such that one or more target sequences of interest are targeted. Thus, for example, in some cases, a method of the present disclosure for modifying a target nucleic acid comprises introducing into a target cell a CasPhi locus, e.g., a nucleic acid obtained from a source cell (e.g., in some cases a cell that in its natural state (the state in which it occurs in nature) comprises a CasPhi locus), where the nucleic acid has a length of from 100 nucleotides (nt) to 5 kb in length (e.g., from 100 nt to 500 nt, from 500 nt to 1 kb, from 1 kb to 1.5 kb, from 1.5 kb to 2 kb, from 2 kb to 2.5 kb, from 2.5 kb to 3 kb, from 3 kb to 3.5 kb, from 3.5 kb to 4 kb, or from 4 kb to 5 kb in length) and comprises a nucleotide sequence encoding a variant CRISPR-Cas effector polypeptide. As noted above, in some such cases, one or more spacer sequences, encoding guide sequences for the encoded crRNA(s), can be modified such that one or more target sequences of interest are targeted. In some cases, the method comprises introducing into a target cell: i) a CasPhi locus; and ii) a donor DNA template. In some cases, the target nucleic acid is in a cell-free composition in vitro. In some cases, the target nucleic acid is present in a target cell. In some cases, the target nucleic acid is present in a target cell, where the target cell is a prokaryotic cell. In some cases, the target nucleic acid is present in a target cell, where the target cell is a eukaryotic cell. In some cases, the target nucleic acid is present in a target cell, where the target cell is a mammalian cell. In some cases, the target nucleic acid is present in a target cell, where the target cell is a plant cell.

In some cases, a method of the present disclosure for modifying a target nucleic acid comprises contacting a target nucleic acid with a variant CRISPR-Cas effector polypeptide of the present disclosure, or with a fusion polypeptide of the present disclosure. In some cases, a method of the present disclosure for modifying a target nucleic acid comprises contacting a target nucleic acid with a variant CRISPR-Cas effector polypeptide and a CasPhi guide RNA. In some cases, a method of the present disclosure for modifying a target nucleic acid comprises contacting a target nucleic acid with a variant CRISPR-Cas effector polypeptide, a first CasPhi guide RNA, and a second CasPhi guide RNA In some cases, a method of the present disclosure for modifying a target nucleic acid comprises contacting a target nucleic acid with a variant CRISPR-Cas effector polypeptide of the present disclosure and a CasPhi guide RNA and a donor DNA template.

### Target Nucleic Acids and Target Cells of Interest

A variant CRISPR-Cas effector polypeptide of the present disclosure, or a fusion polypeptide of the present disclosure, when bound to a CasPhi guide RNA, can bind to a target nucleic acid, and in some cases, can bind to and modify a target nucleic acid. A target nucleic acid can be any nucleic acid (e.g., DNA, RNA), can be double stranded or single stranded, can be any type of nucleic acid (e.g., a chromosome (genomic DNA), derived from a chromosome, chromosomal DNA, plasmid, viral, extracellular, intracellular, mitochondrial, chloroplast, linear, circular, etc.) and can be from any organism (e.g., as long as the CasPhi guide RNA comprises a nucleotide sequence that hybridizes to a target sequence in a target nucleic acid, such that the target nucleic acid can be targeted).

A target nucleic acid can be DNA or RNA. A target nucleic acid can be double stranded (e.g., dsDNA, dsRNA) or single stranded (e.g., ssRNA, ssDNA). In some cases, a target nucleic acid is single stranded. In some cases, a target nucleic acid is a single stranded RNA (ssRNA). In some cases, a target ssRNA (e.g., a target cell ssRNA, a viral ssRNA, etc.) is selected from: mRNA, rRNA, tRNA, non-coding RNA (ncRNA), long non-coding RNA (lncRNA), and microRNA (miRNA). In some cases, a target nucleic acid is a single stranded DNA (ssDNA) (e.g., a viral DNA). As noted above, in some cases, a target nucleic acid is single stranded.

A target nucleic acid can be located anywhere, for example, outside of a cell in vitro, inside of a cell in vitro, inside of a cell in vivo, inside of a cell ex vivo. Suitable target cells (which can comprise target nucleic acids such as genomic DNA) include, but are not limited to: a bacterial cell; an archaeal cell; a cell of a single-cell eukaryotic organism; a plant cell; an algal cell, e.g., Botryococcus braunii, Chlamydomonas reinhardtii, Nannochloropsis gaditana, Chlorella pyrenoidosa, Sargassum patens, C. agardhii, and the like; a fungal cell (e.g., a yeast cell); an animal cell; a cell from an invertebrate animal (e.g. fruit fly, a cnidarian, an echinoderm, a nematode, etc.); a cell of an insect (e.g., a mosquito; a bee; an agricultural pest; etc.); a cell of an arachnid (e.g., a spider; a tick; etc.); a cell from a vertebrate animal (e.g., a fish, an amphibian, a reptile, a bird, a mammal); a cell from a mammal (e.g., a cell from a rodent; a cell from a human; a cell of a non-human mammal; a cell of a rodent (e.g., a mouse, a rat); a cell of a lagomorph (e.g., a rabbit); a cell of an ungulate (e.g., a cow, a horse, a camel, a llama, a vicuña, a sheep, a goat, etc.); a cell of a marine mammal (e.g., a whale, a seal, an elephant seal, a dolphin, a sea lion; etc.) and the like. Any type of cell may be of interest (e.g. a stem cell, e.g. an embryonic stem (ES) cell, an induced pluripotent stem (iPS) cell, a germ cell (e.g., an oocyte, a sperm, an oogonia, a spermatogonia, etc.), an adult stem cell, a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.).

Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures are cultures that may have been passaged 0 times, 1 time, 2 times, 4 times, 5 times, 10 times, or 15 times, but not enough times go through the crisis stage. Typically, the primary cell lines are maintained for fewer than 10 passages in vitro. Target cells can be unicellular organisms and/or can be grown in culture. If the cells are primary cells, they may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. can be conveniently harvested by biopsy.

In some of the above applications, the subject methods may be employed to induce target nucleic acid cleavage, target nucleic acid modification, and/or to bind target nucleic acids (e.g., for visualization, for collecting and/or analyzing, etc.) in mitotic or post-mitotic cells in vivo and/or ex vivo and/or in vitro (e.g., to disrupt production of a protein encoded by a targeted mRNA, to cleave or otherwise modify target DNA, to genetically modify a target cell, and the like). Because the guide RNA provides specificity by hybridizing to target nucleic acid, a mitotic and/or post-mitotic cell of interest in the disclosed methods may include a cell from any organism (e.g. a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens, C. agardhii, and the like, a fungal cell (e.g., a yeast cell), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal, a cell from a rodent, a cell from a human, etc.). In some cases, a subject CasPhi protein (and/or nucleic acid encoding the protein such as DNA and/or RNA), and/or CasPhi guide RNA (and/or a DNA encoding the guide RNA), and/or donor template, and/or RNP can be introduced into an individual (i.e., the target cell can be in vivo) (e.g., a mammal, a rat, a mouse, a pig, a primate, a non-human primate, a human, etc.). In some case, such an administration can be for the purpose of treating and/or preventing a disease, e.g., by editing the genome of targeted cells.

Plant cells include cells of a monocotyledon, and cells of a dicotyledon. The cells can be root cells, leaf cells, cells of the xylem, cells of the phloem, cells of the cambium, apical meristem cells, parenchyma cells, collenchyma cells, sclerenchyma cells, and the like. Plant cells include cells of agricultural crops such as wheat, corn, rice, sorghum, millet, soybean, etc. Plant cells include cells of agricultural fruit and nut plants, e.g., plant that produce apricots, oranges, lemons, apples, plums, pears, almonds, etc.

Additional examples of target cells are listed above in the section titled “Modified cells.” Non-limiting examples of cells (target cells) include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomonas reinhardtii, Nannochloropsis gaditana, Chlorella pyrenoidosa, Sargassum patens, C. agardhii, and the like), seaweeds (e.g. kelp) a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some cases, the cell is a cell that does not originate from a natural organism (e.g., the cell can be a synthetically made cell; also referred to as an artificial cell).

A cell can be an in vitro cell (e.g., established cultured cell line). A cell can be an ex vivo cell (cultured cell from an individual). A cell can be and in vivo cell (e.g., a cell in an individual). A cell can be an isolated cell. A cell can be a cell inside of an organism. A cell can be an organism. A cell can be a cell in a cell culture (e.g., in vitro cell culture). A cell can be one of a collection of cells. A cell can be a prokaryotic cell or derived from a prokaryotic cell. A cell can be a bacterial cell or can be derived from a bacterial cell. A cell can be an archaeal cell or derived from an archaeal cell. A cell can be a eukaryotic cell or derived from a eukaryotic cell. A cell can be a plant cell or derived from a plant cell. A cell can be an animal cell or derived from an animal cell. A cell can be an invertebrate cell or derived from an invertebrate cell. A cell can be a vertebrate cell or derived from a vertebrate cell. A cell can be a mammalian cell or derived from a mammalian cell. A cell can be a rodent cell or derived from a rodent cell. A cell can be a human cell or derived from a human cell. A cell can be a microbe cell or derived from a microbe cell. A cell can be a fungi cell or derived from a fungi cell. A cell can be an insect cell. A cell can be an arthropod cell. A cell can be a protozoan cell. A cell can be a helminth cell.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogeneic cells, allogenic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGC1A.

In some cases, the stem cell is a hematopoietic stem cell (HSC). HSCs are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other embodiments, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

A cell is in some cases a plant cell. A plant cell can be a cell of a monocotyledon. A cell can be a cell of a dicotyledon.

In some cases, the cell is a plant cell. For example, the cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, chinese artichoke (crosnes), chinese cabbage, chinese celery, chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (chinese mustard), gailon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf—green), lettuce (oak leaf—red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams, yu choy, yuca (cassava), and the like.

A cell is in some cases an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopterygota, Plecoptera, Embioptera, Orthoptera, Zoraptera, Dermaptera, Dictyoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

A cell is in some cases an insect cell. For example, in some cases, the cell is a cell of a mosquito, a grasshopper, a true bug, a fly, a flea, a bee, a wasp, an ant, a louse, a moth, or a beetle.

Introducing Components into a Target Cell

A CasPhi guide RNA (or a nucleic acid comprising a nucleotide sequence encoding same), and/or a fusion polypeptide (or a nucleic acid comprising a nucleotide sequence encoding same), and/or a variant CRISPR-Cas effector polypeptide (or a nucleic acid comprising a nucleotide sequence encoding same) and/or a donor polynucleotide can be introduced into a host cell by any of a variety of well-known methods.

Methods of introducing a nucleic acid into a cell are known in the art, and any convenient method can be used to introduce a nucleic acid (e.g., an expression construct) into a target cell (e.g., eukaryotic cell, human cell, stem cell, progenitor cell, and the like). Suitable methods are described in more detail elsewhere herein and include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: S0169-409X(12)00283-9. doi: 10.1016/j.addr.2012.09.023), and the like. Any or all of the components can be introduced into a cell as a composition (e.g., including any convenient combination of: a variant CRISPR-Cas effector polypeptide, a CasPhi guide RNA, a donor polynucleotide, etc.) using known methods, e.g., such as nucleofection.

### Donor Polynucleotide (Donor Template)

Guided by a CasPhi guide RNA, a variant CRISPR-Cas effector protein in some cases generates site-specific double strand breaks (DSBs) or single strand breaks within double-stranded DNA (dsDNA) target nucleic acids, which are repaired either by non-homologous end joining (NHEJ) or homology-directed recombination (HDR).

In some cases, contacting a target DNA (with a variant CRISPR-Cas effector protein and a CasPhi guide RNA) occurs under conditions that are permissive for nonhomologous end joining or homology-directed repair. Thus, in some cases, a subject method includes contacting the target DNA with a donor polynucleotide (e.g., by introducing the donor polynucleotide into a cell), wherein the donor polynucleotide, a portion of the donor polynucleotide, a copy of the donor polynucleotide, or a portion of a copy of the donor polynucleotide integrates into the target DNA. In some cases, the method does not comprise contacting a cell with a donor polynucleotide, and the target DNA is modified such that nucleotides within the target DNA are deleted.

In some cases, CasPhi guide RNA (or DNA encoding same) and a variant CRISPR-Cas effector protein (or a nucleic acid encoding same, such as an RNA or a DNA, e.g., one or more expression vectors) are coadministered (e.g., contacted with a target nucleic acid, administered to cells, etc.) with a donor polynucleotide sequence that includes at least a segment with homology to the target DNA sequence, the subject methods may be used to add, i.e. insert or replace, nucleic acid material to a target DNA sequence (e.g. to “knock in” a nucleic acid, e.g., one that encodes for a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g. promoter, polyadenylation signal, internal ribosome entry sequence (IRES), 2A peptide, start codon, stop codon, splice signal, localization signal, etc.), to modify a nucleic acid sequence (e.g., introduce a mutation, remove a disease causing mutation by introducing a correct sequence), and the like. As such, a complex comprising a CasPhi guide RNA and variant CRISPR-Cas effector protein is useful in any in vitro or in vivo application in which it is desirable to modify DNA in a site-specific, i.e. “targeted”, way, for example gene knock-out, gene knock-in, gene editing, gene tagging, etc., as used in, for example, gene therapy, e.g. to treat a disease or as an antiviral, antipathogenic, or anticancer therapeutic, the production of genetically modified organisms in agriculture, the large scale production of proteins by cells for therapeutic, diagnostic, or research purposes, the induction of iPS cells, biological research, the targeting of genes of pathogens for deletion or replacement, etc.

In applications in which it is desirable to insert a polynucleotide sequence into the genome where a target sequence is cleaved, a donor polynucleotide (a nucleic acid comprising a donor sequence) can also be provided to the cell. By a “donor sequence” or “donor polynucleotide” or “donor template” it is meant a nucleic acid sequence to be inserted at the site cleaved by the variant CRISPR-Cas effector protein (e.g., after dsDNA cleavage, after nicking a target DNA, after dual nicking a target DNA, and the like). The donor polynucleotide can contain sufficient homology to a genomic sequence at the target site, e.g. 70%, 80%, 85%, 90%, 95%, or 100% homology with the nucleotide sequences flanking the target site, e.g. within about 50 bases or less of the target site, e.g. within about 30 bases, within about 15 bases, within about 10 bases, within about 5 bases, or immediately flanking the target site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) can support homology-directed repair. Donor polynucleotides can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair (e.g., for gene correction, e.g., to convert a disease-causing base pair to a non-disease-causing base pair). In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

In some cases, the donor sequence is provided to the cell as single-stranded DNA. In some cases, the donor sequence is provided to the cell as double-stranded DNA. It may be introduced into a cell in linear or circular form. If introduced in linear form, the ends of the donor sequence may be protected (e.g., from exonucleolytic degradation) by any convenient method and such methods are known to those of skill in the art. For example, one or more dideoxynucleotide residues can be added to the 3′ terminus of a linear molecule and/or self-complementary oligonucleotides can be ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by viruses (e.g., adenovirus, AAV), as described elsewhere herein for nucleic acids encoding a CasPhi guide RNA and/or a CasPhi fusion polypeptide and/or donor polynucleotide.

### Detection Methods

A variant CRISPR-Cas effector polypeptide of the present disclosure can promiscuously cleave non-targeted single stranded DNA (ssDNA) once activated by detection of a target DNA (double or single stranded; also referred to herein as an “activator” target nucleic acid or simply “activator” nucleic acid or “activator” ssDNA). Once a variant CRISPR-Cas effector polypeptide of the present disclosure is activated by a guide RNA, which occurs when the guide RNA hybridizes to a target sequence of a target DNA (i.e., the sample includes the targeted DNA), the variant CRISPR-Cas effector polypeptide becomes a nuclease that promiscuously cleaves ssDNAs (i.e., the nuclease cleaves non-target ssDNAs, i.e., ssDNAs to which the guide sequence of the guide RNA does not hybridize). Thus, when the target DNA is present in the sample (e.g., in some cases above a threshold amount), the result is cleavage of ssDNAs in the sample, which can be detected using any convenient detection method (e.g., using a labeled single stranded detector DNA). Cleavage of non-target nucleic acid is referred to as “trans cleavage.” In some cases, a variant CRISPR-Cas effector polypeptide of the present disclosure mediates trans cleavage of ssDNA, but not ssRNA.

Provided are compositions and methods for detecting a target DNA (double stranded or single stranded) in a sample. In some cases, a detector DNA is used that is single stranded (ssDNA) and does not hybridize with the guide sequence of the guide RNA (i.e., the detector ssDNA is a non-target ssDNA). Such methods can include (a) contacting the sample with: (i) a variant CRISPR-Cas effector polypeptide of the present disclosure; (ii) a guide RNA comprising: a region that binds to the variant CRISPR-Cas effector polypeptide, and a guide sequence that hybridizes with the target DNA; and (iii) a detector DNA that is single stranded and does not hybridize with the guide sequence of the guide RNA; and (b) measuring a detectable signal produced by cleavage of the single stranded detector DNA by the variant CRISPR-Cas effector polypeptide, thereby detecting the target DNA. As noted above, once a CasPhi polypeptide of the present disclosure is activated by a guide RNA, which occurs when the sample includes a target DNA to which the guide RNA hybridizes (i.e., the sample includes the targeted target DNA), the variant CRISPR-Cas effector polypeptide is activated and functions as an endoribonuclease that non-specifically cleaves ssDNAs (including non-target ssDNAs) present in the sample. Thus, when the targeted target DNA is present in the sample (e.g., in some cases above a threshold amount), the result is cleavage of ssDNA (including non-target ssDNA) in the sample, which can be detected using any convenient detection method (e.g., using a labeled detector ssDNA).

Also provided are compositions and methods for cleaving single stranded DNAs (ssDNAs) (e.g., non-target ssDNAs). Such methods can include contacting a population of nucleic acids, wherein said population comprises a target DNA and a plurality of non-target ssDNAs, with: (i) a variant CRISPR-Cas effector polypeptide of the present disclosure; and (ii) a guide RNA comprising: a region that binds to the CasPhi polypeptide and a guide sequence that hybridizes with the target DNA, wherein the variant CRISPR-Cas effector polypeptide cleaves non-target ssDNAs of said plurality. Such a method can be used, e.g., to cleave foreign ssDNAs (e.g., viral DNAs) in a cell.

The contacting step of a subject method can be carried out in a composition comprising divalent metal ions. The contacting step can be carried out in an acellular environment, e.g., outside of a cell. The contacting step can be carried out inside a cell. The contacting step can be carried out in a cell in vitro. The contacting step can be carried out in a cell ex vivo. The contacting step can be carried out in a cell in vivo.

The guide RNA can be provided as RNA or as a nucleic acid encoding the guide RNA (e.g., a DNA such as a recombinant expression vector). The variant CRISPR-Cas effector polypeptide can be provided as a protein or as a nucleic acid encoding the protein (e.g., an mRNA, a DNA such as a recombinant expression vector). In some cases, two or more (e.g., 3 or more, 4 or more, 5 or more, or 6 or more) guide RNAs can be provided by (e.g., using a precursor guide RNA array, which can be cleaved by the variant CRISPR-Cas effector protein into individual (“mature”) guide RNAs).

In some cases (e.g., when contacting with a guide RNA and a variant CRISPR-Cas effector polypeptide of the present disclosure, the sample is contacted for 2 hours or less (e.g., 1.5 hours or less, 1 hour or less, 40 minutes or less, 30 minutes or less, 20 minutes or less, 10 minutes or less, or 5 minutes or less, or 1 minute or less) prior to the measuring step. For example, in some cases the sample is contacted for 40 minutes or less prior to the measuring step. In some cases, the sample is contacted for 20 minutes or less prior to the measuring step. In some cases, the sample is contacted for 10 minutes or less prior to the measuring step. In some cases, the sample is contacted for 5 minutes or less prior to the measuring step. In some cases, the sample is contacted for 1 minute or less prior to the measuring step. In some cases, the sample is contacted for from 50 seconds to 60 seconds prior to the measuring step. In some cases, the sample is contacted for from 40 seconds to 50 seconds prior to the measuring step. In some cases, the sample is contacted for from 30 seconds to 40 seconds prior to the measuring step. In some cases, the sample is contacted for from 20 seconds to 30 seconds prior to the measuring step. In some cases, the sample is contacted for from 10 seconds to 20 seconds prior to the measuring step.

A method of the present disclosure for detecting a target DNA (single-stranded or double-stranded) in a sample can detect a target DNA with a high degree of sensitivity. In some cases, a method of the present disclosure can be used to detect a target DNA present in a sample comprising a plurality of DNAs (including the target DNA and a plurality of non-target DNAs), where the target DNA is present at one or more copies per 10⁷ non-target DNAs (e.g., one or more copies per 10⁶ non-target DNAs, one or more copies per 10⁷ non-target DNAs, one or more copies per 10⁴ non-target DNAs, one or more copies per 10³ non-target DNAs, one or more copies per 10² non-target DNAs, one or more copies per 50 non-target DNAs, one or more copies per 20 non-target DNAs, one or more copies per 10 non-target DNAs, or one or more copies per 5 non-target DNAs). In some cases, a method of the present disclosure can be used to detect a target DNA present in a sample comprising a plurality of DNAs (including the target DNA and a plurality of non-target DNAs), where the target DNA is present at one or more copies per 10¹⁸ a non-target DNAs (e.g., one or more copies per 10¹⁵ non-target DNAs, one or more copies per 10¹² non-target DNAs, one or more copies per 10⁹ non-target DNAs, one or more copies per 10⁶ non-target DNAs, one or more copies per 10⁵ non-target DNAs, one or more copies per 10⁴ non-target DNAs, one or more copies per 10³ non-target DNAs, one or more copies per 10² non-target DNAs, one or more copies per 50 non-target DNAs, one or more copies per 20 non-target DNAs, one or more copies per 10 non-target DNAs, or one or more copies per 5 non-target DNAs).

In some cases, a method of the present disclosure can detect a target DNA present in a sample, where the target DNA is present at from one copy per 10⁷ non-target DNAs to one copy per 10 non-target DNAs (e.g., from 1 copy per 10⁷ non-target DNAs to 1 copy per 10² non-target DNAs, from 1 copy per 10⁷ non-target DNAs to 1 copy per 10³ non-target DNAs, from 1 copy per 10⁷ non-target DNAs to 1 copy per 10⁴ non-target DNAs, from 1 copy per 10⁷ non-target DNAs to 1 copy per 10⁵ non-target DNAs, from 1 copy per 10⁷ non-target DNAs to 1 copy per 10⁶ non-target DNAs, from 1 copy per 10⁶ non-target DNAs to 1 copy per 10 non-target DNAs, from 1 copy per 10⁶ non-target DNAs to 1 copy per 10² non-target DNAs, from 1 copy per 10⁶ non-target DNAs to 1 copy per 10³ non-target DNAs, from 1 copy per 10⁶ non-target DNAs to 1 copy per 10⁴ non-target DNAs, from 1 copy per 10⁶ non-target DNAs to 1 copy per 10⁵ non-target DNAs, from 1 copy per 10⁵ non-target DNAs to 1 copy per 10 non-target DNAs, from 1 copy per 10⁵ non-target DNAs to 1 copy per 10² non-target DNAs, from 1 copy per 10⁵ non-target DNAs to 1 copy per 10³ non-target DNAs, or from 1 copy per 10⁵ non-target DNAs to 1 copy per 10⁴ non-target DNAs).

In some cases, a method of the present disclosure can detect a target DNA present in a sample, where the target DNA is present at from one copy per 10¹⁸ a non-target DNAs to one copy per 10 non-target DNAs (e.g., from 1 copy per 10¹⁸ a non-target DNAs to 1 copy per 10² non-target DNAs, from 1 copy per 10¹⁵ non-target DNAs to 1 copy per 10² non-target DNAs, from 1 copy per 10¹² non-target DNAs to 1 copy per 10² non-target DNAs, from 1 copy per 10⁹ non-target DNAs to 1 copy per 10² non-target DNAs, from 1 copy per 10⁷ non-target DNAs to 1 copy per 10² non-target DNAs, from 1 copy per 10⁷ non-target DNAs to 1 copy per 10³ non-target DNAs, from 1 copy per 10⁷ non-target DNAs to 1 copy per 10⁴ non-target DNAs, from 1 copy per 10⁷ non-target DNAs to 1 copy per 10⁵ non-target DNAs, from 1 copy per 10⁷ non-target DNAs to 1 copy per 10⁶ non-target DNAs, from 1 copy per 10⁶ non-target DNAs to 1 copy per 10 non-target DNAs, from 1 copy per 10⁶ non-target DNAs to 1 copy per 10² non-target DNAs, from 1 copy per 10⁶ non-target DNAs to 1 copy per 10³ non-target DNAs, from 1 copy per 10⁶ non-target DNAs to 1 copy per 10⁴ non-target DNAs, from 1 copy per 10⁶ non-target DNAs to 1 copy per 10⁵ non-target DNAs, from 1 copy per 10⁵ non-target DNAs to 1 copy per 10 non-target DNAs, from 1 copy per 10⁵ non-target DNAs to 1 copy per 10² non-target DNAs, from 1 copy per 10⁵ non-target DNAs to 1 copy per 10³ non-target DNAs, or from 1 copy per 10⁵ non-target DNAs to 1 copy per 10⁴ non-target DNAs).

In some cases, a method of the present disclosure can detect a target DNA present in a sample, where the target DNA is present at from one copy per 10⁷ non-target DNAs to one copy per 100 non-target DNAs (e.g., from 1 copy per 10⁷ non-target DNAs to 1 copy per 10² non-target DNAs, from 1 copy per 10⁷ non-target DNAs to 1 copy per 10³ non-target DNAs, from 1 copy per 10⁷ non-target DNAs to 1 copy per 10⁴ non-target DNAs, from 1 copy per 10⁷ non-target DNAs to 1 copy per 10⁵ non-target DNAs, from 1 copy per 10⁷ non-target DNAs to 1 copy per 10⁶ non-target DNAs, from 1 copy per 10⁶ non-target DNAs to 1 copy per 100 non-target DNAs, from 1 copy per 10⁶ non-target DNAs to 1 copy per 10² non-target DNAs, from 1 copy per 10⁶ non-target DNAs to 1 copy per 10³ non-target DNAs, from 1 copy per 10⁶ non-target DNAs to 1 copy per 10⁴ non-target DNAs, from 1 copy per 10⁶ non-target DNAs to 1 copy per 10⁵ non-target DNAs, from 1 copy per 10⁵ non-target DNAs to 1 copy per 100 non-target DNAs, from 1 copy per 10⁵ non-target DNAs to 1 copy per 10² non-target DNAs, from 1 copy per 10⁵ non-target DNAs to 1 copy per 10³ non-target DNAs, or from 1 copy per 10⁵ non-target DNAs to 1 copy per 10⁴ non-target DNAs).

In some cases, the threshold of detection, for a subject method of detecting a target DNA in a sample, is 10 nM or less. The term “threshold of detection” is used herein to describe the minimal amount of target DNA that must be present in a sample in order for detection to occur. Thus, as an illustrative example, when a threshold of detection is 10 nM, then a signal can be detected when a target DNA is present in the sample at a concentration of 10 nM or more. In some cases, a method of the present disclosure has a threshold of detection of 5 nM or less. In some cases, a method of the present disclosure has a threshold of detection of 1 nM or less. In some cases, a method of the present disclosure has a threshold of detection of 0.5 nM or less. In some cases, a method of the present disclosure has a threshold of detection of 0.1 nM or less. In some cases, a method of the present disclosure has a threshold of detection of 0.05 nM or less. In some cases, a method of the present disclosure has a threshold of detection of 0.01 nM or less. In some cases, a method of the present disclosure has a threshold of detection of 0.005 nM or less. In some cases, a method of the present disclosure has a threshold of detection of 0.001 nM or less. In some cases, a method of the present disclosure has a threshold of detection of 0.0005 nM or less. In some cases, a method of the present disclosure has a threshold of detection of 0.0001 nM or less. In some cases, a method of the present disclosure has a threshold of detection of 0.00005 nM or less. In some cases, a method of the present disclosure has a threshold of detection of 0.00001 nM or less. In some cases, a method of the present disclosure has a threshold of detection of 10 pM or less. In some cases, a method of the present disclosure has a threshold of detection of 1 pM or less. In some cases, a method of the present disclosure has a threshold of detection of 500 fM or less. In some cases, a method of the present disclosure has a threshold of detection of 250 fM or less. In some cases, a method of the present disclosure has a threshold of detection of 100 fM or less. In some cases, a method of the present disclosure has a threshold of detection of 50 fM or less. In some cases, a method of the present disclosure has a threshold of detection of 500 aM (attomolar) or less. In some cases, a method of the present disclosure has a threshold of detection of 250 aM or less. In some cases, a method of the present disclosure has a threshold of detection of 100 aM or less. In some cases, a method of the present disclosure has a threshold of detection of 50 aM or less. In some cases, a method of the present disclosure has a threshold of detection of 10 aM or less. In some cases, a method of the present disclosure has a threshold of detection of 1 aM or less.

In some cases, the threshold of detection (for detecting the target DNA in a subject method), is in a range of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM) (where the concentration refers to the threshold concentration of target DNA at which the target DNA can be detected). In some cases, a method of the present disclosure has a threshold of detection in a range of from 800 fM to 100 pM. In some cases, a method of the present disclosure has a threshold of detection in a range of from 1 pM to 10 pM. In some cases, a method of the present disclosure has a threshold of detection in a range of from 10 fM to 500 fM, e.g., from 10 fM to 50 fM, from 50 fM to 100 fM, from 100 fM to 250 fM, or from 250 fM to 500 fM.

In some cases, the minimum concentration at which a target DNA can be detected in a sample is in a range of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM). In some cases, the minimum concentration at which a target DNA can be detected in a sample is in a range of from 800 fM to 100 pM. In some cases, the minimum concentration at which a target DNA can be detected in a sample is in a range of from 1 pM to 10 pM.

In some cases, the threshold of detection (for detecting the target DNA in a subject method), is in a range of from 1 aM to 1 nM (e.g., from 1 aM to 500 pM, from 1 aM to 200 pM, from 1 aM to 100 pM, from 1 aM to 10 pM, from 1 aM to 1 pM, from 100 aM to 1 nM, from 100 aM to 500 pM, from 100 aM to 200 pM, from 100 aM to 100 pM, from 100 aM to 10 pM, from 100 aM to 1 pM, from 250 aM to 1 nM, from 250 aM to 500 pM, from 250 aM to 200 pM, from 250 aM to 100 pM, from 250 aM to 10 pM, from 250 aM to 1 pM, from 500 aM to 1 nM, from 500 aM to 500 pM, from 500 aM to 200 pM, from 500 aM to 100 pM, from 500 aM to 10 pM, from 500 aM to 1 pM, from 750 aM to 1 nM, from 750 aM to 500 pM, from 750 aM to 200 pM, from 750 aM to 100 pM, from 750 aM to 10 pM, from 750 aM to 1 pM, from 1 fM to 1 nM, from 1 fM to 500 pM, from 1 fM to 200 pM, from 1 fM to 100 pM, from 1 fM to 10 pM, from 1 fM to 1 pM, from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM) (where the concentration refers to the threshold concentration of target DNA at which the target DNA can be detected). In some cases, a method of the present disclosure has a threshold of detection in a range of from 1 aM to 800 aM. In some cases, a method of the present disclosure has a threshold of detection in a range of from 50 aM to 1 pM. In some cases, a method of the present disclosure has a threshold of detection in a range of from 50 aM to 500 fM.

In some cases, the minimum concentration at which a target DNA can be detected in a sample is in a range of from 1 aM to 1 nM (e.g., from 1 aM to 500 pM, from 1 aM to 200 pM, from 1 aM to 100 pM, from 1 aM to 10 pM, from 1 aM to 1 pM, from 100 aM to 1 nM, from 100 aM to 500 pM, from 100 aM to 200 pM, from 100 aM to 100 pM, from 100 aM to 10 pM, from 100 aM to 1 pM, from 250 aM to 1 nM, from 250 aM to 500 pM, from 250 aM to 200 pM, from 250 aM to 100 pM, from 250 aM to 10 pM, from 250 aM to 1 pM, from 500 aM to 1 nM, from 500 aM to 500 pM, from 500 aM to 200 pM, from 500 aM to 100 pM, from 500 aM to 10 pM, from 500 aM to 1 pM, from 750 aM to 1 nM, from 750 aM to 500 pM, from 750 aM to 200 pM, from 750 aM to 100 pM, from 750 aM to 10 pM, from 750 aM to 1 pM, from 1 fM to 1 nM, from 1 fM to 500 pM, from 1 fM to 200 pM, from 1 fM to 100 pM, from 1 fM to 10 pM, from 1 fM to 1 pM, from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM). In some cases, the minimum concentration at which a target DNA can be detected in a sample is in a range of from 1 aM to 500 pM. In some cases, the minimum concentration at which a target DNA can be detected in a sample is in a range of from 100 aM to 500 pM.

In some cases, a subject composition or method exhibits an attomolar (aM) sensitivity of detection. In some cases, a subject composition or method exhibits a femtomolar (fM) sensitivity of detection. In some cases, a subject composition or method exhibits a picomolar (pM) sensitivity of detection. In some cases, a subject composition or method exhibits a nanomolar (nM) sensitivity of detection.

### Target DNA

A target DNA can be single stranded (ssDNA) or double stranded (dsDNA). When the target DNA is single stranded, there is no preference or requirement for a PAM sequence in the target DNA. However, when the target DNA is dsDNA, a PAM is usually present adjacent to the target sequence of the target DNA (e.g., see discussion of the PAM elsewhere herein). The source of the target DNA can be the same as the source of the sample, e.g., as described below.

The source of the target DNA can be any source. In some cases, the target DNA is a viral DNA (e.g., a genomic DNA of a DNA virus). As such, subject method can be for detecting the presence of a viral DNA amongst a population of nucleic acids (e.g., in a sample). A subject method can also be used for the cleavage of non-target ssDNAs in the present of a target DNA. For example, if a method takes place in a cell, a subject method can be used to promiscuously cleave non-target ssDNAs in the cell (ssDNAs that do not hybridize with the guide sequence of the guide RNA) when a particular target DNA is present in the cell (e.g., when the cell is infected with a virus and viral target DNA is detected).

Examples of possible target DNAs include, but are not limited to, viral DNAs such as: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus, Pityriasis rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. In some cases, the target DNA is parasite DNA. In some cases, the target DNA is bacterial DNA, e.g., DNA of a pathogenic bacterium.

### Samples

A subject sample includes nucleic acid (e.g., a plurality of nucleic acids). The term “plurality” is used herein to mean two or more. Thus, in some cases, a sample includes two or more (e.g., 3 or more, 5 or more, 10 or more, 20 or more, 50 or more, 100 or more, 500 or more, 1,000 or more, or 5,000 or more) nucleic acids (e.g., DNAs). A subject method can be used as a very sensitive way to detect a target DNA present in a sample (e.g., in a complex mixture of nucleic acids such as DNAs). In some cases, the sample includes 5 or more DNAs (e.g., 10 or more, 20 or more, 50 or more, 100 or more, 500 or more, 1,000 or more, or 5,000 or more DNAs) that differ from one another in sequence. In some cases, the sample includes 10 or more, 20 or more, 50 or more, 100 or more, 500 or more, 10 or more, 5×10³ or more, 10⁴ or more, 5×10⁴ or more, 10⁵ or more, 5×10⁵ or more, 10⁶ or more 5×10⁶ or more, or 10⁷ or more, DNAs. In some cases, the sample comprises from 10 to 20, from 20 to 50, from 50 to 100, from 100 to 500, from 500 to 10³, from 10³ to 5×10³, from 5×10³ to 10⁴, from 10⁴ to 5×10⁴, from 5×10⁴ to 10⁵, from 10⁵ to 5×10⁵, from 5×10⁵ to 10⁶, from 10⁶ to 5×10⁶, or from 5×10⁶ to 10⁷, or more than 10⁷, DNAs. In some cases, the sample comprises from 5 to 10⁷ DNAs (e.g., that differ from one another in sequence) (e.g., from 5 to 10⁶, from 5 to 10⁵, from 5 to 50,000, from 5 to 30,000, from 10 to 10⁶, from 10 to 10⁵, from 10 to 50,000, from 10 to 30,000, from 20 to 10⁶, from 20 to 10⁵, from 20 to 50,000, or from 20 to 30,000 DNAs). In some cases, the sample includes 20 or more DNAs that differ from one another in sequence. In some cases, the sample includes DNAs from a cell lysate (e.g., a eukaryotic cell lysate, a mammalian cell lysate, a human cell lysate, a prokaryotic cell lysate, a plant cell lysate, and the like). For example, in some cases the sample includes DNA from a cell such as a eukaryotic cell, e.g., a mammalian cell such as a human cell.

The term “sample” is used herein to mean any sample that includes DNA (e.g., in order to determine whether a target DNA is present among a population of DNAs). The sample can be derived from any source, e.g., the sample can be a synthetic combination of purified DNAs; the sample can be a cell lysate, an DNA-enriched cell lysate, or DNAs isolated and/or purified from a cell lysate. The sample can be from a patient (e.g., for the purpose of diagnosis). The sample can be from permeabilized cells. The sample can be from crosslinked cells. The sample can be in tissue sections. The sample can be from tissues prepared by crosslinking followed by delipidation and adjustment to make a uniform refractive index. Examples of tissue preparation by crosslinking followed by delipidation and adjustment to make a uniform refractive index have been described in, for example, Shah et al., Development (2016) 143, 2862-2867 doi:10.1242/dev.138560.

A “sample” can include a target DNA and a plurality of non-target DNAs. In some cases, the target DNA is present in the sample at one copy per 10 non-target DNAs, one copy per 20 non-target DNAs, one copy per 25 non-target DNAs, one copy per 50 non-target DNAs, one copy per 100 non-target DNAs, one copy per 500 non-target DNAs, one copy per 103 non-target DNAs, one copy per 5×10³ non-target DNAs, one copy per 10⁴ non-target DNAs, one copy per 5×10⁴ non-target DNAs, one copy per 10⁵ non-target DNAs, one copy per 5×10⁵ non-target DNAs, one copy per 10⁶ non-target DNAs, or less than one copy per 10⁶ non-target DNAs. In some cases, the target DNA is present in the sample at from one copy per 10 non-target DNAs to 1 copy per 20 non-target DNAs, from 1 copy per 20 non-target DNAs to 1 copy per 50 non-target DNAs, from 1 copy per 50 non-target DNAs to 1 copy per 100 non-target DNAs, from 1 copy per 100 non-target DNAs to 1 copy per 500 non-target DNAs, from 1 copy per 500 non-target DNAs to 1 copy per 10³ non-target DNAs, from 1 copy per 10³ non-target DNAs to 1 copy per 5×10³ non-target DNAs, from 1 copy per 5×10³ non-target DNAs to 1 copy per 10⁴ non-target DNAs, from 1 copy per 10⁴ non-target DNAs to 1 copy per 10⁵ non-target DNAs, from 1 copy per 10⁵ non-target DNAs to 1 copy per 10⁶ non-target DNAs, or from 1 copy per 10⁶ non-target DNAs to 1 copy per 10⁷ non-target DNAs.

Suitable samples include but are not limited to saliva, blood, serum, plasma, urine, aspirate, and biopsy samples. Thus, the term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., DNAs. The term “sample” encompasses biological samples such as a clinical sample such as blood, plasma, serum, aspirate, cerebral spinal fluid (CSF), and also includes tissue obtained by surgical resection, tissue obtained by biopsy, cells in culture, cell supernatants, cell lysates, tissue samples, organs, bone marrow, and the like. A “biological sample” includes biological fluids derived therefrom (e.g., cancerous cell, infected cell, etc.), e.g., a sample comprising DNAs that is obtained from such cells (e.g., a cell lysate or other cell extract comprising DNAs).

A sample can comprise, or can be obtained from, any of a variety of cells, tissues, organs, or acellular fluids. Suitable sample sources include eukaryotic cells, bacterial cells, and archaeal cells. Suitable sample sources include single-celled organisms and multi-cellular organisms. Suitable sample sources include single-cell eukaryotic organisms; a plant or a plant cell; an algal cell, e.g., Botryococcus braunii, Chlamydomonas reinhardtii, Nannochloropsis gaditana, Chlorella pyrenoidosa, Sargassum patens, C. agardhii, and the like; a fungal cell (e.g., a yeast cell); an animal cell, tissue, or organ; a cell, tissue, or organ from an invertebrate animal (e.g. fruit fly, cnidarian, echinoderm, nematode, an insect, an arachnid, etc.); a cell, tissue, fluid, or organ from a vertebrate animal (e.g., fish, amphibian, reptile, bird, mammal); a cell, tissue, fluid, or organ from a mammal (e.g., a human; a non-human primate; an ungulate; a feline; a bovine; an ovine; a caprine; etc.). Suitable sample sources include nematodes, protozoans, and the like. Suitable sample sources include parasites such as helminths, malarial parasites, etc.

Suitable sample sources include a cell, tissue, or organism of any of the six kingdoms, e.g., Bacteria (e.g., Eubacteria); Archaebacteria; Protista; Fungi; Plantae; and Animalia. Suitable sample sources include plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g., Apicomplexa, Myxozoa, Microsporidia), and ciliates (e.g., Paramecium).

Suitable sample sources include members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. Suitable sample sources include members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable sample sources include members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). Suitable plants include any monocotyledon and any dicotyledon.

Suitable sources of a sample include cells, fluid, tissue, or organ taken from an organism; from a particular cell or group of cells isolated from an organism; etc. For example, where the organism is a plant, suitable sources include xylem, the phloem, the cambium layer, leaves, roots, etc. Where the organism is an animal, suitable sources include particular tissues (e.g., lung, liver, heart, kidney, brain, spleen, skin, fetal tissue, etc.), or a particular cell type (e.g., neuronal cells, epithelial cells, endothelial cells, astrocytes, macrophages, glial cells, islet cells, T lymphocytes, B lymphocytes, etc.).

In some cases, the source of the sample is a (or is suspected of being a diseased cell, fluid, tissue, or organ. In some cases, the source of the sample is a normal (non-diseased) cell, fluid, tissue, or organ. In some cases, the source of the sample is a (or is suspected of being) a pathogen-infected cell, tissue, or organ. For example, the source of a sample can be an individual who may or may not be infected—and the sample could be any biological sample (e.g., blood, saliva, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, an epithelial cell sample (e.g., epithelial cell scraping), etc.) collected from the individual. In some cases, the sample is a cell-free liquid sample. In some cases, the sample is a liquid sample that can comprise cells. Pathogens include viruses, fungi, helminths, protozoa, malarial parasites, Plasmodium parasites, Toxoplasma parasites, Schistosoma parasites, and the like. “Helminths” include roundworms, heartworms, and phytophagous nematodes (Nematoda), flukes (Tematoda), Acanthocephala, and tapeworms (Cestoda). Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, Plasmodium vivax, Trypanosoma cruzi and Toxoplasma gondii. Fungal pathogens include, but are not limited to: Cryptococcus neoformans, Histoplasma capsulatum, Coccidioides immitis, Blastomyces dermatitidis, Chlamydia trachomatis, and Candida albicans. Pathogenic viruses include, e.g., human immunodeficiency virus (e.g., HIV); influenza virus; dengue; West Nile virus; herpes virus; yellow fever virus; Hepatitis C Virus; Hepatitis A Virus; Hepatitis B Virus; papillomavirus; and the like. Pathogenic viruses can include DNA viruses such as: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), Epstein-Barr virus (EBV), cytomegalovirus (CMV), herpes lymphotropic virus, Pityriasis Rosea, Kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. Pathogens can include, e.g., DNAviruses (e.g.: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), Epstein-Barr virus (EBV), cytomegalovirus (CMV), herpes lymphotropic virus, Pityriasis Rosea, Kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like], Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum:, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae.

### Measuring a Detectable Signal

In some cases, a subject method includes a step of measuring (e.g., measuring a detectable signal produced by variant CRISPR-Cas effector polypeptide-mediated non-target ssDNA cleavage). Because a variant CRISPR-Cas effector polypeptide of the present disclosure cleaves non-targeted ssDNA once activated, which occurs when a guide RNA hybridizes with a target DNA in the presence of a variant CRISPR-Cas effector protein, a detectable signal can be any signal that is produced when ssDNA is cleaved. For example, in some cases, the step of measuring can include one or more of: gold nanoparticle-based detection (e.g., see Xu et al., Angew Chem Int Ed Engl. 2007; 46(19):3468-70; and Xia et al., Proc Natl Acad Sci USA. 2010 Jun. 15; 107(24):10837-41), fluorescence polarization, colloid phase transition/dispersion (e.g., Baksh et al., Nature. 2004 Jan. 8; 427(6970):139-41), electrochemical detection, semiconductor-based sensing (e.g., Rothberg et al., Nature. 2011 Jul. 20; 475(7356):348-52; e.g., one could use a phosphatase to generate a pH change after ssDNA cleavage reactions, by opening 2′-3′ cyclic phosphates, and by releasing inorganic phosphate into solution), and detection of a labeled detector ssDNA (see elsewhere herein for more details). The readout of such detection methods can be any convenient readout. Examples of possible readouts include but are not limited to: a measured amount of detectable fluorescent signal; a visual analysis of bands on a gel (e.g., bands that represent cleaved product versus uncleaved substrate), a visual or sensor based detection of the presence or absence of a color (i.e., color detection method), and the presence or absence of (or a particular amount of) an electrical signal.

The measuring can in some cases be quantitative, e.g., in the sense that the amount of signal detected can be used to determine the amount of target DNA present in the sample. The measuring can in some cases be qualitative, e.g., in the sense that the presence or absence of detectable signal can indicate the presence or absence of targeted DNA (e.g., virus, single nucleotide polymorphism (SNP), etc.). In some cases, a detectable signal will not be present (e.g., above a given threshold level) unless the targeted DNA(s) (e.g., virus, SNP, etc.) is present above a particular threshold concentration. In some cases, the threshold of detection can be titrated by modifying the amount of variant CRISPR-Cas effector polypeptide, guide RNA, sample volume, and/or detector ssDNA (if one is used). As such, for example, as would be understood by one of ordinary skill in the art, a number of controls can be used if desired in order to set up one or more reactions, each set up to detect a different threshold level of target DNA, and thus such a series of reactions could be used to determine the amount of target DNA present in a sample (e.g., one could use such a series of reactions to determine that a target DNA is present in the sample ‘at a concentration of at least X’).

Examples of uses of a detection method of the present disclosure include, e.g., single nucleotide polymorphism (SNP) detection, cancer screening, detection of bacterial infection, detection of antibiotic resistance, detection of viral infection, and the like. The compositions and methods of this disclosure can be used to detect any DNA target. For example, any virus that integrates nucleic acid material into the genome can be detected because a subject sample can include cellular genomic DNA—and the guide RNA can be designed to detect integrated nucleotide sequence.

In some cases, a method of the present disclosure can be used to determine the amount of a target DNA in a sample (e.g., a sample comprising the target DNA and a plurality of non-target DNAs). Determining the amount of a target DNA in a sample can comprise comparing the amount of detectable signal generated from a test sample to the amount of detectable signal generated from a reference sample. Determining the amount of a target DNA in a sample can comprise: measuring the detectable signal to generate a test measurement; measuring a detectable signal produced by a reference sample to generate a reference measurement; and comparing the test measurement to the reference measurement to determine an amount of target DNA present in the sample.

For example, in some cases, a method of the present disclosure for determining the amount of a target DNA in a sample comprises: a) contacting the sample (e.g., a sample comprising the target DNA and a plurality of non-target DNAs) with: (i) a guide RNA that hybridizes with the target DNA, (ii) a variant CRISPR-Cas effector polypeptide of the present disclosure that cleaves RNAs present in the sample, and (iii) a detector ssDNA; b) measuring a detectable signal produced by variant CRISPR-Cas effector polypeptide-mediated ssDNA cleavage (e.g., cleavage of the detector ssDNA), generating a test measurement; c) measuring a detectable signal produced by a reference sample to generate a reference measurement; and d) comparing the test measurement to the reference measurement to determine an amount of target DNA present in the sample.

As another example, in some cases, a method of the present disclosure for determining the amount of a target DNA in a sample comprises: a) contacting the sample (e.g., a sample comprising the target DNA and a plurality of non-target DNAs) with: i) a precursor guide RNA array comprising two or more guide RNAs each of which has a different guide sequence; (ii) a variant CRISPR-Cas effector polypeptide of the present disclosure that cleaves the precursor guide RNA array into individual guide RNAs, and also cleaves RNAs of the sample; and (iii) a detector ssDNA; b) measuring a detectable signal produced by variant CRISPR-Cas effector polypeptide-mediated ssDNA cleavage (e.g., cleavage of the detector ssDNA), generating a test measurement; c) measuring a detectable signal produced by each of two or more reference samples to generate two or more reference measurements; and d) comparing the test measurement to the reference measurements to determine an amount of target DNA present in the sample.

### Amplification of Nucleic Acids in the Sample

In some cases, sensitivity of a subject composition and/or method (e.g., for detecting the presence of a target DNA, such as viral DNA or a SNP, in cellular genomic DNA) can be increased by coupling detection with nucleic acid amplification. In some cases, the nucleic acids in a sample are amplified prior to contact with a variant CRISPR-Cas effector polypeptide of the present disclosure that cleaved ssDNA (e.g., amplification of nucleic acids in the sample can begin prior to contact with a variant CRISPR-Cas effector polypeptide of the present disclosure). In some cases, the nucleic acids in a sample are amplified simultaneously with contact with a variant CRISPR-Cas effector polypeptide of the present disclosure. For example, in some cases, a subject method includes amplifying nucleic acids of a sample (e.g., by contacting the sample with amplification components) prior to contacting the amplified sample with a variant CRISPR-Cas effector polypeptide of the present disclosure. In some cases, a subject method includes contacting a sample with amplification components at the same time (simultaneous with) that the sample is contacted with a variant CRISPR-Cas effector polypeptide of the present disclosure. If all components are added simultaneously (amplification components and detection components such as a variant CRISPR-Cas effector polypeptide of the present disclosure, a guide RNA, and a detector DNA), it is possible that the trans-cleavage activity of the variant CRISPR-Cas effector polypeptide will begin to degrade the nucleic acids of the sample at the same time the nucleic acids are undergoing amplification. However, even if this is the case, amplifying and detecting simultaneously can still increase sensitivity compared to performing the method without amplification.

In some cases, specific sequences (e.g., sequences of a virus, sequences that include a SNP of interest) are amplified from the sample, e.g., using primers. As such, a sequence to which the guide RNA will hybridize can be amplified in order to increase sensitivity of a subject detection method —this could achieve biased amplification of a desired sequence in order to increase the number of copies of the sequence of interest present in the sample relative to other sequences present in the sample. As one illustrative example, if a subject method is being used to determine whether a given sample includes a particular virus (or a particular SNP), a desired region of viral sequence (or non-viral genomic sequence) can be amplified, and the region amplified will include the sequence that would hybridize to the guide RNA if the viral sequence (or SNP) were in fact present in the sample.

As noted, in some cases the nucleic acids are amplified (e.g., by contact with amplification components) prior to contacting the amplified nucleic acids with a variant CRISPR-Cas effector polypeptide of the present disclosure. In some cases, amplification occurs for 10 seconds or more, (e.g., 30 seconds or more, 45 seconds or more, 1 minute or more, 2 minutes or more, 3 minutes or more, 4 minutes or more, 5 minutes or more, 7.5 minutes or more, 10 minutes or more, etc.) prior to contact with a variant CRISPR-Cas effector polypeptide of the present disclosure. In some cases, amplification occurs for 2 minutes or more (e.g., 3 minutes or more, 4 minutes or more, 5 minutes or more, 7.5 minutes or more, 10 minutes or more, etc.) prior to contact with a variant CRISPR-Cas effector polypeptide of the present disclosure. In some cases, amplification occurs for a period of time in a range of from 10 seconds to 60 minutes (e.g., 10 seconds to 40 minutes, 10 seconds to 30 minutes, 10 seconds to 20 minutes, 10 seconds to 15 minutes, 10 seconds to 10 minutes, 10 seconds to 5 minutes, 30 seconds to 40 minutes, 30 seconds to 30 minutes, 30 seconds to 20 minutes, 30 seconds to 15 minutes, 30 seconds to 10 minutes, 30 seconds to 5 minutes, 1 minute to 40 minutes, 1 minute to 30 minutes, 1 minute to 20 minutes, 1 minute to 15 minutes, 1 minute to 10 minutes, 1 minute to 5 minutes, 2 minutes to 40 minutes, 2 minutes to 30 minutes, 2 minutes to 20 minutes, 2 minutes to 15 minutes, 2 minutes to 10 minutes, 2 minutes to 5 minutes, 5 minutes to 40 minutes, 5 minutes to 30 minutes, 5 minutes to 20 minutes, 5 minutes to 15 minutes, or 5 minutes to 10 minutes). In some cases, amplification occurs for a period of time in a range of from 5 minutes to 15 minutes. In some cases, amplification occurs for a period of time in a range of from 7 minutes to 12 minutes.

In some cases, a sample is contacted with amplification components at the same time as contact with a variant CRISPR-Cas effector polypeptide of the present disclosure. In some such cases, the variant CRISPR-Cas effector protein is inactive at the time of contact and is activated once nucleic acids in the sample have been amplified.

Various amplification methods and components will be known to one of ordinary skill in the art and any convenient method can be used (see, e.g., Zanoli and Spoto, Biosensors (Basel). 2013 March; 3(1): 18-43; Gill and Ghaemi, Nucleosides, Nucleotides, and Nucleic Acids, 2008, 27: 224-243; Craw and Balachandrana, Lab Chip, 2012, 12, 2469-2486; which are herein incorporated by reference in their entirety). Nucleic acid amplification can comprise polymerase chain reaction (PCR), reverse transcription PCR (RT-PCR), quantitative PCR (qPCR), reverse transcription qPCR (RT-qPCR), nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR), allele-specific PCR, intersequence-specific PCR (ISS-PCR), whole genome amplification (WGA), inverse PCR, and thermal asymmetric interlaced PCR (TAIL-PCR).

In some cases, the amplification is isothermal amplification. The term “isothermal amplification” indicates a method of nucleic acid (e.g., DNA) amplification (e.g., using enzymatic chain reaction) that can use a single temperature incubation thereby obviating the need for a thermal cycler. Isothermal amplification is a form of nucleic acid amplification which does not rely on the thermal denaturation of the target nucleic acid during the amplification reaction and hence may not require multiple rapid changes in temperature. Isothermal nucleic acid amplification methods can therefore be carried out inside or outside of a laboratory environment. By combining with a reverse transcription step, these amplification methods can be used to isothermally amplify RNA.

Examples of isothermal amplification methods include but are not limited to: loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA).

In some cases, the amplification is recombinase polymerase amplification (RPA) (see, e.g., U.S. Pat. Nos. 8,030,000; 8,426,134; 8,945,845; 9,309,502; and 9,663,820, which are hereby incorporated by reference in their entirety). Recombinase polymerase amplification (RPA) uses two opposing primers (much like PCR) and employs three enzymes—a recombinase, a single-stranded DNA-binding protein (SSB) and a strand-displacing polymerase. The recombinase pairs oligonucleotide primers with homologous sequence in duplex DNA, SSB binds to displaced strands of DNA to prevent the primers from being displaced, and the strand displacing polymerase begins DNA synthesis where the primer has bound to the target DNA. Adding a reverse transcriptase enzyme to an RPA reaction can facilitate detection RNA as well as DNA, without the need for a separate step to produce cDNA. One example of components for an RPA reaction is as follows (see, e.g., U.S. Pat. Nos. 8,030,000; 8,426,134; 8,945,845; 9,309,502; 9,663,820): 50 mM Tris pH 8.4, 80 mM Potassium acetate, 10 mM Magnesium acetate, 2 mM dithiothreitol (DTT), 5% PEG compound (Carbowax-20M), 3 mM ATP, 30 mM Phosphocreatine, 100 ng/μl creatine kinase, 420 ng/μl gp32, 140 ng/μl UvsX, 35 ng/μl UvsY, 2000M dNTPs, 300 nM each oligonucleotide, 35 ng/μl Bsu polymerase, and a nucleic acid-containing sample).

In a transcription mediated amplification (TMA), an RNA polymerase is used to make RNA from a promoter engineered in the primer region, and then a reverse transcriptase synthesizes cDNA from the primer. A third enzyme, e.g., Rnase H can then be used to degrade the RNA target from cDNA without the heat-denatured step. This amplification technique is similar to Self-Sustained Sequence Replication (3SR) and Nucleic Acid Sequence Based Amplification (NASBA), but varies in the enzymes employed. For another example, helicase-dependent amplification (HDA) utilizes a thermostable helicase (Tte-UvrD) rather than heat to unwind dsDNA to create single-strands that are then available for hybridization and extension of primers by polymerase. For yet another example, a loop mediated amplification (LAMP) employs a thermostable polymerase with strand displacement capabilities and a set of four or more specific designed primers. Each primer is designed to have hairpin ends that, once displaced, snap into a hairpin to facilitate self-priming and further polymerase extension. In a LAMP reaction, though the reaction proceeds under isothermal conditions, an initial heat denaturation step is required for double-stranded targets. In addition, amplification yields a ladder pattern of various length products. For yet another example, a strand displacement amplification (SDA) combines the ability of a restriction endonuclease to nick the unmodified strand of its target DNA and an exonuclease-deficient DNA polymerase to extend the 3′ end at the nick and displace the downstream DNA strand.

### Detector DNA

In some cases, a subject method includes contacting a sample (e.g., a sample comprising a target DNA and a plurality of non-target ssDNAs) with: i) a variant CRISPR-Cas effector polypeptide of the present disclosure; ii) a guide RNA (or precursor guide RNA array); and iii) a detector DNA that is single stranded and does not hybridize with the guide sequence of the guide RNA. For example, in some cases, a subject method includes contacting a sample with a labeled single stranded detector DNA (detector ssDNA) that includes a fluorescence-emitting dye pair; the variant CRISPR-Cas effector polypeptide cleaves the labeled detector ssDNA after it is activated (by binding to the guide RNA in the context of the guide RNA hybridizing to a target DNA); and the detectable signal that is measured is produced by the fluorescence-emitting dye pair. For example, in some cases, a subject method includes contacting a sample with a labeled detector ssDNA comprising a fluorescence resonance energy transfer (FRET) pair or a quencher/fluor pair, or both. In some cases, a subject method includes contacting a sample with a labeled detector ssDNA comprising a FRET pair. In some cases, a subject method includes contacting a sample with a labeled detector ssDNA comprising a fluor/quencher pair.

Fluorescence-emitting dye pairs comprise a FRET pair or a quencher/fluor pair. In both cases of a FRET pair and a quencher/fluor pair, the emission spectrum of one of the dyes overlaps a region of the absorption spectrum of the other dye in the pair. As used herein, the term “fluorescence-emitting dye pair” is a generic term used to encompass both a “fluorescence resonance energy transfer (FRET) pair” and a “quencher/fluor pair,” both of which terms are discussed in more detail below. The term “fluorescence-emitting dye pair” is used interchangeably with the phrase “a FRET pair and/or a quencher/fluor pair.”

In some cases (e.g., when the detector ssDNA includes a FRET pair) the labeled detector ssDNA produces an amount of detectable signal prior to being cleaved, and the amount of detectable signal that is measured is reduced when the labeled detector ssDNA is cleaved. In some cases, the labeled detector ssDNA produces a first detectable signal prior to being cleaved (e.g., from a FRET pair) and a second detectable signal when the labeled detector ssDNA is cleaved (e.g., from a quencher/fluor pair). As such, in some cases, the labeled detector ssDNA comprises a FRET pair and a quencher/fluor pair.

In some cases, the labeled detector ssDNA comprises a FRET pair. FRET is a process by which radiationless transfer of energy occurs from an excited state fluorophore to a second chromophore in close proximity. The range over which the energy transfer can take place is limited to approximately 10 nanometers (100 angstroms), and the efficiency of transfer is extremely sensitive to the separation distance between fluorophores. Thus, as used herein, the term “FRET” (“fluorescence resonance energy transfer”; also known as “Forster resonance energy transfer”) refers to a physical phenomenon involving a donor fluorophore and a matching acceptor fluorophore selected so that the emission spectrum of the donor overlaps the excitation spectrum of the acceptor, and further selected so that when donor and acceptor are in close proximity (usually 10 nm or less) to one another, excitation of the donor will cause excitation of and emission from the acceptor, as some of the energy passes from donor to acceptor via a quantum coupling effect. Thus, a FRET signal serves as a proximity gauge of the donor and acceptor; only when they are in close proximity to one another is a signal generated. The FRET donor moiety (e.g., donor fluorophore) and FRET acceptor moiety (e.g., acceptor fluorophore) are collectively referred to herein as a “FRET pair”.

The donor-acceptor pair (a FRET donor moiety and a FRET acceptor moiety) is referred to herein as a “FRET pair” or a “signal FRET pair.” Thus, in some cases, a subject labeled detector ssDNA includes two signal partners (a signal pair), when one signal partner is a FRET donor moiety and the other signal partner is a FRET acceptor moiety. A subject labeled detector ssDNA that includes such a FRET pair (a FRET donor moiety and a FRET acceptor moiety) will thus exhibit a detectable signal (a FRET signal) when the signal partners are in close proximity (e.g., while on the same RNA molecule), but the signal will be reduced (or absent) when the partners are separated (e.g., after cleavage of the RNA molecule by a variant CRISPR-Cas effector polypeptide of the present disclosure).

FRET donor and acceptor moieties (FRET pairs) will be known to one of ordinary skill in the art and any convenient FRET pair (e.g., any convenient donor and acceptor moiety pair) can be used. Examples of suitable FRET pairs include but are not limited to those presented in Table 1. See also: Bajar et al. Sensors (Basel). 2016 Sep. 14; 16(9); and Abraham et al. PLoS One. 2015 Aug. 3; 10(8):e0134436.

TABLE 1
Examples of FRET pairs (donor and acceptor FRET moieties)

| Donor | Acceptor |
|---|---|
| Tryptophan | Dansyl |
| IAEDANS (1) | DDPM (2) |
| BFP | DsRFP |
| Dansyl | Fluorescein |
| | isothiocyanate (FITC) |
| Dansyl | Octadecylrhodamine |
| Cyan fluorescent | Green fluorescent protein |
| protein (CFP) | (GFP) |
| CF (3) | Texas Red |
| Fluorescein | Tetramethylrhodamine |
| Cy3 | Cy5 |
| GFP | Yellow fluorescent |
| | protein (YFP) |
| BODIPY FL (4) | BODIPY FL (4) |
| Rhodamine 110 | Cy3 |
| Rhodamine 6G | Malachite Green |
| FITC | Eosin Thiosemicarbazide |
| B-Phycoerythrin | Cy5 |
| Cy5 | Cy5.5 |

(1) 5-(2-iodoacetylaminoethyl)aminonaphthalene-1-sulfonic acid
(2) N-(4-dimethylamino-3,5-dinitrophenyl)maleimide
(3) carboxyfluorescein succinimidyl ester
(4) 4,4-difluoro-4-bora-3a,4a-diaza-s-indacene

In some cases, a detectable signal is produced when the labeled detector ssDNA is cleaved (e.g., in some cases, the labeled detector ssDNA comprises a quencher/fluor pair). One signal partner of a signal quenching pair produces a detectable signal and the other signal partner is a quencher moiety that quenches the detectable signal of the first signal partner (i.e., the quencher moiety quenches the signal of the signal moiety such that the signal from the signal moiety is reduced (quenched) when the signal partners are in proximity to one another, e.g., when the signal partners of the signal pair are in close proximity).

For example, in some cases, an amount of detectable signal increases when the labeled detector ssDNA is cleaved. For example, in some cases, the signal exhibited by one signal partner (a signal moiety) is quenched by the other signal partner (a quencher signal moiety), e.g., when both are present on the same ssDNA molecule prior to cleavage by a variant CRISPR-Cas effector polypeptide of the present disclosure). Such a signal pair is referred to herein as a “quencher/fluor pair”, “quenching pair”, or “signal quenching pair.” For example, in some cases, one signal partner (e.g., the first signal partner) is a signal moiety that produces a detectable signal that is quenched by the second signal partner (e.g., a quencher moiety). The signal partners of such a quencher/fluor pair will thus produce a detectable signal when the partners are separated (e.g., after cleavage of the detector ssDNA by a variant CasPhi polypeptide of the present disclosure), but the signal will be quenched when the partners are in close proximity (e.g., prior to cleavage of the detector ssDNA by a variant CRISPR-Cas effector polypeptide of the present disclosure).

A quencher moiety can quench a signal from the signal moiety (e.g., prior to cleave of the detector ssDNA by a variant CRISPR-Cas effector polypeptide of the present disclosure) to various degrees. In some cases, a quencher moiety quenches the signal from the signal moiety where the signal detected in the presence of the quencher moiety (when the signal partners are in proximity to one another) is 95% or less of the signal detected in the absence of the quencher moiety (when the signal partners are separated). For example, in some cases, the signal detected in the presence of the quencher moiety can be 90% or less, 80% or less, 70% or less, 60% or less, 50% or less, 40% or less, 30% or less, 20% or less, 15% or less, 10% or less, or 5% or less of the signal detected in the absence of the quencher moiety. In some cases, no signal (e.g., above background) is detected in the presence of the quencher moiety.

In some cases, the signal detected in the absence of the quencher moiety (when the signal partners are separated) is at least 1.2 fold greater (e.g., at least 1.3 fold, at least 1.5 fold, at least 1.7 fold, at least 2 fold, at least 2.5 fold, at least 3 fold, at least 3.5 fold, at least 4 fold, at least 5 fold, at least 7 fold, at least 10 fold, at least 20 fold, or at least 50 fold greater) than the signal detected in the presence of the quencher moiety (when the signal partners are in proximity to one another).

In some cases, the signal moiety is a fluorescent label. In some such cases, the quencher moiety quenches the signal (the light signal) from the fluorescent label (e.g., by absorbing energy in the emission spectra of the label). Thus, when the quencher moiety is not in proximity with the signal moiety, the emission (the signal) from the fluorescent label is detectable because the signal is not absorbed by the quencher moiety. Any convenient donor acceptor pair (signal moiety/quencher moiety pair) can be used and many suitable pairs are known in the art.

In some cases, the quencher moiety absorbs energy from the signal moiety (also referred to herein as a “detectable label”) and then emits a signal (e.g., light at a different wavelength). Thus, in some cases, the quencher moiety is itself a signal moiety (e.g., a signal moiety can be 6-carboxyfluorescein while the quencher moiety can be 6-carboxy-tetramethylrhodamine), and in some such cases, the pair could also be a FRET pair. In some cases, a quencher moiety is a dark quencher. A dark quencher can absorb excitation energy and dissipate the energy in a different way (e.g., as heat). Thus, a dark quencher has minimal to no fluorescence of its own (does not emit fluorescence). Examples of dark quenchers are further described in U.S. Pat. Nos. 8,822,673 and 8,586,718; U.S. patent publications 20140378330, 20140349295, and 20140194611; and international patent applications: WO200142505 and WO200186001, all if which are hereby incorporated by reference in their entirety.

Examples of fluorescent labels include, but are not limited to: an Alexa Fluor® dye, an ATO dye (e.g., ATTO 390, ATO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein isothiocyanate (FITC), tetramethylrhodamine (TRITC), Texas Red, Oregon Green, Pacific Blue, Pacific Green, Pacific Orange, quantum dots, and a tethered fluorescent protein.

In some cases, a detectable label is a fluorescent label selected from: an Alexa Fluor® dye, an ATTO dye (e.g., ATTO 390, ATO 425, ATO 465, ATTO 488, ATO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein (FITC), tetramethylrhodamine (TRITC), Texas Red, Oregon Green, Pacific Blue, Pacific Green, and Pacific Orange.

In some cases, a detectable label is a fluorescent label selected from: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein (FITC), tetramethylrhodamine (TRITC), Texas Red, Oregon Green, Pacific Blue, Pacific Green, Pacific Orange, a quantum dot, and a tethered fluorescent protein.

Examples of ATO dyes include, but are not limited to: ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, and ATTO 740.

Examples of AlexaFluor dyes include, but are not limited to: Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, Alexa Fluor® 790, and the like.

Examples of quencher moieties include, but are not limited to: a dark quencher, a Black Hole Quencher® (BHQ®) (e.g., BHQ-0, BHQ-1, BHQ-2, BHQ-3), a Qxl quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and metal clusters such as gold nanoparticles, and the like.

In some cases, a quencher moiety is selected from: a dark quencher, a Black Hole Quencher® (BHQ®) (e.g., BHQ-0, BHQ-1, BHQ-2, BHQ-3), a Qxl quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and a metal cluster.

Examples of an ATTO quencher include, but are not limited to: ATTO 540Q, ATTO 580Q, and ATTO 612Q. Examples of a Black Hole Quencher® (BHQ®) include, but are not limited to: BHQ-0 (493 nm), BHQ-1 (534 nm), BHQ-2 (579 nm) and BHQ-3 (672 nm).

For examples of some detectable labels (e.g., fluorescent dyes) and/or quencher moieties, see, e.g., Bao et al., Annu Rev Biomed Eng. 2009; 11:25-47; as well as U.S. Pat. Nos. 8,822,673 and 8,586,718; U.S. patent publications 20140378330, 20140349295, 20140194611, 20130323851, 20130224871, 20110223677, 20110190486, 20110172420, 20060179585 and 20030003486; and international patent applications: WO200142505 and WO200186001, all of which are hereby incorporated by reference in their entirety.

In some cases, cleavage of a labeled detector ssDNA can be detected by measuring a colorimetric read-out. For example, the liberation of a fluorophore (e.g., liberation from a FRET pair, liberation from a quencher/fluor pair, and the like) can result in a wavelength shift (and thus color shift) of a detectable signal. Thus, in some cases, cleavage of a subject labeled detector ssDNA can be detected by a color-shift. Such a shift can be expressed as a loss of an amount of signal of one color (wavelength), a gain in the amount of another color, a change in the ration of one color to another, and the like.

### Transgenic, Non-Human Organisms

As described above, in some cases, a nucleic acid (e.g., a recombinant expression vector) of the present disclosure (e.g., a nucleic acid comprising a nucleotide sequence encoding a variant CRISPR-Cas effector polypeptide of the present disclosure; a nucleic acid comprising a nucleotide sequence encoding a fusion polypeptide of the present disclosure; etc.), is used as a transgene to generate a transgenic non-human organism that produces a variant CRISPR-Cas effector polypeptide, or a fusion polypeptide, of the present disclosure. The present disclosure provides a transgenic-non-human organism comprising a nucleotide sequence encoding a variant CRISPR-Cas effector polypeptide, or a fusion polypeptide, of the present disclosure.

### Transgenic, Non-Human Animals

The present disclosure provides a transgenic non-human animal, which animal comprises a transgene comprising a nucleic acid comprising a nucleotide sequence encoding a variant CRISPR-Cas effector polypeptide or a fusion polypeptide. In some cases, the genome of the transgenic non-human animal comprises a nucleotide sequence encoding a variant CRISPR-Cas effector polypeptide or a fusion polypeptide, of the present disclosure. In some cases, the transgenic non-human animal is homozygous for the genetic modification. In some cases, the transgenic non-human animal is heterozygous for the genetic modification. In some embodiments, the transgenic non-human animal is a vertebrate, for example, a fish (e.g., salmon, trout, zebra fish, gold fish, puffer fish, cave fish, etc.), an amphibian (frog, newt, salamander, etc.), a bird (e.g., chicken, turkey, etc.), a reptile (e.g., snake, lizard, etc.), a non-human mammal (e.g., an ungulate, e.g., a pig, a cow, a goat, a sheep, etc.; a lagomorph (e.g., a rabbit); a rodent (e.g., a rat, a mouse); a non-human primate; etc.), etc. In some cases, the transgenic non-human animal is an invertebrate. In some cases, the transgenic non-human animal is an insect (e.g., a mosquito; an agricultural pest; etc.). In some cases, the transgenic non-human animal is an arachnid.

Nucleotide sequences encoding a variant CRISPR-Cas effector polypeptide, or a fusion polypeptide, of the present disclosure can be under the control of (i.e., operably linked to) an unknown promoter (e.g., when the nucleic acid randomly integrates into a host cell genome) or can be under the control of (i.e., operably linked to) a known promoter. Suitable known promoters can be any known promoter and include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc.

### Transgenic Plants

As described above, in some cases, a nucleic acid (e.g., a recombinant expression vector) of the present disclosure (e.g., a nucleic acid comprising a nucleotide sequence encoding a variant CRISPR-Cas effector polypeptide of the present disclosure; a nucleic acid comprising a nucleotide sequence encoding a fusion polypeptide of the present disclosure; etc.), is used as a transgene to generate a transgenic plant that produces a variant CRISPR-Cas effector polypeptide, or a fusion polypeptide, of the present disclosure. The present disclosure provides a transgenic plant comprising a nucleotide sequence encoding a variant CRISPR-Cas effector polypeptide, or a fusion polypeptide, of the present disclosure. In some cases, the genome of the transgenic plant comprises a subject nucleic acid. In some embodiments, the transgenic plant is homozygous for the genetic modification. In some embodiments, the transgenic plant is heterozygous for the genetic modification.

In some cases, a transgenic plant of the present disclosure comprises a genetic modification produced using a variant CRISPR-Cas effector polypeptide of the present disclosure (and a guide nucleic acid), where the genetic modification is in only one allele of a target gene. In some cases, a transgenic plant of the present disclosure comprises a genetic modification produced using a variant CRISPR-Cas effector polypeptide of the present disclosure (and a guide nucleic acid), where the genetic modification is in both alleles of a target gene. In some cases, where the plant species is polyploid, a transgenic plant of the present disclosure comprises a genetic modification produced using a variant CRISPR-Cas effector polypeptide of the present disclosure (and a guide nucleic acid), where the genetic modification is in one allele, two alleles, more than two alleles, or all alleles of a target gene. In some cases, a transgenic plant of the present disclosure is a T0 plant. The present disclosure provides T1 seeds from the T0 plant. In some cases, the transgenic plant is a T1 plant grown from the T1 seeds. The present disclosure provides T2 seeds from the T1 plant. In some cases, the transgenic plant is a T2 plant grown from the T2 seeds. In some cases, the genetic modification made using a variant CRISPR-Cas effector polypeptide of the present disclosure (and a guide nucleic acid) is present in one or both alleles of a T0 plant, a T1 plant, a T2 plant, and subsequent generations of the plant and in seeds of the T0 plant, the T1 plant, the T2 plant, and in the subsequent generations of the plant.

Methods of introducing exogenous nucleic acids into plant cells are well known in the art. Such plant cells are considered “transformed,” as defined above. Suitable methods include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation and the like. The choice of method is generally dependent on the type of cell being transformed and the circumstances under which the transformation is taking place (i.e. in vitro, ex vivo, or in vivo).

Transformation methods based upon the soil bacterium Agrobacterium tumefaciens are particularly useful for introducing an exogenous nucleic acid molecule into a vascular plant. The wild type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors is well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art. See, e.g., Glick and Thompson, (eds.), Methods in Plant Molecular Biology and Biotechnology, Boca Raton, Fla.: CRC Press (1993).

Microprojectile-mediated transformation also can be used to produce a subject transgenic plant. This method, first described by Klein et al. (Nature 327:70-73 (1987)), relies on microprojectiles such as gold or tungsten that are coated with the desired nucleic acid molecule by precipitation with calcium chloride, spermidine or polyethylene glycol. The microprojectile particles are accelerated at high speed into an angiosperm tissue using a device such as the BIOLISTIC PD-1000 (Biorad; Hercules Calif.).

A nucleic acid of the present disclosure (e.g., a nucleic acid (e.g., a recombinant expression vector) comprising a nucleotide sequence encoding a variant CRISPR-Cas effector polypeptide, or a fusion polypeptide, of the present disclosure) may be introduced into a plant in a manner such that the nucleic acid is able to enter a plant cell(s), e.g., via an in vivo or ex vivo protocol. By “in vivo,” it is meant in the nucleic acid is administered to a living body of a plant e.g. infiltration. By “ex vivo” it is meant that cells or explants are modified outside of the plant, and then such cells or organs are regenerated to a plant. A number of vectors suitable for stable transformation of plant cells or for the establishment of transgenic plants have been described, including those described in Weissbach and Weissbach, (1989) Methods for Plant Molecular Biology Academic Press, and Gelvin et al., (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303: 209, Bevan (1984) Nucl Acid Res. 12: 8711-8721, Klee (1985) Bio/Technolo 3: 637-642. Alternatively, non-Ti vectors can be used to transfer the DNA into plants and cells by using free DNA delivery techniques. By using these methods transgenic plants such as wheat, rice (Christou (1991) Bio/Technology 9:957-9 and 4462) and corn (Gordon-Kamm (1990) Plant Cell 2: 603-618) can be produced. An immature embryo can also be a good target tissue for monocots for direct DNA delivery techniques by using the particle gun (Weeks et al. (1993) Plant Physiol 102: 1077-1084; Vasil (1993) Bio/Technolo 10: 667-674; Wan and Lemeaux (1994) Plant Physiol 104: 37-48 and for Agrobacterium-mediated DNA transfer (Ishida et al. (1996) Nature Biotech 14: 745-750). Exemplary methods for introduction of DNA into chloroplasts are biolistic bombardment, polyethylene glycol transformation of protoplasts, and microinjection (Danieli et al Nat. Biotechnol 16:345-348, 1998; Staub et al Nat. Biotechnol 18: 333-338, 2000; O'Neill et al Plant J. 3:729-738, 1993; Knoblauch et al Nat. Biotechnol 17: 906-909; U.S. Pat. Nos. 5,451,513, 5,545,817, 5,545,818, and 5,576,198; in Intl. Application No. WO 95/16783; and in Boynton et al., Methods in Enzymology 217: 510-536 (1993), Svab et al., Proc. Natl. Acad. Sci. USA 90: 913-917 (1993), and McBride et al., Proc. Natl. Acad. Sci. USA 91: 7301-7305 (1994)). Any vector suitable for the methods of biolistic bombardment, polyethylene glycol transformation of protoplasts and microinjection will be suitable as a targeting vector for chloroplast transformation. Any double stranded DNA vector may be used as a transformation vector, especially when the method of introduction does not utilize Agrobacterium.

Plants which can be genetically modified include grains, forage crops, fruits, vegetables, oil seed crops, palms, forestry, and vines. Specific examples of plants which can be modified follow: maize, banana, peanut, field peas, sunflower, tomato, canola, tobacco, wheat, barley, oats, potato, soybeans, cotton, carnations, sorghum, lupin and rice.

The present disclosure provides transformed plant cells, tissues, plants and products that contain the transformed plant cells. A feature of the subject transformed cells, and tissues and products that include the same is the presence of a subject nucleic acid integrated into the genome, and production by plant cells of a variant CRISPR-Cas effector polypeptide, or a fusion polypeptide, of the present disclosure. Recombinant plant cells of the present invention are useful as populations of recombinant cells, or as a tissue, seed, whole plant, stem, fruit, leaf, root, flower, stem, tuber, grain, animal feed, a field of plants, and the like.

Nucleotide sequences encoding a variant CRISPR-Cas effector polypeptide, or a fusion polypeptide, of the present disclosure can be under the control of (i.e., operably linked to) an unknown promoter (e.g., when the nucleic acid randomly integrates into a host cell genome) or can be under the control of (i.e., operably linked to) a known promoter. Suitable known promoters can be any known promoter and include constitutively active promoters, inducible promoters, spatially restricted and/or temporally restricted promoters, etc.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects.

This is intended to provide support for all such combinations of aspects and is not limited to combinations of aspects explicitly provided below:

Aspect 1. A variant CRISPR-Cas effector polypeptide comprising an amino acid sequence having at least 50% amino acid sequence identity to any one of the amino acid sequences depicted in FIG. 9A-9R, wherein the variant CRISPR-Cas effector polypeptide comprises a deletion or a substitution of one or more amino acids in the alpha-7 helix of the Rec I domain, compared to the amino acid sequence depicted in FIG. 6, or a corresponding region of another CasPhi polypeptide, and wherein the variant CRISPR-Cas effector polypeptide exhibits at least a 10% increased cis- and/or trans-cleavage activity compared to the cis- and/or trans-cleavage activity of a CasPhi polypeptide comprising the amino acid sequence depicted in FIG. 6.

Aspect 2. The variant CRISPR-Cas effector polypeptide of aspect 1, wherein the variant CRISPR-Cas effector polypeptide comprises amino acid substitutions of amino acids E159, S160, S164, D167, and E168, compared to the amino acid sequence depicted in FIG. 6, or corresponding amino acids in another CasPhi polypeptide.

Aspect 3. The variant CRISPR-Cas effector polypeptide of aspect 2, wherein the variant CRISPR-Cas effector polypeptide comprises E159A, S160A, S164A, D167A, and E168A substitutions, compared to the amino acid sequence depicted in FIG. 6.

Aspect 4. The variant CRISPR-Cas effector polypeptide of aspect 1, wherein the variant CRISPR-Cas effector polypeptide comprises a replacement of from 15 amino acids to 52 amino acids within amino acids 144-195 of the amino acid sequence depicted in FIG. 6, or a corresponding stretch of amino acids in the alpha-7 helix of another CasPhi polypeptide, with a heterologous polypeptide.

Aspect 5. The variant CRISPR-Cas effector polypeptide of aspect 4, wherein the variant CRISPR-Cas effector polypeptide comprises a replacement of amino acids 155-176 of the amino acid sequence depicted in FIG. 6, or a corresponding stretch of amino acids in another CasPhi polypeptide.

Aspect 6. The variant CRISPR-Cas effector polypeptide of aspect 4 or aspect 5, wherein the heterologous polypeptide comprises Gly, Ser, or a combination of Gly and Ser, and wherein the heterologous polypeptide has a length of from 4 amino acids to about 25 amino acids.

Aspect 7. The variant CRISPR-Cas effector polypeptide of aspect 4 or aspect 5, wherein the heterologous polypeptide exhibits an enzymatic activity.

Aspect 8. The variant CRISPR-Cas effector polypeptide of aspect 7, wherein the heterologous polypeptide is a base editor.

Aspect 9. The variant CRISPR-Cas effector polypeptide of aspect 4 or aspect 5, wherein the heterologous polypeptide comprises a protein-binding domain.

Aspect 10. The variant CRISPR-Cas effector polypeptide of aspect 4 or aspect 5, wherein the heterologous polypeptide is a nucleic acid-binding polypeptide, a nucleic acid modifying polypeptide, or a protein-binding polypeptide.

Aspect 11. The variant CRISPR-Cas effector polypeptide of any one of aspects 1-10, wherein the variant CRISPR-Cas effector polypeptide exhibits at least 2-fold increased cis- and/or trans-cleavage activity compared to the cis- and/or trans-cleavage activity of a CasPhi polypeptide comprising the amino acid sequence depicted in FIG. 6.

Aspect 12. The variant CRISPR-Cas effector polypeptide of any one of aspects 1-10, wherein the variant CRISPR-Cas effector polypeptide exhibits at least 5-fold increased cis- and/or trans-cleavage activity compared to the cis- and/or trans-cleavage activity of a CasPhi polypeptide comprising the amino acid sequence depicted in FIG. 6.

Aspect 13. The variant CRISPR-Cas effector polypeptide of any one of aspects 1-10, wherein the variant CRISPR-Cas effector polypeptide exhibits at least 10-fold increased cis- and/or trans-cleavage activity compared to the cis- and/or trans-cleavage activity of a CasPhi polypeptide comprising the amino acid sequence depicted in FIG. 6.

Aspect 14. The variant CRISPR-Cas effector polypeptide of any one of aspects 1-10, wherein the variant CRISPR-Cas effector polypeptide exhibits at least 15-fold increased cis- and/or trans-cleavage activity compared to the cis- and/or trans-cleavage activity of a CasPhi polypeptide comprising the amino acid sequence depicted in FIG. 6.

Aspect 15. A fusion polypeptide comprising:
a) a variant CRISPR-Cas effector polypeptide of any one of aspects 1-14; and
b) one or more heterologous polypeptides.

Aspect 16. The fusion polypeptide of aspect 15, wherein the one or more heterologous polypeptides is fused to the N-terminus and/or the C-terminus of the variant CRISPR-Cas effector polypeptide.

Aspect 17. The fusion polypeptide of aspect 15 or aspect 16, wherein at least one of the one or more heterologous polypeptides comprises a nuclear localization signal (NLS).

Aspect 18. The fusion polypeptide of any one of aspects 15-17, wherein at least one of the one or more heterologous polypeptides is a targeting polypeptide that provides for binding to a cell surface moiety on a target cell or target cell type.

Aspect 19. The fusion polypeptide of any one of aspects 15-17, wherein at least one of the one or more heterologous polypeptides exhibits an enzymatic activity that modifies target DNA.

Aspect 20. The fusion polypeptide of any one of aspects 15-17, wherein at least one of the one or more heterologous polypeptides exhibits an enzymatic activity that modifies a target polypeptide associated with a target nucleic acid.

Aspect 21. The fusion polypeptide of any one of aspects 15-20, wherein at least one of the one or more heterologous polypeptides is an endosomal escape polypeptide.

Aspect 22. The fusion polypeptide of any one of aspects 15-20, wherein at least one of the one or more heterologous polypeptides is a chloroplast transit peptide.

Aspect 23. The fusion polypeptide of any one of aspects 15-20, wherein at least one of the one or more heterologous polypeptides comprises a protein transduction domain.

Aspect 24. The fusion polypeptide of any one of aspects 15-20, wherein at least one of the one or more heterologous polypeptides is a protein binding domain.

Aspect 25. A composition comprising:
a1) a variant CRISPR-Cas effector polypeptide of any one of aspects 1-14, or a nucleic acid comprising a nucleotide sequence encoding the variant CRISPR-Cas effector polypeptide; and
b1) a CasPhi guide RNA, or one or more DNA molecules encoding the CasPhi guide RNA; or
a2) a fusion polypeptide of any one of aspects 15-24 or a nucleic acid comprising a nucleotide sequence encoding the fusion polypeptide; and
b2) a CasPhi guide RNA, or one or more DNA molecules encoding the CasPhi guide RNA.

Aspect 26. The composition of aspect 25, wherein the CasPhi guide RNA comprises a nucleotide sequence having 80%, 90%, 95%, 98%, 99%, or 100%, nucleotide sequence identity with any one of the crRNA sequences depicted in FIG. 10, or the reverse complement of any one of the sequences depicted in FIG. 10 or FIG. 11.

Aspect 27. The composition of aspect 25 or aspect 26, wherein the composition comprises a DNA molecule comprising a nucleotide sequence encoding the CasPhi guide RNA, and wherein the nucleotide sequence encoding the CasPhi guide RNA is operably linked to a Pol II promoter or a Pol III promoter.

Aspect 28. The composition of aspect 27, wherein the nucleotide sequence encoding the CasPhi guide RNA is operably linked to a Pol II promoter, and wherein the Pol II promoter is a UBQ10 promoter or a CmYLCV promoter.

Aspect 29. The composition of aspect 28, wherein the nucleotide sequence encoding the guide RNA is flanked by a nucleotide sequence encoding a first ribozyme stem loop and a nucleotide sequence encoding a second ribozyme stem loop.

Aspect 30. The composition of any one of aspects 25-29, wherein the guide RNA is a single-molecule guide RNA.

Aspect 31. The composition of any one of aspects 25-30, wherein the composition comprises a lipid.

Aspect 32. The composition of any one of aspects 25-31, wherein a) and b) are within a liposome.

Aspect 33. The composition of any one of aspects 25-30, wherein a) and b) are within a particle.

Aspect 34. The composition of any one of aspects 25-33, comprising one or more of: a buffer, a nuclease inhibitor, and a protease inhibitor.

Aspect 35. The composition of any one of aspects 25-34, further comprising a DNA donor template.

Aspect 36. The composition of any one of aspects 25-35, comprising a pharmaceutically acceptable excipient.

Aspect 37. A nucleic acid comprising a nucleotide sequence encoding the variant CRISPR-Cas effector polypeptide of any one of aspects 1-14, or the fusion polypeptide of any one of aspects 15-24.

Aspect 38. The nucleic acid of aspect 37, wherein the nucleotide sequence encoding the variant CRISPR-Cas effector polypeptide, or the nucleotide sequence encoding the fusion polypeptide, is operably linked to a promoter.

Aspect 39. The nucleic acid of aspect 38, wherein the promoter is functional in a eukaryotic cell.

Aspect 40. The nucleic acid of aspect 39, wherein the promoter is functional in one or more of: a plant cell, a fungal cell, an animal cell, cell of an invertebrate, a fly cell, a cell of a vertebrate, a mammalian cell, a primate cell, a non-human primate cell, and a human cell.

Aspect 41. The nucleic acid of any one of aspects 28-40, wherein the promoter is one or more of: a constitutive promoter, an inducible promoter, a cell type-specific promoter, and a tissue-specific promoter.

Aspect 42. The nucleic acid of any one of aspects 37-41, wherein the nucleic acid is a recombinant expression vector.

Aspect 43. The nucleic acid of aspect 42, wherein the recombinant expression vector is a recombinant adeno-associated viral vector, a recombinant retroviral vector, or a recombinant lentiviral vector.

Aspect 44. A composition comprising: a) the nucleic acid of any one of aspects 37-43; and b) one or more of: a buffer, a nuclease inhibitor, a salt, a lipid, and a pharmaceutically acceptable excipient.

Aspect 45. One or more nucleic acids comprising: (a) a nucleotide sequence encoding a CasPhi guide RNA; and (b) a nucleotide sequence encoding: i) a variant CRISPR-Cas polypeptide of any one of aspects 1-14; or ii) a fusion polypeptide of any one of aspects 15-24.

Aspect 46. The one or more nucleic acids of aspect 45, wherein the CasPhi guide RNA comprises a nucleotide sequence having 80% or more nucleotide sequence identity with any one of the crRNA sequences depicted in FIG. 10, or the reverse complement of any one of the sequences depicted in FIG. 10, or the reverse complement of any one of the sequences depicted in FIG. 11.

Aspect 47. The one or more nucleic acids of aspect 45 or aspect 46, wherein the nucleotide sequence encoding the CasPhi guide RNA is operably linked to a promoter.

Aspect 48. The one or more nucleic acids of aspect 47, wherein the promoter is a Pol-II promoter.

Aspect 49. The one or more nucleic acids of any one of aspects 45-48, wherein the nucleotide sequence encoding the variant CRISPR-Cas polypeptide, or the nucleotide sequence encoding the fusion polypeptide, is operably linked to a promoter.

Aspect 50. The one or more nucleic acids of aspect 49, wherein the promoter is a promoter that is functional in a eukaryotic cell.

Aspect 51. The one or more nucleic acids of aspect 49 or aspect 50, wherein the promoter is an inducible promoter.

Aspect 52. A composition comprising: a) the one or more nucleic acids of any one of aspects 45-51; and b) one or more of: a buffer, a nuclease inhibitor, a salt, a lipid, and a pharmaceutically acceptable excipient.

Aspect 53. A cell comprising one or more of: a) a variant CRISPR-Cas polypeptide of any one of aspects 1-14, or a nucleic acid comprising a nucleotide sequence encoding the variant CRISPR-Cas polypeptide; b) a fusion polypeptide of any one of aspects 15-24, or a nucleic acid comprising a nucleotide sequence encoding the fusion polypeptide, and c) a CasPhi guide RNA, or a nucleic acid comprising a nucleotide sequence encoding the CasPhi guide RNA.

Aspect 54. The cell of aspect 53, comprising the nucleic acid comprising a nucleotide sequence encoding the variant CRISPR-Cas polypeptide, or comprising the nucleic acid comprising a nucleotide sequence encoding the fusion polypeptide, wherein said nucleic acid is integrated into the genomic DNA of the cell.

Aspect 55. The cell of aspect 53 or aspect 54, wherein the cell is a eukaryotic cell.

Aspect 56. The cell of aspect 55, wherein the eukaryotic cell is a plant cell, a mammalian cell, an insect cell, an arachnid cell, a fungal cell, a bird cell, a reptile cell, an amphibian cell, an invertebrate cell, a mouse cell, a rat cell, a primate cell, a non-human primate cell, or a human cell.

Aspect 57. The cell of aspect 53 or aspect 54, wherein the cell is a prokaryotic cell.

Aspect 58. The cell of any one of aspects 53-57, wherein the cell is in vitro.

Aspect 59. The cell of any one of aspects 53-57, wherein the cell is in vivo.

Aspect 60. A method of modifying a target nucleic acid, the method comprising contacting the target nucleic acid with: a) a variant CRISPR-Cas effector polypeptide of any one of aspects 1-14; and b) a CasPhi guide RNA comprising a guide sequence that hybridizes to a target sequence of the target nucleic acid, wherein said contacting results in modification of the target nucleic acid by the variant CRISPR-Cas effector polypeptide.

Aspect 61. The method of aspect 60, wherein said modification is cleavage of the target nucleic acid.

Aspect 62. The method of aspect 60 or aspect 61, wherein the target nucleic acid is selected from: double stranded DNA, single stranded DNA, RNA, genomic DNA, and extrachromosomal DNA.

Aspect 63. The method of any one of aspects 60-62, wherein the target nucleic acid is present in repressive and compact chromatin.

Aspect 64. The method of any one of aspects 60-62, wherein the target nucleic acid is present in active and accessible chromatin.

Aspect 65. The method of any of aspects 60-64, wherein said contacting takes place in vitro outside of a cell.

Aspect 66. The method of any of aspects 60-64, wherein said contacting takes place inside of a cell in vitro.

Aspect 67. The method of any of aspects 60-65, wherein said contacting takes place inside of a cell in vivo.

Aspect 68. The method of aspect 67, wherein the cell is a eukaryotic cell.

Aspect 69. The method of aspect 68, wherein the cell is selected from: a plant cell, a fungal cell, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell.

Aspect 70. The method of aspect 66, wherein the cell is a prokaryotic cell.

Aspect 71. The method of any one of aspects 66-70, wherein said contacting results in genome editing.

Aspect 72. The method of any one of aspects 66-71, wherein said contacting comprises: introducing into a cell: (a) the variant CRISPR-Cas effector polypeptide, or a nucleic acid comprising a nucleotide sequence encoding the variant CRISPR-Cas effector polypeptide, and (b) the CasPhi guide RNA, or a nucleic acid comprising a nucleotide sequence encoding the CasPhi guide RNA.

Aspect 73. The method of aspect 72, wherein said contacting further comprises introducing a DNA donor template into the cell.

Aspect 74. A transgenic, multicellular, non-human organism whose genome comprises a transgene comprising a nucleotide sequence encoding one or more of: a) a variant CRISPR-Cas effector polypeptide of any one of aspects 1-14; b) a fusion polypeptide of any one of aspects 15-24; and c) a CasPhi guide RNA.

Aspect 75. The transgenic, multicellular, non-human organism of aspect 74, wherein the organism is a plant, an invertebrate animal, an insect, an arthropod, an arachnid, a parasite, a worm, a cnidarian, a vertebrate animal, a fish, a reptile, an amphibian, an ungulate, a bird, a pig, a horse, a sheep, a rodent, a mouse, a rat, or a non-human primate.

Aspect 76. The transgenic, multicellular, non-human organism of aspect 75, wherein the organism is a monocotyledon plant or a dicotyledon plant.

Aspect 77. A system comprising one of: a) a variant CRISPR-Cas effector polypeptide of any one of aspects 1-14 and a CasPhi guide RNA; b) a variant CRISPR-Cas effector polypeptide of any one of aspects 1-14, a CasPhi guide RNA, and a DNA donor template; c) a fusion polypeptide of any one of aspects 15-24 and a CasPhi guide RNA; d) a fusion polypeptide of any one of aspects 15-24, a CasPhi guide RNA, and a DNA donor template; e) an mRNA encoding a variant CRISPR-Cas effector polypeptide of any one of aspects 1-14, and a CasPhi guide RNA; f) an mRNA encoding a variant CRISPR-Cas effector polypeptide of any one of aspects 1-14; a CasPhi guide RNA, and a DNA donor template; g) an mRNA encoding a fusion polypeptide of any one of aspects 15-24, and a CasPhi guide RNA; h) an mRNA encoding a fusion polypeptide of any one of aspects 15-24, a CasPhi guide RNA, and a DNA donor template; i) one or more recombinant expression vectors comprising: i) a nucleotide sequence encoding a variant CRISPR-Cas effector polypeptide of any one of aspects 1-14; and ii) a nucleotide sequence encoding a CasPhi guide RNA; j) one or more recombinant expression vectors comprising: i) a nucleotide sequence encoding a variant CRISPR-Cas effector polypeptide of any one of aspects 1-14; ii) a nucleotide sequence encoding a CasPhi guide RNA; and iii) a DNA donor template; k) one or more recombinant expression vectors comprising: i) a nucleotide sequence encoding a fusion polypeptide of any one of aspects 15-24; and ii) a nucleotide sequence encoding a CasPhi guide RNA; and 1) one or more recombinant expression vectors comprising: i) a nucleotide sequence encoding a fusion polypeptide of any one of aspects 15-24; ii) a nucleotide sequence encoding a CasPhi guide RNA; and a DNA donor template.

Aspect 78. A composition comprising the system of aspect 77.

Aspect 79. The composition of aspect 78, comprising one or more of: a buffer, a nuclease inhibitor, a protease inhibitor, a salt, a lipid, and a pharmaceutically acceptable excipient.

Aspect 80. A kit comprising the system of aspect 77 or the composition of aspect 78 or 79.

Aspect 81. The kit of aspect 80, wherein the components of the kit are in the same container.

Aspect 82. The kit of aspect 80, wherein the components of the kit are in separate containers.

Aspect 83. A sterile container comprising the system of aspect 77 or the composition of aspect 78 or 79.

Aspect 84. The sterile container of aspect 83, wherein the container is a syringe.

Aspect 85. An implantable device comprising the system of aspect 77 or the composition of aspect 78 or 79.

Aspect 86. The implantable device of aspect 85, wherein the system is within a matrix.

Aspect 87. The implantable device of aspect 85, wherein the system is in a reservoir.

Aspect 88. A method of detecting a target DNA in a sample, the method comprising: (a) contacting the sample with: (i) a variant CRISPR-Cas effector polypeptide of any one of aspects 1-14 or a fusion polypeptide of any one of aspects 15-24; (ii) a guide RNA comprising: a region that binds to the variant CRISPR-Cas effector polypeptide of any one of aspects 1-14, and a guide sequence that hybridizes with the target DNA; and (iii) a detector DNA that is single stranded and does not hybridize with the guide sequence of the guide RNA; and (b) measuring a detectable signal produced by cleavage of the single stranded detector DNA by the variant CRISPR-Cas effector polypeptide or the fusion polypeptide, thereby detecting the target DNA.

Aspect 89. The method of aspect 88, wherein the target DNA is single stranded.

Aspect 90. The method of aspect 88, wherein the target DNA is double stranded.

Aspect 91. The method of any one of aspects 88-90, wherein the target DNA is bacterial DNA.

Aspect 92. The method of any one of aspects 88-90, wherein the target DNA is viral DNA.

Aspect 93. The method of aspect 92, wherein the target DNA is papovavirus, human papillomavirus (HPV), hepadnavirus, Hepatitis B Virus (HBV), herpesvirus, varicella zoster virus (VZV), Epstein-Barr virus (EBV), Kaposi's sarcoma-associated herpesvirus, adenovirus, poxvirus, or parvovirus DNA.

Aspect 94. The method of any one of aspects 88-90, wherein the target DNA is from a human cell.

Aspect 95. The method of any one of aspects 88-90, wherein the target DNA is human fetal or cancer cell DNA.

Aspect 96. The method of aspect 94 or 95, wherein the sample comprises DNA from a cell lysate.

Aspect 97. The method of aspect 94 or 95, wherein the sample comprises cells.

Aspect 98. The method of aspect 97, wherein the sample is a blood, serum, plasma, urine, aspirate, or biopsy sample.

Aspect 99. The method of any one of aspects 88-98, further comprising determining an amount of the target DNA present in the sample.

Aspect 100. The method of aspect 99, wherein said measuring a detectable signal comprises one or more of: visual based detection, sensor-based detection, color detection, gold nanoparticle-based detection, fluorescence polarization, colloid phase transition/dispersion, electrochemical detection, and semiconductor-based sensing.

Aspect 101. The method of any one of aspects 88-100, wherein the labeled detector DNA comprises a modified nucleobase, a modified sugar moiety, and/or a modified nucleic acid linkage.

Aspect 102. The method of any one of aspects 88-101, further comprising detecting a positive control target DNA in a positive control sample, the detecting comprising:
(c) contacting the positive control sample with:
(i) the variant CRISPR-Cas polypeptide or the fusion polypeptide;
(ii) a positive control guide RNA comprising: a region that binds to the variant CRISPR-Cas polypeptide or the fusion polypeptide, and a positive control guide sequence that hybridizes with the positive control target DNA; and
(iii) a labeled detector DNA that is single stranded and does not hybridize with the positive control guide sequence of the positive control guide RNA; and
(d) measuring a detectable signal produced by cleavage of the labeled detector DNA by the variant CRISPR-Cas polypeptide or the fusion polypeptide, thereby detecting the positive control target DNA.

Aspect 103. The method of any one of aspects 88-102, wherein the detectable signal is detectable in less than 45 minutes.

Aspect 104. The method of any one of aspects 88-102, wherein the detectable signal is detectable in less than 30 minutes.

Aspect 105. The method of any one of aspects 88-104, further comprising amplifying the target DNA in the sample by loop-mediated isothermal amplification (LAMP), helicase-dependent amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR), or isothermal multiple displacement amplification (IMDA).

Aspect 106. The method of any one of aspects 88-105, wherein target DNA in the sample is present at a concentration of less than 10 aM.

Aspect 107. The method according to any one of aspect 88-106, wherein the single stranded detector DNA comprises a fluorescence-emitting dye pair.

Aspect 108. The method according to any one of aspects 88-107, wherein the fluorescence-emitting dye pair is a fluorescence resonance energy transfer (FRET) pair.

Aspect 109. The method according to any one of aspects 88-107, wherein the fluorescence-emitting dye pair is a quencher/fluor pair.

Aspect 110. The method according to any one of aspects 88-109, wherein the single stranded detector DNA comprises two or more fluorescence-emitting dye pairs.

Aspect 111. The method according to aspect 110, wherein said two or more fluorescence-emitting dye pairs include a fluorescence resonance energy transfer (FRET) pair and a quencher/fluor pair.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Generation and Characterization of Variant CRISPR-Cas Effector Polypeptides

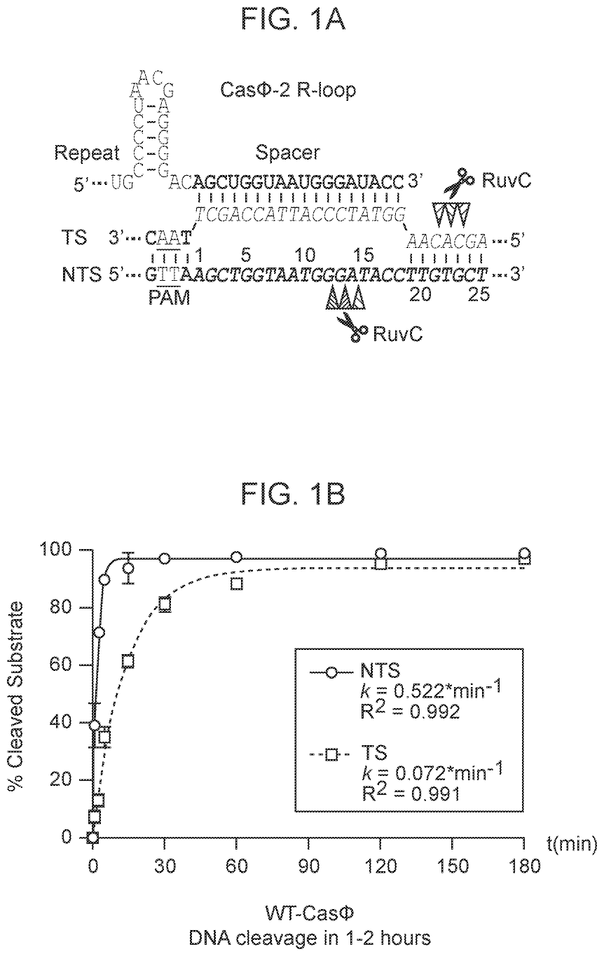

As depicted in FIG. 1A-1B, Wild-type (WT) CasΦ cleaves DNA within hours. FIG. 1A: schematic drawing of the DNA bound to the crRNA spacer in a Replication loop (R-loop) conformation. The scissor icons indicate cleavage positions within in the DNA, introduced by the CasΦ RuvC active site. FIG. 1B: Time course DNA cleavage assay, tracking the cleavage kinetics of the NTS and TS strands. WT CasΦ cleaves DNA within hours. The wild-type CasPhi polypeptide comprises the amino acid sequence depicted in FIG. 6.

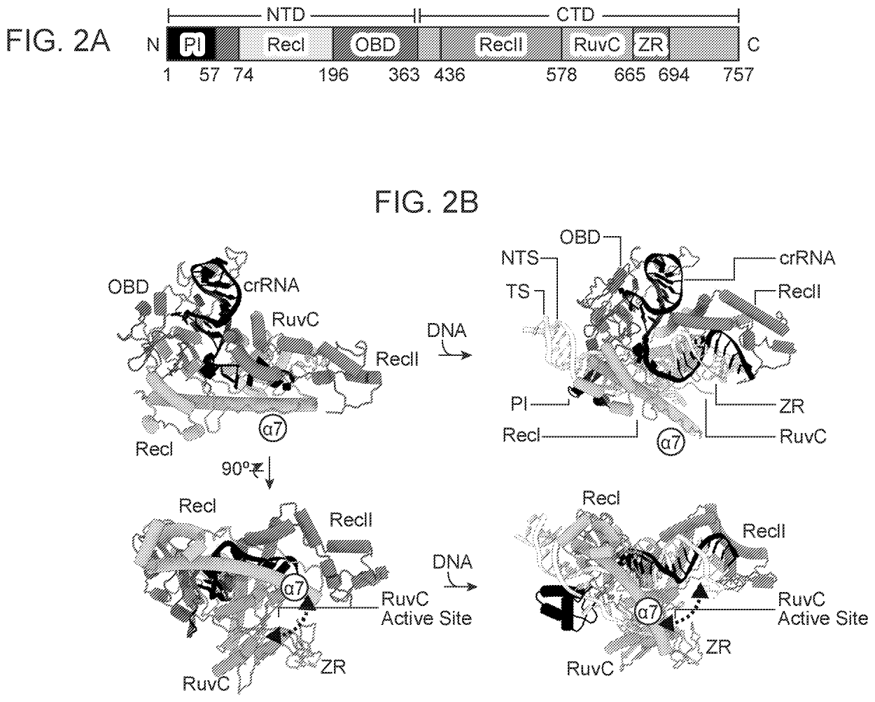

Cryo-EM structures of WT CasΦ in the crRNA and DNA bound states are depicted in FIG. 2A-2B. FIG. 2A: Scheme illustrating the domain architecture of WT CasΦ. FIG. 2B: Structures of WT CasΦ in the crRNA bound binary state (left) and DNA bound ternary state (right) in two 90° rotated conformations (upper and lower panels). Domain coloring as in A. Helix alpha 7 (yellow circle) rotates (arrow) between the binary and ternary states above the RuvC (green) active site. Amino acid modifications in this region allow for the generation of CasΦ variants with improved DNA cleavage kinetics.

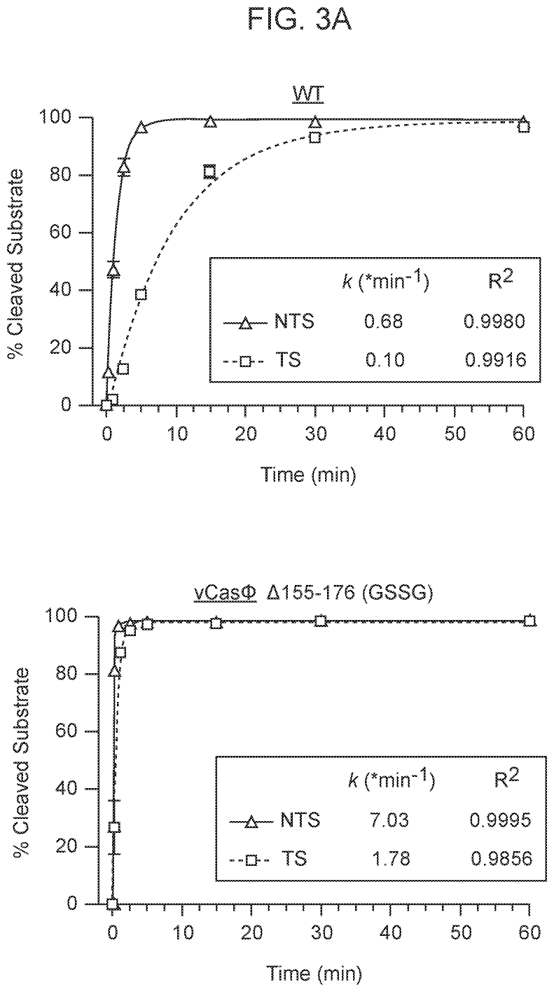

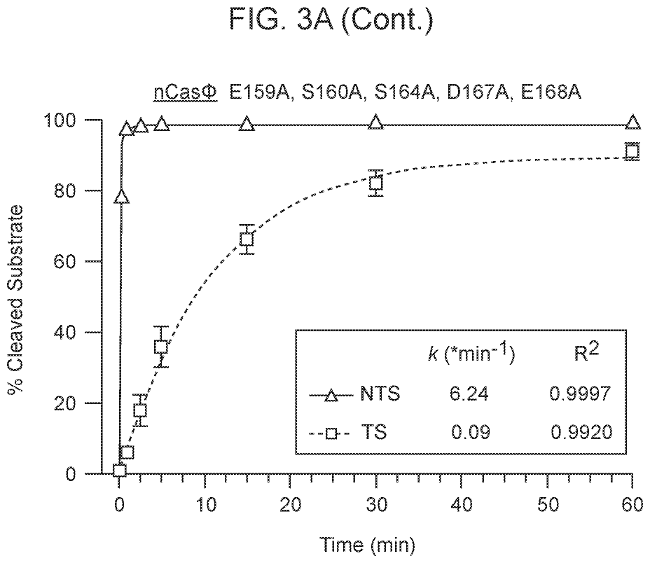

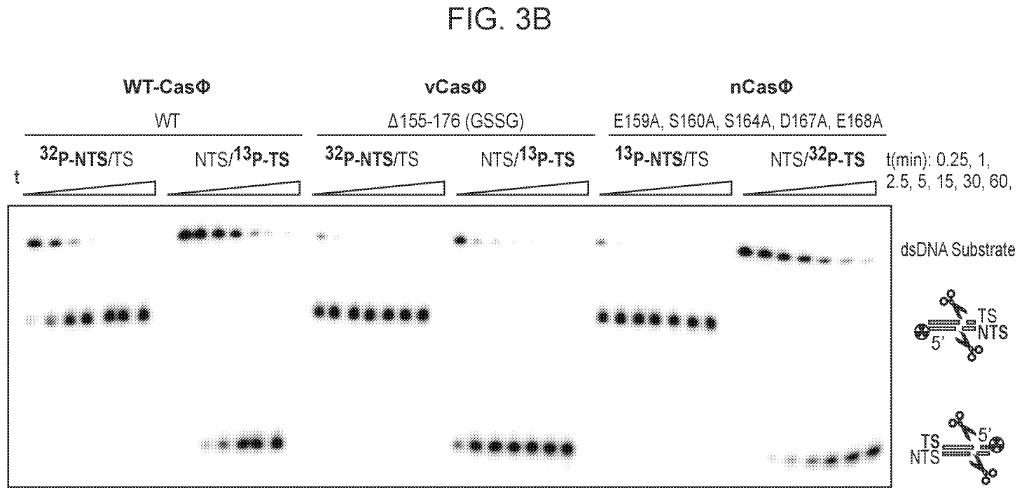

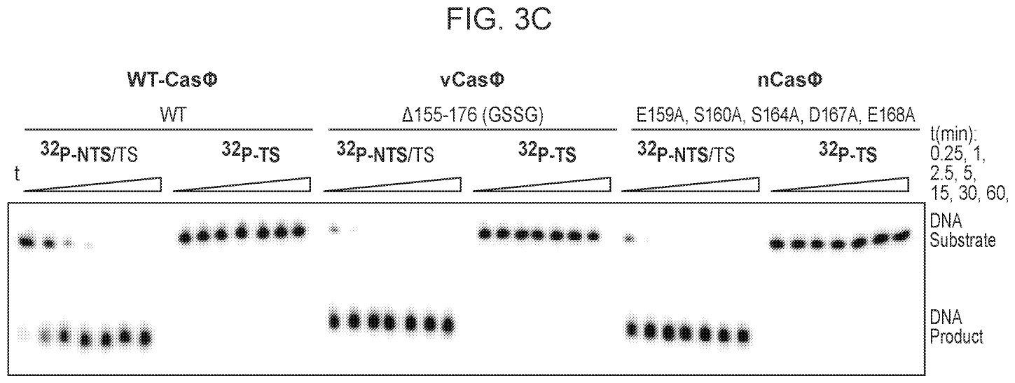

Two variant CasPhi polypeptides were engineered, in which helix alpha 7 was modified, either by replacement of amino acids 155-176, or by substitution of E159, S160, S164, D167, and E168. These variant CasPhi polypeptides were designated “vCasPhi” and “nCasPhi.” The amino acid sequence of vCasPhi is provided in FIG. 7; as shown in FIG. 7, a GSSG (SEQ ID NO:160) peptide replaces amino acids 155-176 of the wild-type CasPhi-2 amino acid sequence depicted in FIG. 6. The amino acid sequence of nCasPhi is provided in FIG. 8; as shown in FIG. 8, E159, S160, S164, D167, and E168 of the amino acid sequence depicted in FIG. 6 are replaced with Ala. The two variants were characterized. The results are depicted in FIG. 3.

As shown in FIG. 3A-3C, the variants vCasPhi and nCasPhi cleave DNA faster than wild-type CasPhi (where the wild-type CasPhi comprises the amino acid sequence depicted in FIG. 6). FIG. 3A: Time course DNA cleavage assay, tracking both, the cleavage kinetics of the NTS and TS strands. WT CasΦ cleaves DNA within hours. vCasΦD cleaves the same DNA 17.8 times faster, within minutes. nCasΦ cleaves the NTS 10× faster than WT. The cleavage rate of the respective strands is given in the panels below the graphs. FIG. 3B: Data for the graphs shown in A. Only one replicate is shown. Experiments were performed in three individual technical replicates. FIG. 3C: Engineered vCasΦ and nCasΦ cleave DNA only when a crRNA guide complementary TS is present in the reaction, demonstrating that the variants are highly specific and only become activated upon TS recognition.

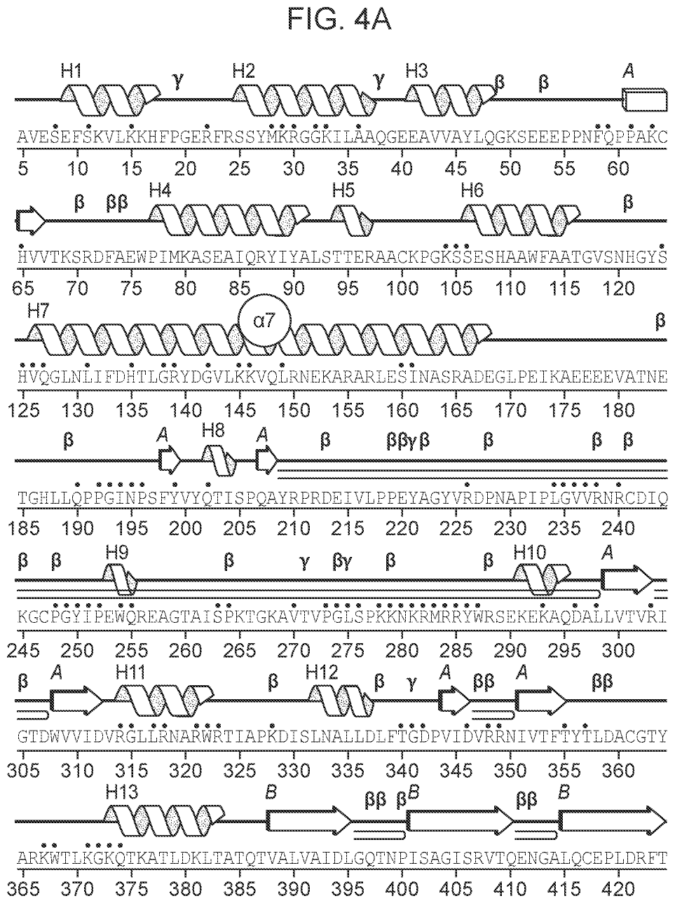

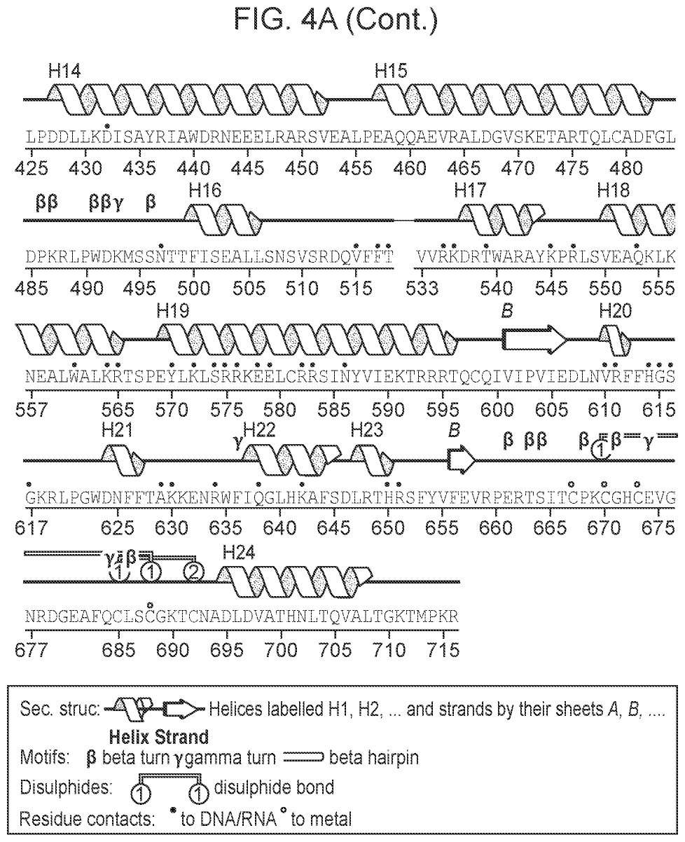

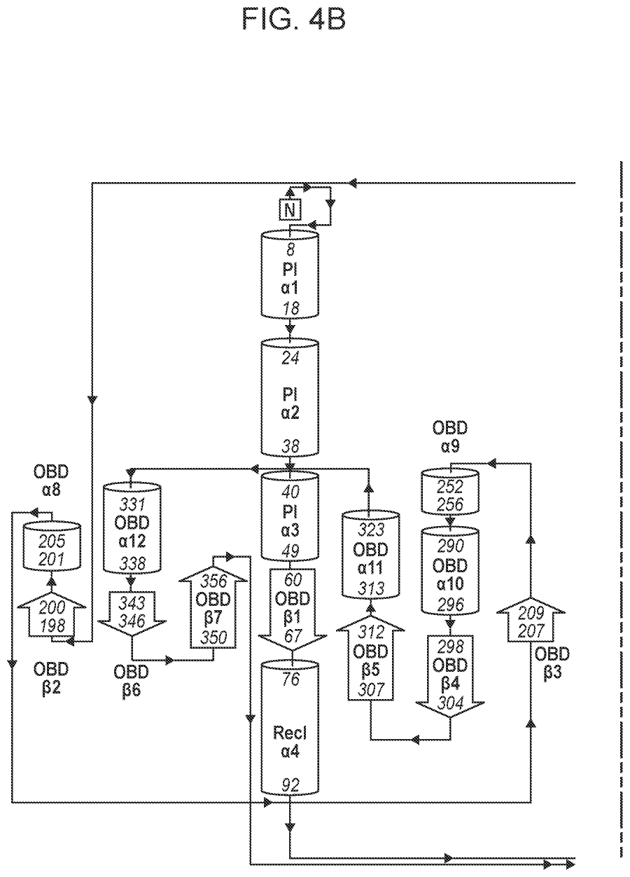

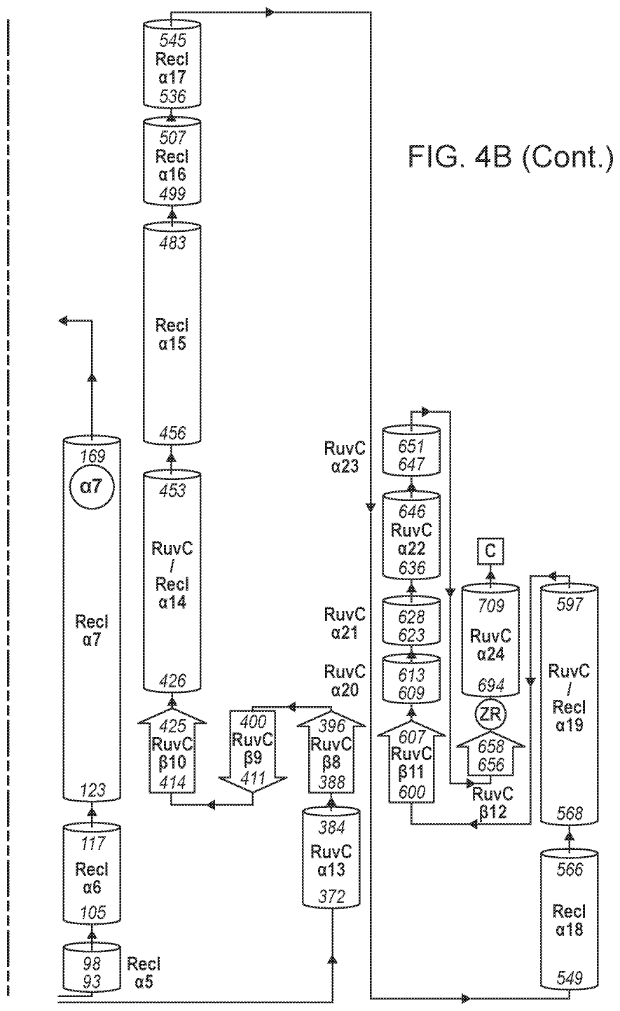

FIG. 4A-4B depict secondary structure of WT CasΦ illustrating the position of helix alpha 7. FIG. 4A: Sequence of CasΦ-2 including the secondary structure of CasΦ (sheets and helices). Alpha 7 is highlighted as a yellow circle. FIG. 4B: Secondary structure arrangement of CasΦ-2. Alpha 7 is highlighted as a yellow circle.

FIG. 5A-5C provide an amino acid sequence alignment of several CasΦ proteins, illustrating the position of helix alpha 7. Modifications in the highlighted region (black frame) can be introduced to increase the specific DNA cleavage activity.

### Example 2: CasΦ Variants Exhibit Cleavage of Non-Target ssDNA in Trans

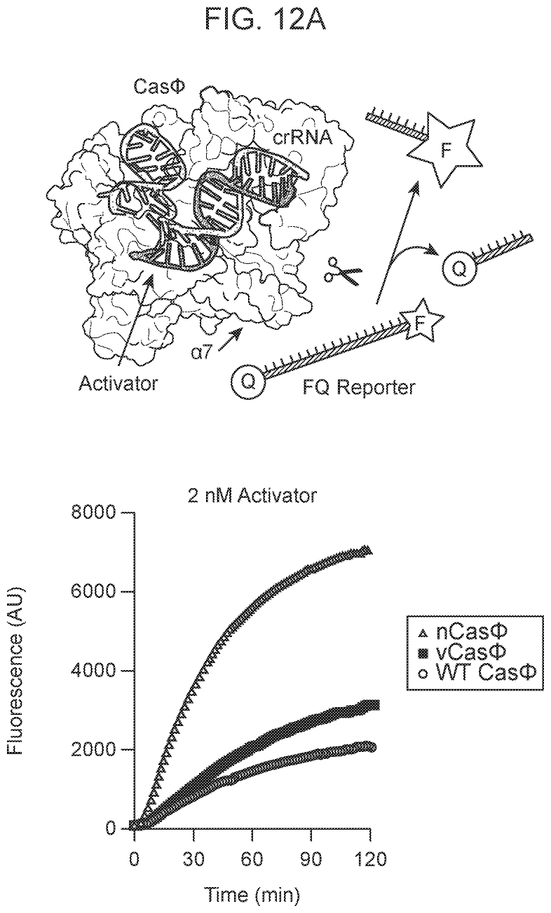

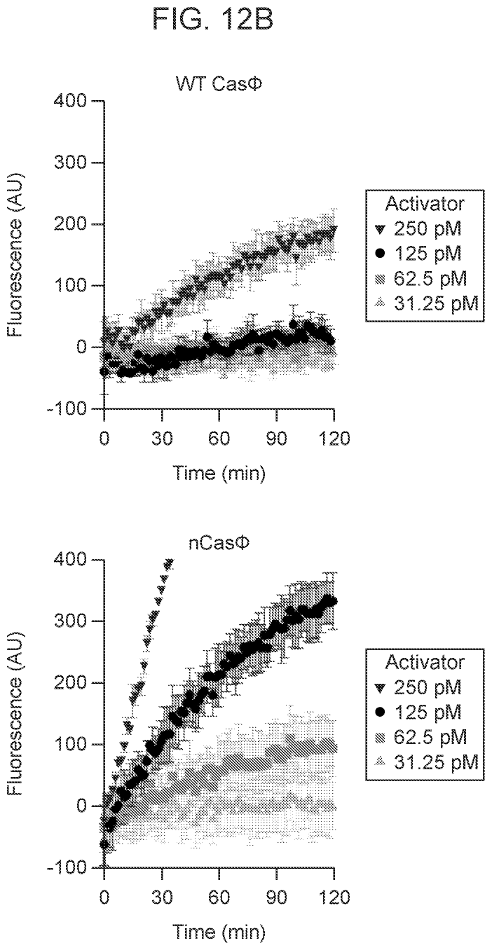

A fluorophore quencher (FQ)-assay was employed to assess the ability of engineered CasΦ variants to non-specifically degrade the fluorophore reporter DNA after activation. The FQ assay is depicted schematically in FIG. 12A.

### Materials and Methods

Plasmids were cloned and mutagenized via Golden Gate assembly as previously described. Pausch et al. (2020) Science 369:333. In brief, the pRSFDuet-1 derived casΦ-2 overexpression vector pPP085 (Pausch et al. (2020) supra) was amplified around the horn using primers containing the desired mutation and AarI Golden Gate cloning sites. The resulting fragment was circularized using the restriction enzyme AarI (Thermo Fisher Scientific) and T4 ligase (NEB). Plasmids were propagated in Escherichia coli MachI. Generated plasmids were sequenced across the coding sequence of casΦ-2.

### CasΦ-2 Protein Production and Purification

C-terminally hexa-histidine tagged CasΦ-2 was produced by heterologous expression in E. coli and purified as previously described (Pausch et al. (2020) supra). In brief, overexpression plasmids were transformed into E. coli BL21 (DE3)-Star. Expression cultures were grown shaking vigorously at 37° C. in 1.5 L TB-Kan (50 μg/mL Kanamycin) media to an OD₆₀₀ of 0.6. Subsequently, cultures were cooled down on ice for 15 min and gene expression was induced with 0.5 mM IPTG before incubation overnight at 16° C. Cells were harvested by centrifugation and resuspended in wash buffer (50 mM HEPES-Na pH 7.5 RT, 1 M NaCl, 20 mM imidazole, 5% glycerol and 0.5 mM TCEP), subsequently lysed by sonication, followed by lysate clarification by centrifugation. The soluble fraction was loaded on a 5 mL Ni-NTA Superflow Cartridge (Qiagen) pre-equilibrated in wash buffer. Bound proteins were washed with 20 column volumes (CV) wash buffer and subsequently eluted in 4 CV elution buffer (50 mM HEPES-Na pH 7.5 RT, 500 mM NaCl, 500 mM imidazole, 5% glycerol and 0.5 mM TCEP). The eluted proteins were concentrated to 1-2 mL before injection into a HiLoad 16/600 Superdex 200 pg column (GE Healthcare) pre-equilibrated in size-exclusion chromatography (SEC) buffer (20 mM HEPES-Na pH 7.5 RT, 500 mM NaCl, 5% glycerol and 0.5 mM TCEP). Peak fractions were concentrated to 1 mL and concentrations were determined based on the absorbance at 280 nm using a NanoDrop 8000 Spectrophotometer (Thermo Scientific). Proteins were purified at a constant temperature of 4° C. and concentrated proteins were kept on ice to prevent aggregation, snap frozen in liquid nitrogen and stored at −80° C.

### RNP Complex Reconstitution

CasΦ-2 was produced as described above. The crRNA guide (Sequence (5′->3′): HO-CAACGAUUGCCCCUCACGAGGGGACAGCUGGUAAUGGGAUACCUU (SEQ ID NO:161); where the repeat sequence is underlined) was ordered as a synthetic RNA oligonucleotide from IDT (Integrated DNA Technologies) and dissolved in diethylpyrocarbonate (DEPC)-treated ddH₂0 to a concentration of 0.5 mM. Subsequently, the crRNA was heated to 65° C. for 3 min and cooled down to RT to allow for hairpin formation. CasΦ-2 RNP complexes were reconstituted at a concentration of 10 μM by incubation of 10 μM CasΦ-2 and 12 μM crRNA for 10 min at RT in 2× cleavage buffer (2×CB) (20 mM Hepes-Na pH 7.5, 300 mM KCl, 10 mM MgCl₂, 20% glycerol, 1 mM TCEP). Formed RNPs were aliquoted to a volume of 10 μL, flash frozen in liquid nitrogen and stored at −80° C. Before usage, RNP aliquots were thawed on ice.

### Fluorophore Quencher Assay

CasΦ RNP were assembled as described above. Reactions were initiated by combining 100 nM RNP (100 nM CasΦ, 120 nM crRNA), 100 nM DNase Alert (IDT) FQ probe, with and without activator ssDNA (Sequence (5′->3′): HO-AAGGTATCCCATTACCAGCT; SEQ ID NO:162) in cleavage buffer (10 mM Hepes-Na pH 7.5, 150 mM KCl, 5 mM MgCl₂, 10% glycerol, 0.5 mM TCEP) in a 384 well flat bottom black polystyrene assay plate (#3820, Corning). Three replicates for each reaction were monitored (λₑₓ: 530 nm; λₑₓ: 590 nm) in a Cytation 5 plate reader (BioTek) at 37° C. every 1.5 min. The data were background-subtracted using the mean values of the measurements taken at the respective time point in absence of the activator. Data were plotted in Prism 6 (graphpad).

### Results

The data indicate that, after target DNA recognition and cleavage, CasΦ variants remain in a nuclease activated state to non-specifically degrade ssDNA in trans.

The data are depicted in FIG. 12A-12B. Incubation of the CasΦ RNPs in presence of a crRNA spacer complementary ssDNA oligonucleotide target revealed that nCasΦ catalyzes fluorophore reporter degradation faster than vCasΦ and WT CasΦ (FIG. 12A). The nCasΦ variant allows detection of an activator nucleic acid when the activator nucleic acid is present in the low picomolar range, as depicted in FIG. 12B.

FIG. 12A-12B. nCasΦ and vCasΦ detect DNA more efficiently than WT CasΦ. (FIG. 12A) Left panel: Scheme illustrating the in vitro nucleic acid detection assay. Right panel: FQ assay for detection of 2 nM ssDNA activator by WT and engineered CasΦ. (n=3 each; means±SD). (FIG. 12B) FQ assay for detection of pico-molar ssDNA activator concentrations by WT (left) and nCasΦ (right). (n=3 each; means±SD).

### Example 3

Target gene editing efficiencies in planta were compared between wild type CasΦ (WTCasΦ), vCasΦ and nCasΦ, by transfection of RNPs or plasmids into Arabidopsis mesophyll protoplasts. Consistent with the in vitro data, higher target gene editing efficiencies were observed with the vCasΦ and nCasΦ variants compared to the WTCasΦ.

### Materials and Methods

### RNP Reconstitution

Guide RNAs were synthesized (25 nt repeat+20 nt spacer as shown in Table 2) by Synthego. Dry RNA was dissolved by adding DEPC-treated H2O to a concentration of 0.5 mM. The dissolved RNA was incubated at 65° C. for 3 min, then cooled down to RT. For RNP reconstitution, heated and cooled RNA was added to 2×CB buffer for a final concentration of 5 uM and vortexed to mix. Then, WTCasΦ, vCasΦ or nCasΦ proteins were added to a final concentration of 4 μM and mixed by pipetting. This solution was then incubated at room temperature for 30 min. The resulting solution contains 4 μM of RNP in 2×CB buffer. 2×CB buffer: 20 mM Hepes-Na, 300 mM KCl, 10 mM MgCl₂, 20% glycerol, 1 mM TCEP, PH 7.5. Special care was taken to keep all reagents RNase free.

Table 2: Sequences of guide RNAs used for Arabidopsis protoplast transfections. Guide RNAs are composed of two parts: repeat and spacer, with spacer at the 3′ side of the repeat.

TABLE 2

| | CasΦ Guide RNA repeat sequence | | |
|---|---|---|---|
| Purpose | (common to all guides) | | |
| For plasmid | GTCGGAACGCTCAACGATTGCCCCTCACGAGGGGAC | | |
| vectors | (SEQ ID NO: 23) | | |
| For RNPs | CAACGATTGCCCCTCACGAGGGGAC | | |
| | (SEQ ID NO: 163) | | |
| | Guide RNA spacer | | Direction |
| Guide | sequence (Denoted | | relative to |
| RNA name | in DNA sequence) | PAM | target gene |
| AtPDS3 gR8 | TTGTTCCGCAAAATAGCCCA | TCG | reverse |
| | (SEQ ID NO: 164) | | |
| AtPDS3 gR10 | CAGTTGACAATCCAGCCAAT | TTG | reverse |
| | (SEQ ID NO: 165) | | |
| FWA gR1 | TCCCATTCAACATTCATACG | TTA | forward |
| | (SEQ ID NO: 166) | | |
| FWA gR4 | TGGTTCTATACTAATATCAA | TTA | forward |
| | (SEQ ID NO: 167) | | |
| FWA gR5 | ATATTAGTATAGAACCATAA | TTG | reverse |
| | (SEQ ID NO: 168) | | |
| FWA gR6 | GTATAGAACCATAACAAAAG | TTA | reverse |
| | (SEQ ID NO: 169) | | |
| scramble | GCGACACGACTCATTATAAC | NONE | |
| control | (SEQ ID NO: 170) | | |

### Plasmid Cloning

pCAMBIA1300 vector with the UBQ10 promoter driving the expression of WTCasΦ and without the guide RNA cassette was previous constructed (named as pCAMBIA1300 pUBQ10 pco-WTCasΦDMCS). To build the corresponding vectors expressing the vCasΦD and nCasΦD variants, the pCAMBIA1300 pUBQ10 pco-WTCas4D MCS plasmid was digested with KpnI to remove the UBQ10 promoter and DNA sequence encoding the N-terminal of the CasΦ. The removed DNA sequence included the sequence to be mutated for the vCasΦD and nCasΦD variants. The following two fragments were PCR amplified using the pCAMBIA1300 pUBQ10 pco-WTCasΦDMCS vector as template: (1) The UBQ10 promoter and the CasΦ sequence before the mutation site. (2) The CasΦ sequence after the mutation site before the KpnI digestion site. The primers used for these amplifications contained overlapping sequences with the vector backbone on corresponding end. Also, the primers between fragment (1) and (2) had overlapping sequences containing the desired mutation to generate vCasΦD and nCasΦD. Then, the vector backbone and the PCR fragments were assembled by TAKARA in-fusion HD cloning kit (cat639650) to generate pCAMBIA pUBQ10 pco-vCasΦDMCS and pCAMBIA pUBQ10 pco-nCasΦD MCS vectors.

To clone the guide RNA cassettes, the pCAMBIA1300 pUBQ10 pco-WTCasΦDMCS, pCAMBIA pUBQ10 pco-vCasΦD MCS and pCAMBIA pUBQ10 pco-nCasΦ MCS vectors were linearized with SpeI digestion. For AtPDS3 gRNA8 and gRNA10 driven by the U6 promoter, as well as the ribozyme-AtPDS3 gRNA10 driven by Pol-II promoters, the whole guide RNA cassettes were amplified from previously built vectors with overlapping sequence on the corresponding ends to the linearized vector backbones. For the FWA gRNA1, gRNA4, gRNA5 and gRNA6, the guide RNA cassettes with the U6 promoter were amplified as two PCR fragments, with the overlapping sequences between the two fragments as the specific FWA gRNA spacer sequences added by primers. Then, the linearized vectors and the corresponding guide RNA transcription cassettes (as one or two PCR fragments) were assembled by TAKARA in-fusion HD cloning kit (cat639650) to generate the final vectors. Qiagen Plasmid Maxi Kit (Cat12163) was used to maxiprep these final vectors for protoplast transfections.

### Protoplast Isolation and Transfection

Wild type (Col-0 ecotype) and the fwa-4 epi-mutant plants were grown under a 12 h light/12 h dark photoperiod and with a relatively low light condition in an incubator. Protoplast isolation was performed according to the following publication: PMID: 17585298. Special care was taken to maintain a sterile environment when preparing protoplasts.

For RNP transfection, 26 μl of 4 μM RNP was first added to a round bottom 2 ml tube, followed by 200 μl of protoplasts (2×10⁵ cells/ml). Then, 2 μl of 5 μg/μl salmon sperm DNA was added and mixed gently by tapping the tube 3-4 times. Finally, 228 μl of fresh, sterile and RNase free PEG-CaCl₂ solution (PMID: 17585298) was added to the protoplast-plasmid mixture and mixed well by gently tapping the tube. The protoplasts with PEG solution were incubated at RT for 10 min, then, 880 μl of W5 solution (PMID: 17585298) was added and mixed with the protoplasts by inverting the tube 2-3 times to stop the transfection. Protoplasts were harvested by centrifuging the tubes at 100 rcf for 2 min and resuspended in 1 ml of WI solution. They were then plated in 6-well plates pre-coated with 5% calf serum. These 6-well plates were then incubated at room temperature for 48 h.

For plasmid transfections, the concentrations of plasmids were determined by nanodrop. Then the same amount of plasmids were added to the bottom of each transfection tube, and the volume of the plasmids was supplemented with H₂O to reach 20 ul. 200 ul of protoplasts were added followed by 220 μl of fresh and sterile PEG-CaCl₂ solution (PMID: 17585298). The mixture was mixed well by gently tapping tubes and incubated at room temperature for 10 min. 880 μl of W5 solution (PMID: 17585298) was added and mixed with the protoplasts by inverting the tube 2-3 times to stop the transfection. Protoplasts were harvested by centrifuging the tubes at 100 rcf for 2 min and resuspended in 1 ml of WI solution. They were then plated in 6-well plates pre-coated with 5% calf serum. These 6-well plates were then incubated at room temperature for 48 h.

At the end of the incubations, the protoplasts were harvested by centrifugation at 100 rcf for 2-3 min. The resulting supernatant was moved to another tube and went through another centrifugation at 3000 rcf for 3 min to collect any residual protoplasts. Pellets from these two centrifugations were combined and flash frozen for further analysis.

### Amplicon Sequencing

DNA was extracted from protoplast samples with Qiagen DNeasy plant mini kit (Cat. No. 69106). The amplicon was obtained using two rounds of polymerase chain reaction (PCR). Amplification primers for the first round of PCR were designed to have the 3′ sequence of the primers flanking a 200-300 bp fragment of the genomic area targeted by the guide RNA of interest. The 5′ part of the primer contained a sequence which will be bound by common sequencing primers. After 25 cycles of the first round of PCR amplification, the reaction was cleaned using 1× Ampure XP beads (BECKmen Coulter A63881). The eluate was used as template for the second round of PCR using the Phusion enzyme and 12 cycles of amplification. The second round of PCR was designed so that indexes were added to each sample. The samples were then purified using 0.8× Ampure XP beads. Part of the purified libraries were run on a 2% agarose gel to check for size and absence of primer dimer (fragments below 200 bp considered as primer dimer). Then amplicons were sent for next generation sequencing.

### Amplicon Sequencing Result Analysis

Reads were first quality and adaptor trimmed with trim-galore and then mapped to the target genomic region by BWA aligner. Sorted and indexed bam files were used as input files for further analysis by the CrispRvariants R package. Each mutation pattern with corresponding read counts was exported by the CrispRvariants R package. After assessing all control samples, a criterion to classify reads as edited reads was established: only reads with a >=3 bp deletion or insertion (indel, mainly as deletions) of the same pattern (indels of same size starting at the same location) with >=100 read counts from a sample are counted as edited reads. This criterion is established due to the observation of 1 bp indels and occasionally 2 bp indels with read numbers >100 in control samples. Also, larger indels that happen at very low frequencies (much lower than 100 reads) were observed in control samples. These observations indicate that occasional PCR inaccuracy and low-quality sequencing in a small fraction of reads can result in the indel patterns with corresponding read number ranges as stated above in control samples with the typical sequencing depth in our experiments (1-5 million reads/sample). By employing such stringent criteria, it is believed that the editing signals counted are true signal indicating editing events. Additionally, for FWA gRNA6 targeted regions, there are long stretches of adenines a few nucleotides just after these target regions. Due to the high error rate of polymerases amplifying long stretches of adenines, reads with indels only within these stretches of adenines were not counted as real edited reads.

### Results

To compare the target gene editing efficiencies of the wild type CasΦ and the CasΦ variants, Arabidopsis mesophyll protoplasts were transfected with plasmids expressing WTCasΦ, vCasΦD, or nCasΦD, as well as the desired guide RNAs. Also, RNPs reconstituted with the WTCasΦ, vCasΦ, or nCasΦ and the desired guide RNAs were used to transfect the protoplasts.

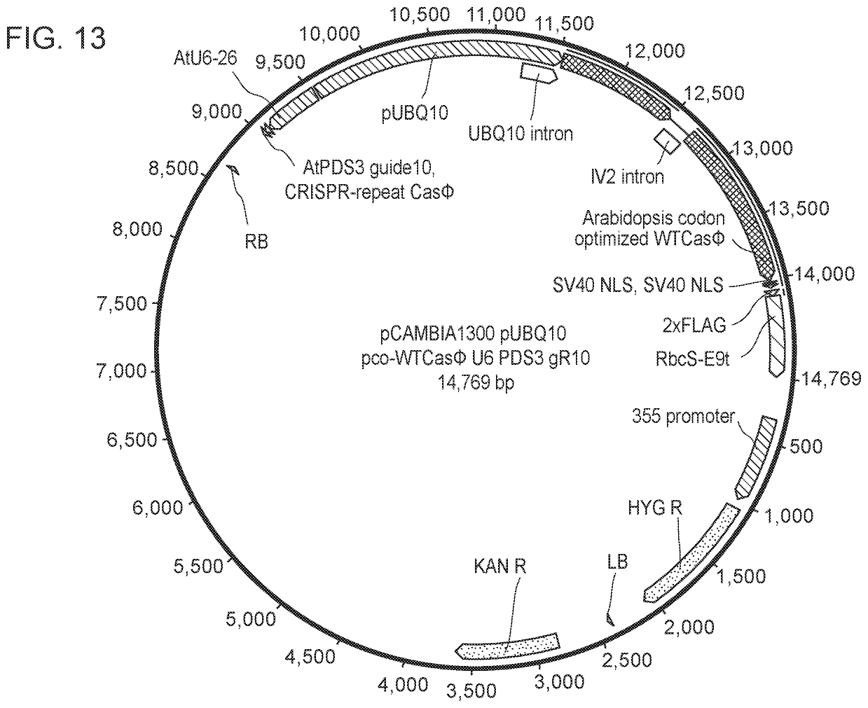

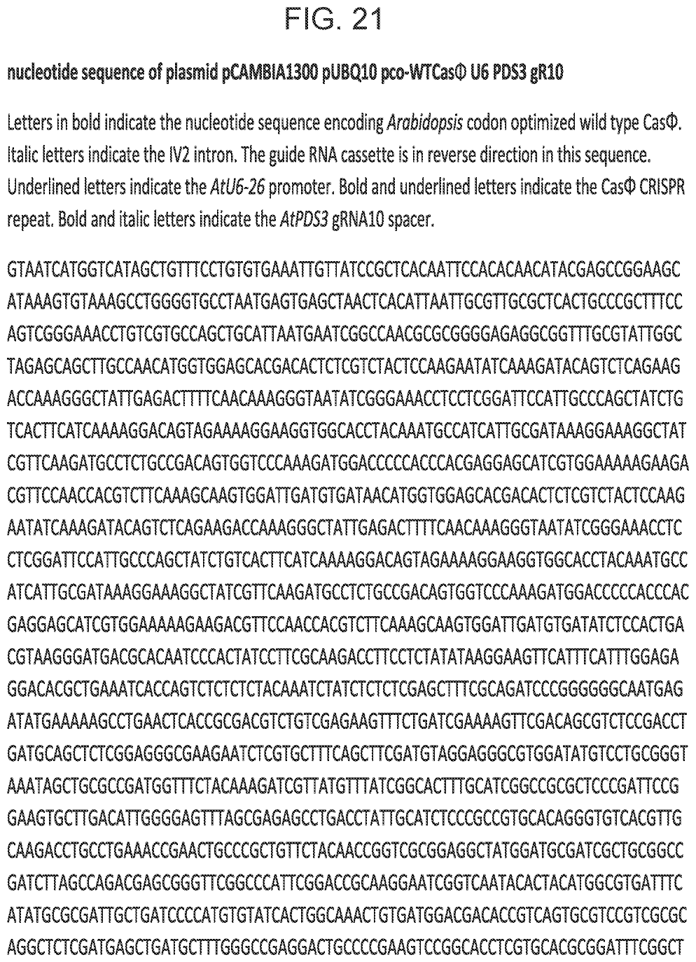

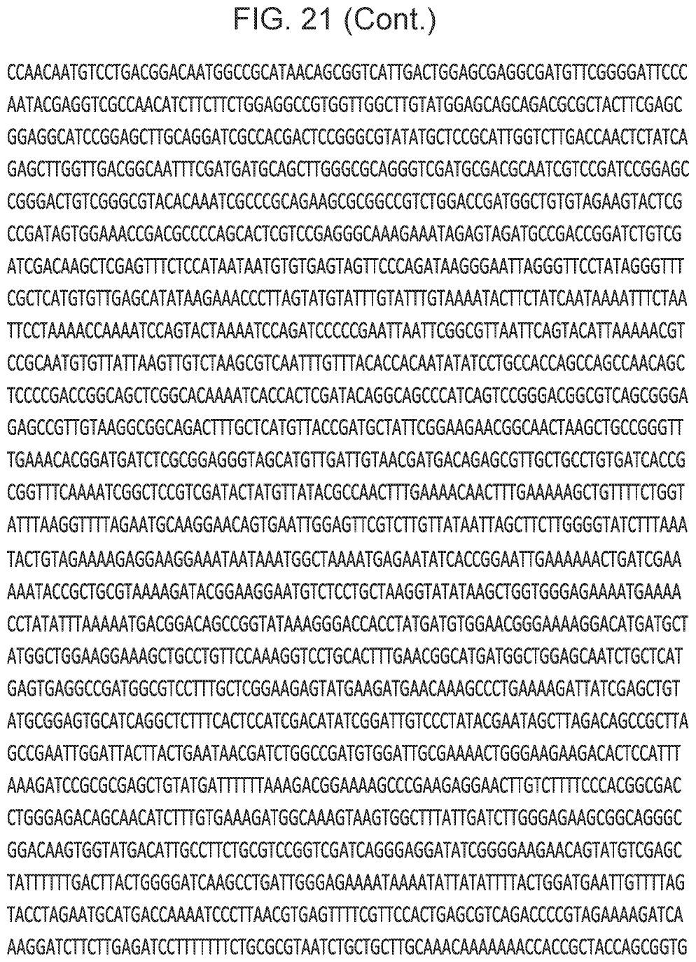

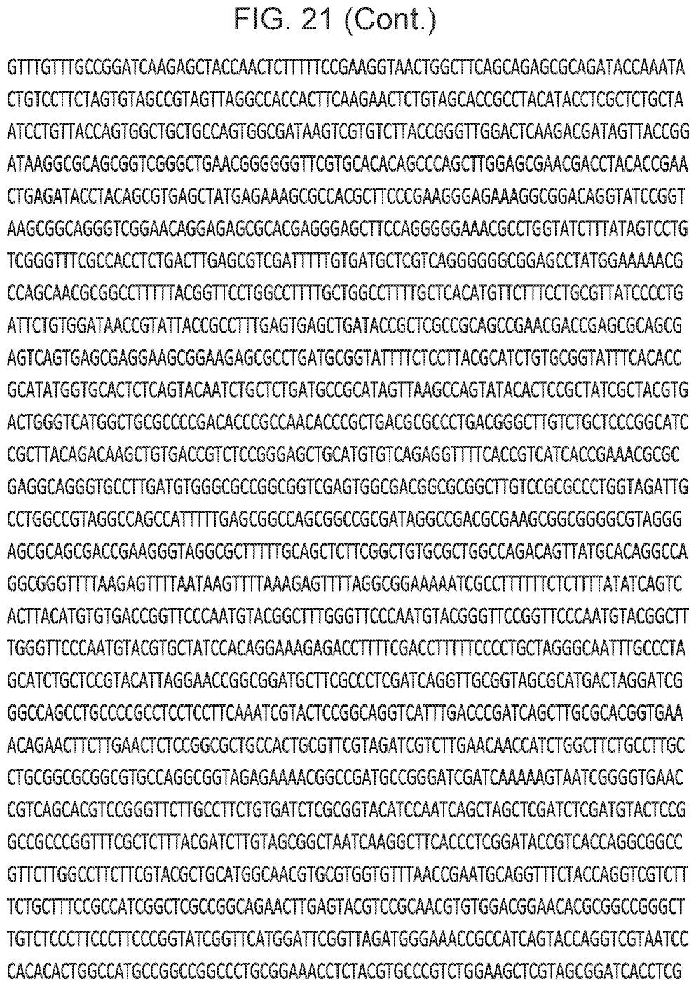

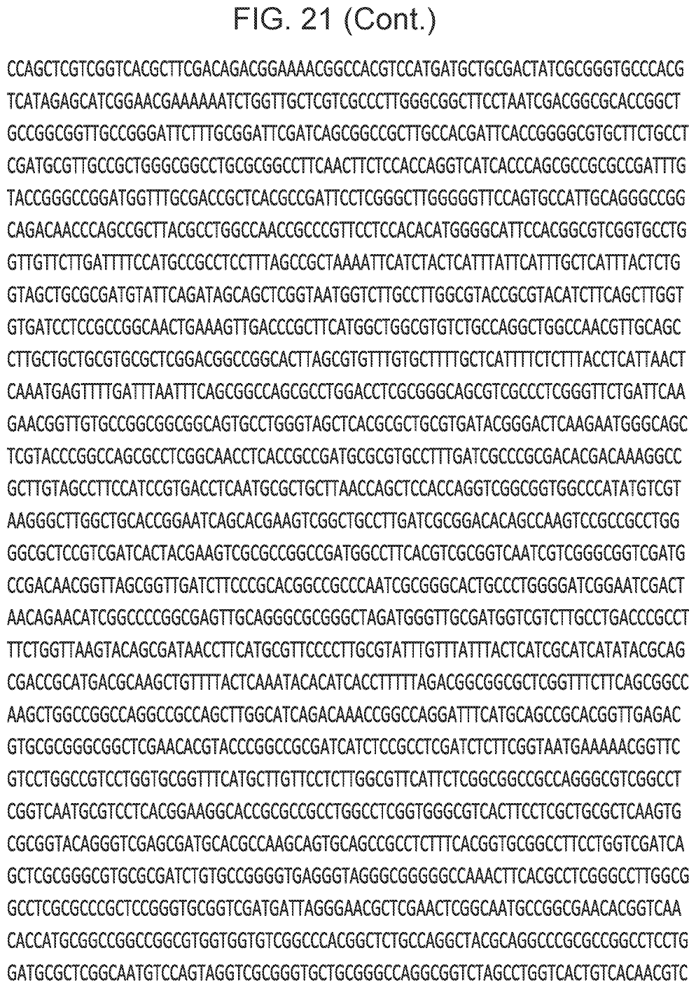

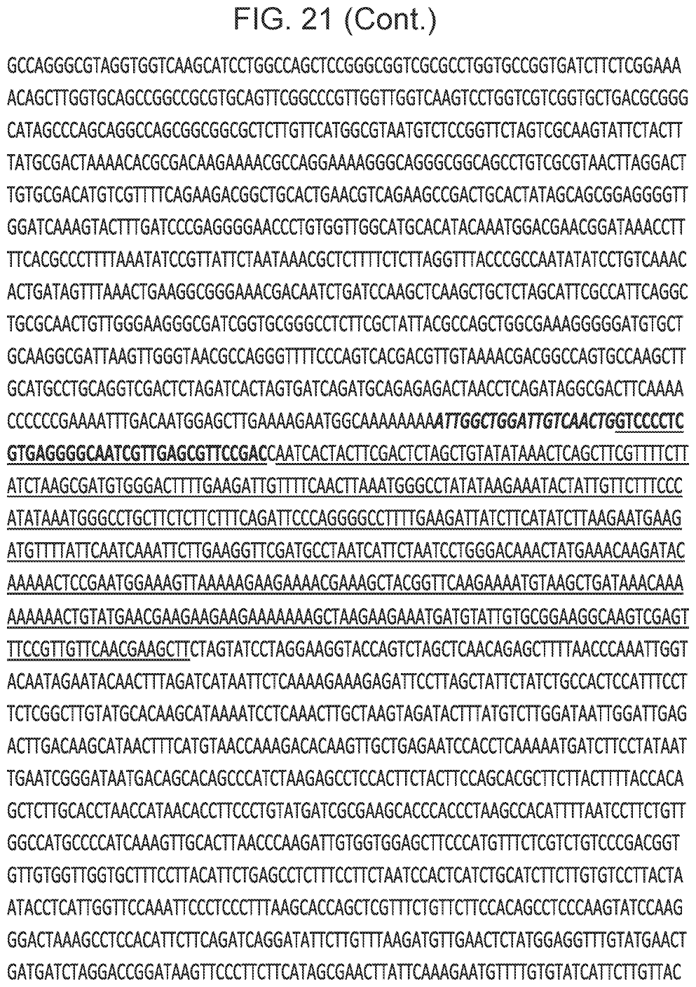

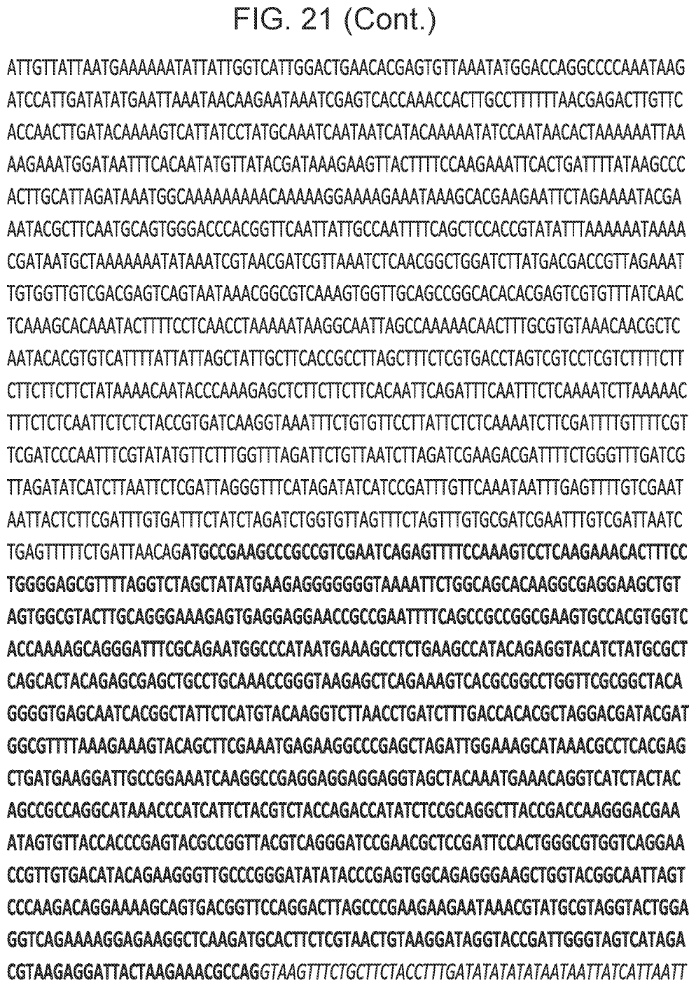

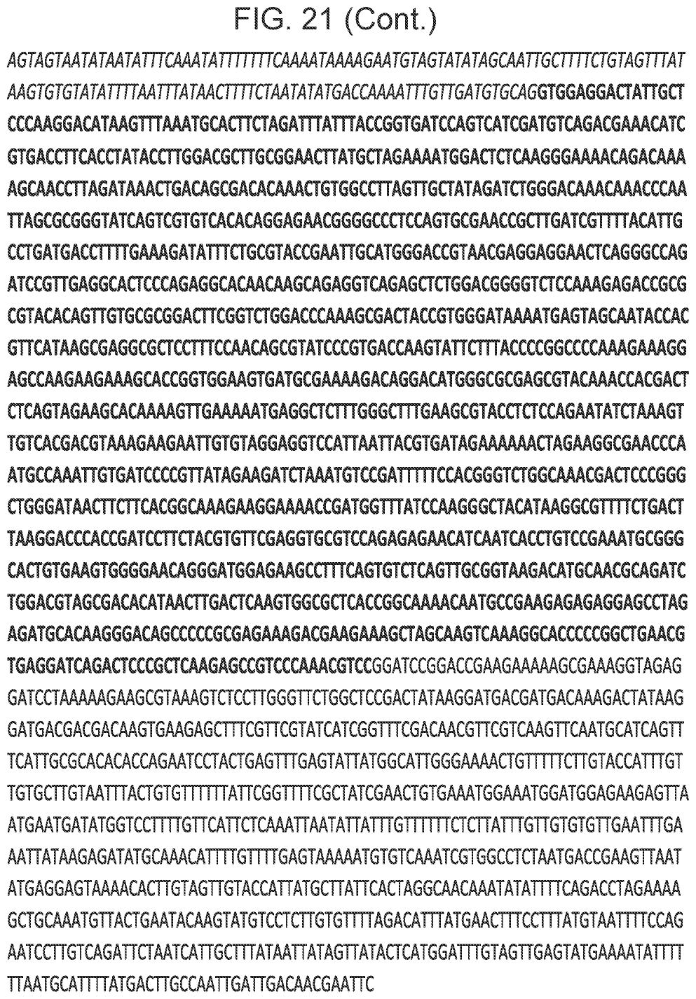

In the plasmids used for transfections, the UBQ10 promoter was used to drive the Arabidopsis codon optimized CasΦ expression and the U6 promoter (AtU6-26) was used to drive transcription of the guide RNAs. A detailed map of the plasmid expressing the WTCasΦ and the AtPDS3 gRNA10 is shown in FIG. 13; the sequence of the plasmid depicted in FIG. 13 is provided in FIG. 21. In FIG. 21, letters in bold indicate the nucleotide sequence encoding Arabidopsis codon optimized wild type CasΦ; and italic letters indicate the IV2 intron; the guide RNA cassette is in reverse direction in this sequence; underlined letters indicate the AtU6-26 promoter; bold and underlined letters indicate the CasΦ CRISPR repeat; and bold and italic letters indicate the AtPDS3 gRNA10 spacer.

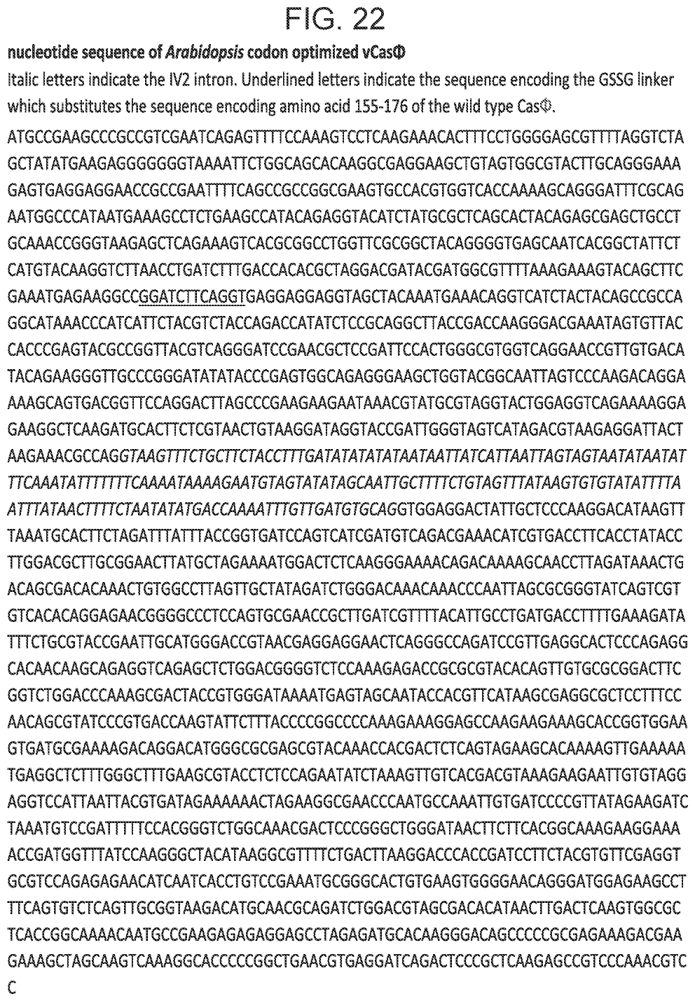

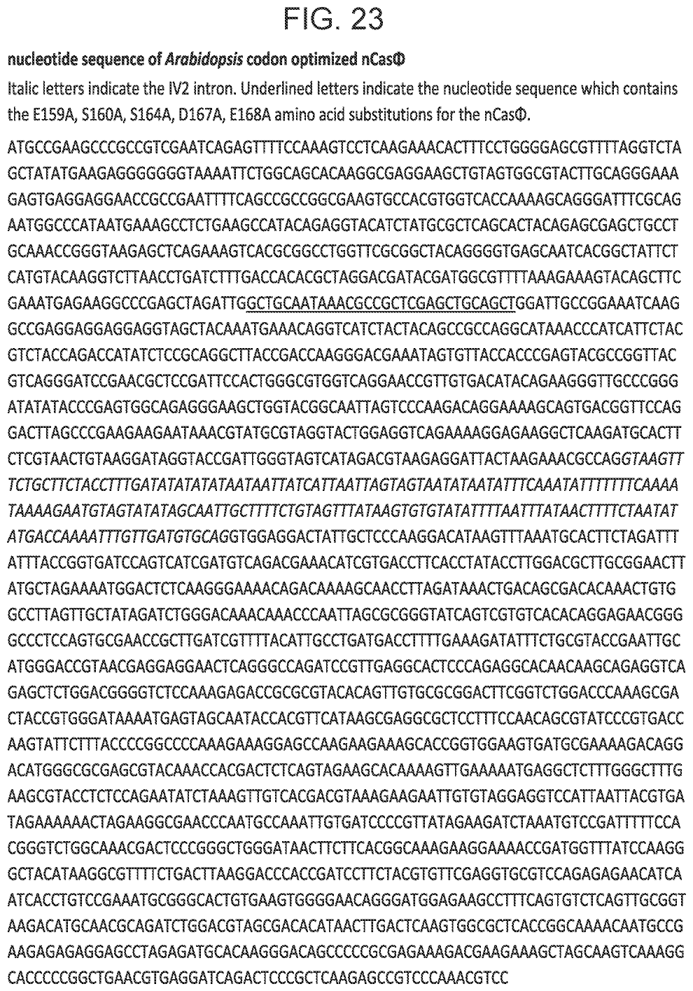

The plasmids expressing the vCasΦ and nCasΦ variants are only different from the WTCasΦ plasmids in the sequences encoding the CasΦ protein (sequences provided in FIG. 22 and FIG. 23). For RNP transfection, the same amount of WTCasΦ, vCasΦ and nCasΦ proteins were used to reconstitute RNPs with the desired guide RNAs. 6 guide RNAs were used for the tests in this example: (1) AtPDS3 gRNA8 and gRNA10 (PMID: 32675376) targeting the AtPDS3 gene and (2) FWA gRNA1, gRNA4, gRNA5 and gRNA6, targeting the promoter region of the FWA gene (Table 2).

In FIG. 22, italic letters indicate the IV2 intron; and underlined letters indicate the sequence encoding the GSSG (SEQ ID NO:160) linker which substitutes the sequence encoding amino acid 155-176 of the wild type CasΦ. In FIG. 23, italic letters indicate the IV2 intron; and underlined letters indicate the nucleotide sequence which contains the E159A, S160A, S164A, D167A, E168A amino acid substitutions for the nCasΦ.

FIG. 13. Plasmid map of pCAMBIA1300 pUBQ10 pco-WTCasΦ U6 PDS3 gR10. In this plasmid, Arabidopsis codon optimized wild type CasΦ was driven by the UBQ10 promoter and RbcS-E9 terminator. The AtU6-26 promoter was used to drive the transcription of the CasΦ CRISPR repeat sequence followed by the spacer sequence for AtPDS3 gRNA10.

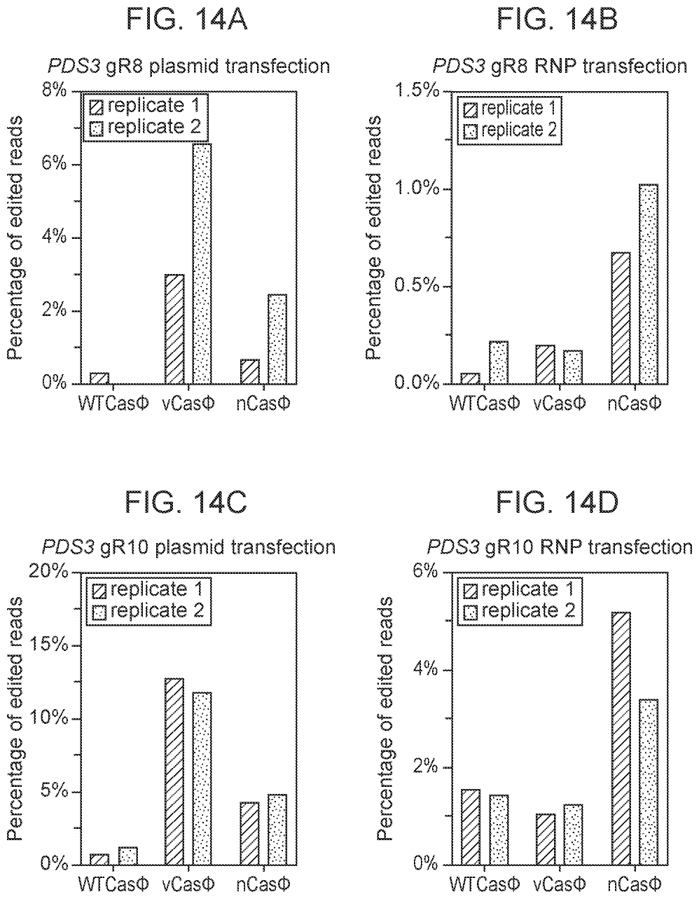

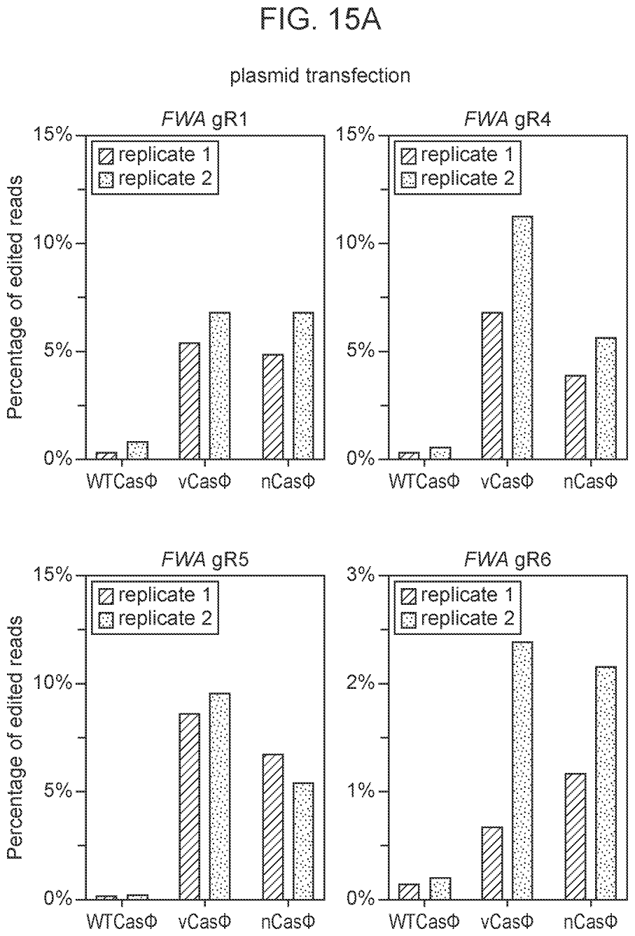

For AtPDS3 gRNA8 and gRNA10, compared to transfections of the WTCasΦ, higher editing efficiencies were observed with plasmid transfections of the vCasΦ and nCasΦ variants (FIG. 14A and FIG. 14C), and with RNP transfections of the nCasΦ variant (FIG. 14B and FIG. 14D).

FIG. 14A-14D. The CasΦ variants have higher target gene editing efficiencies than WTCasΦ for AtPDS3 gRNA8 and gRNA10. Plasmid transfections (A and C) and RNP transfections (B and D) were performed with Arabidopsis mesophyll protoplasts prepared from wild type plants (Col-0) and with the same amount of plasmids and RNPs, respectively. AtU6-26 promoter was used to drive the transcription of AtPDS3 gRNA8 (A) and AtPDS3 gRNA10 (C) in the plasmids used. Two replicate transfections were performed. For the AtPDS3 gR8 WTCasΦ plasmid transfection (A), editing efficiency was obtained for only one replicate due to library preparation failure for the other replicate.

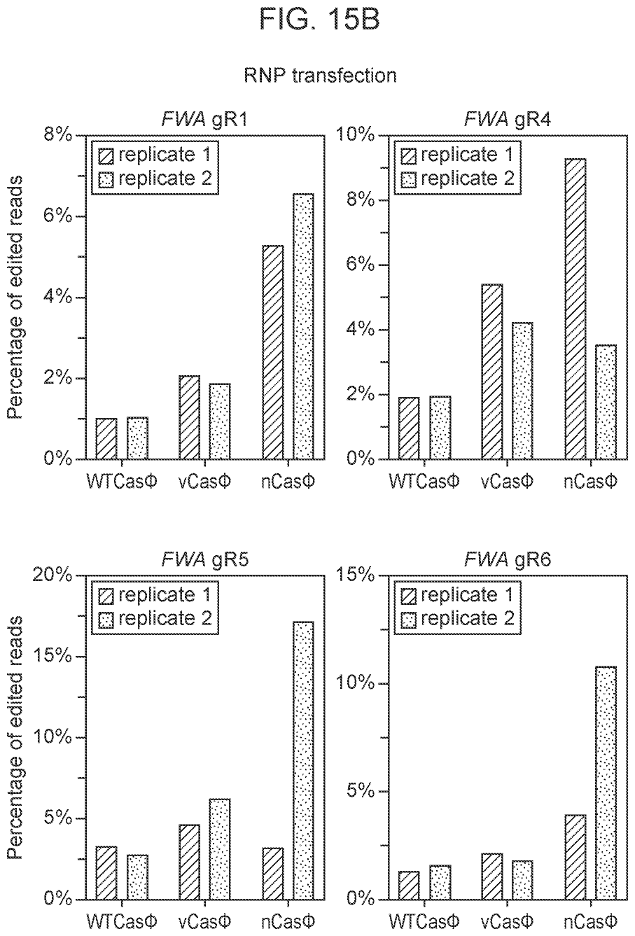

To further confirm these results, plasmid and RNP transfections were performed for FWA gRNA1, gRNA4, gRNA5 and gRNA6, with protoplasts prepared from the epi-mutant fwa-4. In the fwa-4 epi-mutant, the FWA gene promoter is unmethylated and less compact compared to the wild type plants (PMID: 14631047 and PMID: 11090618), which is potentially more accessible for gene editing machineries. For all four FWA gRNAs tested, similar to the AtPDS3 gRNA8 and gRNA10, plasmid transfections with both CasΦ variants yielded higher editing efficiencies (FIG. 15A), and RNP transfections with the nCasΦ variant yielded higher editing efficiency compared to the WTCasΦ (FIG. 15B).

FIG. 15A-15B. The CasΦ variants have higher target gene editing efficiencies than WTCasΦ for FWA gRNA1, gRNA4, gRNA5 and gRNA6. Plasmid transfections (A) and RNP transfections (B) were performed with Arabidopsis mesophyll protoplasts prepared from the fwa-4 epi-mutant plants and with the same amount of plasmids and RNPs, respectively. AtU6-26 promoter was used to drive the transcription of guide RNAs in the plasmids used. Two replicate transfections were performed.

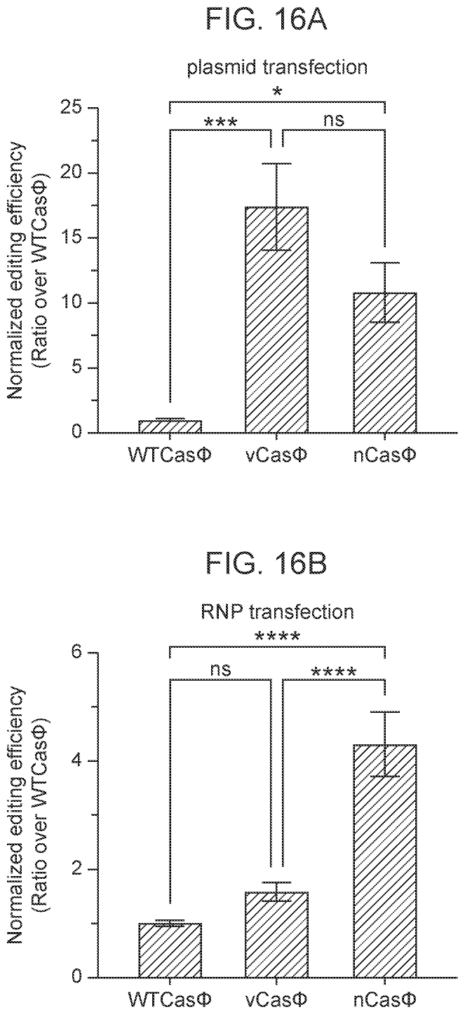

To statistically evaluate the differences between the editing efficiencies, for the 6 gRNAs tested, normalized editing efficiencies were calculated (ratio over WTCasΦ efficiency) and pooled for significance tests (FIG. 16A-16B). In the plasmid transfections, both the vCasΦ and nCasΦ variants yielded significantly higher editing efficiencies than the WTCasΦ (FIG. 16A). However, in the RNP transfections, only the nCasΦ variant yielded significantly higher editing efficiencies than the WTCasΦ (FIG. 16B). One possible reason for the differences observed between the plasmid and RNP transfections could be the differences in the production and degradation kinetics of the guide RNAs. In the plasmid transfections, guide RNAs could be continuously produced by transcription, while in the case of the RNP transfections, degraded guide RNA cannot be replaced. Overall, these results suggest that the vCasΦ and nCasΦ variants have higher target gene editing efficiencies than the WTCasΦ in the plant cells.

FIG. 16A-16B. The target gene editing efficiencies of CasΦ variants are significantly higher than that of WTCasΦ. For plasmid transfections (A) and RNP transfections (B), the target gene editing efficiencies of each guide RNAs used in FIG. 14A-14D and FIG. 15A-15B were normalized by calculating the ratio of editing efficiency over that of WTCasΦ. Then, the normalized editing efficiencies for different guide RNAs were pooled together for analysis. Mean and SEM of the normalized editing efficiencies were plotted and one-way ANOVA followed by Tukey's multiple comparisons tests were used to detect significant differences. *, 0.01<P<0.05, ***, 0.0001<P<0.001, ****, P<0.0001.

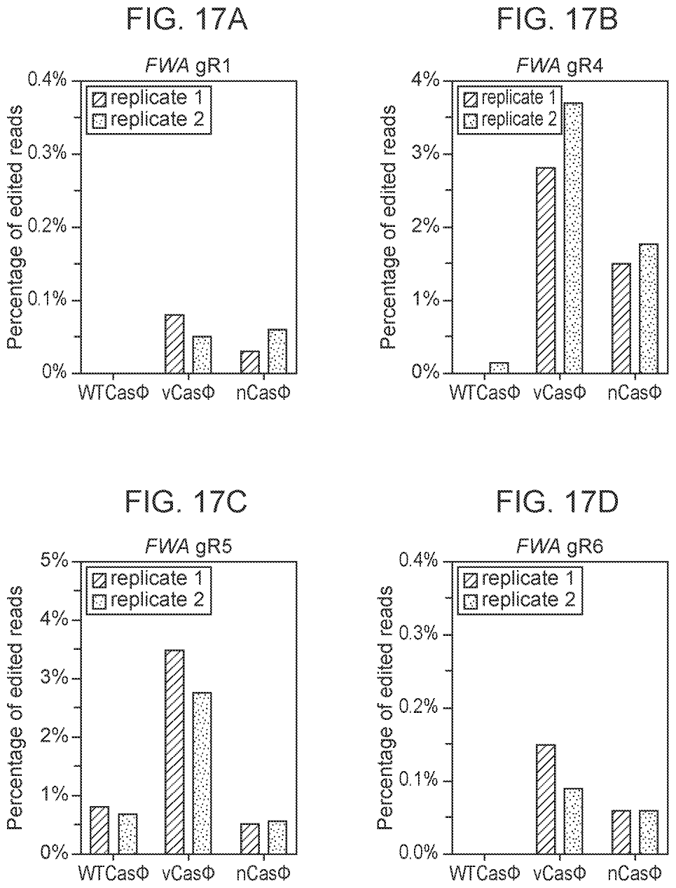

Since the FWA gene in the fwa-4 epi-mutant and the AtPDS3 gene are both actively transcribed genes, which have relatively more accessible chromatin environment, it was tested if the vCasΦ and nCasΦ variants were able to enhance the editing efficiency under repressive and compact chromatin conditions. To test this, protoplasts prepared from the wild type Arabidopsis plants were used for the plasmid transfections with FWA gRNA1, gRNA4, gRNA5 and gRNA6. As expected, very low editing efficiencies or no editing events by the WTCasΦ were observed for the FWA guide RNAs tested with the wild type protoplasts (FIG. 17A-17D). The vCasΦ and nCasΦ variants yielded readily detectable and higher editing efficiencies than the WTCasΦ for all four FWA guide RNAs tested (FIG. 17A-17D). These results suggest that under repressive and compact chromatin environments, the vCasΦ and nCasΦ variants are also able to dramatically enhance the editing efficiency compared to WTCasΦ.

FIG. 17A-17D. The CasΦ variants have higher target gene editing efficiencies than WTCasΦ under repressive and compact chromatin state. Plasmid transfections were performed with Arabidopsis mesophyll protoplasts prepared from wild type plants (Col-0) and with the same amount of plasmids. In the plasmids used, AtU6-26 promoter was used to drive the transcription of FWA gRNA1 (A), gRNA4 (B), gRNA5 (C) and gRNA6 (D). Two replicate transfections were performed.

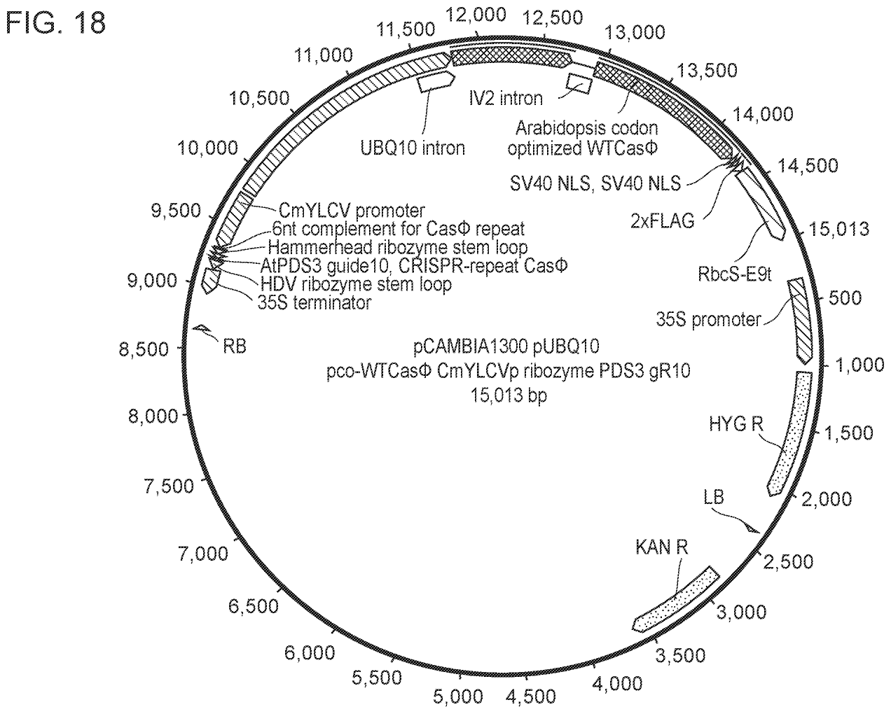

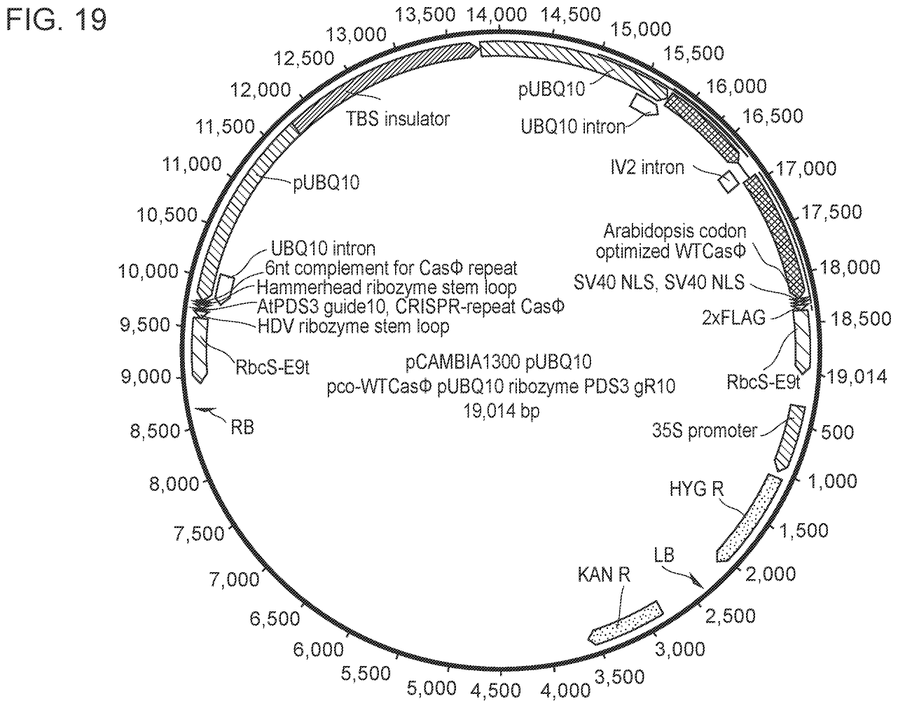

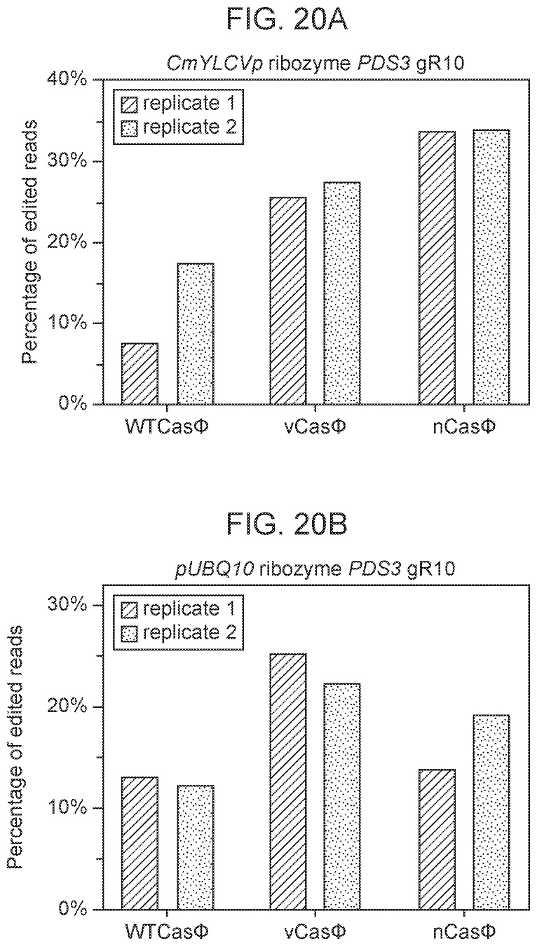

In addition to using Pol-III promoters for guide RNA transcription, Pol-II promoters in combination with proper guide RNA processing machineries can also be used, which offers tunable transcriptional strength and tissue specificity of the guide RNA production. The vCasΦ and nCasΦ variants were able to enhance the editing efficiency when the guide RNA was driven by the U6 promoter (Pol-III promoter). It was thought that the vCasΦ and nCasΦ variants might also increase the editing efficiency when guide RNA transcription is driven by Pol-II promoters. To test this hypothesis, two Pol-II promoters were used to for guide RNA transcription: the CmYLCV promoter and the UBQ10 promoter. The Hammerhead type ribozyme and hepatitis delta virus (HDV) ribozymes were cloned in to flank the AtPDS3 gRNA10 sequence for proper processing and release of the guide RNA. Plasmid maps with WTCasΦ are provided in FIG. 18 and FIG. 19, and the DNA sequences for Pol-II gRNA cassettes are provided in FIG. 24 and FIG. 25. For both the CmYLCV promoter and the UBQ10 promoter, the editing efficiencies of the vCasΦ and nCasΦ variants were higher than that of the WTCasΦ (FIG. 20A-20B). These results suggest that when guide RNA transcription is driven by Pol-II promoter, the vCasΦ and nCasΦ variants are also able to increase the target gene editing efficiency.

In FIG. 24, italic letters indicate the CmYLCV promoter; the first and second patches of letters in bold indicate the nucleotide sequences of the Hammerhead ribozyme stem loop and HDV ribozyme stem loop, respectively; underlined letters indicate the 35S terminator; and the underlined and italic letters indicate the CasΦ CRISPR repeat sequence and the spacer sequence for AtPDS3 gR10. In FIG. 25, italic letters indicate the UBQ10 promoter; the first and second patches of letters in bold indicate the nucleotide sequences of the Hammerhead ribozyme stem loop and HDV ribozyme stem loop, respectively; underlined letters indicate the RbcS-E9 terminator; and the underlined and italic letters indicate the CasΦ CRISPR repeat sequence and the spacer sequence for AtPDS3 gRNA10.

FIG. 18. Plasmid map of pCAMBIA1300 pUBQ10 pco-WTCasΦ CmYLCVp ribozyme PDS3 gR10. In this plasmid, Arabidopsis codon optimized wild type CasΦ was driven by the UBQ10 promoter and RbcS-E9 terminator. The CmYLCV promoter was used to drive the transcription of the CasΦ CRISPR repeat sequence followed by the spacer sequence for AtPDS3 gRNA10. The CRISPR repeat and the spacer sequence were flanked by Hammerhead ribozyme on the 5′ end and the HDV ribozyme on the 3′ end.

FIG. 19. Plasmid map of pCAMBIA1300 pUBQ10 pco-WTCasΦ pUBQ10 ribozyme PDS3 gR10. In this plasmid, Arabidopsis codon optimized wild type CasΦ was driven by the UBQ10 promoter and RbcS-E9 terminator. The UBQ10 promoter was used to drive the transcription of the CasΦ CRISPR repeat sequence followed by the spacer sequence for AtPDS3 gRNA10. The CRISPR repeat and the spacer sequence were flanked by Hammerhead ribozyme on the 5′ end and the HDV ribozyme on the 3′ end.

FIG. 20A-20B. The CasΦ variants have higher target gene editing efficiencies than WTCasΦ, with guide RNA driven by Pol-II promoters. Plasmid transfections were performed with Arabidopsis mesophyll protoplasts prepared from wild type plants (Col-0) and with the same amount of plasmids. In the plasmids used, CmYLCV promoter (A) and UBQ10 promoter (B) were used to drive the transcription of AtPDS3 gRNA10. The guide RNA sequence was flanked by Hammerhead ribozyme on the 5′ end and the HDV ribozyme on the 3′ end. Two replicate transfections were performed.

Consistent with the in vitro data that the vCasΦ and nCasΦ variants cleaved target DNA with faster kinetics, from the assays in this example, it was shown that these variants edit target genes with higher efficiency in the plant cells, under both active and repressive chromatin environments. The vCasΦ and nCasΦ variants, similar to the WTCasΦ, are also compatible with the Pol-II guide RNA transcription cassettes, yielding higher editing efficiency than the WTCasΦ.

### Example 4: The Engineered Variants vCasΦ and nCasΦ Enhance Target Gene Editing Efficiency in Arabidopsis Transgenic Plants

In Example 3, it was shown that the engineered variants vCasΦ and nCasΦ enhance target gene editing efficiency in Arabidopsis mesophyll protoplasts at multiple target loci. In this example, target gene editing efficiencies were examined in transgenic T1 plants for PDS3 gRNA10. Higher editing efficiencies were observed in the transgenic plants with the variant forms of CasΦ. Albino seedlings were observed in T2 populations of vCasΦ and nCasΦ PDS3 gRNA10 transgenic plants, indicating that homozygous mutants arose in T2 populations. Furthermore, transgene-free seedlings were identified from these albino seedlings, indicating that the mutation generated by the vCasΦ and nCasΦ variants are heritable.

### Methods

### Agrobacterium-Mediated Transformation

Transformation of Arabidopsis was performed with Agrobacterium strain AGL0 following the protocol described in PMID: 17406292 (Zhang et al. (2006) Nat. Protoc. 1:641). The Arabidopsis rdr6-15 mutant (PMID 15565108; Allen et al. (2004) Nat. Genet. 36:1282) plants were used for transformation to avoid potential transgene silencing. Plasmid constructed in example 3 with AtPDS3 gRNA10 were used for Agrobacterium-mediated transformation.

### Selection of Transgenic TI Plants

Seeds of Agrobacterium transformed plants were sterilized and plated onto 1/2 MS medium plates with 40 μg/ml hygromycin B (ThermoFisher 10687010). Then the seeds were stratified in the dark at 4° C. for 48-72 hours. Plates were then placed into a growth room at room temperature. Transgenic T1 hygromycin resistant plants were transferred from plates to soil when they could be clearly distinguished from plants that were not resistant to hygromycin. On hygromycin MS plates, resistant plants are able to develop normal long roots and true leaves while non-resistant plants have roots that do not elongate and do not develop true leaves.

### Growth of Transgenic 72 Populations

Transgenic T2 seeds were harvested from individual T1 plants and surface sterilized. Then the seeds were plated on half MS medium with 3% sucrose to support the growth of potential albino seedlings.

### DNA Extraction

To extract DNA from the transgenic plants for amplicon sequencing, 2-3 leaves were collected from each T1 plant. The leaves from the same T1 plant were pooled together for DNA extraction. DNeasy Plant Mini Kit (Qiagen cat. 69109) was used to extract DNA from albino seedlings.

### Amplicon Sequencing and Amplicon Sequencing Result Analysis

The amplicon sequencing and corresponding result analysis were performed as described in Example 3.

### Results

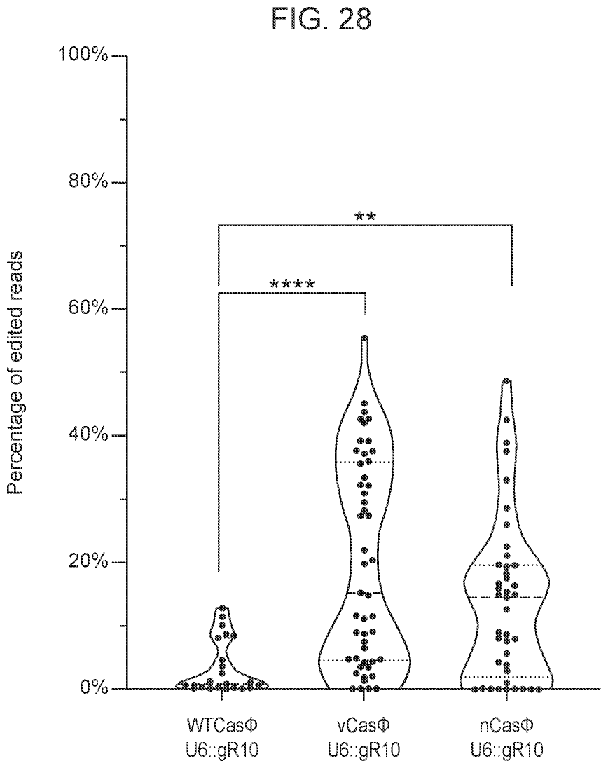

In Example 3, evidence was shown that the engineered variants vCasΦ and nCasΦ exhibit higher editing efficiencies on target loci in Arabidopsis mesophyll protoplasts. Here, it was tested if the engineered variants vCasΦ and nCasΦ could perform target gene editing with higher efficiency in transgenic plants, where the DNA cassettes encoding the CasΦ protein and the guide RNA are inserted in the plant genome. To test this, the pCAMBIA1300 pUBQ10 pco-vCasΦ U6 PDS3 gRNA10 and pCAMBIA1300 pUBQ10 pco-nCasΦ U6 PDS3 gRNA10 plasmid were transformed into the rdr6-15 mutant plants by Agrobacterium-mediated transformation. The plasmid pCAMBIA1300 pUBQ10 pco-WTCasΦ U6 PDS3 gRNA10 plasmid was also transformed into the rdr6-15 mutant plants as the control. Between these constructs, the only variable is the CasΦ protein encoded, while the guide RNA was driven by the same AtU6-26 promoter. Significantly higher editing efficiencies were observed in the T1 plants with the DNA cassette encoding the vCasΦ and nCasΦ variants (FIG. 28), confirming the ability of these variants to enhance the target gene editing efficiencies in transgenic plants.

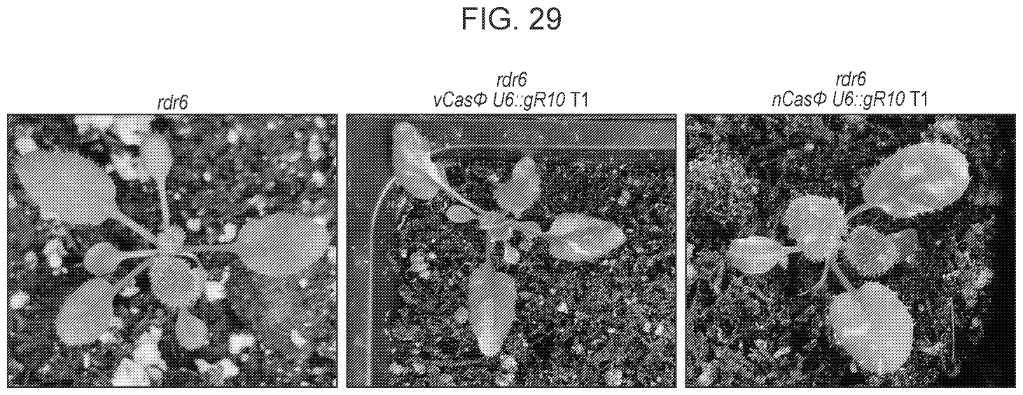

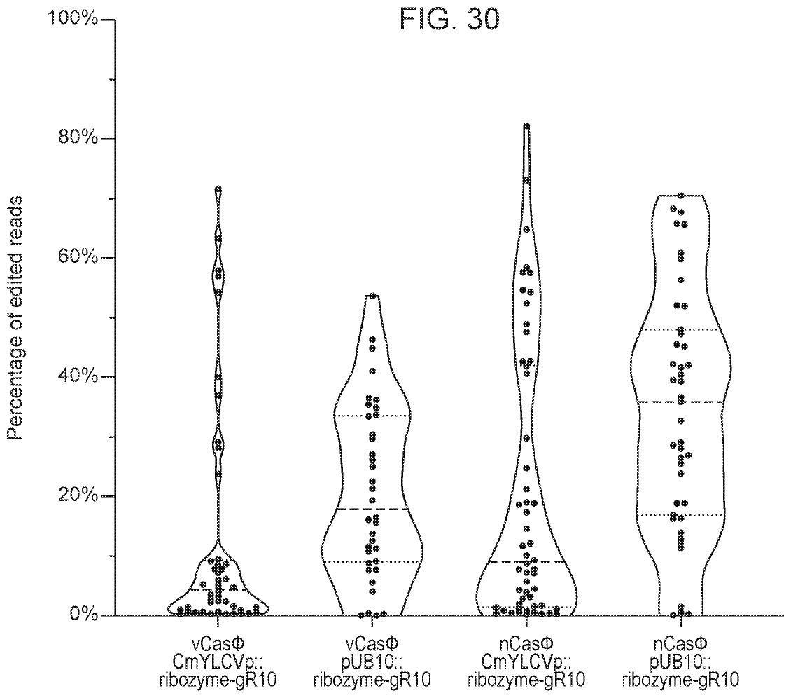

The AtPDS3 gene is essential for early chloroplast biogenesis and homozygous mutations of the AtPDS3 gene exhibit albino and dwarf phenotypes (PMID 17486124; Qin et al. (2007) Cell Res. 17:471). Thus, in the T1 transgenic plants in which the AtPDS3 gene is edited with high efficiency, it is expected that some cells will have both alleles of the AtPDS3 gene edited, leading to an albino phenotype. Indeed, T1 plants with white sectors were observed from the vCasΦ and nCasΦexpressing T1 populations (FIG. 29), indicating strong editing activity in somatic cells. These white sectors were not observed in transgenic T1 plants expressing wild type CasΦ.

In Example 3, it was shown that Pol-II promoters in combination with ribozyme-mediated gRNA processing can also be used together with the engineered CasΦ variants in mesophyll protoplasts. To test if this is also functional in transgenic plants, pCAMBIA1300 pUBQ10 pco-vCasΦ CmYLCVp ribozyme-PDS3 gRNA10, pCAMBIA1300 pUBQ10 pco-vCasΦ pUBQ10 ribozyme-PDS3 gRNA10, pCAMBIA1300 pUBQ10 pco-nCasΦ CmYLCVp ribozyme-PDS3 gRNA10 and pCAMBIA1300 pUBQ10 pco-nCasΦ pUBQ10 ribozyme-PDS3 gRNA10 plasmids were transformed into rdr6-15 mutant plants by Agrobacterium-mediated transformation. Very high target gene editing efficiencies were observed in the leaves of the transgenic T1 plants with vCasΦ and nCasΦ combined with the CmYLCV promoter or UBQ10 promoter driven PDS3 gRNA10 flanked by ribozymes (FIG. 30), indicating that the combinations of the engineered CasΦ variants and the Pol-II promoter driven gRNA cassettes were also functional in transgenic plants. In certain T1 plants, the target gene editing was very efficient with editing efficiencies reaching 50-80% (FIG. 30). These results suggest that when combined with proper Pol-II promoter guide RNA transcription and processing machineries, the engineered CasΦ variants were able to edit the target gene with high efficiency, which renders them a powerful and tunable tool for practical editing applications.

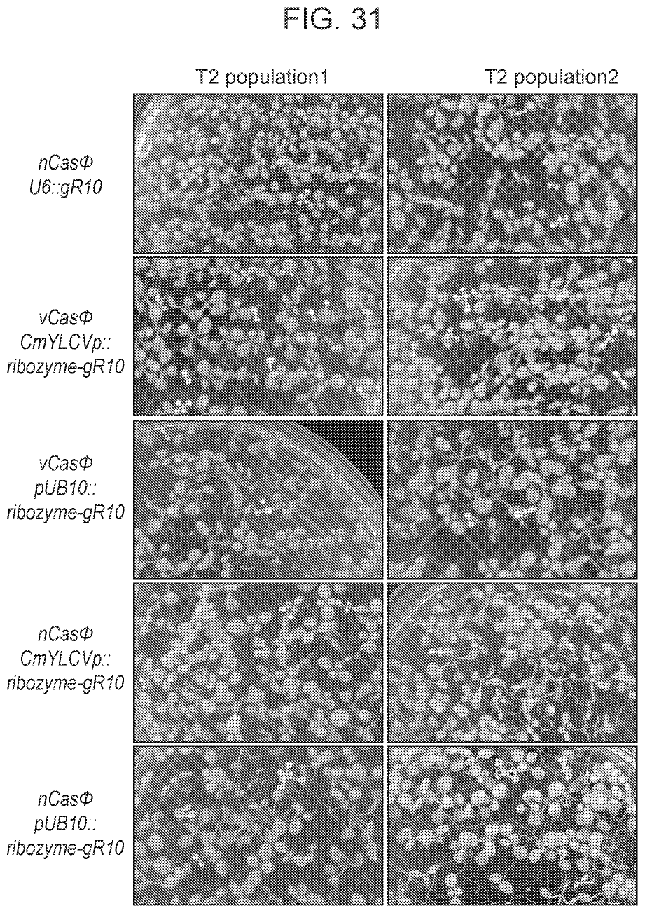

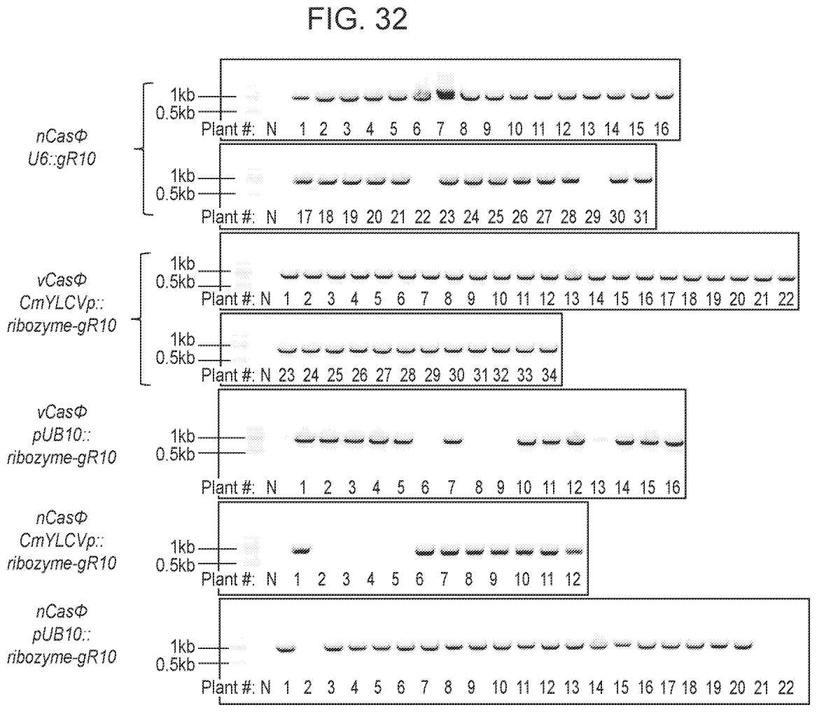

When the target gene is edited on both the maternal and the paternal chromosomes of germline cells, the offspring plants can be homozygous for the target gene mutation. If the target gene is the PDS3 gene, such offspring plants with homozygous PDS3 gene mutations inherited from the germline cells will appear as albino in the whole plant, instead of the mosaic pattern with white sectors in T1 plants as shown in FIG. 29. Indeed, consistent with the high editing efficiency of T1 plants observed by amplicon sequencing, albino seedlings were readily observed in the T2 populations of the engineered CasΦ variant transgenic plants (FIG. 31). The highest frequency lines showed 13 albino seedlings out of a total of 214 seedlings for the pCAMBIA1300 pUBQ10 pco-nCasΦ U6 PDS3 gRNA10 T2 lines; 24 albino seedlings out of a total of 197 seedlings for the pCAMBIA1300 pUBQ10 pco-vCasΦ CmYLCVp ribozyme-PDS3 gRNA10 T2 lines; 5 albino seedlings out of a total of 169 seedlings for the pCAMBIA1300 pUBQ10 pco-vCasΦ pUBQ10 ribozyme-PDS3 gRNA10 T2 lines; 6 albino seedlings out of a total of 210 seedlings for the pCAMBIA1300 pUBQ10 pco-nCasΦ CmYLCVp ribozyme-PDS3 gRNA10 lines and 4 albino seedlings out of a total of 163 seedlings for the pCAMBIA1300 pUBQ10 pco-nCasΦ pUBQ10 ribozyme-PDS3 gRNA10 lines. DNA from albino T2 seedlings were extracted and genotyped for the presence of the CasΦ transgene by the PCR amplification of a fragment of DNA encoding the CasΦ proteins. Transgene-free albino seedlings were identified from the T2 populations of pCAMBIA1300 pUBQ10 pco-nCasΦ U6 PDS3 gRNA10, pCAMBIA1300 pUBQ10 pco-vCasΦ pUBQ10 ribozyme-PDS3 gRNA10, pCAMBIA1300 pUBQ10 pco-nCasΦ CmYLCVp ribozyme-PDS3 gRNA10 and pCAMBIA1300 pUBQ10 pco-nCasΦ pUBQ10 ribozyme-PDS3 gRNA10 in the rdr6-15 background (FIG. 32). All 34 albino seedlings from pCAMBIA1300 pUBQ10 pco-vCasΦ CmYLCVp ribozyme-PDS3 gRNA10 T2 lines contain ed CasΦ transgenes, likely because of multiple T-DNA insertions. These results indicate that gene edits produced by the engineered CasΦ variants can be inherited into offspring generations.

From this example, it is shown that the engineered variants vCasΦ and nCasΦ enhanced the target gene editing efficiencies in transgenic plants and the edited gene products can be inherited to offspring plants. The engineered variants vCasΦ and nCasΦ are also compatible with the Pol-II promoter driven guide RNA transcription and ribozyme guide RNA processing machineries. With the wide variety of available Pol-II promoters, this observation allows for more controllable and varied applications.

FIG. 28. Target gene editing efficiencies of CasΦ variants are significantly higher than that of the wild type CasΦ in T1 transgenic plants. Leaf tissue of T1 transgenic plants of indicated plasmids in the rdr6-15 background were harvested for DNA extraction and amplicon sequencing analysis. Truncated violin plots and all data points representing the percentage of edited reads from each individual T1 plant are shown. Mann-Whitney test were used to detect significant differences. *, 0.01<P<0.05, **, 0.001<P<0.01, ***, 0.0001<P<0.001, ****, P<0.0001.

FIG. 29. White sectors were observed in leaves of vCASΦ and nCASΦ PDS3 gR10 transgenic T1 plants. A control plant (rdr6-15 mutant) is shown on the left, with leaves appearing as uniformly green. T1 transgenic plants of vCasΦ U6::PDS3 gR10 (middle) and nCasΦ U6::PDS3 gR10 (right) are shown, with white sectors on their leaves.

FIG. 30. Target gene editing efficiencies of CasΦ variants combined with Pol-II promoters and PDS3 gRNA10 flanked by ribozymes, in T1 transgenic plants. Leaf tissue of T1 transgenic plants of indicated plasmids on rdr6 background were harvested for DNA extraction and amplicon sequencing analysis. Truncated violin plot and all data points representing the percentage of edited reads from each individual T1 plant are shown. Mann-Whitney test were used to detect significant differences. *, 0.01<P<0.05, ***, 0.0001<P<0.001, ****, P<0.0001.

FIG. 31. Albino seedlings were observed in T2 populations of vCASΦ and nCASΦ PDS3 gR10 transgenic plants. T2 populations of vCASΦ and nCASΦ PDS3 gR10 transgenic plants were plated on half MS medium with 3% sucrose. Completely albino seedlings were observed from these populations.

FIG. 32. Albino seedlings which are transgene free were identified in T2 populations of vCASΦ and nCASΦ PDS3 gR10 transgenic plants. Albino seedlings from multiple T2 populations of vCASΦ and nCASΦ PDS3 gR10 transgenic plants were individually collected for DNA extraction. PCR amplification of a fragment of DNA encoding the CasΦ protein were performed. N, DNA of rdr6-15 plant were used as template for PCR as negative control. Lanes with no band represent plants in which the transgene has been segregated away.

### Example 5: The Engineered Variants vCasΦ and nCasΦ Enhance Target Gene Editing Efficiency in T2 Populations of Arabidopsis Transgenic Plants

In Example 4, it was shown that the engineered variants vCasΦ and nCasΦ enhance target gene editing efficiency in Arabidopsis transgenic plants in the T1 generation. It was also shown that albino seedlings were observed in T2 populations of vCasΦ and nCasΦ PDS3 gRNA10 transgenic plants. In this example, the number of albino seedlings in multiple T2 populations was quantified for each transgene of WTCasΦ, vCasΦ or nCasΦ with PDS3 gRNA10. It was observed that, compared to the WTCasΦ, the CasΦ variants led to significantly more albino seedlings in T2 populations.

### Methods

### Growth of Transgenic T2 Populations

Transgenic T2 seeds were harvested from individual T1 plants without pre-selection of T1 plants by target gene editing efficiency. Then the seeds were surface sterilized and plated on half MS medium with 3% sucrose to support the growth of potential albino seedlings. Total number of seedlings and the number of albino seedlings grown from the plated seeds of each T2 population were counted.

### Results

In example 4, it was shown that albino seedlings were observed in T2 populations of vCasΦ and nCasΦ PDS3 gRNA10 transgenic plants. As opposed to the white sectors observed in the T1 plants that reflect the editing of the AtPDS3 gene in both alleles in somatic cells, completely albino seedlings reveal homozygous/biallelic mutations in both alleles of the AtPDS3 gene inherited from a heterozygous parental plant and/or new editing events occurring in germline cells of the T1 plants. Thus, the number of albino seedlings in the T2 populations reveals germline PDS3 gene editing efficiency and the heritability of the resulting mutations.

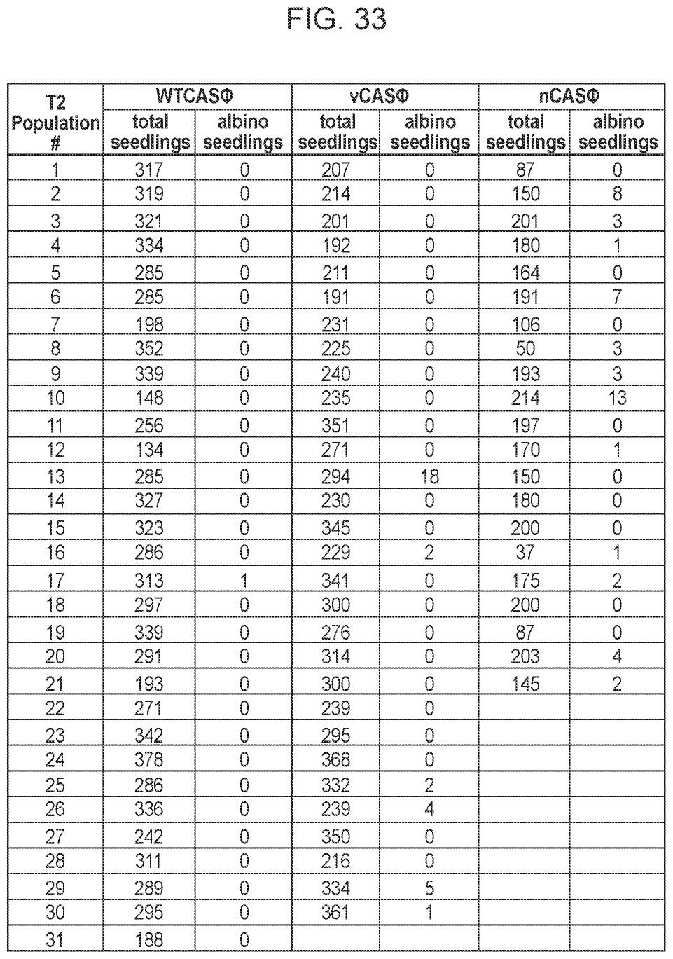

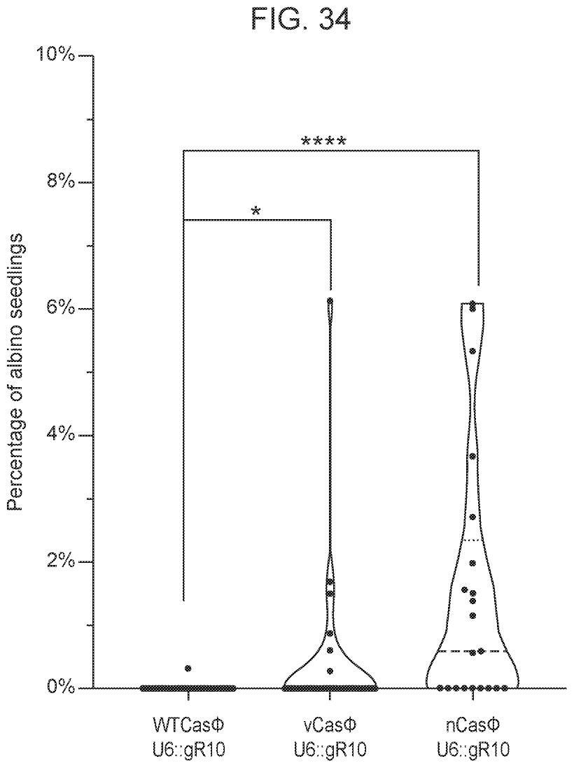

To compare the number of albino seedlings, T2 seeds were harvested from T1 transgenic plants of WTCasΦ U6::PDS3 gR10, vCasΦ U6::PDS3 gR10 and nCasΦ U6::PDS3 gR10 in the rdr6-15 mutant background. After surface sterilization and plating of the seeds, for each T2 population, the total number of seedlings grown from the plated seeds and the number of albino seedlings were counted (FIG. 33). Among the T2 populations of WTCasΦ U6::PDS3 gR10 tested, only 1 T2 population (T2 population #17) had 1 albino seedlings out of a total number of 313 seedlings from this T2 population. In contrast, among the T2 populations of vCasΦ U6::PDS3 gR10 and nCasΦ U6::PDS3 gR10, multiple T2 populations had albino seedlings of various numbers (FIG. 33). FIG. 34 displays the frequency of albino seedlings in the T2 populations, showing that the vCasΦ and nCasΦ variants had a significantly higher percentage of albino seedlings in T2 populations compared to the WTCasΦ. These results indicate that the CasΦ variants are more potent than the WTCasΦ at generating offspring plants where both alleles of the target gene are edited.

FIG. 33. Total seedling number and albino seedling number from the T2 populations of WTCASΦ U6::PDS3 gR10, vCASΦ U6::PDS3 gR10 and nCASΦ U6::PDS3 gR10. T2 seed populations were collected from T1 plants of each transgene without pre-selection of T1 plants by editing efficiency in T1 generation. Seeds from each T2 population were surface sterilized and plated on half MS medium with 3% sucrose. Total number of seedlings grown and the number of albino seedlings from each T2 populations were counted.

FIG. 34. The percentage of albino seedlings in T2 transgenic populations of CasΦ variants are significantly higher than that of the wild type CasΦ with PDS3 gRNA10 driven by the U6 promoter. T2 populations of WTCASΦ U6::PDS3 gR10, vCASΦ U6::PDS3 gR10 and nCASΦ U6::PDS3 gR10 in the rdr6-15 mutant background were plated. Total seedling and albino seedling number from each population were counted. Truncated violin plots and all data points representing the percentage of albino seedlings from each T2 population are shown. Mann-Whitney test were used to detect significant differences. *, 0.01<P<0.05, **, 0.001<P<0.01, ***, 0.0001<P<0.001, ****, P<0.0001.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A variant CRISPR-Cas effector polypeptide comprising an amino acid sequence having at least 50% amino acid sequence identity to any one of the amino acid sequences depicted in FIG. 9A-9R, wherein the variant CRISPR-Cas effector polypeptide comprises a deletion or a substitution of one or more amino acids in the alpha-7 helix of the Rec I domain, compared to the amino acid sequence depicted in FIG. 6, or a corresponding region of another CasPhi polypeptide, and
wherein the variant CRISPR-Cas effector polypeptide exhibits at least a 10% increased cis- and/or trans-cleavage activity compared to the cis- and/or trans-cleavage activity of a CasPhi polypeptide comprising the amino acid sequence depicted in FIG. 6.

2. The variant CRISPR-Cas effector polypeptide of claim 1, wherein the variant CRISPR-Cas effector polypeptide comprises amino acid substitutions of amino acids E159, S160, S164, D167, and E168, compared to the amino acid sequence depicted in FIG. 6, or corresponding amino acids in another CasPhi polypeptide.

3. The variant CRISPR-Cas effector polypeptide of claim 2, wherein the variant CRISPR-Cas effector polypeptide comprises E159A, S160A, S164A, D167A, and E168A substitutions, compared to the amino acid sequence depicted in FIG. 6.

4. The variant CRISPR-Cas effector polypeptide of claim 1, wherein the variant CRISPR-Cas effector polypeptide comprises a replacement of from 15 amino acids to 52 amino acids within amino acids 144-195 of the amino acid sequence depicted in FIG. 6, or a corresponding stretch of amino acids in the alpha-7 helix of another CasPhi polypeptide, with a heterologous polypeptide.

5. The variant CRISPR-Cas effector polypeptide of claim 4, wherein the variant CRISPR-Cas effector polypeptide comprises a replacement of amino acids 155-176 of the amino acid sequence depicted in FIG. 6, or a corresponding stretch of amino acids in another CasPhi polypeptide.

6. The variant CRISPR-Cas effector polypeptide of claim 4 or claim 5, wherein the heterologous polypeptide comprises Gly, Ser, or a combination of Gly and Ser, and wherein the heterologous polypeptide has a length of from 4 amino acids to about 25 amino acids.

7. The variant CRISPR-Cas effector polypeptide of claim 4 or claim 5, wherein the heterologous polypeptide exhibits an enzymatic activity.

8. The variant CRISPR-Cas effector polypeptide of claim 7, wherein the heterologous polypeptide is a base editor.

9. The variant CRISPR-Cas effector polypeptide of claim 4 or claim 5, wherein the heterologous polypeptide comprises a protein-binding domain.

10. The variant CRISPR-Cas effector polypeptide of claim 4 or claim 5, wherein the heterologous polypeptide is a nucleic acid-binding polypeptide, a nucleic acid modifying polypeptide, or a protein-binding polypeptide.

11. The variant CRISPR-Cas effector polypeptide of any one of claims 1-10, wherein the variant CRISPR-Cas effector polypeptide exhibits at least 2-fold increased cis- and/or trans-cleavage activity compared to the cis- and/or trans-cleavage activity of a CasPhi polypeptide comprising the amino acid sequence depicted in FIG. 6.

12. The variant CRISPR-Cas effector polypeptide of any one of claims 1-10, wherein the variant CRISPR-Cas effector polypeptide exhibits at least 5-fold increased cis- and/or trans-cleavage activity compared to the cis- and/or trans-cleavage activity of a CasPhi polypeptide comprising the amino acid sequence depicted in FIG. 6.

13. The variant CRISPR-Cas effector polypeptide of any one of claims 1-10, wherein the variant CRISPR-Cas effector polypeptide exhibits at least 10-fold increased cis- and/or trans-cleavage activity compared to the cis- and/or trans-cleavage activity of a CasPhi polypeptide comprising the amino acid sequence depicted in FIG. 6.

14. The variant CRISPR-Cas effector polypeptide of any one of claims 1-10, wherein the variant CRISPR-Cas effector polypeptide exhibits at least 15-fold increased cis- and/or trans-cleavage activity compared to the cis- and/or trans-cleavage activity of a CasPhi polypeptide comprising the amino acid sequence depicted in FIG. 6.

15. A fusion polypeptide comprising:
a) a variant CRISPR-Cas effector polypeptide of any one of claims 1-14; and
b) one or more heterologous polypeptides.

16. The fusion polypeptide of claim 15, wherein the one or more heterologous polypeptides is fused to the N-terminus and/or the C-terminus of the variant CRISPR-Cas effector polypeptide.

17. The fusion polypeptide of claim 15 or claim 16, wherein at least one of the one or more heterologous polypeptides comprises a nuclear localization signal (NLS).

18. The fusion polypeptide of any one of claims 15-17, wherein at least one of the one or more heterologous polypeptides is a targeting polypeptide that provides for binding to a cell surface moiety on a target cell or target cell type.

19. The fusion polypeptide of any one of claims 15-17, wherein at least one of the one or more heterologous polypeptides exhibits an enzymatic activity that modifies target DNA.

20. The fusion polypeptide of any one of claims 15-17, wherein at least one of the one or more heterologous polypeptides exhibits an enzymatic activity that modifies a target polypeptide associated with a target nucleic acid.

21. The fusion polypeptide of any one of claims 15-20, wherein at least one of the one or more heterologous polypeptides is an endosomal escape polypeptide.

22. The fusion polypeptide of any one of claims 15-20, wherein at least one of the one or more heterologous polypeptides is a chloroplast transit peptide.

23. The fusion polypeptide of any one of claims 15-20, wherein at least one of the one or more heterologous polypeptides comprises a protein transduction domain.

24. The fusion polypeptide of any one of claims 15-20, wherein at least one of the one or more heterologous polypeptides is a protein binding domain.

25. A composition comprising:
a1) a variant CRISPR-Cas effector polypeptide of any one of claims 1-14, or a nucleic acid comprising a nucleotide sequence encoding the variant CRISPR-Cas effector polypeptide; and
b1) a CasPhi guide RNA, or one or more DNA molecules comprising nucleotide sequence(s) encoding the CasPhi guide RNA; or
a2) a fusion polypeptide of any one of claims 15-24 or a nucleic acid comprising a nucleotide sequence encoding the fusion polypeptide; and
b2) a CasPhi guide RNA, or one or more DNA molecules comprising nucleotide sequence(s) encoding the CasPhi guide RNA.

26. The composition of claim 25, wherein the CasPhi guide RNA comprises a nucleotide sequence having 80%, 90%, 95%, 98%, 99%, or 100%, nucleotide sequence identity with any one of the crRNA sequences depicted in FIG. 10, or the reverse complement of any one of the sequences depicted in FIG. 10, or the reverse complement of any one of the sequences depicted in FIG. 11.

27. The composition of claim 25 or claim 26, wherein the composition comprises a DNA molecule comprising a nucleotide sequence encoding the CasPhi guide RNA, and wherein the nucleotide sequence encoding the CasPhi guide RNA is operably linked to a Pol II promoter or a Pol III promoter.

28. The composition of claim 27, wherein the nucleotide sequence encoding the CasPhi guide RNA is operably linked to a Pol II promoter, and wherein the Pol II promoter is a UBQ10 promoter or a CmYLCV promoter.

29. The composition of claim 28, wherein the nucleotide sequence encoding the guide RNA is flanked by a nucleotide sequence encoding a first ribozyme stem loop and a nucleotide sequence encoding a second ribozyme stem loop.

30. The composition of any one of claims 25-29, wherein the guide RNA is a single-molecule guide RNA.

31. The composition of any one of claims 25-30, wherein the composition comprises a lipid.

32. The composition of any one of claims 25-31, wherein a) and b) are within a liposome.

33. The composition of any one of claims 25-30, wherein a) and b) are within a particle.

34. The composition of any one of claims 25-33, comprising one or more of: a buffer, a nuclease inhibitor, and a protease inhibitor.

35. The composition of any one of claims 25-34, further comprising a DNA donor template.

36. The composition of any one of claims 25-35, comprising a pharmaceutically acceptable excipient.

37. A nucleic acid comprising a nucleotide sequence encoding the variant CRISPR-Cas effector polypeptide of any one of claims 1-14, or the fusion polypeptide of any one of claims 15-24.

38. The nucleic acid of claim 37, wherein the nucleotide sequence encoding the variant CRISPR-Cas effector polypeptide, or the nucleotide sequence encoding the fusion polypeptide, is operably linked to a promoter.

39. The nucleic acid of claim 38, wherein the promoter is functional in a eukaryotic cell.

40. The nucleic acid of claim 39, wherein the promoter is functional in one or more of: a plant cell, a fungal cell, an animal cell, cell of an invertebrate, a fly cell, a cell of a vertebrate, a mammalian cell, a primate cell, a non-human primate cell, and a human cell.

41. The nucleic acid of any one of claims 28-40, wherein the promoter is one or more of: a constitutive promoter, an inducible promoter, a cell type-specific promoter, and a tissue-specific promoter.

42. The nucleic acid of any one of claims 37-41, wherein the nucleic acid is a recombinant expression vector.

43. The nucleic acid of claim 42, wherein the recombinant expression vector is a recombinant adeno-associated viral vector, a recombinant retroviral vector, or a recombinant lentiviral vector.

44. A composition comprising:
a) the nucleic acid of any one of claims 37-43; and
b) one or more of: a buffer, a nuclease inhibitor, a salt, a lipid, and a pharmaceutically acceptable excipient.

45. One or more nucleic acids comprising:
(a) a nucleotide sequence encoding a CasPhi guide RNA; and
(b) a nucleotide sequence encoding:
i) a variant CRISPR-Cas polypeptide of any one of claims 1-14; or
ii) a fusion polypeptide of any one of claims 15-24.

46. The one or more nucleic acids of claim 45, wherein the CasPhi guide RNA comprises a nucleotide sequence having 80% or more nucleotide sequence identity with any one of the crRNA sequences depicted in FIG. 10, or the reverse complement of any one of the sequences depicted in FIG. 10, or the reverse complement of any one of the sequences depicted in FIG. 11.

47. The one or more nucleic acids of claim 45 or claim 46, wherein the nucleotide sequence encoding the CasPhi guide RNA is operably linked to a promoter.

48. The one or more nucleic acids of claim 47, wherein the promoter is a Pol-II promoter.

49. The one or more nucleic acids of any one of claims 45-48, wherein the nucleotide sequence encoding the variant CRISPR-Cas polypeptide, or the nucleotide sequence encoding the fusion polypeptide, is operably linked to a promoter.

50. The one or more nucleic acids of claim 49, wherein the promoter is a promoter that is functional in a eukaryotic cell.

51. The one or more nucleic acids of claim 49 or claim 50, wherein the promoter is an inducible promoter.

52. A composition comprising:
a) the one or more nucleic acids of any one of claims 45-51; and
b) one or more of: a buffer, a nuclease inhibitor, a salt, a lipid, and a pharmaceutically acceptable excipient.

53. A cell comprising one or more of:
a) a variant CRISPR-Cas polypeptide of any one of claims 1-14, or a nucleic acid comprising a nucleotide sequence encoding the variant CRISPR-Cas polypeptide,
b) a fusion polypeptide of any one of claims 15-24, or a nucleic acid comprising a nucleotide sequence encoding the fusion polypeptide, and
c) a CasPhi guide RNA, or a nucleic acid comprising a nucleotide sequence encoding the CasPhi guide RNA.

54. The cell of claim 53, comprising the nucleic acid comprising a nucleotide sequence encoding the variant CRISPR-Cas polypeptide, or comprising the nucleic acid comprising a nucleotide sequence encoding the fusion polypeptide, wherein said nucleic acid is integrated into the genomic DNA of the cell.

55. The cell of claim 53 or claim 54, wherein the cell is a eukaryotic cell.

56. The cell of claim 55, wherein the eukaryotic cell is a plant cell, a mammalian cell, an insect cell, an arachnid cell, a fungal cell, a bird cell, a reptile cell, an amphibian cell, an invertebrate cell, a mouse cell, a rat cell, a primate cell, a non-human primate cell, or a human cell.

57. The cell of claim 53 or claim 54, wherein the cell is a prokaryotic cell.

58. The cell of any one of claims 53-57, wherein the cell is in vitro.

59. The cell of any one of claims 53-57, wherein the cell is in vivo.

60. A method of modifying a target nucleic acid, the method comprising contacting the target nucleic acid with:
a) a variant CRISPR-Cas effector polypeptide of any one of claims 1-14; and
b) a CasPhi guide RNA comprising a guide sequence that hybridizes to a target sequence of the target nucleic acid,
wherein said contacting results in modification of the target nucleic acid by the variant CRISPR-Cas effector polypeptide.

61. The method of claim 60, wherein said modification is cleavage of the target nucleic acid.

62. The method of claim 60 or claim 61, wherein the target nucleic acid is selected from: double stranded DNA, single stranded DNA, RNA, genomic DNA, and extrachromosomal DNA.

63. The method of any one of claims 60-62, wherein the target nucleic acid is present in repressive and compact chromatin.

64. The method of any one of claims 60-62, wherein the target nucleic acid is present in active and accessible chromatin.

65. The method of any of claims 60-64, wherein said contacting takes place in vitro outside of a cell.

66. The method of any of claims 60-64, wherein said contacting takes place inside of a cell in vitro.

67. The method of any of claims 60-65, wherein said contacting takes place inside of a cell in vivo.

68. The method of claim 67, wherein the cell is a eukaryotic cell.

69. The method of claim 68, wherein the cell is selected from: a plant cell, a fungal cell, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell.

70. The method of claim 66, wherein the cell is a prokaryotic cell.

71. The method of any one of claims 66-70, wherein said contacting results in genome editing.

72. The method of any one of claims 66-71, wherein said contacting comprises: introducing into a cell: (a) the variant CRISPR-Cas effector polypeptide, or a nucleic acid comprising a nucleotide sequence encoding the variant CRISPR-Cas effector polypeptide, and (b) the CasPhi guide RNA, or a nucleic acid comprising a nucleotide sequence encoding the CasPhi guide RNA.

73. The method of claim 72, wherein said contacting further comprises introducing a DNA donor template into the cell.

74. A transgenic, multicellular, non-human organism whose genome comprises a transgene comprising a nucleotide sequence encoding one or more of:
a) a variant CRISPR-Cas effector polypeptide of any one of claims 1-14,
b) a fusion polypeptide of any one of claims 15-24, and
c) a CasPhi guide RNA.

75. The transgenic, multicellular, non-human organism of claim 74, wherein the organism is a plant, an invertebrate animal, an insect, an arthropod, an arachnid, a parasite, a worm, a cnidarian, a vertebrate animal, a fish, a reptile, an amphibian, an ungulate, a bird, a pig, a horse, a sheep, a rodent, a mouse, a rat, or a non-human primate.

76. The transgenic, multicellular, non-human organism of claim 75, wherein the organism is a monocotyledon plant or a dicotyledon plant.

77. A system comprising one of:
a) a variant CRISPR-Cas effector polypeptide of any one of claims 1-14 and a CasPhi guide RNA;
b) a variant CRISPR-Cas effector polypeptide of any one of claims 1-14, a CasPhi guide RNA, and a DNA donor template;
c) a fusion polypeptide of any one of claims 15-24 and a CasPhi guide RNA;
d) a fusion polypeptide of any one of claims 15-24, a CasPhi guide RNA, and a DNA donor template;
e) an mRNA encoding a variant CRISPR-Cas effector polypeptide of any one of claims 1-14, and a CasPhi guide RNA;
f) an mRNA encoding a variant CRISPR-Cas effector polypeptide of any one of claims 1-14; a CasPhi guide RNA, and a DNA donor template;
g) an mRNA encoding a fusion polypeptide of any one of claims 15-24, and a CasPhi guide RNA;
h) an mRNA encoding a fusion polypeptide of any one of claims 15-24, a CasPhi guide RNA, and a DNA donor template;
i) one or more recombinant expression vectors comprising: i) a nucleotide sequence encoding a variant CRISPR-Cas effector polypeptide of any one of claims 1-14; and ii) a nucleotide sequence encoding a CasPhi guide RNA;
j) one or more recombinant expression vectors comprising: i) a nucleotide sequence encoding a variant CRISPR-Cas effector polypeptide of any one of claims 1-14; ii) a nucleotide sequence encoding a CasPhi guide RNA; and iii) a DNA donor template;
k) one or more recombinant expression vectors comprising: i) a nucleotide sequence encoding a fusion polypeptide of any one of claims 15-24; and ii) a nucleotide sequence encoding a CasPhi guide RNA; and
l) one or more recombinant expression vectors comprising: i) a nucleotide sequence encoding a fusion polypeptide of any one of claims 15-24; ii) a nucleotide sequence encoding a CasPhi guide RNA;
and a DNA donor template.

78. A composition comprising the system of claim 77.

79. The composition of claim 78, comprising one or more of: a buffer, a nuclease inhibitor, a protease inhibitor, a salt, a lipid, and a pharmaceutically acceptable excipient.

80. A kit comprising the system of claim 77 or the composition of claim 78 or 79.

81. The kit of claim 80, wherein the components of the kit are in the same container.

82. The kit of claim 80, wherein the components of the kit are in separate containers.

83. A sterile container comprising the system of claim 77 or the composition of claim 78 or 79.

84. The sterile container of claim 83, wherein the container is a syringe.

85. An implantable device comprising the system of claim 77 or the composition of claim 78 or 79.

86. The implantable device of claim 85, wherein the system is within a matrix.

87. The implantable device of claim 85, wherein the system is in a reservoir.

88. A method of detecting a target DNA in a sample, the method comprising:
(a) contacting the sample with:
(i) a variant CRISPR-Cas effector polypeptide of any one of claims 1-14 or a fusion polypeptide of any one of claims 15-24;
(ii) a guide RNA comprising: a region that binds to the variant CRISPR-Cas effector polypeptide of any one of claims 1-14, and a guide sequence that hybridizes with the target DNA; and
(iii) a detector DNA that is single stranded and does not hybridize with the guide sequence of the guide RNA; and
(b) measuring a detectable signal produced by cleavage of the single stranded detector DNA by the variant CRISPR-Cas effector polypeptide or the fusion polypeptide, thereby detecting the target DNA.

89. The method of claim 88, wherein the target DNA is single stranded.

90. The method of claim 88, wherein the target DNA is double stranded.

91. The method of any one of claims 88-90, wherein the target DNA is bacterial DNA.

92. The method of any one of claims 88-90, wherein the target DNA is viral DNA.

93. The method of claim 92, wherein the target DNA is papovavirus, human papillomavirus (HPV), hepadnavirus, Hepatitis B Virus (HBV), herpesvirus, varicella zoster virus (VZV), Epstein-Barr virus (EBV), Kaposi's sarcoma-associated herpesvirus, adenovirus, poxvirus, or parvovirus DNA.

94. The method of any one of claims 88-90, wherein the target DNA is from a human cell.

95. The method of any one of claims 88-90, wherein the target DNA is human fetal or cancer cell DNA.

96. The method of claim 94 or 95, wherein the sample comprises DNA from a cell lysate.

97. The method of claim 94 or 95, wherein the sample comprises cells.

98. The method of claim 97, wherein the sample is a blood, serum, plasma, urine, aspirate, or biopsy sample.

99. The method of any one of claims 88-98, further comprising determining an amount of the target DNA present in the sample.

100. The method of claim 99, wherein said measuring a detectable signal comprises one or more of: visual based detection, sensor-based detection, color detection, gold nanoparticle-based detection, fluorescence polarization, colloid phase transition/dispersion, electrochemical detection, and semiconductor-based sensing.

101. The method of any one of claims 88-100, wherein the labeled detector DNA comprises a modified nucleobase, a modified sugar moiety, and/or a modified nucleic acid linkage.

102. The method of any one of claims 88-101, further comprising detecting a positive control target DNA in a positive control sample, the detecting comprising:
(c) contacting the positive control sample with:
(i) the variant CRISPR-Cas polypeptide or the fusion polypeptide;
(ii) a positive control guide RNA comprising: a region that binds to the variant CRISPR-Cas polypeptide or the fusion polypeptide, and a positive control guide sequence that hybridizes with the positive control target DNA; and
(iii) a labeled detector DNA that is single stranded and does not hybridize with the positive control guide sequence of the positive control guide RNA; and
(d) measuring a detectable signal produced by cleavage of the labeled detector DNA by the variant CRISPR-Cas polypeptide or the fusion polypeptide, thereby detecting the positive control target DNA.

103. The method of any one of claims 88-102, wherein the detectable signal is detectable in less than 45 minutes.

104. The method of any one of claims 88-102, wherein the detectable signal is detectable in less than 30 minutes.

105. The method of any one of claims 88-104, further comprising amplifying the target DNA in the sample by loop-mediated isothermal amplification (LAMP), helicase-dependent amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR), or isothermal multiple displacement amplification (IMDA).

106. The method of any one of claims 88-105, wherein target DNA in the sample is present at a concentration of less than 10 aM.

107. The method according to any one of claim 88-106, wherein the single stranded detector DNA comprises a fluorescence-emitting dye pair.

108. The method according to any one of claims 88-107, wherein the fluorescence-emitting dye pair is a fluorescence resonance energy transfer (FRET) pair.

109. The method according to any one of claims 88-107, wherein the fluorescence-emitting dye pair is a quencher/fluor pair.

110. The method according to any one of claims 88-109, wherein the single stranded detector DNA comprises two or more fluorescence-emitting dye pairs.

111. The method according to claim 110, wherein said two or more fluorescence-emitting dye pairs include a fluorescence resonance energy transfer (FRET) pair and a quencher/fluor pair.


SEQUENCE LISTING (170 sequences)

SEQ ID NO: 1 - RNA, 36 nt, Artificial sequence
  synthetic sequence
ugccccucacgaggggacagcugguaaugggauacc

SEQ ID NO: 2 - DNA, 29 bp, Artificial sequence
  synthetic sequence
agcacaaggtatcccattaccagcttaac

SEQ ID NO: 3 - DNA, 29 bp, Artificial sequence
  synthetic sequence
gttaagctggtaatgggataccttgtgct

SEQ ID NO: 4 - protein, 698 aa, Artificial sequence
  synthetic sequence
  NON_CONS (514)..(515)
AVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKR

SEQ ID NO: 5 - protein, 441 aa, Artificial sequence
  synthetic sequence
MNKIEKEKTPLAKLMNENFAGLRFPFAIIKQAGKKLLKEGELKTIEYMTGKGSIEPLPNFKPPVKCLIVAKRRDLKYFPICKASCEIQSYVYSLNYKDFMDYFSTPMTSQKQHEEFFKKSGLNIEYQNVAGLNLIFNNVKNTYNGVILKVKNRNEKLKKKAIKNNYEFEEIKTFNDDGCLINKPGINNVIYCFQSISPKILKNITHLPKEYNDYDCSVDRNIIQKYVSRLDIPESQPGHVPEWQRKLPEFNNTNNPRRRRKWYSNGRNISKGYSVDQVNQAKIEDSLLAQIKIGEDWIILDIRGLLRDLNRRELISYKNKLTIKDVLGFFSDYPIIDIKKNLVTFCYKEGVIQVVSQKSIGNKKSKQLLEKLIENKPIALVSIDLGQTNPVSVKISKLNKINNKISIESFTYRFLNEEILKEIEKYRKDYDKLELKLINEA

SEQ ID NO: 6 - protein, 772 aa, Artificial sequence
  synthetic sequence
MSNTAVSTREHMSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 7 - protein, 793 aa, Artificial sequence
  synthetic sequence
MSSLPTPLELLKQKHADLFKGLQFSSKDNKMAGKVLKKDGEEAALAFLSERGVSRGELPNFRPPAKTLVVAQSRPFEEFPIYRVSEAIQLYVYSLSVKELETVPSGSSTKKEHQRFFQDSSVPDFGYTSVQGLNKIFGLARGIYLGVITRGENQLQKAKSKHEALNKKRRASGEAETEFDPTPYEYMTPERKLAKPPGVNHSIMCYVDISVDEFDFRNPDGIVLPSEYAGYCREINTAIEKGTVDRLGHLKGGPGYIPGHQRKESTTEGPKINFRKGRIRRSYTALYAKRDSRRVRQGKLALPSYRHHMMRLNSNAESAILAVIFFGKDWVVFDLRGLLRNVRWRNLFVDGSTPSTLLGMFGDPVIDPKRGVVAFCYKEQIVPVVSKSITKMVKAPELLNKLYLKSEDPLVLVAIDLGQTNPVGVGVYRVMNASLDYEVVTRFALESELLREIESYRQRTNAFEAQIRAETFDAMTSEEQEEITRVRAFSASKAKENVCHRFGMPVDAVDWATMGSNTIHIAKWVMRHGDPSLVEVLEYRKDNEIKLDKNGVPKKVKLTDKRIANLTSIRLRFSQETSKHYNDTMWELRRKHPVYQKLSKSKADFSRRVVNSIIRRVNHLVPRARIVFIIEDLKNLGKVFHGSGKRELGWDSYFEPKSENRWFIQVLHKAFSETGKHKGYYIIECWPNWTSCTCPKCSCCDSENRHGEVFRCLACGYTCNTDFGTAPDNLVKIATTGKGLPGPKKRCKGSSKGKNPKIARSSETGVSVTESGAPKVKKSSPTQTSQSSSQSAP

SEQ ID NO: 8 - protein, 717 aa, Artificial sequence
  synthetic sequence
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 9 - protein, 765 aa, Artificial sequence
  synthetic sequence
MYSLEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 10 - protein, 766 aa, Artificial sequence
  synthetic sequence
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 11 - protein, 793 aa, Artificial sequence
  synthetic sequence
MRSSREIGDKILMRQPAEKTAFQVFRQEVIGTQKLSGGDAKTAGRLYKQGKMEAAREWLLKGARDDVPPNFQPPAKCLVVAVSHPFEEWDISKTNHDVQAYIYAQPLQAEGHLNGLSEKWEDTSADQHKLWFEKTGVPDRGLPVQAINKIAKAAVNRAFGVVRKVENRNEKRRSRDNRIAEHNRENGLTEVVREAPEVATNADGFLLHPPGIDPSILSYASVSPVPYNSSKHSFVRLPEEYQAYNVEPDAPIPQFVVEDRFAIPPGQPGYVPEWQRLKCSTNKHRRMRQWSNQDYKPKAGRRAKPLEFQAHLTRERAKGALLVVMRIKEDWVVFDVRGLLRNVEWRKVLSEEAREKLTLKGLLDLFTGDPVIDTKRGIVTFLYKAEITKILSKRTVKTKNARDLLLRLTEPGEDGLRREVGLVAVDLGQTHPIAAAIYRIGRTSAGALESTVLHRQGLREDQKEKLKEYRKRHTALDSRLRKEAFETLSVEQQKEIVTVSGSGAQITKDKVCNYLGVDPSTLPWEKMGSYTHFISDDFLRRGGDPNIVHFDRQPKKGKVSKKSQRIKRSDSQWVGRMRPRLSQETAKARMEADWAAQNENEEYKRLARSKQELARWCVNTLLQNTRCITQCDEIVVVIEDLNVKSLHGKGAREPGWDNFFTPKTENRWFIQILHKTFSELPKHRGEHVIEGCPLRTSITCPACSYCDKNSRNGEKFVCVACGATFHADFEVATYNLVRLATTGMPMPKSLERQGGGEKAGGARKARKKAKQVEKIVVQANANVTMNGASLHSP

SEQ ID NO: 12 - protein, 707 aa, Artificial sequence
  synthetic sequence
MADTPTLFTQFLRHHLPGQRFRKDILKQAGRILANKGEDATIAFLRGKSEESPPDFQPPVKCPIIACSRPLTEWPIYQASVAIQGYVYGQSLAEFEASDPGCSKDGLLGWFDKTGVCTDYFSVQGLNLIFQNARKRYIGVQTKVTNRNEKRHKKLKRINAKRIAEGLPELTSDEPESALDETGHLIDPPGLNTNIYCYQQVSPKPLALSEVNQLPTAYAGYSTSGDDPIQPMVTKDRLSISKGQPGYIPEHQRALLSQKKHRRMRGYGLKARALLVIVRIQDDWAVIDLRSLLRNAYWRRIVQTKEPSTITKLLKLVTGDPVLDATRMVATFTYKPGIVQVRSAKCLKNKQGSKLFSERYLNETVSVTSIDLGSNNLVAVATYRLVNGNTPELLQRFTLPSHLVKDFERYKQAHDTLEDSIQKTAVASLPQGQQTEIRMWSMYGFREAQERVCQELGLADGSIPWNVMTATSTILTDLFLARGGDPKKCMFTSEPKKKKNSKQVLYKIRDRAWAKMYRTLLSKETREAWNKALWGLKRGSPDYARLSKRKEELARRCVNYTISTAEKRAQCGRTIVALEDLNIGFFHGRGKQEPGWVGLFTRKKENRWLMQALHKAFLELAHHRGYHVIEVNPAYTSQTCPVCRHCDPDNRDQHNREAFHCIGCGFRGNADLDVATHNIAMVAITGESLKRARGSVASKTPQPLAAE

SEQ ID NO: 13 - protein, 757 aa, Artificial sequence
  synthetic sequence
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 14 - protein, 739 aa, Artificial sequence
  synthetic sequence
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKAGSSGEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 15 - protein, 757 aa, Artificial sequence
  synthetic sequence
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLAAINAARAAAGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 16 - protein, 765 aa, Artificial sequence
  synthetic sequence
MYILEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 17 - protein, 812 aa, Artificial sequence
  synthetic sequence
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 18 - protein, 812 aa, Artificial sequence
  synthetic sequence
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHKGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 19 - protein, 812 aa, Artificial sequence
  synthetic sequence
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 20 - protein, 812 aa, Artificial sequence
  synthetic sequence
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 21 - protein, 766 aa, Artificial sequence
  synthetic sequence
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 22 - DNA, 36 bp, Artificial sequence
  synthetic sequence
gtctcgactaatcgagcaatcgtttgagatctctcc

SEQ ID NO: 23 - DNA, 36 bp, Artificial sequence
  synthetic sequence
gtcggaacgctcaacgattgcccctcacgaggggac

SEQ ID NO: 24 - DNA, 36 bp, Artificial sequence
  synthetic sequence
gtcccagcgtactgggcaatcaatagtcgttttggt

SEQ ID NO: 25 - DNA, 37 bp, Artificial sequence
  synthetic sequence
ggatccaatcctttttgattgcccaattcgttgggac

SEQ ID NO: 26 - DNA, 36 bp, Artificial sequence
  synthetic sequence
ggatctgaggatcattattgctcgttacgacgagac

SEQ ID NO: 27 - DNA, 36 bp, Artificial sequence
  synthetic sequence
gtctcgtcgtaacgagcaataatgatcctcagatcc

SEQ ID NO: 28 - DNA, 36 bp, Artificial sequence
  synthetic sequence
gtctcagcgtactgagcaatcaaaaggtttcgcagg

SEQ ID NO: 29 - DNA, 36 bp, Artificial sequence
  synthetic sequence
gtctcctcgtaaggagcaatctattagtcttgaaag

SEQ ID NO: 30 - DNA, 36 bp, Artificial sequence
  synthetic sequence
gtctcggcgcaccgagcaatcagcgaggtcttctac

SEQ ID NO: 31 - DNA, 36 bp, Artificial sequence
  synthetic sequence
accaaaacgactattgattgcccagtacgctgggac

SEQ ID NO: 32 - DNA, 37 bp, Artificial sequence
  synthetic sequence
gtcccaacgaattgggcaatcaaaaaggattggatcc

SEQ ID NO: 33 - DNA, 36 bp, Artificial sequence
  synthetic sequence
gtcgcggcgtaccgcgcaatgagagtctgttgccat

SEQ ID NO: 34 - RNA, 36 nt, Artificial sequence
  synthetic sequence
  misc_feature (27)..(27): n is a, c, g, or u
  misc_feature (33)..(33): n is a, c, g, or u
gucycrdcgwahygrgcaaucrdwrrnkduukndrb

SEQ ID NO: 35 - RNA, 36 nt, Artificial sequence
  synthetic sequence
gucccaacgaauugggcaaucaaaaaggauuggauc

SEQ ID NO: 36 - RNA, 36 nt, Artificial sequence
  synthetic sequence
gucucagcguacugagcaaucaaaagguuucgcagg

SEQ ID NO: 37 - RNA, 36 nt, Artificial sequence
  synthetic sequence
gucucgacuaaucgagcaaucguuugagaucucucc

SEQ ID NO: 38 - RNA, 36 nt, Artificial sequence
  synthetic sequence
guccccucgugaggggcaaucguugagcguuccgac

SEQ ID NO: 39 - RNA, 36 nt, Artificial sequence
  synthetic sequence
gucccagcguacugggcaaucaauagucguuuuggu

SEQ ID NO: 40 - RNA, 36 nt, Artificial sequence
  synthetic sequence
gucgcggcguaccgcgcaaugagagucuguugccau

SEQ ID NO: 41 - RNA, 36 nt, Artificial sequence
  synthetic sequence
gucuccucguaaggagcaaucuauuagucuugaaag

SEQ ID NO: 42 - RNA, 36 nt, Artificial sequence
  synthetic sequence
gucucggcgcaccgagcaaucagcgaggucuucuac

SEQ ID NO: 43 - DNA, 14769 bp, Artificial sequence
  synthetic sequence
gtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagatcactagtgatcagatgcagagagactaacctcagataggcgacttcaaaaccccccgaaaatttgacaatggagcttgaaaagaatggcaaaaaaaaattggctggattgtcaactggtcccctcgtgaggggcaatcgttgagcgttccgaccaatcactacttcgactctagctgtatataaactcagcttcgttttcttatctaagcgatgtgggacttttgaagattgttttcaacttaaatgggcctatataagaaatactattgttctttcccatataaatgggcctgcttctcttctttcagattcccaggggccttttgaagattatcttcatatcttaagaatgaagatgttttattcaatcaaattcttgaaggttcgatgcctaatcattctaatcctgggacaaactatgaaacaagatacaaaaactccgaatggaaagttaaaaagaagaaaacgaaagctacggttcaagaaaatgtaagctgataaacaaaaaaaaactgtatgaacgaagaagaagaaaaaaagctaagaagaaatgatgtattgtgcggaaggcaagtcgagtttccgttgttcaacgaagcttctagtatcctaggaaggtaccagtctagctcaacagagcttttaacccaaattggtacaatagaatacaactttagatcataattctcaaaagaaagagattccttagctattctatctgccactccatttccttctcggcttgtatgcacaagcataaaatcctcaaacttgctaagtagatactttatgtcttggataattggattgagacttgacaagcataactttcatgtaaccaaagacacaagttgctgagaatccacctcaaaaatgatcttcctataattgaatcgggataatgacagcacagcccatctaagagcctccacttctacttccagcacgcttcttacttttaccacagctcttgcacctaaccataacaccttccctgtatgatcgcgaagcacccaccctaagccacattttaatccttctgttggccatgccccatcaaagttgcacttaacccaagattgtggtggagcttcccatgtttctcgtctgtcccgacggtgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgccgaagcccgccgtcgaatcagagttttccaaagtcctcaagaaacactttcctggggagcgttttaggtctagctatatgaagagggggggtaaaattctggcagcacaaggcgaggaagctgtagtggcgtacttgcagggaaagagtgaggaggaaccgccgaattttcagccgccggcgaagtgccacgtggtcaccaaaagcagggatttcgcagaatggcccataatgaaagcctctgaagccatacagaggtacatctatgcgctcagcactacagagcgagctgcctgcaaaccgggtaagagctcagaaagtcacgcggcctggttcgcggctacaggggtgagcaatcacggctattctcatgtacaaggtcttaacctgatctttgaccacacgctaggacgatacgatggcgttttaaagaaagtacagcttcgaaatgagaaggcccgagctagattggaaagcataaacgcctcacgagctgatgaaggattgccggaaatcaaggccgaggaggaggaggtagctacaaatgaaacaggtcatctactacagccgccaggcataaacccatcattctacgtctaccagaccatatctccgcaggcttaccgaccaagggacgaaatagtgttaccacccgagtacgccggttacgtcagggatccgaacgctccgattccactgggcgtggtcaggaaccgttgtgacatacagaagggttgcccgggatatatacccgagtggcagagggaagctggtacggcaattagtcccaagacaggaaaagcagtgacggttccaggacttagcccgaagaagaataaacgtatgcgtaggtactggaggtcagaaaaggagaaggctcaagatgcacttctcgtaactgtaaggataggtaccgattgggtagtcatagacgtaagaggattactaagaaacgccaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggaggactattgctcccaaggacataagtttaaatgcacttctagatttatttaccggtgatccagtcatcgatgtcagacgaaacatcgtgaccttcacctataccttggacgcttgcggaacttatgctagaaaatggactctcaagggaaaacagacaaaagcaaccttagataaactgacagcgacacaaactgtggccttagttgctatagatctgggacaaacaaacccaattagcgcgggtatcagtcgtgtcacacaggagaacggggccctccagtgcgaaccgcttgatcgttttacattgcctgatgaccttttgaaagatatttctgcgtaccgaattgcatgggaccgtaacgaggaggaactcagggccagatccgttgaggcactcccagaggcacaacaagcagaggtcagagctctggacggggtctccaaagagaccgcgcgtacacagttgtgcgcggacttcggtctggacccaaagcgactaccgtgggataaaatgagtagcaataccacgttcataagcgaggcgctcctttccaacagcgtatcccgtgaccaagtattctttaccccggccccaaagaaaggagccaagaagaaagcaccggtggaagtgatgcgaaaagacaggacatgggcgcgagcgtacaaaccacgactctcagtagaagcacaaaagttgaaaaatgaggctctttgggctttgaagcgtacctctccagaatatctaaagttgtcacgacgtaaagaagaattgtgtaggaggtccattaattacgtgatagaaaaaactagaaggcgaacccaatgccaaattgtgatccccgttatagaagatctaaatgtccgatttttccacgggtctggcaaacgactcccgggctgggataacttcttcacggcaaagaaggaaaaccgatggtttatccaagggctacataaggcgttttctgacttaaggacccaccgatccttctacgtgttcgaggtgcgtccagagagaacatcaatcacctgtccgaaatgcgggcactgtgaagtggggaacagggatggagaagcctttcagtgtctcagttgcggtaagacatgcaacgcagatctggacgtagcgacacataacttgactcaagtggcgctcaccggcaaaacaatgccgaagagagaggagcctagagatgcacaagggacagcccccgcgagaaagacgaagaaagctagcaagtcaaaggcacccccggctgaacgtgaggatcagactcccgctcaagagccgtcccaaacgtccggatccggaccgaagaaaaagcgaaaggtagaggatcctaaaaagaagcgtaaagtctccttgggttctggctccgactataaggatgacgatgacaaagactataaggatgacgacgacaagtgaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacgaattc

SEQ ID NO: 44 - DNA, 2406 bp, Artificial sequence
  synthetic sequence
atgccgaagcccgccgtcgaatcagagttttccaaagtcctcaagaaacactttcctggggagcgttttaggtctagctatatgaagagggggggtaaaattctggcagcacaaggcgaggaagctgtagtggcgtacttgcagggaaagagtgaggaggaaccgccgaattttcagccgccggcgaagtgccacgtggtcaccaaaagcagggatttcgcagaatggcccataatgaaagcctctgaagccatacagaggtacatctatgcgctcagcactacagagcgagctgcctgcaaaccgggtaagagctcagaaagtcacgcggcctggttcgcggctacaggggtgagcaatcacggctattctcatgtacaaggtcttaacctgatctttgaccacacgctaggacgatacgatggcgttttaaagaaagtacagcttcgaaatgagaaggccggatcttcaggtgaggaggaggtagctacaaatgaaacaggtcatctactacagccgccaggcataaacccatcattctacgtctaccagaccatatctccgcaggcttaccgaccaagggacgaaatagtgttaccacccgagtacgccggttacgtcagggatccgaacgctccgattccactgggcgtggtcaggaaccgttgtgacatacagaagggttgcccgggatatatacccgagtggcagagggaagctggtacggcaattagtcccaagacaggaaaagcagtgacggttccaggacttagcccgaagaagaataaacgtatgcgtaggtactggaggtcagaaaaggagaaggctcaagatgcacttctcgtaactgtaaggataggtaccgattgggtagtcatagacgtaagaggattactaagaaacgccaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggaggactattgctcccaaggacataagtttaaatgcacttctagatttatttaccggtgatccagtcatcgatgtcagacgaaacatcgtgaccttcacctataccttggacgcttgcggaacttatgctagaaaatggactctcaagggaaaacagacaaaagcaaccttagataaactgacagcgacacaaactgtggccttagttgctatagatctgggacaaacaaacccaattagcgcgggtatcagtcgtgtcacacaggagaacggggccctccagtgcgaaccgcttgatcgttttacattgcctgatgaccttttgaaagatatttctgcgtaccgaattgcatgggaccgtaacgaggaggaactcagggccagatccgttgaggcactcccagaggcacaacaagcagaggtcagagctctggacggggtctccaaagagaccgcgcgtacacagttgtgcgcggacttcggtctggacccaaagcgactaccgtgggataaaatgagtagcaataccacgttcataagcgaggcgctcctttccaacagcgtatcccgtgaccaagtattctttaccccggccccaaagaaaggagccaagaagaaagcaccggtggaagtgatgcgaaaagacaggacatgggcgcgagcgtacaaaccacgactctcagtagaagcacaaaagttgaaaaatgaggctctttgggctttgaagcgtacctctccagaatatctaaagttgtcacgacgtaaagaagaattgtgtaggaggtccattaattacgtgatagaaaaaactagaaggcgaacccaatgccaaattgtgatccccgttatagaagatctaaatgtccgatttttccacgggtctggcaaacgactcccgggctgggataacttcttcacggcaaagaaggaaaaccgatggtttatccaagggctacataaggcgttttctgacttaaggacccaccgatccttctacgtgttcgaggtgcgtccagagagaacatcaatcacctgtccgaaatgcgggcactgtgaagtggggaacagggatggagaagcctttcagtgtctcagttgcggtaagacatgcaacgcagatctggacgtagcgacacataacttgactcaagtggcgctcaccggcaaaacaatgccgaagagagaggagcctagagatgcacaagggacagcccccgcgagaaagacgaagaaagctagcaagtcaaaggcacccccggctgaacgtgaggatcagactcccgctcaagagccgtcccaaacgtcc

SEQ ID NO: 45 - DNA, 2460 bp, Artificial sequence
  synthetic sequence
atgccgaagcccgccgtcgaatcagagttttccaaagtcctcaagaaacactttcctggggagcgttttaggtctagctatatgaagagggggggtaaaattctggcagcacaaggcgaggaagctgtagtggcgtacttgcagggaaagagtgaggaggaaccgccgaattttcagccgccggcgaagtgccacgtggtcaccaaaagcagggatttcgcagaatggcccataatgaaagcctctgaagccatacagaggtacatctatgcgctcagcactacagagcgagctgcctgcaaaccgggtaagagctcagaaagtcacgcggcctggttcgcggctacaggggtgagcaatcacggctattctcatgtacaaggtcttaacctgatctttgaccacacgctaggacgatacgatggcgttttaaagaaagtacagcttcgaaatgagaaggcccgagctagattggctgcaataaacgccgctcgagctgcagctggattgccggaaatcaaggccgaggaggaggaggtagctacaaatgaaacaggtcatctactacagccgccaggcataaacccatcattctacgtctaccagaccatatctccgcaggcttaccgaccaagggacgaaatagtgttaccacccgagtacgccggttacgtcagggatccgaacgctccgattccactgggcgtggtcaggaaccgttgtgacatacagaagggttgcccgggatatatacccgagtggcagagggaagctggtacggcaattagtcccaagacaggaaaagcagtgacggttccaggacttagcccgaagaagaataaacgtatgcgtaggtactggaggtcagaaaaggagaaggctcaagatgcacttctcgtaactgtaaggataggtaccgattgggtagtcatagacgtaagaggattactaagaaacgccaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggaggactattgctcccaaggacataagtttaaatgcacttctagatttatttaccggtgatccagtcatcgatgtcagacgaaacatcgtgaccttcacctataccttggacgcttgcggaacttatgctagaaaatggactctcaagggaaaacagacaaaagcaaccttagataaactgacagcgacacaaactgtggccttagttgctatagatctgggacaaacaaacccaattagcgcgggtatcagtcgtgtcacacaggagaacggggccctccagtgcgaaccgcttgatcgttttacattgcctgatgaccttttgaaagatatttctgcgtaccgaattgcatgggaccgtaacgaggaggaactcagggccagatccgttgaggcactcccagaggcacaacaagcagaggtcagagctctggacggggtctccaaagagaccgcgcgtacacagttgtgcgcggacttcggtctggacccaaagcgactaccgtgggataaaatgagtagcaataccacgttcataagcgaggcgctcctttccaacagcgtatcccgtgaccaagtattctttaccccggccccaaagaaaggagccaagaagaaagcaccggtggaagtgatgcgaaaagacaggacatgggcgcgagcgtacaaaccacgactctcagtagaagcacaaaagttgaaaaatgaggctctttgggctttgaagcgtacctctccagaatatctaaagttgtcacgacgtaaagaagaattgtgtaggaggtccattaattacgtgatagaaaaaactagaaggcgaacccaatgccaaattgtgatccccgttatagaagatctaaatgtccgatttttccacgggtctggcaaacgactcccgggctgggataacttcttcacggcaaagaaggaaaaccgatggtttatccaagggctacataaggcgttttctgacttaaggacccaccgatccttctacgtgttcgaggtgcgtccagagagaacatcaatcacctgtccgaaatgcgggcactgtgaagtggggaacagggatggagaagcctttcagtgtctcagttgcggtaagacatgcaacgcagatctggacgtagcgacacataacttgactcaagtggcgctcaccggcaaaacaatgccgaagagagaggagcctagagatgcacaagggacagcccccgcgagaaagacgaagaaagctagcaagtcaaaggcacccccggctgaacgtgaggatcagactcccgctcaagagccgtcccaaacgtcc

SEQ ID NO: 46 - DNA, 839 bp, Artificial sequence
  synthetic sequence
tggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttgcctcagctccgacctgatgagtccgtgaggacgaaacgagtaagctcgtcgtcggaacgctcaacgattgcccctcacgaggggaccagttgacaatccagccaatggccggcatggtcccagcctcctcgctggcgccggctgggcaacatgcttcggcatggcgaatgggacttaattaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaatta

SEQ ID NO: 47 - DNA, 2804 bp, Artificial sequence
  synthetic sequence
agtctagctcaacagagcttttaacccaaattggtacaatagaatacaactttagatcataattctcaaaagaaagagattccttagctattctatctgccactccatttccttctcggcttgtatgcacaagcataaaatcctcaaacttgctaagtagatactttatgtcttggataattggattgagacttgacaagcataactttcatgtaaccaaagacacaagttgctgagaatccacctcaaaaatgatcttcctataattgaatcgggataatgacagcacagcccatctaagagcctccacttctacttccagcacgcttcttacttttaccacagctcttgcacctaaccataacaccttccctgtatgatcgcgaagcacccaccctaagccacattttaatccttctgttggccatgccccatcaaagttgcacttaacccaagattgtggtggagcttcccatgtttctcgtctgtcccgacggtgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggcctcagctccgacctgatgagtccgtgaggacgaaacgagtaagctcgtcgtcggaacgctcaacgattgcccctcacgaggggaccagttgacaatccagccaatggccggcatggtcccagcctcctcgctggcgccggctgggcaacatgcttcggcatggcgaatgggacttaattaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaac

SEQ ID NO: 48 - DNA, 1986 bp, Artificial sequence
  synthetic sequence
agtctagctcaacagagcttttaacccaaattggtacaatagaatacaactttagatcataattctcaaaagaaagagattccttagctattctatctgccactccatttccttctcggcttgtatgcacaagcataaaatcctcaaacttgctaagtagatactttatgtcttggataattggattgagacttgacaagcataactttcatgtaaccaaagacacaagttgctgagaatccacctcaaaaatgatcttcctataattgaatcgggataatgacagcacagcccatctaagagcctccacttctacttccagcacgcttcttacttttaccacagctcttgcacctaaccataacaccttccctgtatgatcgcgaagcacccaccctaagccacattttaatccttctgttggccatgccccatcaaagttgcacttaacccaagattgtggtggagcttcccatgtttctcgtctgtcccgacggtgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacag

SEQ ID NO: 49 - DNA, 465 bp, Artificial sequence
  synthetic sequence
tggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagctt

SEQ ID NO: 50 - protein, 10 aa, Artificial sequence
  synthetic sequence
  VARIANT (1)..(1): The amino acid at position 1 is any amino acid other than Glu
  VARIANT (2)..(2): The amino acid at position 2 is any amino acid other than Ser
  VARIANT (3)..(3): The amino acid at position 3 is Ile, Arg, Lys, Leu, or Asn
  VARIANT (4)..(4): The amino acid at position 4 is Asn, Lys, or Ala
  VARIANT (5)..(5): The amino acid at position 5 is Ala, Glu, His, or Lys
  VARIANT (6)..(6): The amino acid at position 6 is any amino acid other than Ser
  VARIANT (7)..(7): The amino acid at position 7 is Arg, Asn, Ala, or Cys
  VARIANT (8)..(8): The amino acid at position 8 is Ala, Ile, Arg, Ser, Leu, or Lys
  VARIANT (9)..(9): The amino acid at position 9 is any amino acid other than Asp
  VARIANT (10)..(10): The amino acid at position 10 is any amino acid other than Glu
XXXXXXXXXX

SEQ ID NO: 51 - protein, 10 aa, Artificial sequence
  synthetic sequence
  VARIANT (1)..(1): The amino acid at position 1 is any amino acid other than Glu
  VARIANT (2)..(2): The amino acid at position 2 is any amino acid other than Ser
  VARIANT (6)..(6): The amino acid at position 6 is any amino acid other than Ser
  VARIANT (9)..(9): The amino acid at position 9 is any amino acid other than Asp
  VARIANT (10)..(10): The amino acid at position 10 is any amino acid other than Glu
XXINSXRAXX

SEQ ID NO: 52 - protein, 10 aa, Artificial sequence
  synthetic sequence
AAINAARAAA

SEQ ID NO: 53 - protein, 25 aa, Artificial sequence
  synthetic sequence
  VARIANT (5)..(25): Each amino acid at postions 5 to 25 may either be present or absent
GGGGGGGGGGGGGGGGGGGGGGGGG

SEQ ID NO: 54 - protein, 25 aa, Artificial sequence
  synthetic sequence
  VARIANT (5)..(25): Each amino acid at positions 5 to 25 may either be present or absent
SSSSSSSSSSSSSSSSSSSSSSSSS

SEQ ID NO: 55 - protein, 24 aa, Artificial sequence
  synthetic sequence
  VARIANT (5)..(24): Each copy of Gly Ser Ser Gly at positions 5 to 24 may either be present or absent
GSSGGSSGGSSGGSSGGSSGGSSG

SEQ ID NO: 56 - protein, 25 aa, Artificial sequence
  synthetic sequence
  VARIANT (6)..(25): Each copy of Gly Gly Gly Gly Ser at positions 6 to 25 may either be present or absent
GGGGSGGGGSGGGGSGGGGSGGGGS

SEQ ID NO: 57 - protein, 13 aa, Artificial sequence
  synthetic sequence
SRLEEELRRRLTE

SEQ ID NO: 58 - protein, 15 aa, Artificial sequence
  synthetic sequence
GLNDIFEAQKIEWHE

SEQ ID NO: 59 - protein, 4 aa, Artificial sequence
  synthetic sequence
EPEA

SEQ ID NO: 60 - protein, 26 aa, Artificial sequence
  synthetic sequence
KRRWKKNFIAVSAANRFKKISSSGAL

SEQ ID NO: 61 - protein, 10 aa, Artificial sequence
  synthetic sequence
  VARIANT (5)..(10): Each amino acid at positions 5 to 10 may either be present or absent
EEEEEEEEEE

SEQ ID NO: 62 - protein, 9 aa, Artificial sequence
  synthetic sequence
  VARIANT (6)..(9): Each amino acid at positions 6 to 9 may either be present or absent
RRRRRRRRR

SEQ ID NO: 63 - protein, 13 aa, Artificial sequence
  synthetic sequence
GAPVPYPDPLEPR

SEQ ID NO: 64 - protein, 8 aa, Artificial sequence
  synthetic sequence
DYKDDDDK

SEQ ID NO: 65 - protein, 9 aa, Artificial sequence
  synthetic sequence
YPYDVPDYA

SEQ ID NO: 66 - protein, 10 aa, Artificial sequence
  synthetic sequence
  VARIANT (6)..(10): Each amino acid at positions 6 to 10 may either be present or absent
HHHHHHHHHH

SEQ ID NO: 67 - protein, 10 aa, Artificial sequence
  synthetic sequence
EQKLISEEDL

SEQ ID NO: 68 - protein, 18 aa, Artificial sequence
  synthetic sequence
TKENPRSNQEESYDDNES

SEQ ID NO: 69 - protein, 9 aa, Artificial sequence
  synthetic sequence
TETSQVAPA

SEQ ID NO: 70 - protein, 15 aa, Artificial sequence
  synthetic sequence
KETAAAKFERQHMDS

SEQ ID NO: 71 - protein, 38 aa, Artificial sequence
  synthetic sequence
MDEKTTGWRGGHVVEGLAGELEQLRARLEHHPQGQREP

SEQ ID NO: 72 - protein, 13 aa, Artificial sequence
  synthetic sequence
SLAELLNAGLGGS

SEQ ID NO: 73 - protein, 8 aa, Artificial sequence
  synthetic sequence
TQDPSRVG

SEQ ID NO: 74 - protein, 8 aa, Artificial sequence
  synthetic sequence
WSHPQFEK

SEQ ID NO: 75 - protein, 11 aa, Artificial sequence
  synthetic sequence
MASMTGGQQMG

SEQ ID NO: 76 - protein, 6 aa, Artificial sequence
  synthetic sequence
CCPGCC

SEQ ID NO: 77 - protein, 10 aa, Artificial sequence
  synthetic sequence
EVHTNQDPLD

SEQ ID NO: 78 - protein, 14 aa, Artificial sequence
  synthetic sequence
GKPIPNPLLGLDST

SEQ ID NO: 79 - protein, 11 aa, Artificial sequence
  synthetic sequence
YTDIEMNRLGK

SEQ ID NO: 80 - protein, 8 aa, Artificial sequence
  synthetic sequence
DLYDDDDK

SEQ ID NO: 81 - protein, 84 aa, Artificial sequence
  synthetic sequence
MASMISSSAVTTVSRASRGQSAAMAPFGGLKSMTGFPVRKVNTDITSITSNGGRVKCMQVWPPIGKKKFETLSYLPPLTRDSRA

SEQ ID NO: 82 - protein, 57 aa, Artificial sequence
  synthetic sequence
MASMISSSAVTTVSRASRGQSAAMAPFGGLKSMTGFPVRKVNTDITSITSNGGRVKS

SEQ ID NO: 83 - protein, 85 aa, Artificial sequence
  synthetic sequence
MASSMLSSATMVASPAQATMVAPFNGLKSSAAFPATRKANNDITSITSNGGRVNCMQVWPPIEKKKFETLSYLPDLTDSGGRVNC

SEQ ID NO: 84 - protein, 76 aa, Artificial sequence
  synthetic sequence
MAQVSRICNGVQNPSLISNLSKSSQRKSPLSVSLKTQQHPRAYPISSSWGLKKSGMTLIGSELRPLKVMSSVSTAC

SEQ ID NO: 85 - protein, 76 aa, Artificial sequence
  synthetic sequence
MAQVSRICNGVWNPSLISNLSKSSQRKSPLSVSLKTQQHPRAYPISSSWGLKKSGMTLIGSELRPLKVMSSVSTAC

SEQ ID NO: 86 - protein, 72 aa, Artificial sequence
  synthetic sequence
MAQINNMAQGIQTLNPNSNFHKPQVPKSSSFLVFGSKKLKNSANSMLVLKKDSIFMQLFCSFRISASVATAC

SEQ ID NO: 87 - protein, 69 aa, Artificial sequence
  synthetic sequence
MAALVTSQLATSGTVLSVTDRFRRPGFQGLRPRNPADAALGMRTVGASAAPKQSRKPHRFDRRCLSMVV

SEQ ID NO: 88 - protein, 77 aa, Artificial sequence
  synthetic sequence
MAALTTSQLATSATGFGIADRSAPSSLLRHGFQGLKPRSPAGGDATSLSVTTSARATPKQQRSVQRGSRRFPSVVVC

SEQ ID NO: 89 - protein, 57 aa, Artificial sequence
  synthetic sequence
MASSVLSSAAVATRSNVAQANMVAPFTGLKSAASFPVSRKQNLDITSIASNGGRVQC

SEQ ID NO: 90 - protein, 65 aa, Artificial sequence
  synthetic sequence
MESLAATSVFAPSRVAVPAARALVRAGTVVPTRRTSSTSGTSGVKCSAAVTPQASPVISRSAAAA

SEQ ID NO: 91 - protein, 72 aa, Artificial sequence
  synthetic sequence
MGAAATSMQSLKFSNRLVPPSRRLSPVPNNVTCNNLPKSAAPVRTVKCCASSWNSTINGAAATTNGASAASS

SEQ ID NO: 92 - protein, 20 aa, Artificial sequence
  synthetic sequence
  VARIANT (4)..(4): The amino acid at position 4 is selected from lysine, histidine, and arginine
  VARIANT (8)..(8): The amino acid at position 8 is selected from lysine, histidine, and arginine
  VARIANT (11)..(11): The amino acid at position 11 is selected from lysine, histidine, and arginine
  VARIANT (15)..(15): The amino acid at position 15 is selected from lysine, histidine, and arginine
  VARIANT (19)..(19): The amino acid at position 19 is selected from lysine, histidine, and arginine
GLFXALLXLLXSLWXLLLXA

SEQ ID NO: 93 - protein, 20 aa, Artificial sequence
  synthetic sequence
GLFHALLHLLHSLWHLLLHA

SEQ ID NO: 94 - protein, 167 aa, Artificial sequence
  synthetic sequence
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 95 - protein, 178 aa, Artificial sequence
  synthetic sequence
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 96 - protein, 160 aa, Staphylococcus aureus
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 97 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 98 - protein, 183 aa, Salmonella typhimurium
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 99 - protein, 164 aa, Shewanella putrefaciens
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 100 - protein, 173 aa, Haemophilus influenzae
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 101 - protein, 161 aa, Caulobacter crescentus
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 102 - protein, 179 aa, Geobacter sulfurreducens
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 103 - protein, 198 aa, Artificial sequence [sequence omitted]

SEQ ID NO: 104 - protein, 188 aa, Artificial sequence
  synthetic sequence
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKENHERTFKAWEGLHENSVRLSRQLRRILLPLYEVDDLRDAFRTLGL

SEQ ID NO: 105 - protein, 7 aa, SV40 virus
PKKKRKV

SEQ ID NO: 106 - protein, 16 aa, Unknown
  nucleoplasmin bipartite NLS
KRPAATKKAGQAKKKK

SEQ ID NO: 107 - protein, 9 aa, Unknown
  c-myc NLS
PAAKRVKLD

SEQ ID NO: 108 - protein, 11 aa, Unknown
  c-myc NLS
RQRRNELKRSP

SEQ ID NO: 109 - protein, 38 aa, Unknown
  hRNPA1 M9 NLS
NQSSNFGPMKGGNFGGRSSGPYGGGGQYFAKPRNQGGY

SEQ ID NO: 110 - protein, 42 aa, Unknown
  IBB domain from importin-alpha
RMRIZFKNKGKDTAELRRRRVEVSVELRKAKKDEQILKRRNV

SEQ ID NO: 111 - protein, 8 aa, Unknown
  myoma T protein
VSRKRPRP

SEQ ID NO: 112 - protein, 8 aa, Unknown
  myoma T protein
PPKKARED

SEQ ID NO: 113 - protein, 8 aa, Homo sapiens
PQPKKKPL

SEQ ID NO: 114 - protein, 12 aa, Mus musculus
SALIKKKKKMAP

SEQ ID NO: 115 - protein, 5 aa, Influenza virus
DRLRR

SEQ ID NO: 116 - protein, 7 aa, Influenza virus
PKQKKRK

SEQ ID NO: 117 - protein, 10 aa, Hepatitis virus
RKLKKKIKKL

SEQ ID NO: 118 - protein, 10 aa, Mus musculus
REKKKFLKRR

SEQ ID NO: 119 - protein, 20 aa, Homo sapiens
KRKGDEVDGVDEVAKKKSKK

SEQ ID NO: 120 - protein, 17 aa, Homo sapiens
RKCLQAGMNLEARKTKK

SEQ ID NO: 121 - protein, 11 aa, Human immunodeficiency virus 1
YGRKKRRQRRR

SEQ ID NO: 122 - protein, 12 aa, Artificial sequence
  synthetic sequence
RRQRRTSKLMKR

SEQ ID NO: 123 - protein, 27 aa, Unknown
  Transportan
GWTLNSAGYLLGKINLKALAALAKKIL

SEQ ID NO: 124 - protein, 33 aa, Artificial sequence
  synthetic sequence
KALAWEAKLAKALAKALAKHLAKALAKALKCEA

SEQ ID NO: 125 - protein, 16 aa, Artificial sequence
  synthetic sequence
RQIKIWFQNRRMKWKK

SEQ ID NO: 126 - protein, 9 aa, Artificial sequence
  synthetic sequence
RKKRRQRRR

SEQ ID NO: 127 - protein, 8 aa, Artificial sequence
  synthetic sequence
RKKRRQRR

SEQ ID NO: 128 - protein, 11 aa, Artificial sequence
  synthetic sequence
YARAAARQARA

SEQ ID NO: 129 - protein, 11 aa, Artificial sequence
  synthetic sequence
THRLPRRRRRR

SEQ ID NO: 130 - protein, 11 aa, Artificial sequence
  synthetic sequence
GGRRARRRRRR

SEQ ID NO: 131 - protein, 5 aa, Artificial sequence
  synthetic sequence
GSGGS

SEQ ID NO: 132 - protein, 6 aa, Artificial sequence
  synthetic sequence
GGSGGS

SEQ ID NO: 133 - protein, 5 aa, Artificial sequence
  synthetic sequence
GGGGS

SEQ ID NO: 134 - protein, 4 aa, Artificial sequence
  synthetic sequence
GGGS

SEQ ID NO: 135 - protein, 4 aa, Artificial sequence
  synthetic sequence
GGSG

SEQ ID NO: 136 - protein, 5 aa, Artificial sequence
  synthetic sequence
GGSGG

SEQ ID NO: 137 - protein, 5 aa, Artificial sequence
  synthetic sequence
GSGSG

SEQ ID NO: 138 - protein, 5 aa, Artificial sequence
  synthetic sequence
GSGGG

SEQ ID NO: 139 - protein, 5 aa, Artificial sequence
  synthetic sequence
GGGSG

SEQ ID NO: 140 - protein, 5 aa, Artificial sequence
  synthetic sequence
GSSSG

SEQ ID NO: 141 - protein, 5 aa, Artificial sequence
  synthetic sequence
GGGGS

SEQ ID NO: 142 - RNA, 36 nt, Artificial sequence
  synthetic sequence
gucggaacgcucaacgauugccccucacgaggggac

SEQ ID NO: 143 - DNA, 36 bp, Artificial sequence
  synthetic sequence
gucccagcguacugggcaaucaauagtcguuuuggu

SEQ ID NO: 144 - RNA, 40 nt, Artificial sequence
  synthetic sequence
cacaggagagaucucaaacgauugcucgauuagucgagac

SEQ ID NO: 145 - RNA, 40 nt, Artificial sequence
  synthetic sequence
uaaugucggaacgcucaacgauugccccucacgaggggac

SEQ ID NO: 146 - RNA, 40 nt, Artificial sequence
  synthetic sequence
auuaaccaaaacgacuauugauugcccaguacgcugggac

SEQ ID NO: 147 - RNA, 71 nt, Artificial sequence
  synthetic sequence
  misc_feature (1)..(35): n is a, c, g, or u
nnnnnnnnnnnnnnnnnnnnnnnnnnnnnnnnnnngucucgacuaaucgagcaaucguuugagaucucucc

SEQ ID NO: 148 - RNA, 71 nt, Artificial sequence
  synthetic sequence
  misc_feature (1)..(35): n is a, c, g, or u
nnnnnnnnnnnnnnnnnnnnnnnnnnnnnnnnnnngucggaacgcucaacgauugccccucacgaggggac

SEQ ID NO: 149 - RNA, 71 nt, Artificial sequence
  synthetic sequence
  misc_feature (37)..(71): n is a, c, g, or u
gucucgacuaaucgagcaaucguuugagaucucuccnnnnnnnnnnnnnnnnnnnnnnnnnnnnnnnnnnn

SEQ ID NO: 150 - RNA, 36 nt, Artificial sequence
  synthetic sequence
ggagagaucucaaacgauugcucgauuagucgagac

SEQ ID NO: 151 - RNA, 71 nt, Artificial sequence
  synthetic sequence
  misc_feature (37)..(71): n is a, c, g, or u
ggagagaucucaaacgauugcucgauuagucgagacnnnnnnnnnnnnnnnnnnnnnnnnnnnnnnnnnnn

SEQ ID NO: 152 - RNA, 71 nt, Artificial sequence
  synthetic sequence
  misc_feature (37)..(71): n is a, c, g, or u
gucggaacgcucaacgauugccccucacgaggggacnnnnnnnnnnnnnnnnnnnnnnnnnnnnnnnnnnn

SEQ ID NO: 153 - RNA, 71 nt, Artificial sequence
  synthetic sequence
  misc_feature (37)..(71): n is a, c, g, or u
guccccucgugaggggcaaucguugagcguuccgacnnnnnnnnnnnnnnnnnnnnnnnnnnnnnnnnnnn

SEQ ID NO: 154 - RNA, 75 nt, Artificial sequence
  synthetic sequence
  misc_feature (41)..(75): n is a, c, g, or u
cacaggagagaucucaaacgauugcucgauuagucgagacnnnnnnnnnnnnnnnnnnnnnnnnnnnnnnnnnnn

SEQ ID NO: 155 - RNA, 75 nt, Artificial sequence
  synthetic sequence
  misc_feature (41)..(75): n is a, c, g, or u
uaaugucggaacgcucaacgauugccccucacgaggggacnnnnnnnnnnnnnnnnnnnnnnnnnnnnnnnnnnn

SEQ ID NO: 156 - RNA, 75 nt, Artificial sequence
  synthetic sequence
  misc_feature (41)..(75): n is a, c, g, or u
auuaaccaaaacgacuauugauugcccaguacgcugggacnnnnnnnnnnnnnnnnnnnnnnnnnnnnnnnnnnn

SEQ ID NO: 157 - DNA, 257 bp, Artificial sequence
  synthetic sequence
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacg

SEQ ID NO: 158 - DNA, 448 bp, Artificial sequence
  synthetic sequence
aagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgatt

SEQ ID NO: 159 - DNA, 633 bp, Artificial sequence
  synthetic sequence
cgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtagattaatctgagtttttctgattaaca

SEQ ID NO: 160 - protein, 4 aa, Artificial sequence
  synthetic sequence
GSSG

SEQ ID NO: 161 - RNA, 45 nt, Artificial sequence
  synthetic sequence
caacgauugccccucacgaggggacagcugguaaugggauaccuu

SEQ ID NO: 162 - DNA, 20 bp, Artificial sequence
  synthetic sequence
aaggtatcccattaccagct

SEQ ID NO: 163 - DNA, 25 bp, Artificial sequence
  synthetic sequence
caacgattgcccctcacgaggggac

SEQ ID NO: 164 - DNA, 20 bp, Artificial sequence
  synthetic sequence
ttgttccgcaaaatagccca

SEQ ID NO: 165 - DNA, 20 bp, Artificial sequence
  synthetic sequence
cagttgacaatccagccaat

SEQ ID NO: 166 - DNA, 20 bp, Artificial sequence
  synthetic sequence
tcccattcaacattcatacg

SEQ ID NO: 167 - DNA, 20 bp, Artificial sequence
  synthetic sequence
tggttctatactaatatcaa

SEQ ID NO: 168 - DNA, 20 bp, Artificial sequence
  synthetic sequence
atattagtatagaaccataa

SEQ ID NO: 169 - DNA, 20 bp, Artificial sequence
  synthetic sequence
gtatagaaccataacaaaag

SEQ ID NO: 170 - DNA, 20 bp, Artificial sequence
  synthetic sequence
gcgacacgactcattataac